(19) United States
(12) Patent Application Publication
(10) Pub. No.: US 2023/0408921 A1
(43) Pub. Date: Dec. 21, 2023

(54) Polymerizable Monomer, Polymer Compound, Resist Composition, And Patterning Process

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 18/328,507

(22) Filed: Jun. 2, 2023

(30) Foreign Application Priority Data
Jun. 20, 2022 (JP) 2022-98948

(51) Int. Cl. G03F 7/039; C08F 116/14; G03F 7/004; G03F 7/20

(52) U.S. Cl. CPC G03F 7/0397; C08F 116/14; G03F 7/0045; G03F 7/0048; G03F 7/2006

(57) ABSTRACT

The present invention is a polymerizable monomer represented by the following general formula (1),

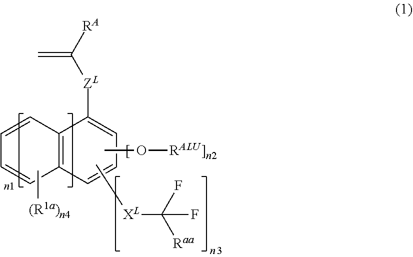

wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a hydrocarbyl group having 1 to 20 carbon atoms; n1 is an integer of 0 to 2; n2 is an integer of 1 or 2; n3 is an integer of 1 or 2; and n4 is an integer of 0 to 4.

### TECHNICAL FIELD

The present invention relates to a polymerizable monomer, a polymer compound, a resist composition, and a patterning process.

### BACKGROUND ART

Along with high integration and high processing speed of LSI, miniaturization of pattern rule is rapidly advancing. This is attributable to the spreads of high speed communication of 5G and artificial intelligence (AI), which require high-performance devices for the processing. As a leading edge miniaturization technology, mass production of devices at the 5-nm node by way of lithography using extreme ultraviolet (EUV) having a wavelength of 13.5 nm has been conducted. Further, the use of EUV lithography in the next-generation devices at 3-nm node and next-next-generation devices at 2-nm node has also been considered.

With the progress of miniaturization, image blur due to acid diffusion has become a problem. In order to ensure the resolution of a fine pattern having a size of 45 nm or less, it is proposed that not only the improvement of the dissolution contrast, which has been conventionally proposed, but also the control of acid diffusion is important (Non-Patent Document 1). However, since the sensitivity and the contrast of a chemically-amplified resist composition are enhanced by acid diffusion, attempts to minimize acid diffusion by decreasing the temperature or the time of post-exposure bake (PEB) results in a significant decrease in the sensitivity and the contrast.

A triangle trade-off among the sensitivity, the resolution, and the edge roughness (LER, LWR) has been known. In order to improve the resolution, it is necessary to suppress acid diffusion; however, as the acid diffusion distance becomes shorter, the sensitivity decreases.

It is effective to suppress acid diffusion by adding an acid generator that generates a bulky acid. Therefore, it is proposed to incorporate a repeating unit derived from an onium salt having a polymerizable unsaturated bond into a polymer. In this case, the polymer also functions as an acid generator (polymer-bound acid generator). Patent Document 1 proposes a sulfonium salt or an iodonium salt having a polymerizable unsaturated bond which generates a specific sulfonic acid. Patent Document 2 proposes a sulfonium salt in which a sulfonic acid is directly bonded to the main chain (backbone).

The structure of the acid-labile group in the base polymer is important as a component that contributes to the performance of the positive resist composition. Related to this matter, a tertiary ester-type acid-labile group bonded to an aromatic group substituted with a fluorine atom has been proposed (Patent Documents 3 and 4). Since a tertiary ester-type acid-labile group bonded to an aromatic group has very high elimination reactivity by an acid, it has been difficult to control acid diffusion; however, the elimination reactivity can be appropriately suppressed by introducing a fluorine atom into an aromatic group.

Fluorine atom is the second sterically smallest element next to hydrogen atom, and is excellent in hydrophobicity and lipophilicity. In particular, a trifluoromethoxy group is known as a substituent having remarkably excellent hydrophobicity as compared with a corresponding methoxy group (Non-Patent Document 2). Tertiary ester-type acid-labile groups obtained by substituting them with an aromatic group have also been proposed (Patent Document 5).

On the other hand, the olefin-containing tertiary ester-type acid-labile group exemplified in paragraph of Patent Document 6 has very high elimination reactivity by an acid, and it was difficult to control the acid diffusion. In addition, the olefin-containing secondary ester-type acid-labile group exemplified in paragraph [0188] of Patent Document 7 has low elimination reactivity by an acid, and has a problem in which a high dissolution contrast is not obtained.

Such a tertiary ester-type acid-labile group generates a carboxylic acid after a deprotection reaction by an acid. The carboxylic acid has a swelling behavior in an alkaline developer, and the swelling causes pattern collapse during the fine pattern formation. In order to meet the demand for further miniaturization, development of an acid-labile monomer having a desirable acid elimination reactivity with respect to an acid and being capable of suppressing swelling with respect to an alkaline developer even after the elimination reaction has been required.

### CITATION LIST

### Patent Literature

Patent Document JP 2006-045311 A
Patent Document JP 2006-178317 A
Patent Document JP 3832564 B
Patent Document JP 5655754 B
Patent Document JP 2019-214554 A
Patent Document JP 2001-302728 A
Patent Document JP 2022-025610 A

### Non-Patent Literature

Non-patent Document 1: SPIE Vol. 6520 65203L-1 (2007)
Non-patent Document 2: Fusso Kagaku Nyumon 2010-Kiso to Oyo no Saizensen: edited by 155th Fluorine Chemistry Committee, Japan Society for the Promotion of Science, Sankyo Shuppan Co., Ltd., 2010

### SUMMARY OF INVENTION

### Technical Problem

The present invention has been made in view of the above circumstances, and an object thereof is to provide, in particular, a polymerizable monomer having sensitivity and resolution higher than those of the previously-known positive resist compositions, and exhibiting small edge roughness and size variation, and a desirable pattern shape after exposure, as well as a polymer compound, a resist composition, and a patterning process using the polymerizable monomer.

In particular, an object of the present invention is to provide a polymerizable monomer used for a chemically-amplified resist composition having excellent solvent solubility, high sensitivity and high contrast, excellent lithography performance such as exposure latitude (EL) and LWR, and an excellent pattern shape in photolithography using high-energy beam such as KrF or ArF excimer laser beam, electron beam (EB) or EUV, as well as a polymer compound and a chemically-amplified resist composition obtained by using the polymerizable monomer, and a patterning process using the chemically-amplified resist composition.

### SOLUTION TO PROBLEM

In order to achieve the above objects, the present invention provides: a polymerizable monomer represented by the following general formula (1),

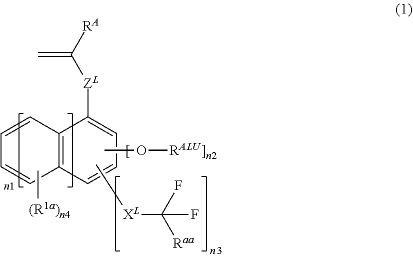

wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a linear, branched or cyclic hydrocarbyl group having 1 to 20 carbon atoms which may contain a heteroatom; n1 is an integer of 0 to 2; n2 is an integer of 1 or 2; n3 is an integer of 1 or 2; and n4 is an integer of 0 to 4; when n2=1, each of —O—R^{ALU} and —X^{L}—CF₂(R^{aa}) is bonded to each of carbon atoms which are adjacent to each other on an aromatic ring; when n2=2, one of two —O—R^{ALU} is bonded to a carbon atom adjacent to a carbon atom on the aromatic ring to which —X^{L}—CF₂(R^{aa}) is bonded.

Such a polymerizable monomer can be used as a raw material of a resist composition having sensitivity and resolution higher than those of the previously-known positive resist materials, and exhibiting small edge roughness and size variation and a desirable pattern shape after exposure.

The general formula (1) above is preferably that represented by the following formula (1-A),

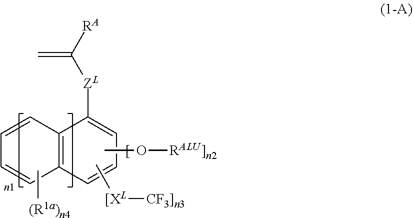

wherein R^{A}, Z^{L}, X^{L}, R^{1a}, R^{ALU}, n1, n2, n3, and n4 are as defined above.

Such a monomer is more preferable as the polymerizable monomer described above.

The R^{ALU} is preferably that represented by the following formula (AL-1) or (AL-2) together with the adjacent oxygen atom.

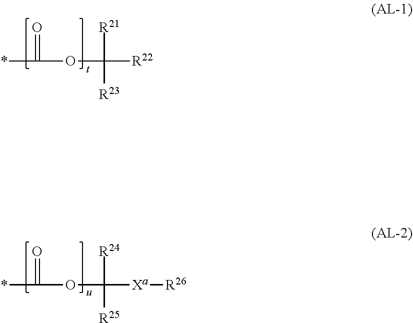

In the formula (AL-1), R²¹, R²², and R²³ independently represent a hydrocarbyl group having 1 to 12 carbon atoms which may contain a heteroatom; any two of R²¹, R²², and R²³ may be bonded to each other to form a ring; and t is an integer of 0 or 1. In the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

Such a group has a desirable function as an acid-labile group.

The present invention also provides a polymer compound comprising a repeating unit obtained from the polymerizable monomer described above, in which the repeating unit is represented by the following general formula (Ia),

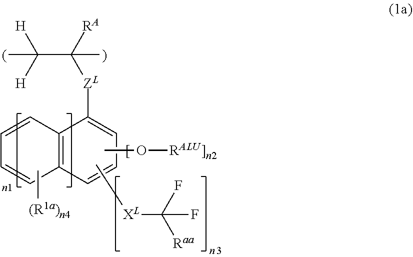

wherein R^{A}, Z^{L}, X^{L}, R^{1a}, R^{ALU}, R^{aa}, n1, n2, n3, and n4 are as defined above.

By containing such a polymer compound, it is usable as a resist composition having sensitivity and resolution higher than those of the previously-known positive resist compositions, and exhibiting small edge roughness and size variation and a desirable pattern shape after exposure.

The polymer compound is preferably a polymer compound comprising a repeating unit obtained from the polymerizable monomer described above, in which the repeating unit is represented by the following general formula (1-Aa),

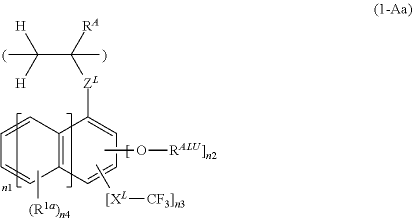

wherein R^{A}, Z^{L}, X^{L}, R^{1a}, R^{ALU}, n1, n2, n3, and n4 are as defined above.

Such a compound is more preferable as the polymer compound described above.

In the polymer compound, the R^{ALU} is preferably represented by the following formula (AL-1) or (AL-2)

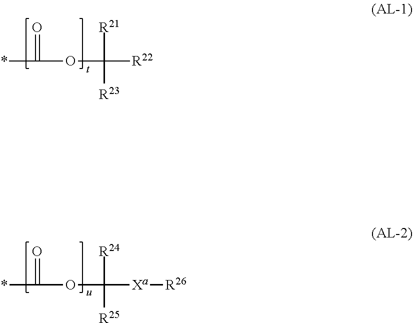

wherein R²¹, R²², R²³, t, R²⁴, R²⁵, R²⁶, X^{a} and u are defined above.

Such a compound has a desirable function as an acid-labile group.

It is preferable that the polymer compound further contain at least one selected from repeating units represented by the following general formula (a1) or (a2),

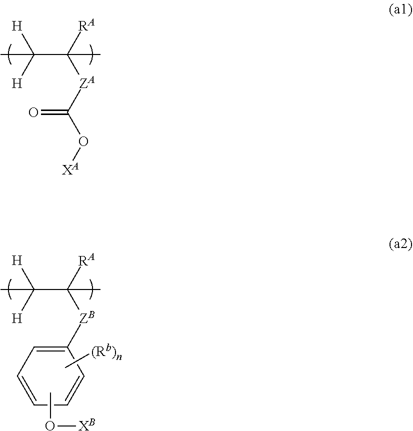

wherein R^{A} independently represents a hydrogen atom, a fluorine atom, a methyl group, or a trifluoromethyl group; Z^{A} is a single bond, an alkoxy group optionally substituted with a halogen atom, a phenylene group or a naphthylene group optionally substituted with a halogen atom, or (backbone) —C(═O)—O—Z^{A1}—; Z^{A1} is a heteroatom, an alkoxy group having 1 to 10 carbon atoms which may contain a fluorine atom, a linear, branched or cyclic alkanediyl group having 1 to 20 carbon atoms which may contain a hydroxy group, an ether bond, an ester bond, a lactone ring, a thioether bond, a sulfonyl group, or a sulfonamide structure, or a phenylene group or a naphthylene group; Z^{B} is a single bond or (backbone) —C(═O)—O—Z^{B1}—; Z^{B1} is a single bond or an alkanediyl group having 1 to 10 carbon atoms which may contain an ester and/or an ether bond; X^{A} and X^{B} independently represent an acid-labile group; R^{b} represents a halogen atom, a nitro group, or a linear, branched or cyclic monovalent hydrocarbon group having 1 to 20 carbon atoms which may contain a heteroatom; n is an integer of 0 to 4.

Such a compound has a desirable function as the polymer compound described above.

It is preferable that the polymer compound further contain at least one selected from repeating units represented by the following general formula (b1) or (b2),

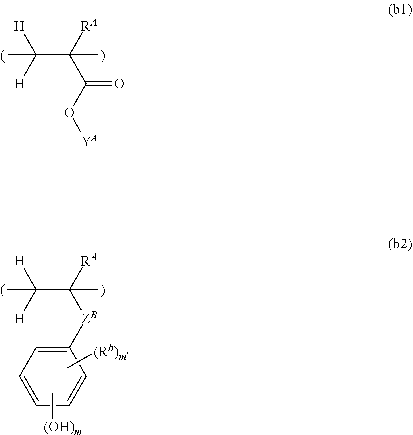

wherein R^{A} independently represents a hydrogen atom, a fluorine atom, a methyl group, or a trifluoromethyl group; YA is a hydrogen atom, or a polar group comprising at least one or more structures selected from the group consisting of a hydroxy group, a cyano group, a carbonyl group, a carboxy group, an ether bond, an ester bond, a sulfonic acid ester bond, a sulfonic acid amide bond, a carbonate bond, a lactone ring, a sultone ring, a sulfur atom, and a carboxylic acid anhydride; Z^{B} is a single bond or (backbone) —C(═O)—O—Z^{B1}—; Z^{B1} is a single bond or an alkanediyl group having 1 to 10 carbon atoms which may contain an ester and/or an ether bond; R^{b} represents a halogen atom, a nitro group, or a linear, branched or cyclic monovalent hydrocarbon group having 1 to 20 carbon atoms which may contain a heteroatom; m is an integer of 1 to 4; and m′ is an integer of 0 to 4.

Such a compound has a more desirable function as the polymer compound described above.

It is preferable that the polymer compound further contain at least one selected from repeating units represented by the following general formulae (C1) to (C4),

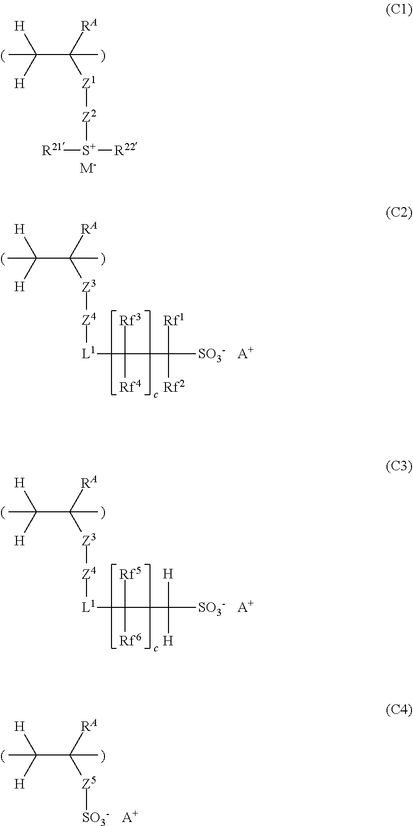

wherein R^{A} independently represents a hydrogen atom, a fluorine atom, a methyl group, or a trifluoromethyl group; Z¹ is a single bond or a phenylene group; Z² is a single bond, *—C(═O)—O—Z²¹—, *—C(═O) —NH—Z²¹—, or *—O—Z²¹—; Z²¹ is an aliphatic hydrocarbylene group having 1 to 12 carbon atoms, a phenylene group, or a divalent group obtained by combining these groups, and may contain a carbonyl group, an ester bond, an ether bond or a hydroxy group; Z³ is a single bond, a phenylene group, a naphthylene group, or *—C(═O)—O—Z³¹—; Z³¹ is a single bond, an aliphatic hydrocarbylene group having 1 to 14 carbon atoms which may contain a hydroxy group, an ether bond, an ester bond, or a lactone ring, or a phenylene group or a naphthylene group; Z⁴ is a single bond, a methylene group, or *—Z⁴¹—C(═O)—O—; Z⁴¹ represents a hydrocarbylene group having 1 to 20 carbon atoms which may contain an ether bond, an ester bond, or a heteroatom; Z⁵ is a single bond, a methylene group, an ethylene group, a phenylene group, a fluorinated phenylene group, a phenylene group substituted with a trifluoromethyl group, *—C(═O)—O—Z⁵¹—, *—C(═O)—N(H)—Z⁵¹—, or *—O—Z⁵¹—; Z⁵¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond or a hydroxy group; and * represents a bonding arm to a carbon atom in the backbone; R^{21′} and R^{22′} independently represent a hydrocarbyl group having 1 to 20 carbon atoms which may contain a heteroatom; R^{21′} and R^{22′} may also be bonded to each other to form a ring with a sulfur atom to which R^{21′} and R^{22′} are bonded; L¹ is a single bond, an ether bond, an ester bond, a carbonyl group, a sulfonic acid ester bond, a carbonate bond or a carbamate bond; Rf¹ and Rf² independently represent a fluorine atom or a fluorinated alkyl group having 1 to 6 carbon atoms; Rf³ and Rf⁴ independently represent a hydrogen atom, a fluorine atom, or a fluorinated alkyl group having 1 to 6 carbon atoms; Rf⁵ and Rf⁶ independently represent a hydrogen atom, a fluorine atom, or a fluorinated alkyl group having 1 to 6 carbon atoms, provided that not all of Rf⁵ and Rf⁶ are hydrogen atoms at the same time; M⁻ is a non-nucleophilic counter ion; A⁺ is an onium cation; and c is an integer of 0 to 3.

Such a compound has further desirable function as the polymer compound described above.

The present invention also provides a resist composition containing a base resin formed from the polymer compound described above, and an organic solvent.

By incorporating the polymer compound and an organic solvent, a desirable resist composition is obtained.

It is preferable that the resist composition further contains one or more selected from a quencher and an acid generator.

Such a composition has more desirable function as a resist composition.

It is preferable that the resist composition further contain a surfactant which is insoluble or substantially insoluble in water and soluble in an alkaline developer, and/or a surfactant which is insoluble or substantially insoluble in water and an alkaline developer.

Such a composition has further desirable function as a resist composition.

Further, the present invention provides a patterning process, comprising the steps of forming a resist film on a substrate by using the resist composition, exposing the resist film to high-energy beam, and developing the exposed resist film by using a developer.

Such a patterning process is capable of forming a desirable pattern.

Further, the high-energy beam is preferably KrF excimer laser beam, ArF excimer laser beam, an electron beam, or extreme ultraviolet ray having a wavelength of 3 to 15 nm.

By using such a high-energy beam, more desirable pattern can be obtained.

### Advantageous Effects of Invention

When a pattern is formed by using a chemically-amplified resist composition containing, as a base polymer, the polymer compound obtained by using the polymerizable monomer of the present invention, a resist pattern having a high contrast and desirable sensitivity, ensuring excellent lithography performance such as DOF (depth of focus) and LWR, and having suppressed pattern collapse can be formed.

### DESCRIPTION OF EMBODIMENTS

There has been demanded a polymerizable monomer having sensitivity and resolution higher than those of the previously-known positive resist compositions, and exhibiting small edge roughness and size variation, and a desirable pattern shape after exposure.

As a result of intensive study to achieve the objects described above, the present inventors found that a polymerizable monomer having a specific structure is excellent in solvent solubility and that a chemically-amplified resist composition containing, as a base polymer, a polymer compound obtained by using the monomer has high sensitivity and a high contrast and excellent lithography performance such as EL (exposure latitude) and LWR, and is very effective in suppressing pattern collapse during formation of a fine pattern. With these findings, the present inventors completed the present invention.

Specifically, the present invention is a polymerizable monomer represented by the following general formula (1),

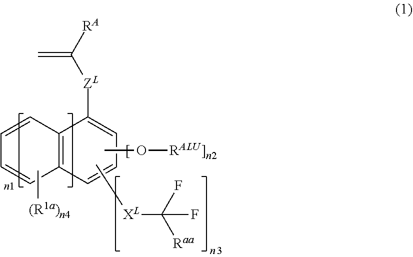

wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a linear, branched or cyclic hydrocarbyl group having 1 to 20 carbon atoms which may contain a heteroatom; n1 is an integer of 0 to 2; n2 is an integer of 1 or 2; n3 is an integer of 1 or 2; and n4 is an integer of 0 to 4; when n2=1, each of —O—R^{ALU} and —X^{L}—CF₂(R^{aa}) is bonded to each of carbon atoms which are adjacent to each other on an aromatic ring; when n2=2, one of two —O—R^{ALU} is bonded to a carbon atom adjacent to a carbon atom on the aromatic ring to which —X^{L}—CF₂(R^{aa}) is bonded.

Further, the present invention is also a polymer compound comprising a repeating unit obtained from the polymerizable monomer described above, a resist composition containing a base resin formed from the polymer compound and an organic solvent, and a patterning process using the resist composition.

Hereinafter, embodiments of the present invention will be described in detail, but the present invention is not limited thereto.

### [Polymerizable Monomer]

The polymerizable monomer of the present invention is represented by the following formula (1),

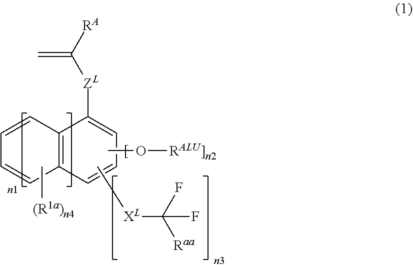

wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a linear, branched or cyclic hydrocarbyl group having 1 to 20 carbon atoms which may contain a heteroatom; n1 is an integer of 0 to 2; n2 is an integer of 1 or 2; n3 is an integer of 1 or 2; and n4 is an integer of 0 to 4; when n2=1, each of —O—R^{ALU} and —X^{L}—CF₂(R^{aa}) is bonded to each of carbon atoms which are adjacent to each other on an aromatic ring; when n2=2, one of two —O—R^{ALU} is bonded to a carbon atom adjacent to a carbon atom on the aromatic ring to which —X^{L}—CF₂(R^{aa}) is bonded.

In the formula (1), R^{A} represents a hydrogen atom, a fluorine atom, a methyl group, or a trifluoromethyl group. Among these, a hydrogen atom and a methyl group are preferable.

Z^{L} is a single bond or (backbone) —C(═O)—O—. Among these, single bond is preferable.

In the formula (1), R^{1a} independently represents a linear, branched or cyclic hydrocarbyl group having 1 to 20 carbon atoms which may contain a heteroatom. The hydrocarbyl group may be any of a saturated, unsaturated, linear, branched, or cyclic hydrocarbyl group. Examples thereof include alkyl groups having 1 to 20 carbon atoms such as a methyl group, an ethyl group, a n-propyl group, an isopropyl group, a n-butyl group, and a tert-butyl group; cyclic saturated hydrocarbyl groups having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms such as a vinyl group, an allyl group, a propenyl group, a butenyl group, a hexenyl group; cyclic unsaturated hydrocarbyl groups having 3 to 20 carbon atoms such as a cyclohexenyl group; aryl groups having 6 to 20 carbon atoms such as a phenyl group, and a naphthyl group; aralkyl groups having 7 to 20 carbon atoms such as a benzyl group, a 1-phenylethyl group, and a 2-phenylethyl group; and groups obtained by combining these groups. Among them, aryl groups are preferable. Further, in these groups, a part or all of the hydrogen atoms of the hydrocarbyl group may be substituted with a group containing a heteroatom such as an oxygen atom, a sulfur atom, a nitrogen atom and a halogen atom, and a part of the —CH₂— constituting the hydrocarbyl group may be substituted with a group containing a heteroatom such as an oxygen atom, a sulfur atom, and a nitrogen atom, so that the groups contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic acid anhydride, a haloalkyl group, or the like.

In the formula (1), X^{L} represents an oxygen atom or a sulfur atom, and is preferably an oxygen atom in view of easy raw material procurement.

In the formula (1), R^{aa} represents a hydrogen atom or a fluorine atom, and is preferably a hydrogen atom in view of easy raw material procurement.

In the formula (1), R^{ALU} represents an acid-labile group that is formed together with the adjacent oxygen atom; specifically, a structure represented by the following formula (AL-1) or (AL-2) is preferable.

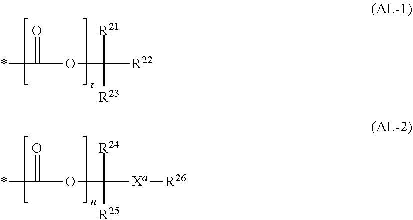

In the formula (AL-1), R²¹, R²², and R²³ independently represent a hydrocarbyl group having 1 to 12 carbon atoms which may contain a heteroatom; any two of R²¹, R²², and R²³ may be bonded to each other to form a ring; and t is an integer of 0 or 1. In the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

In the formula (AL-1), R²¹, R²², and R²³ preferably independently represent a hydrocarbyl group having 1 to 10 carbon atoms; any two of R²¹, R²², and R²³ may be bonded to each other to form a ring; and t is an integer of 0 or 1. In the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

Examples of the acid-labile group represented by the formula (AL-1) include, but are not limited thereto. * represents a bond to the adjacent oxygen atom.

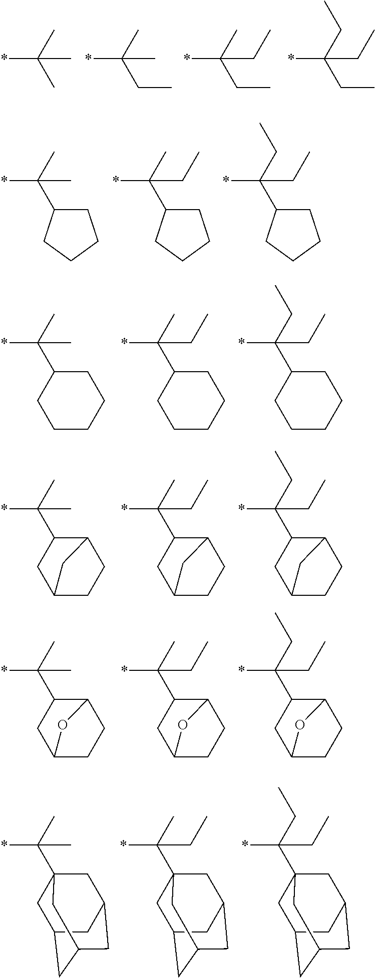

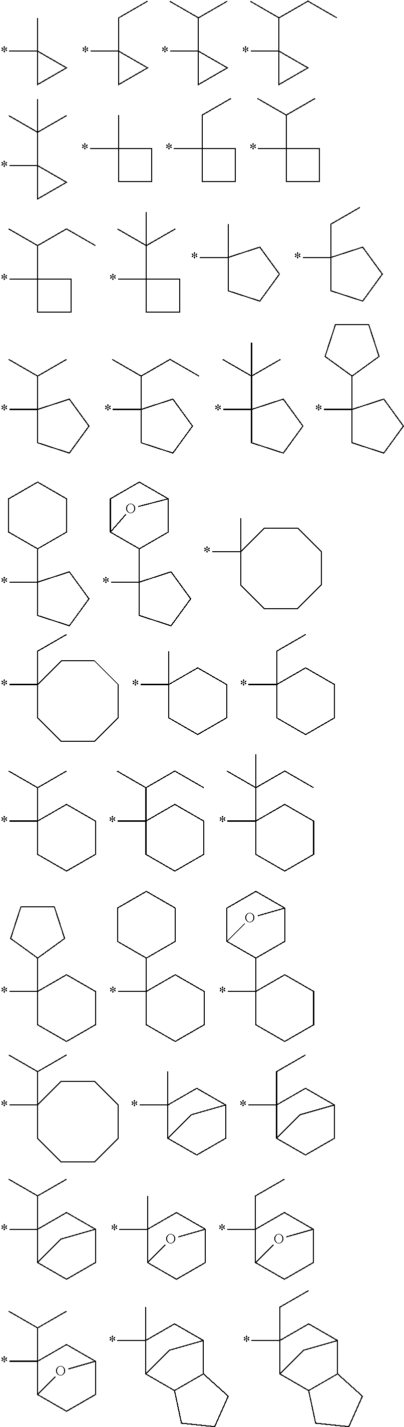

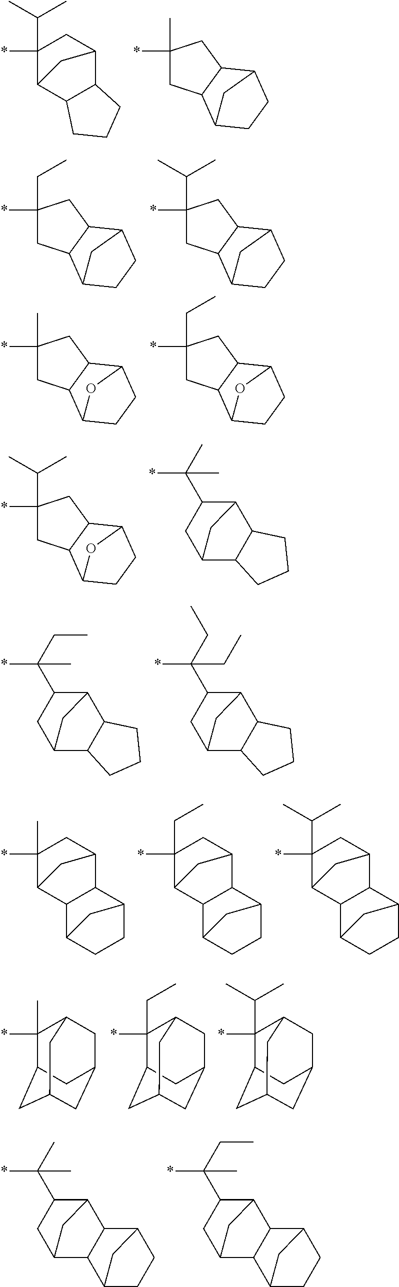

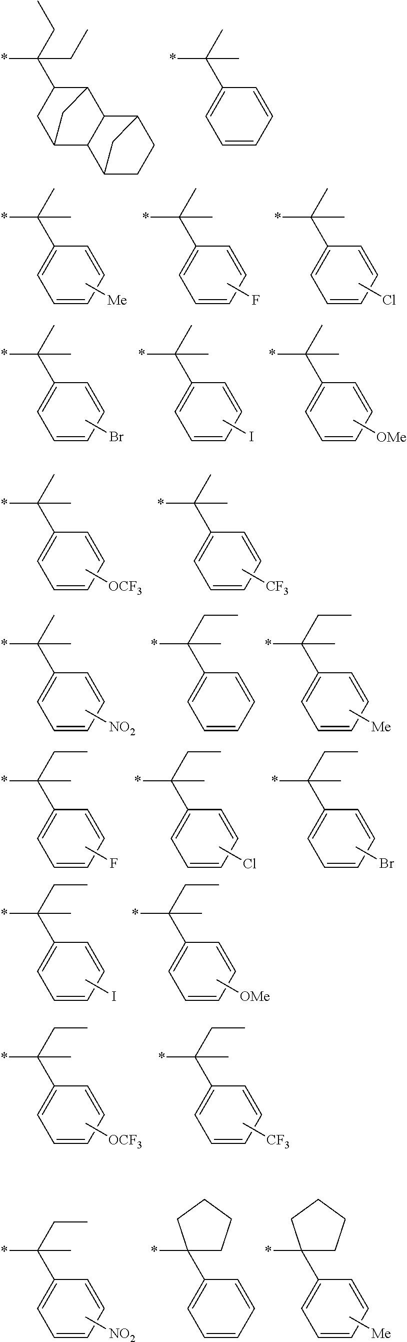

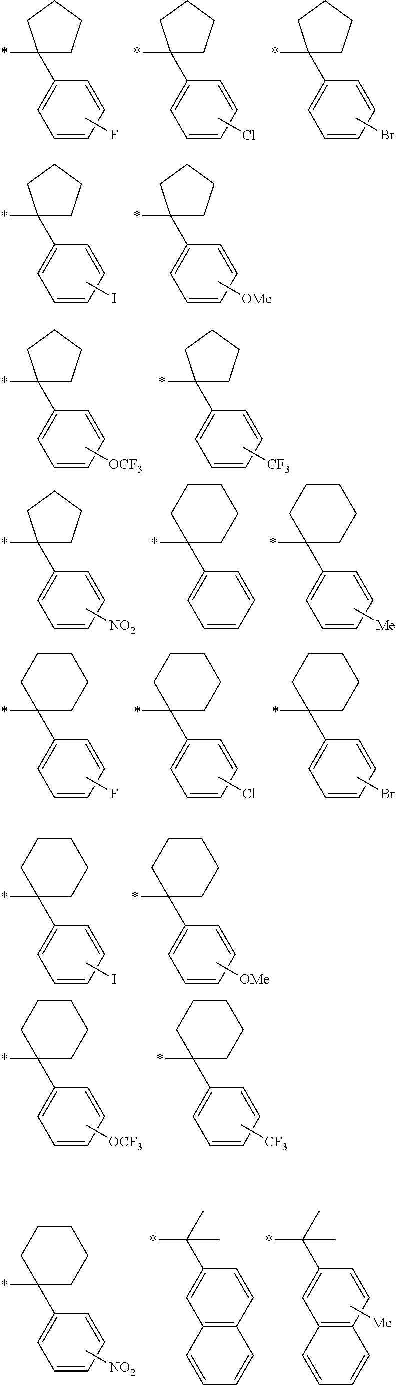

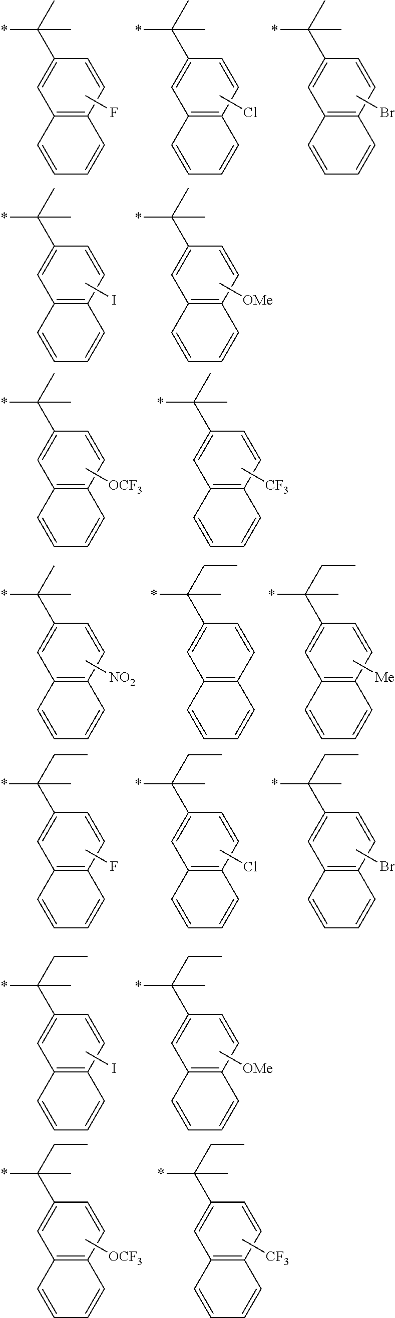

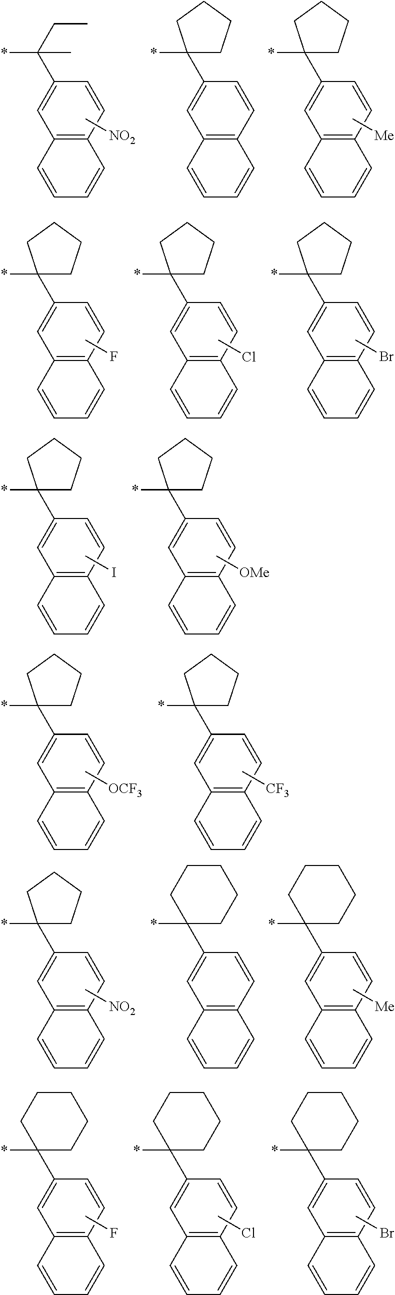

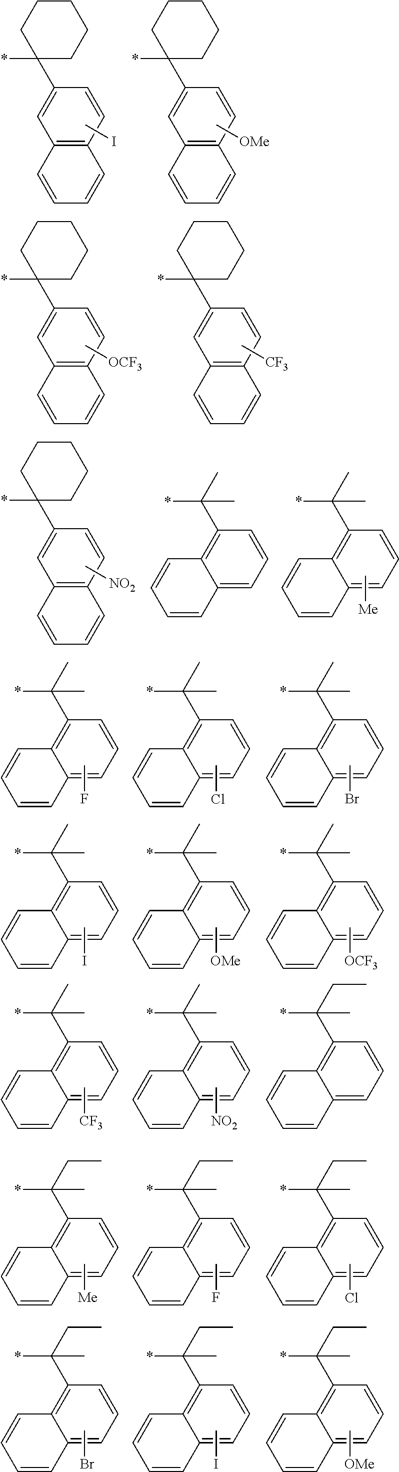

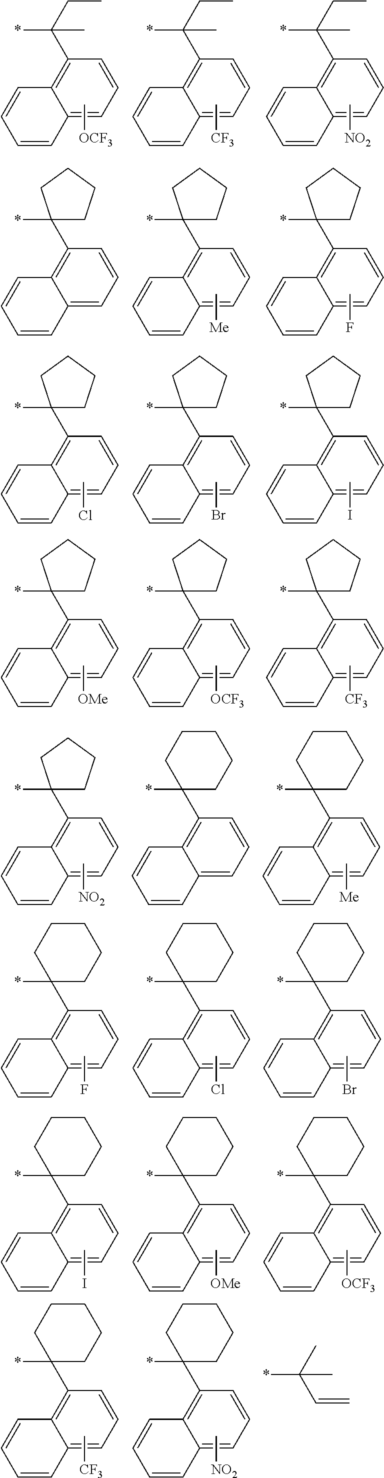

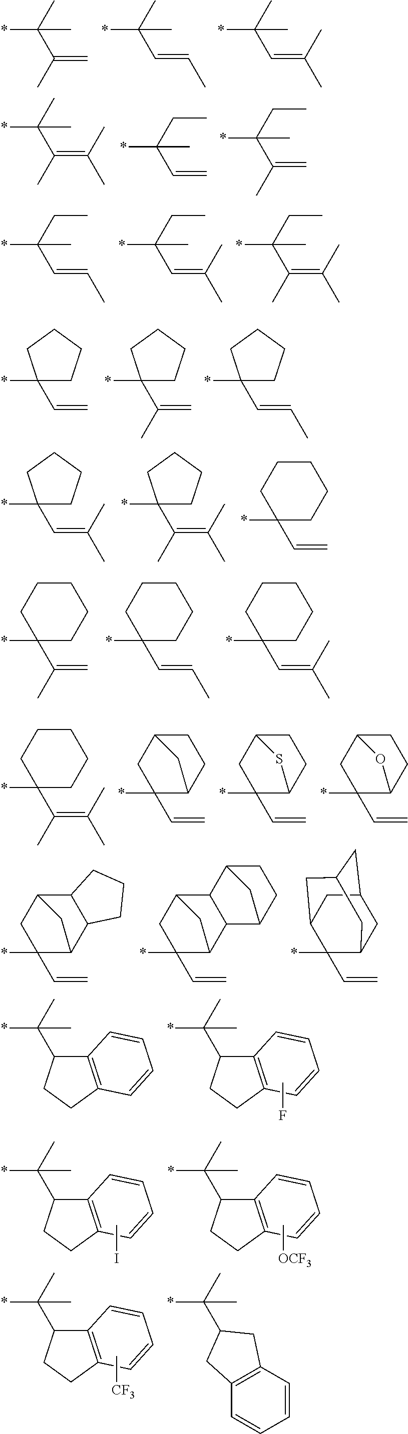

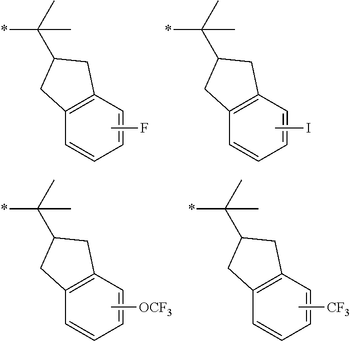

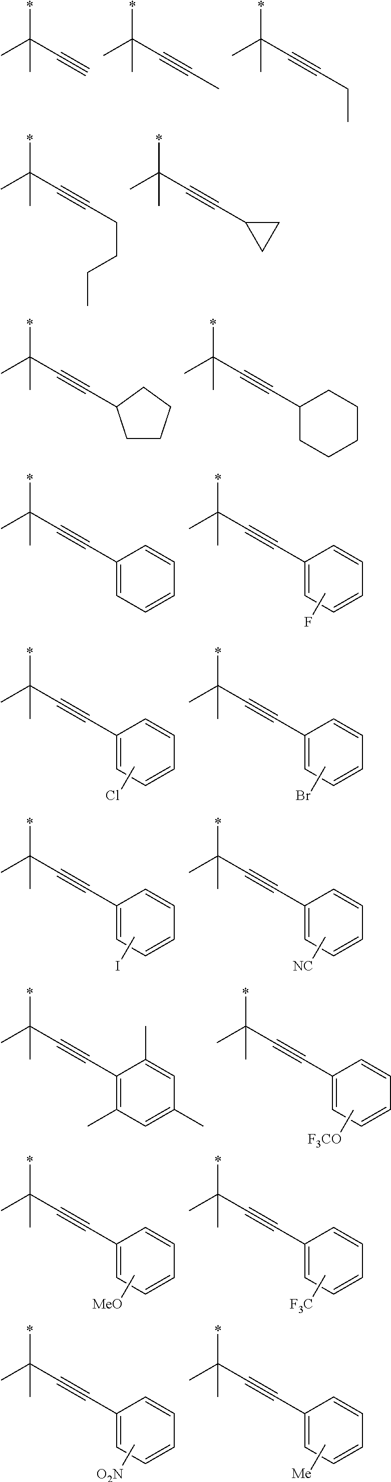

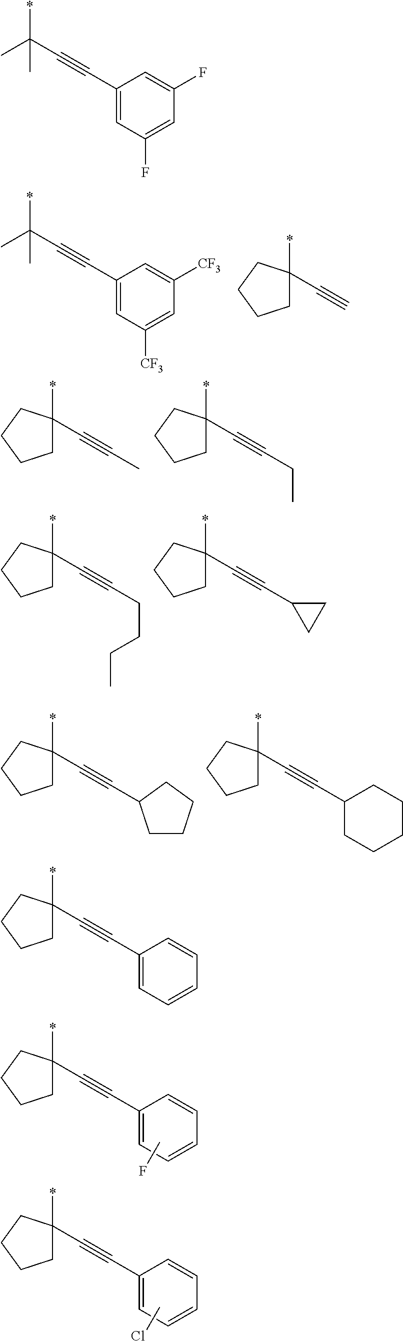

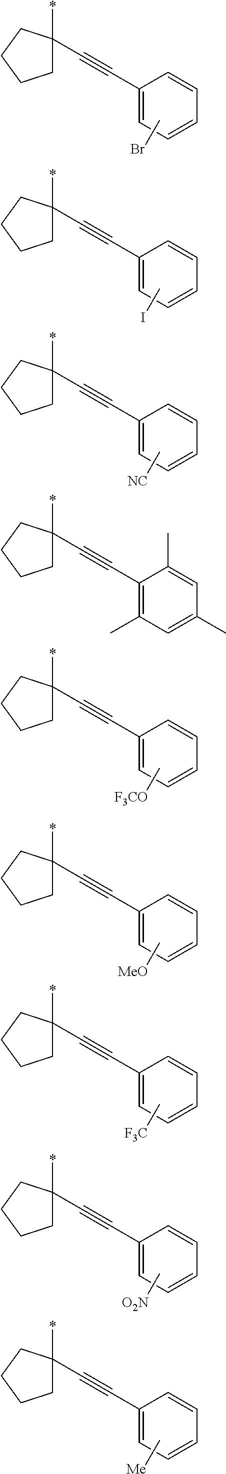

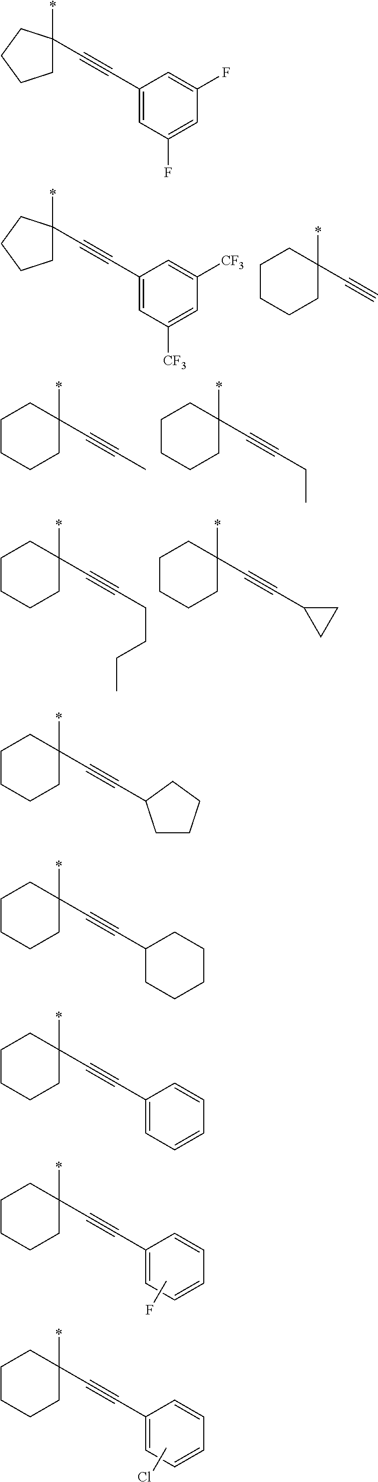

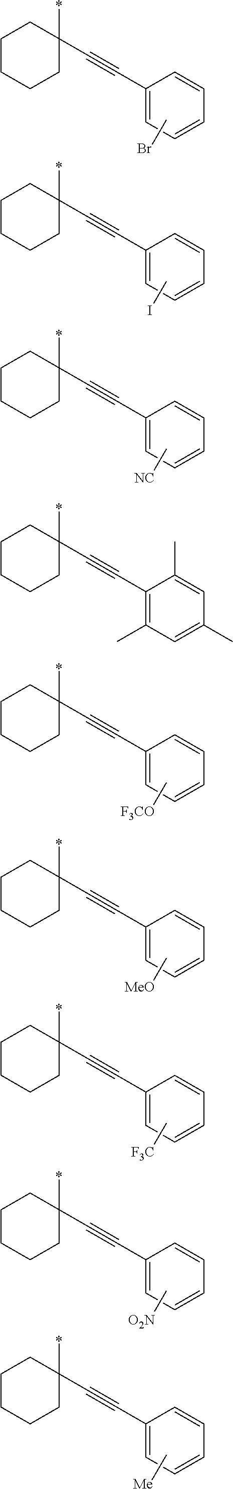

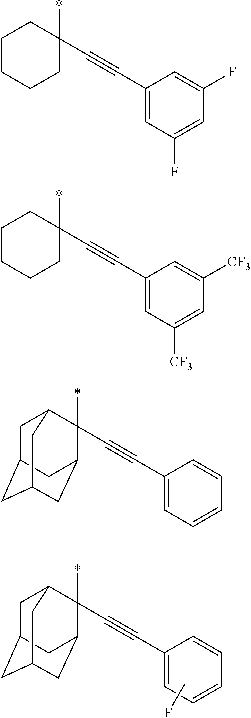

Examples of the acid-labile group represented by the formula (AL-2) include, but are not limited thereto. * represents a bond to the adjacent oxygen atom.

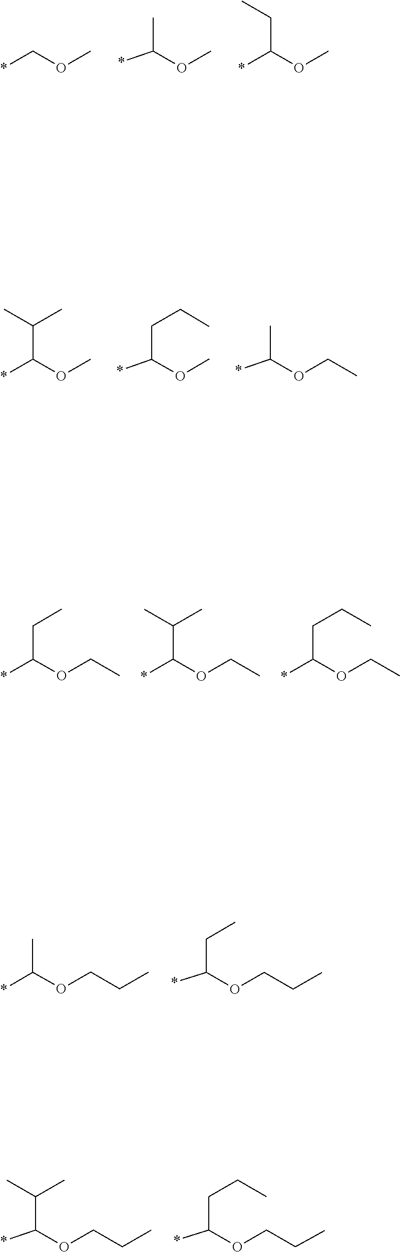

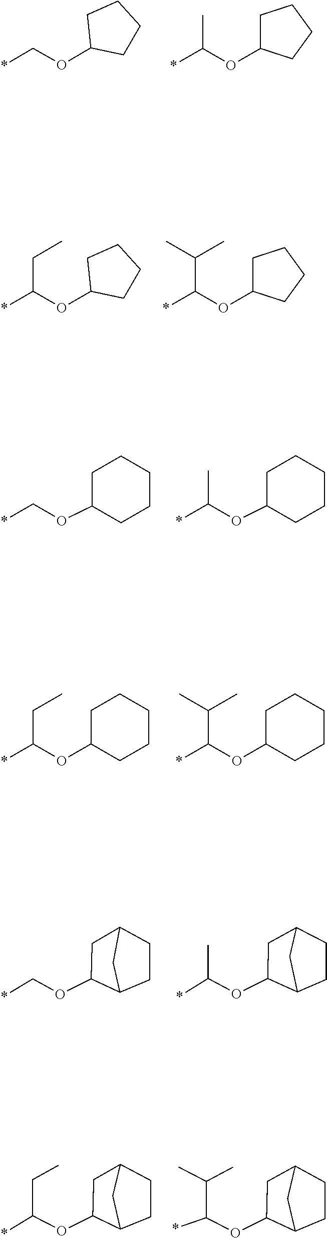

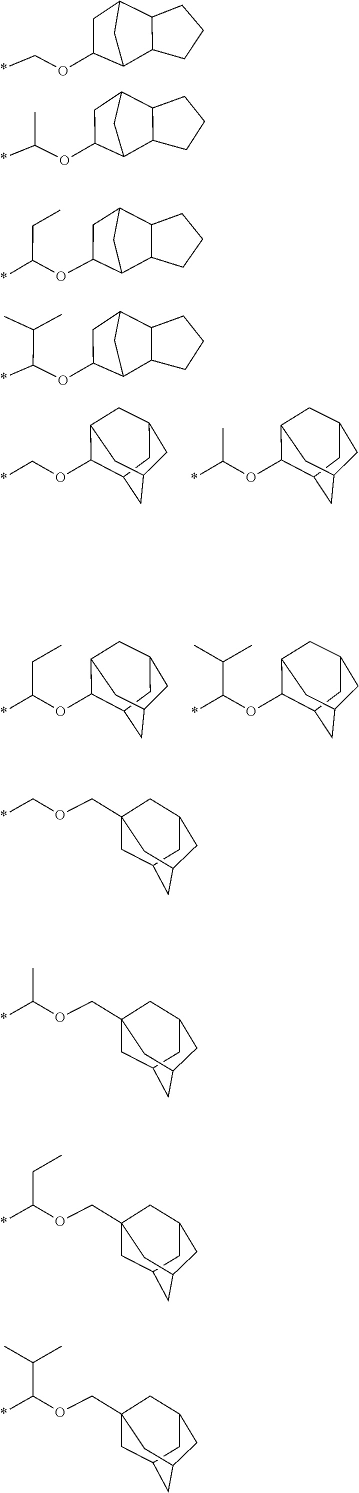

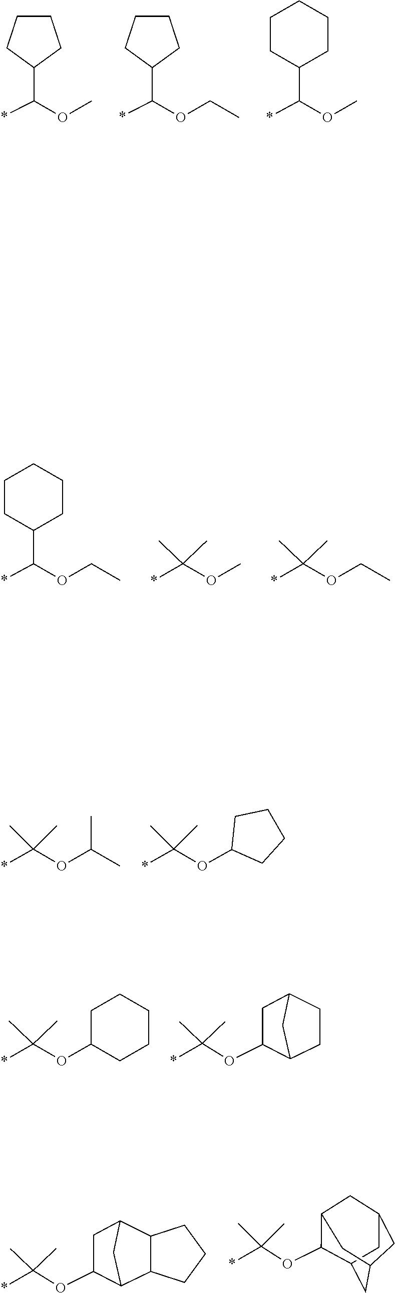

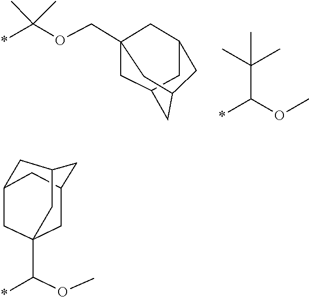

In the formula (1), there must be at least one pair of —O—R^{ALU} and —X^{L}—CF₂ (R^{aa}) each of which is bonded to each of two carbon atoms which are adjacent to each other on the aromatic ring. Being adjacent to each other improves the acidity of the aromatic alcohol that is generated after deprotection of R^{ALU}.

In the formula (1), n1 is an integer of 0 to 2. When n1 is 0, it represents a benzene ring, when n1 is 1, it represents a naphthalene ring, and when n1 is 2, it represents an anthracene ring. A benzene ring, i.e., the case where n1 is 0, is preferable in view of solvent solubility.

In the formula (1), n2 is an integer of 1 or 2. In view of easy raw material procurement, n2 is preferably 1.

In the formula (1), n3 is an integer of 1 or 2. In view of easy raw material procurement, n3 is preferably 1.

In the formula (1), n4 is an integer of 0 to 4. In view of easy raw material procurement, n4 is preferably 0 to 2.

The polymerizable monomer represented by the formula (1) is further preferably that represented by the following formula (1-A),

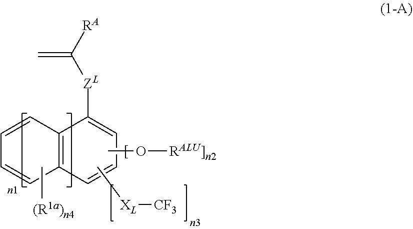

wherein R^{A}, Z^{L}, X^{L}, R^{1a}, R^{ALU}, n1, n2, n3, and n4 are as defined above.

Examples of the polymerizable monomers represented by the formula (1) and the formula (1-A) include, but are not limited thereto. The substitution position of the substituent on the aromatic ring is also not limited to the case above insofar as the groups bracketed by n2 and n3 are adjacent to each other.

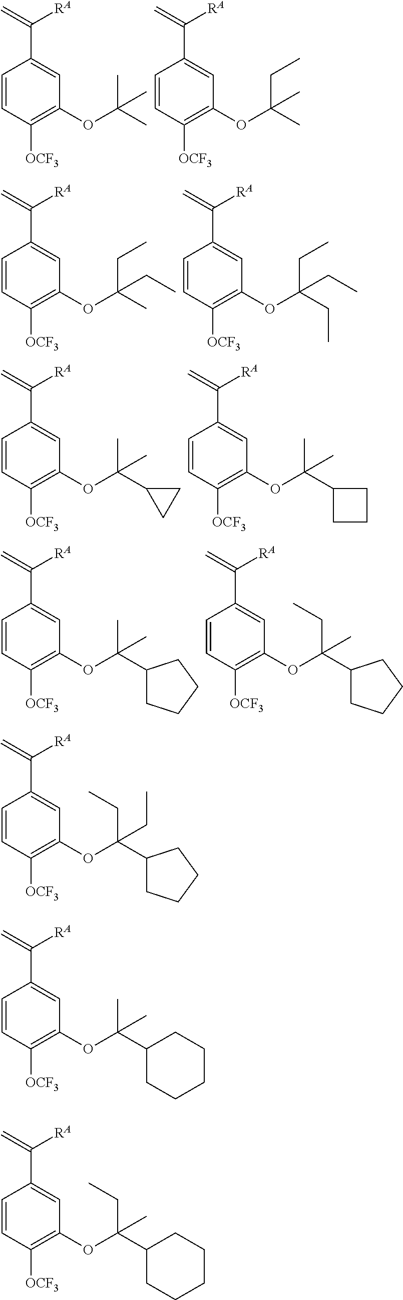

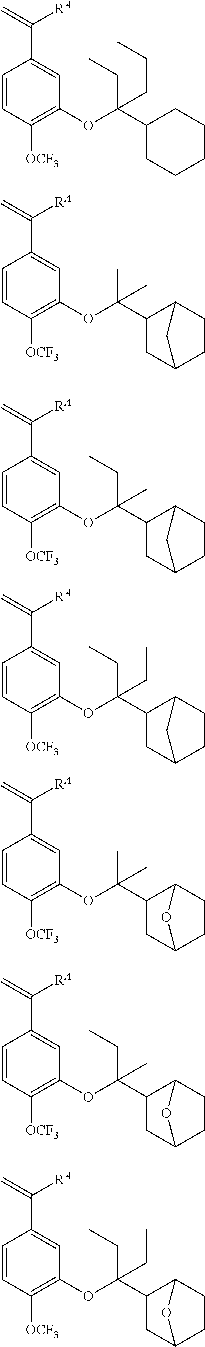

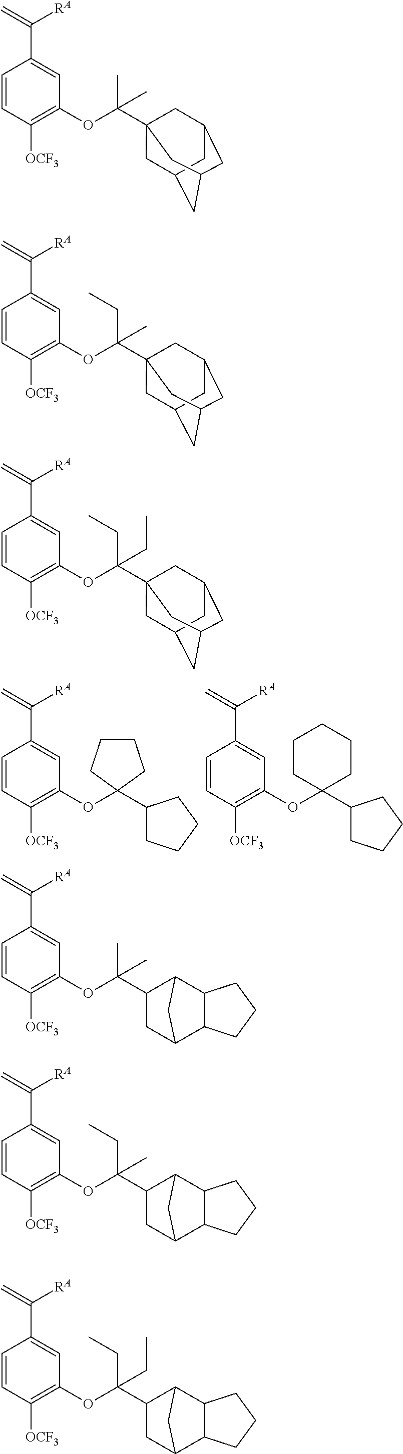

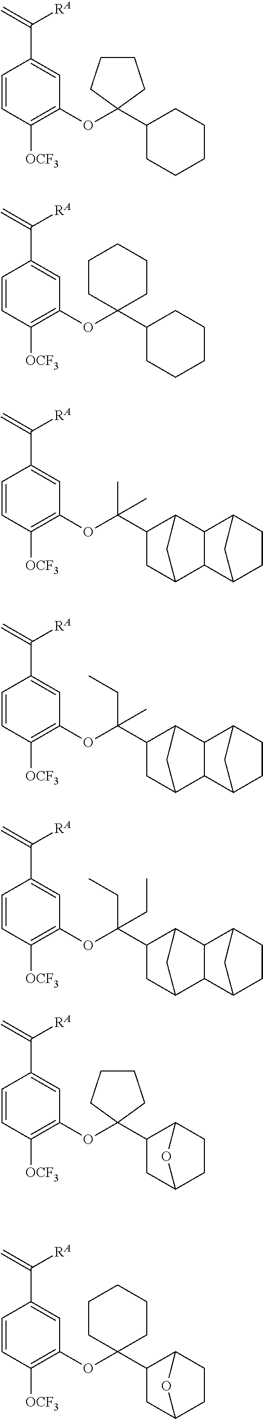

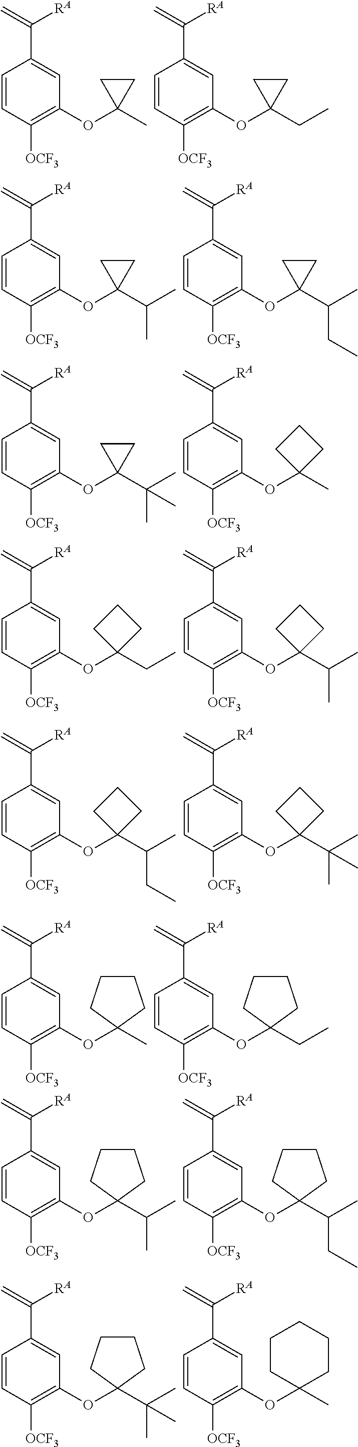

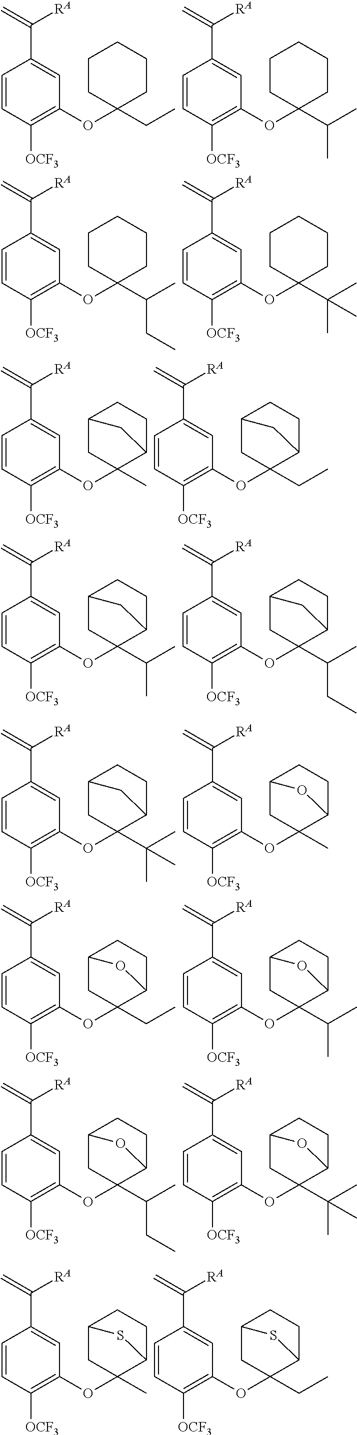

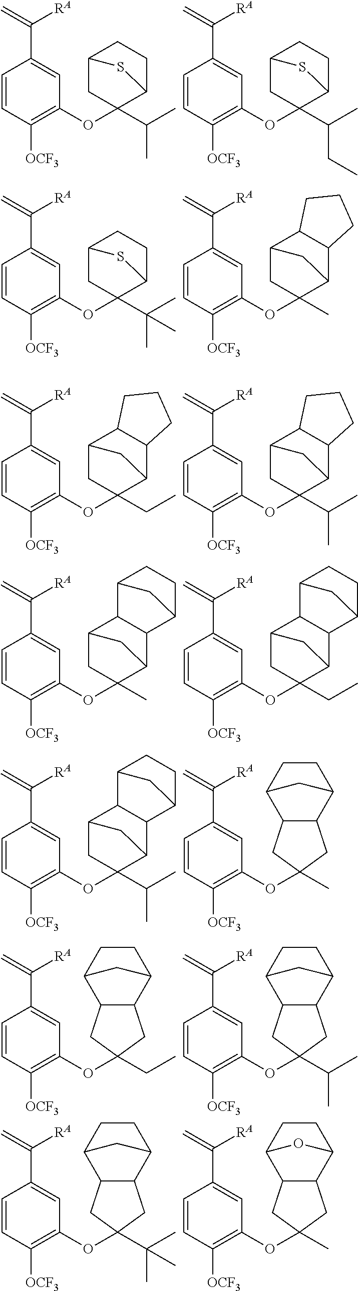

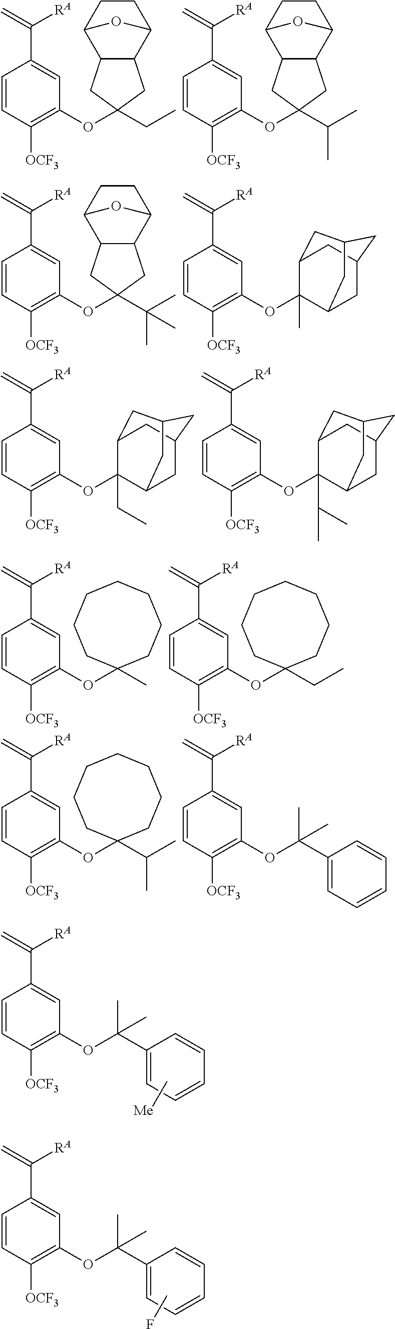

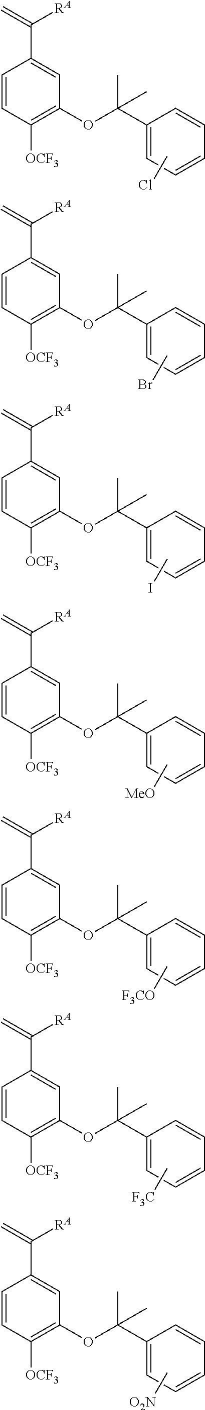

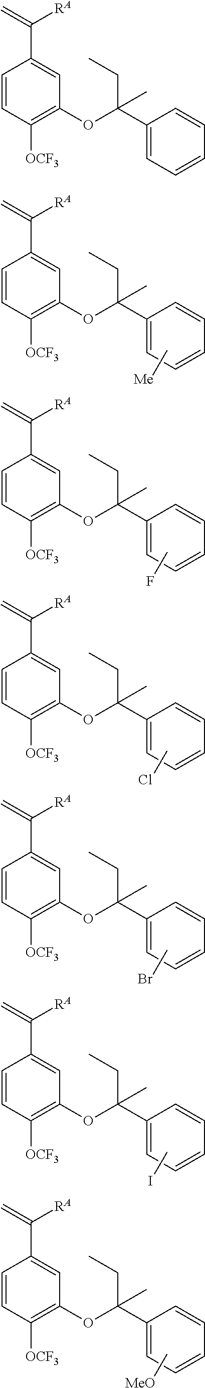

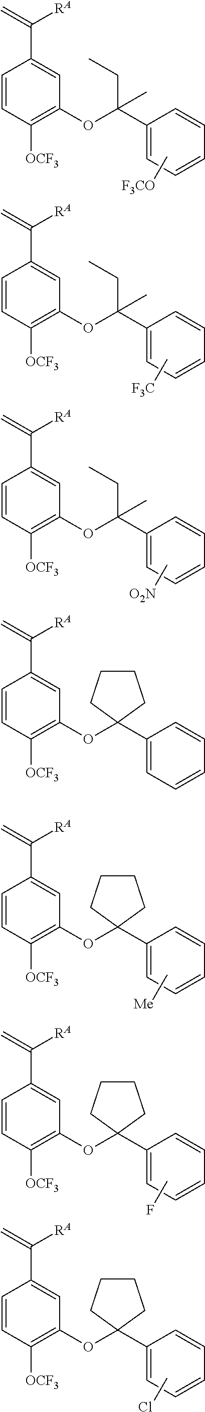

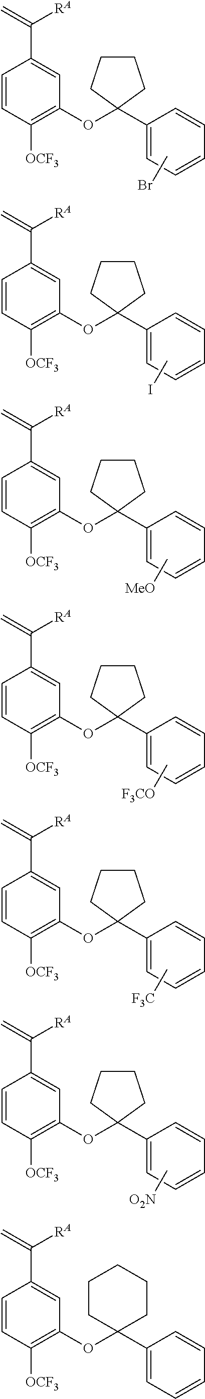

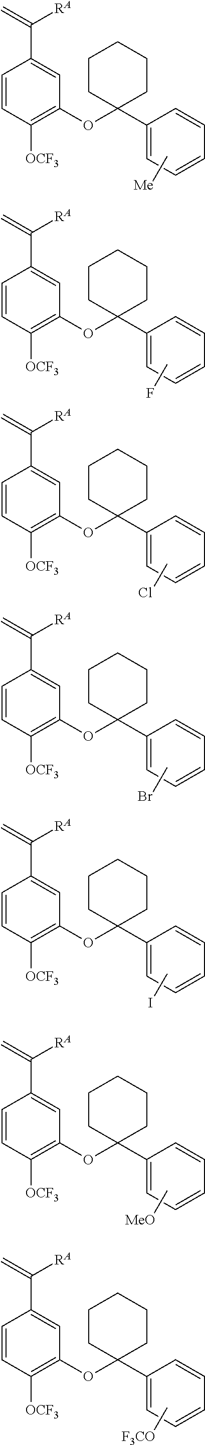

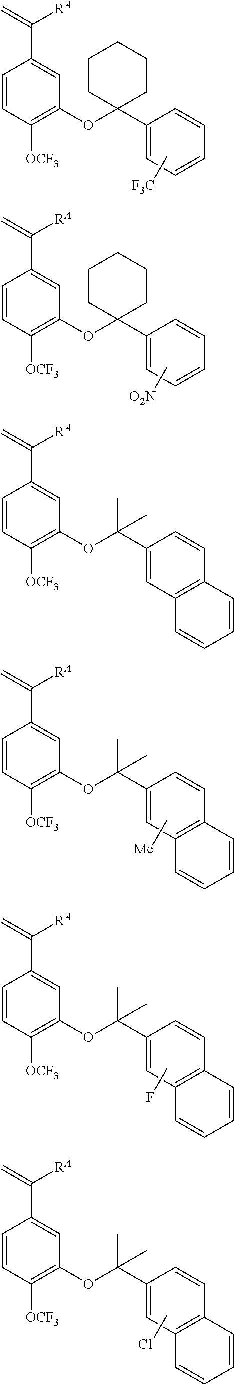

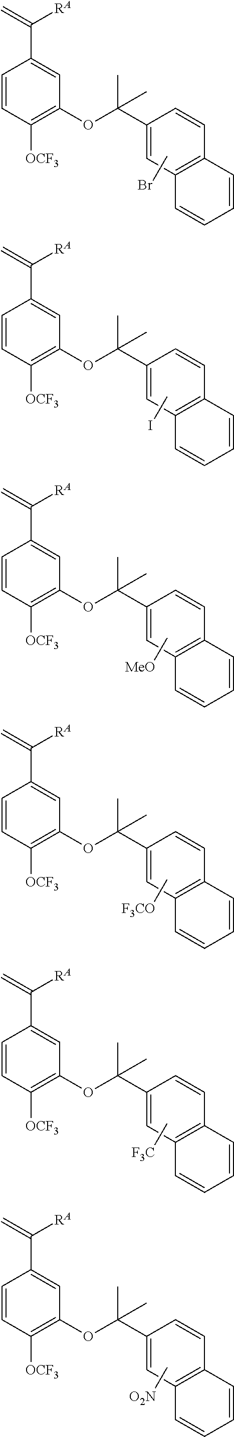

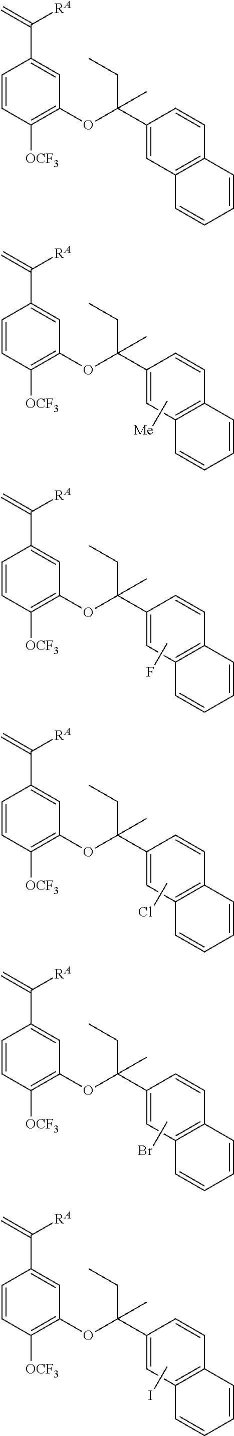

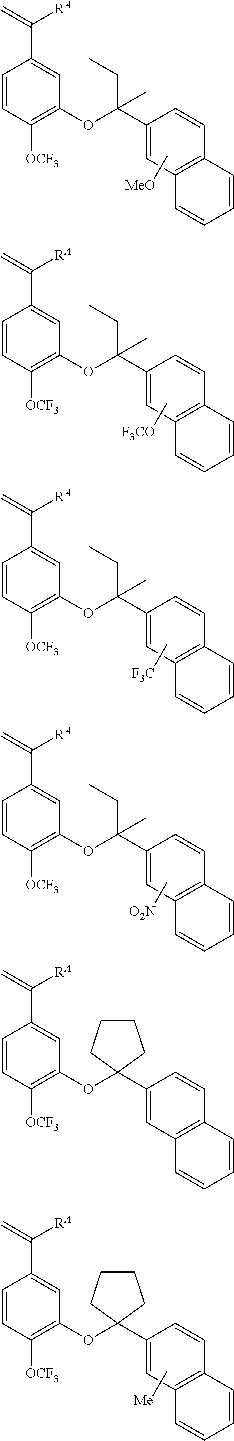

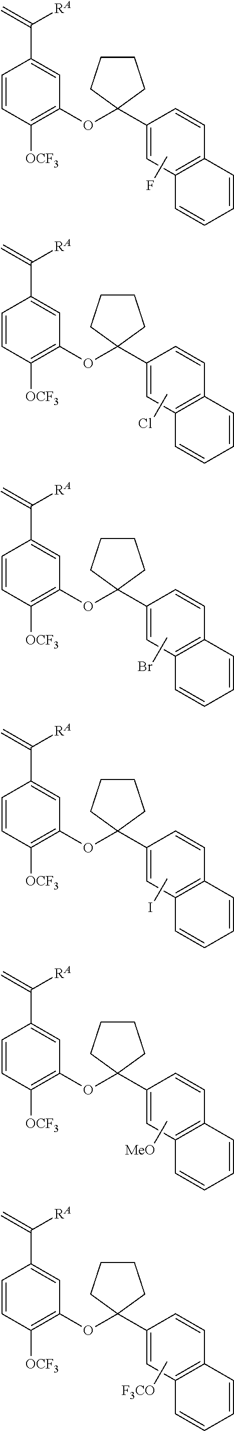

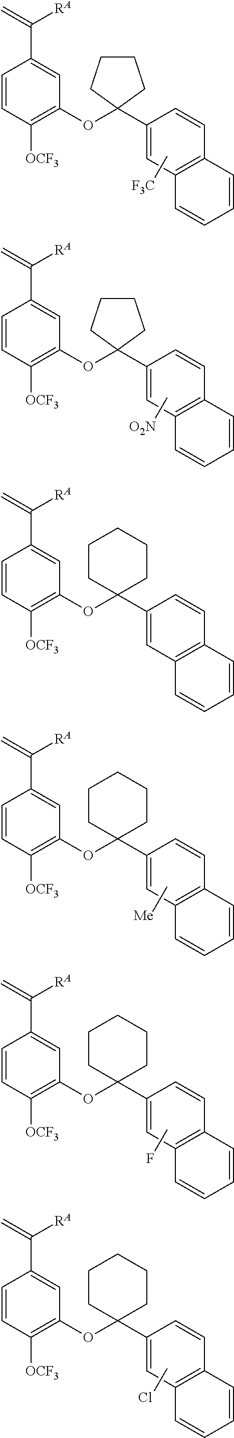

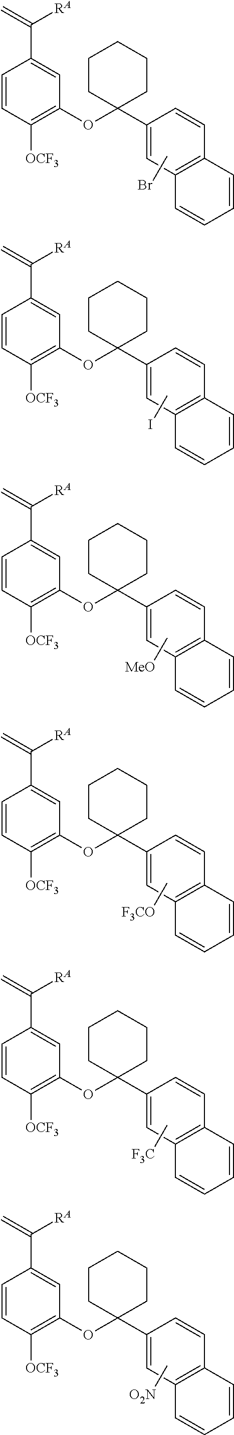

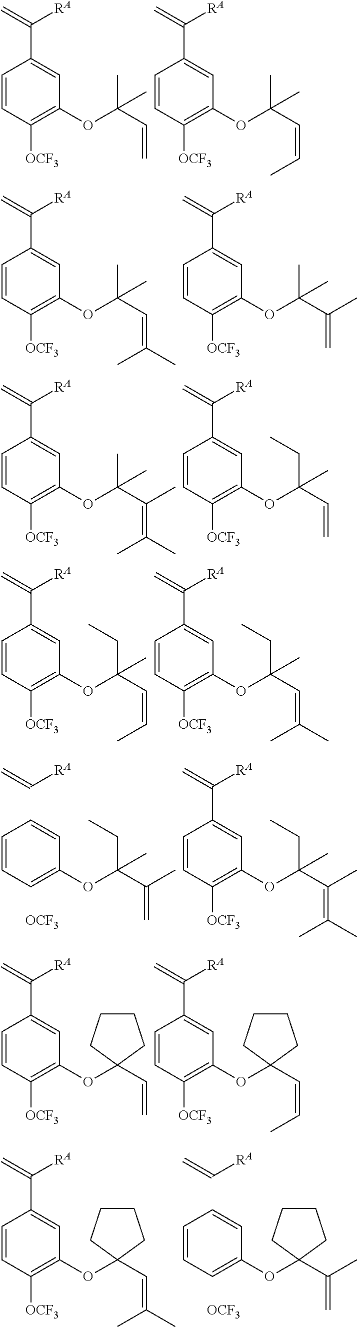

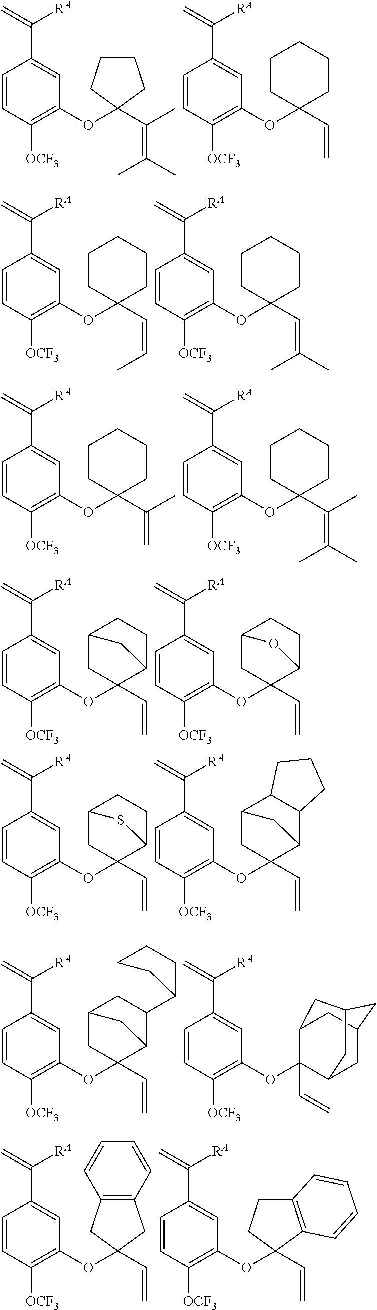

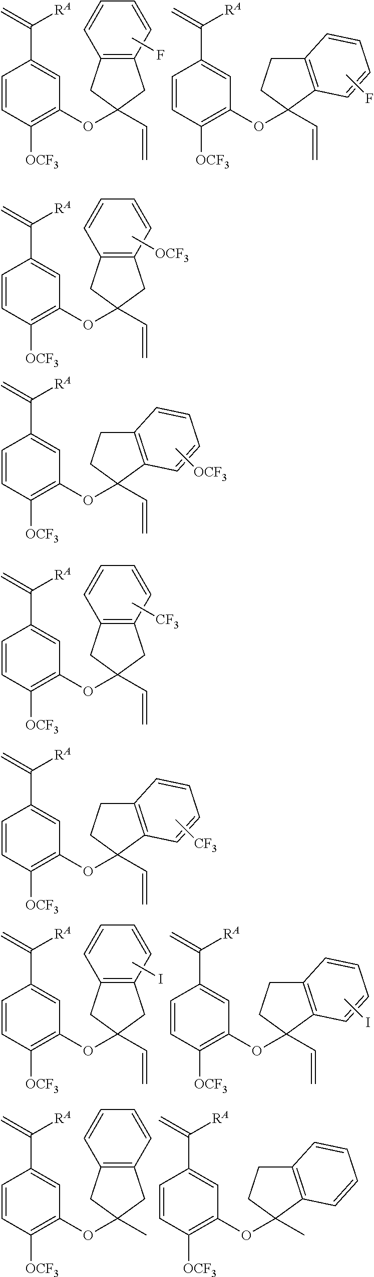

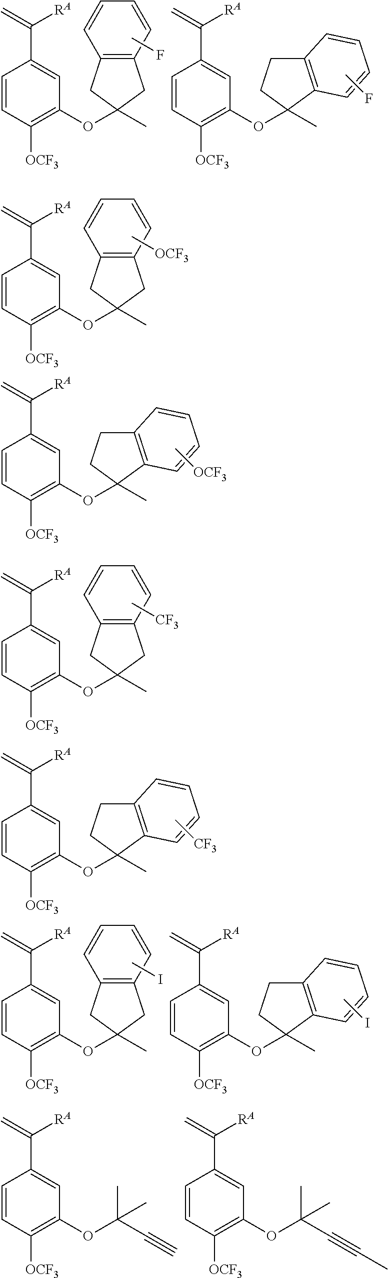

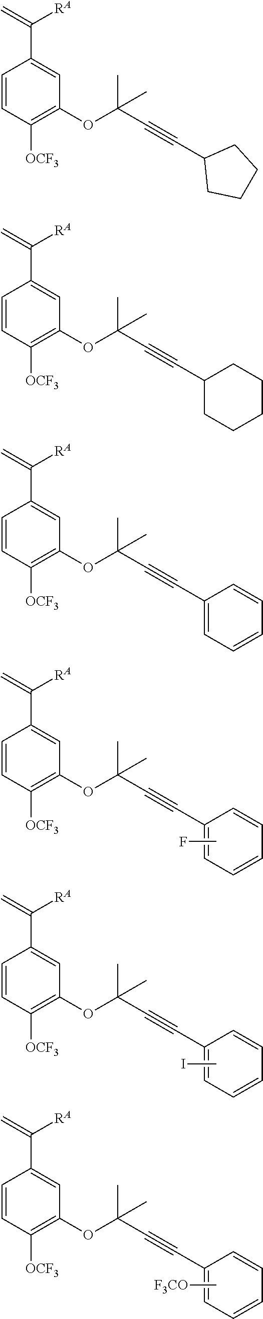

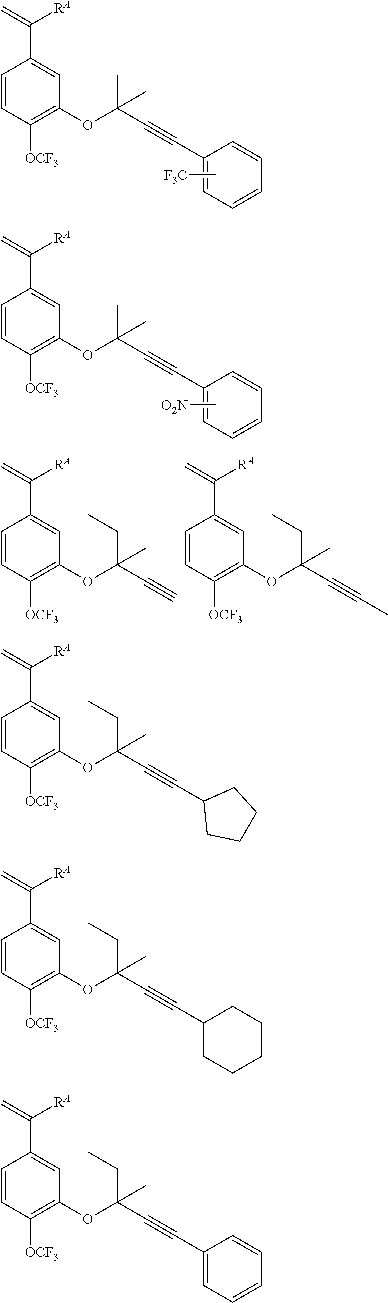

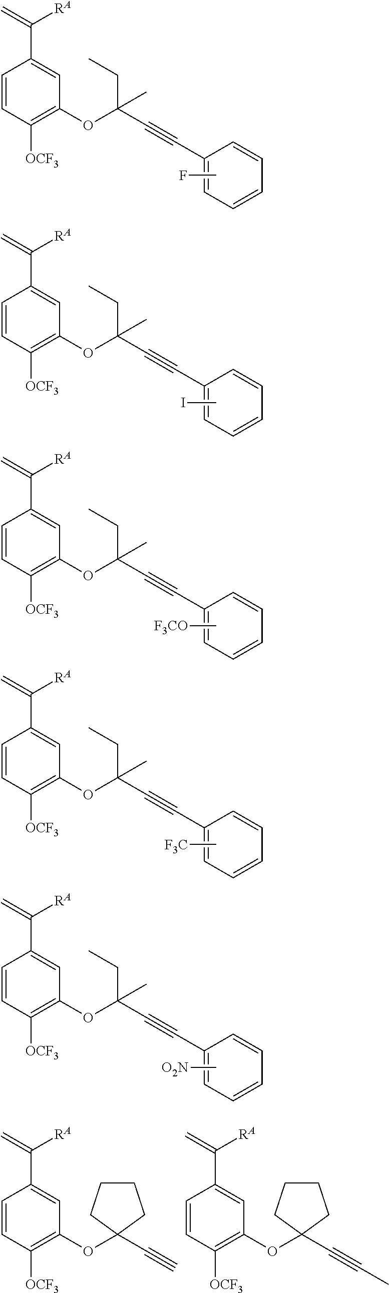

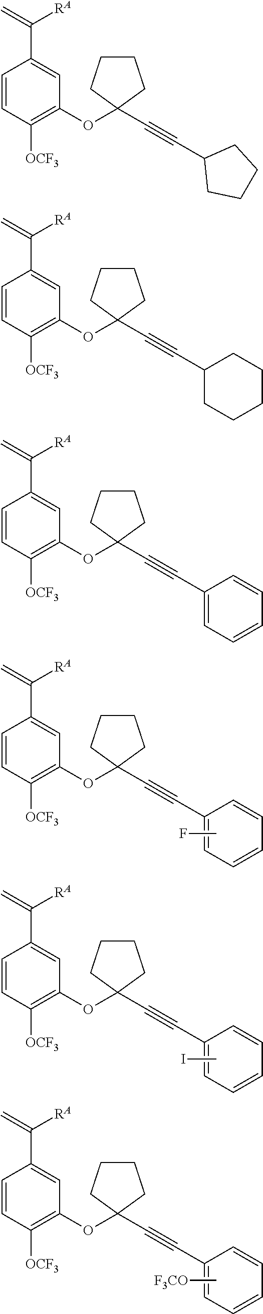

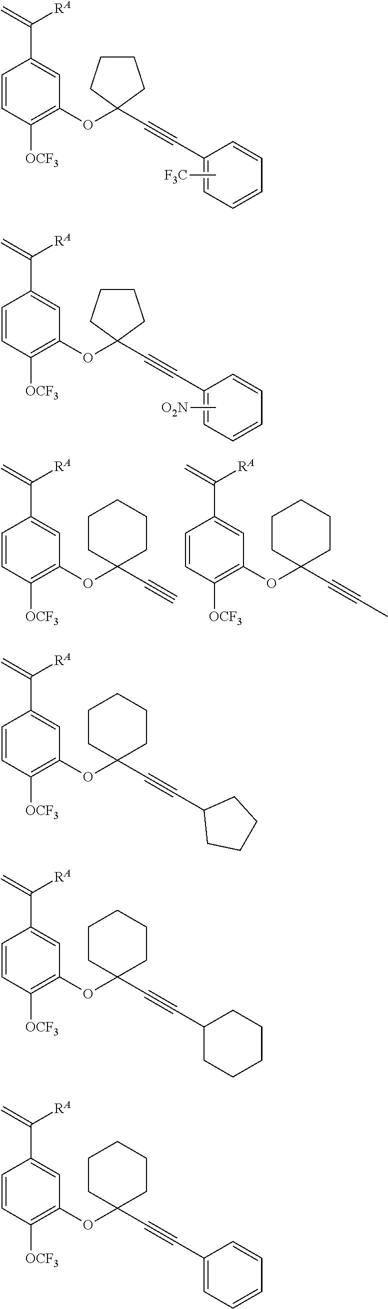

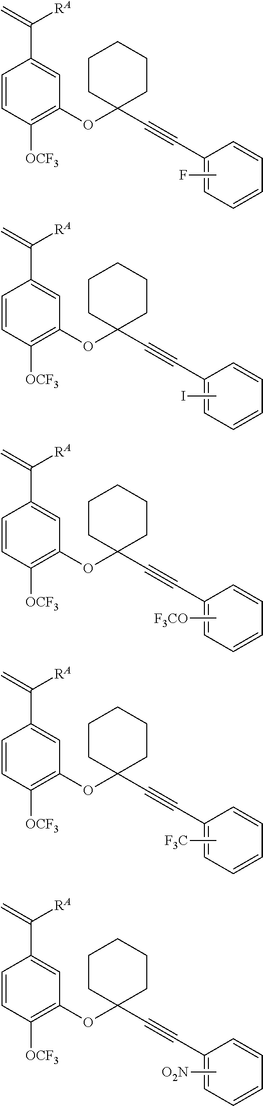

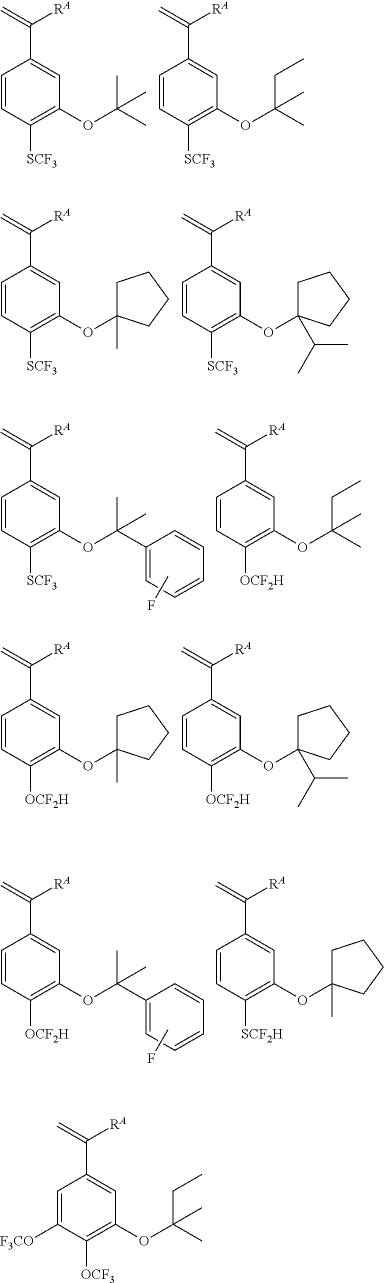

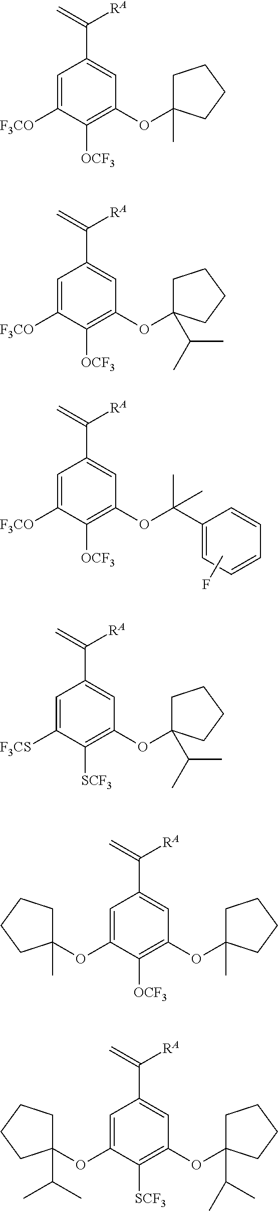

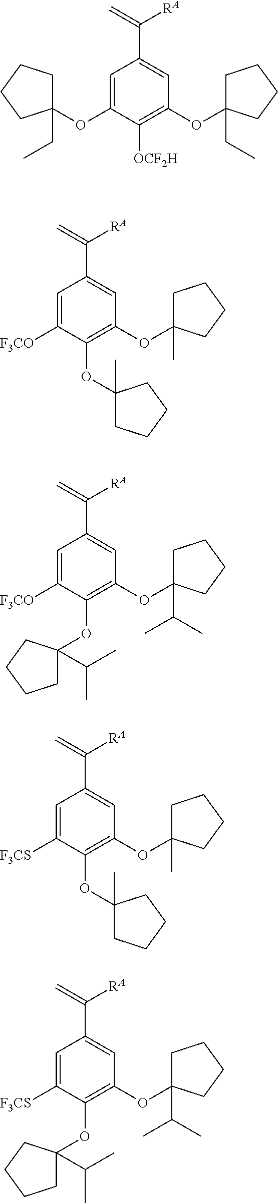

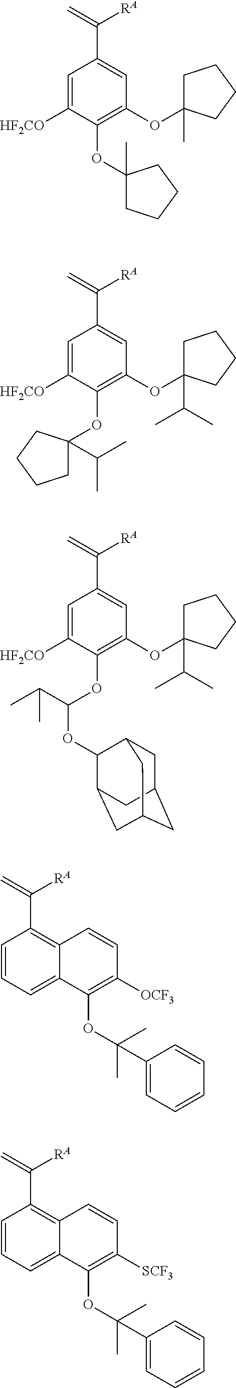

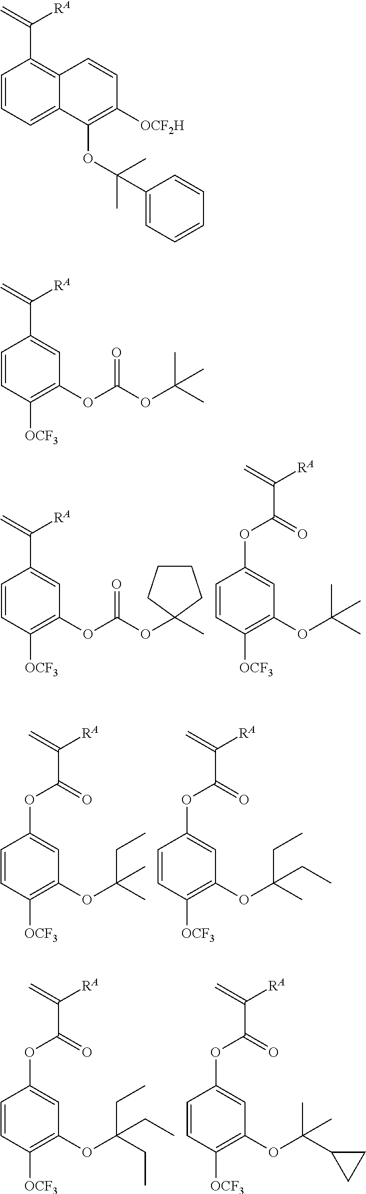

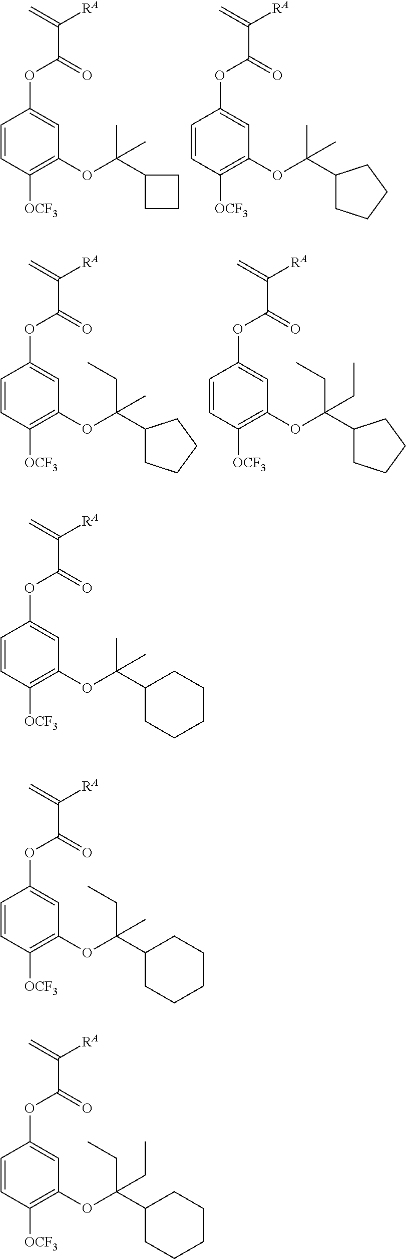

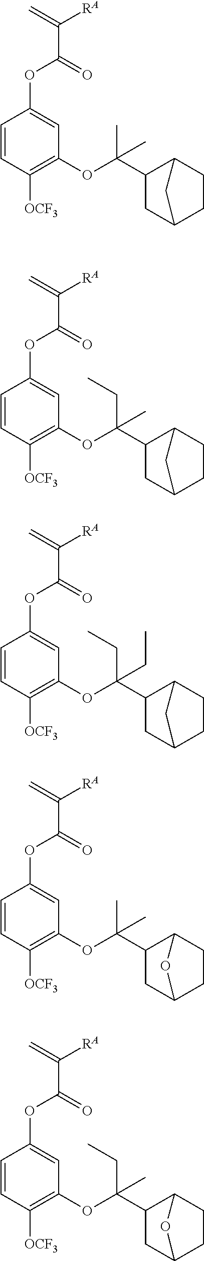

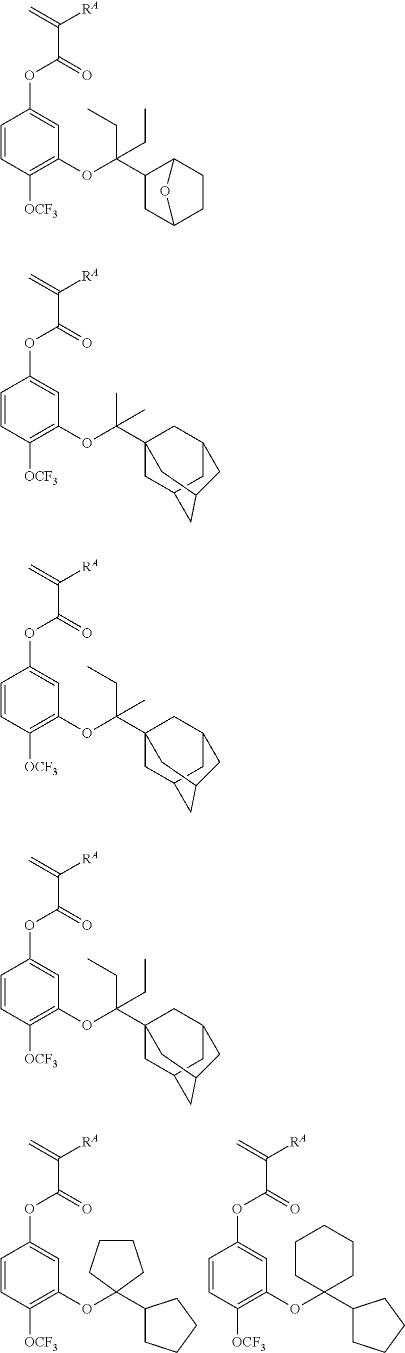

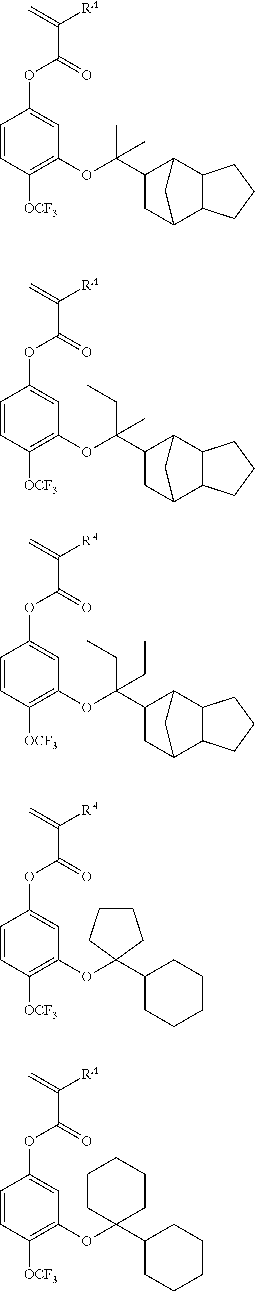

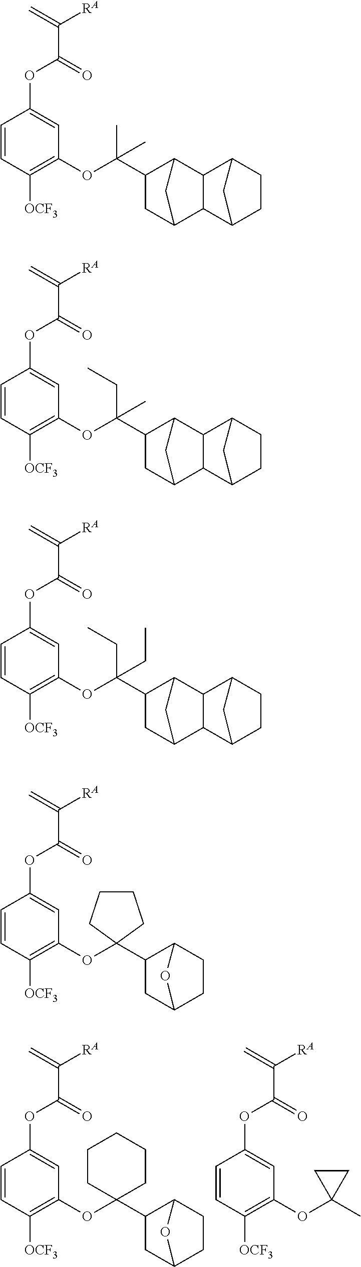

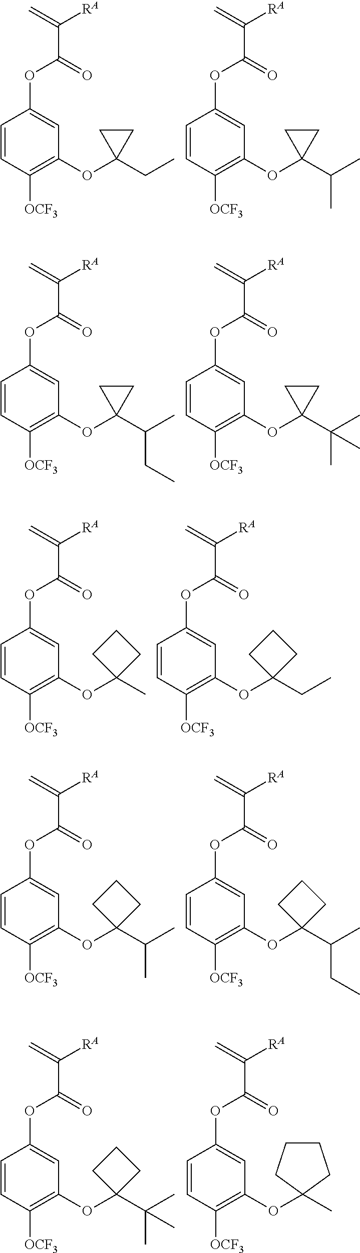

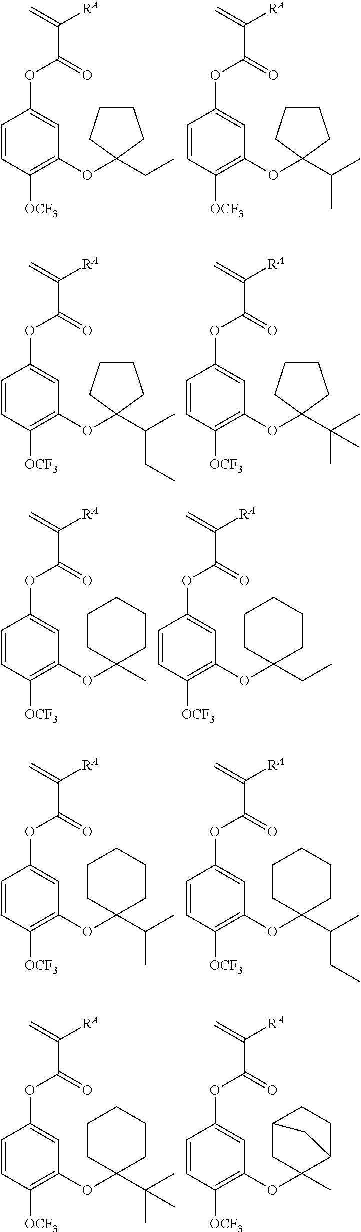

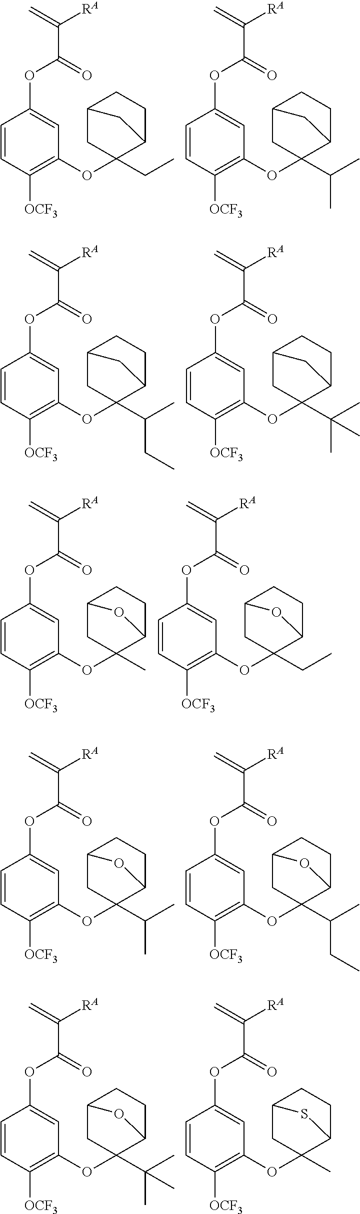

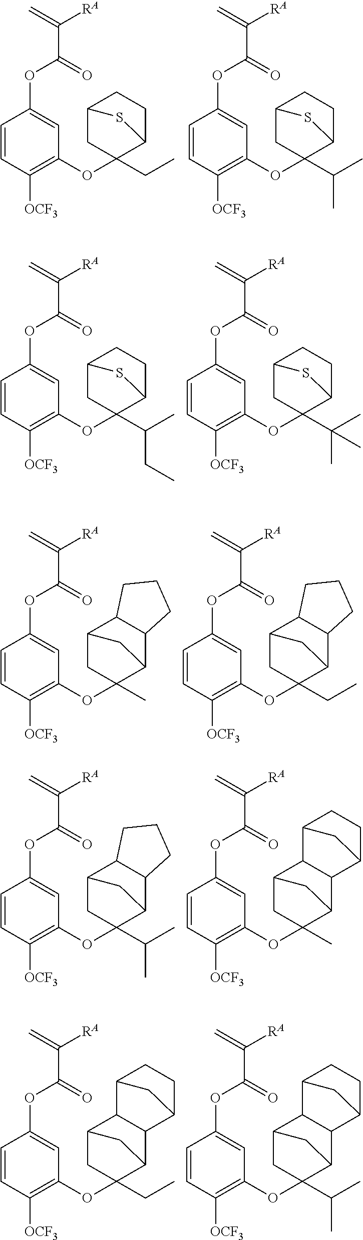

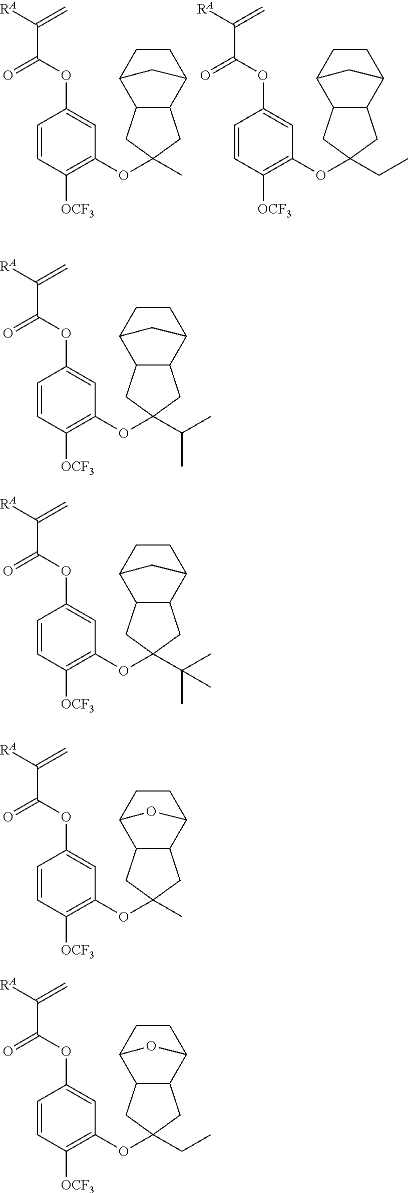

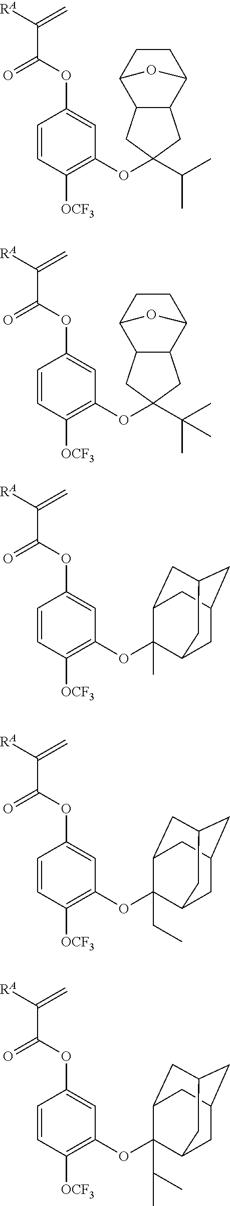

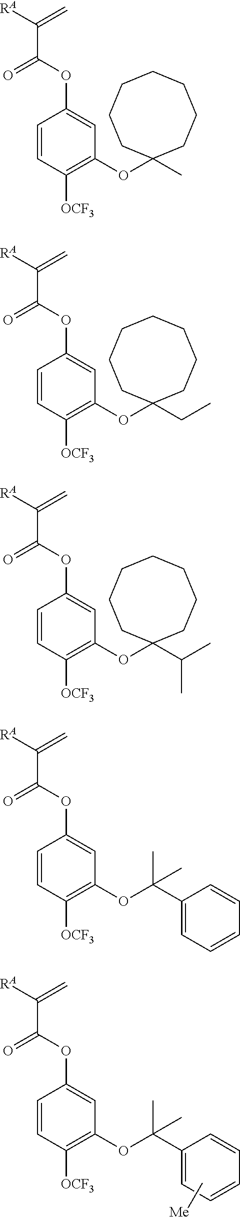

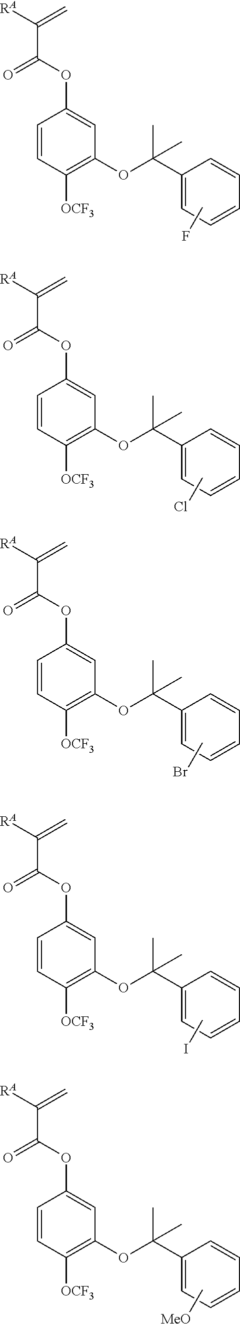

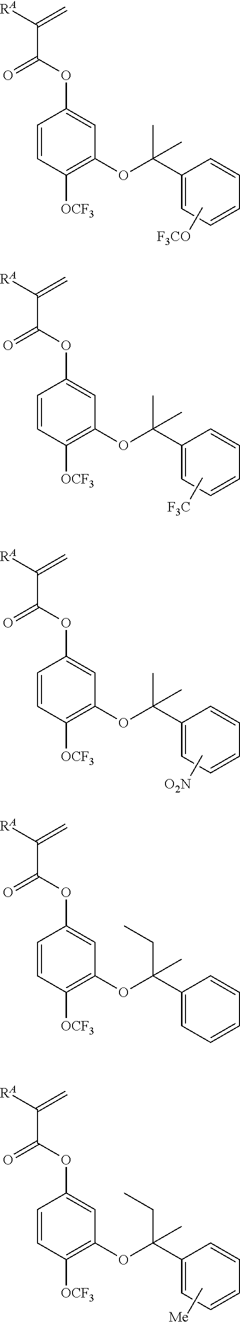

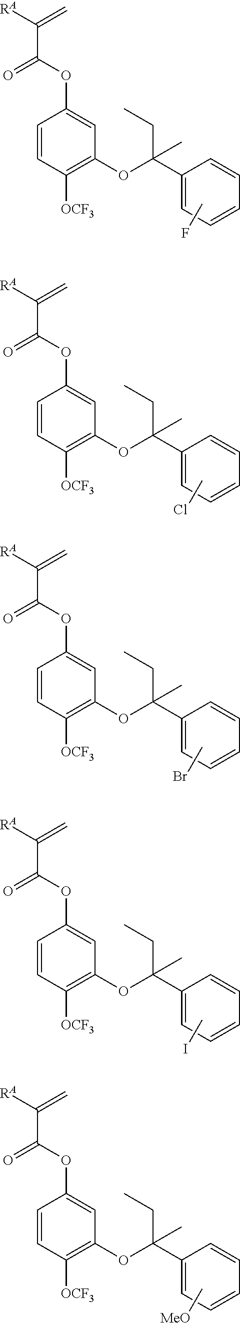

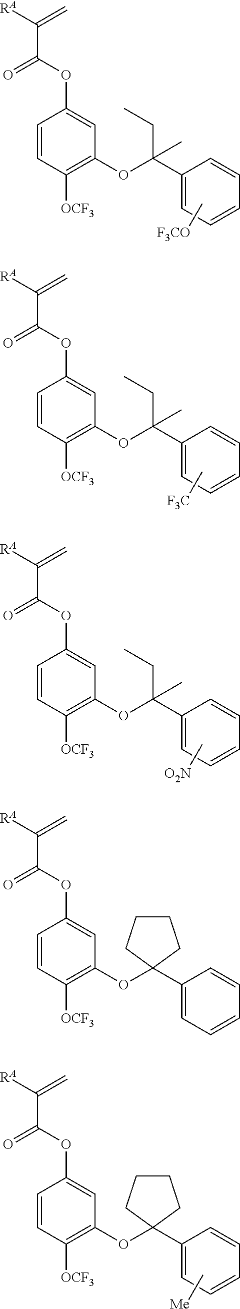

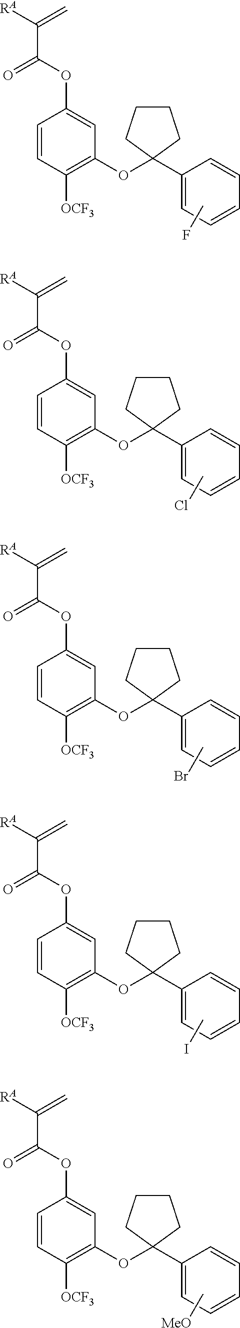

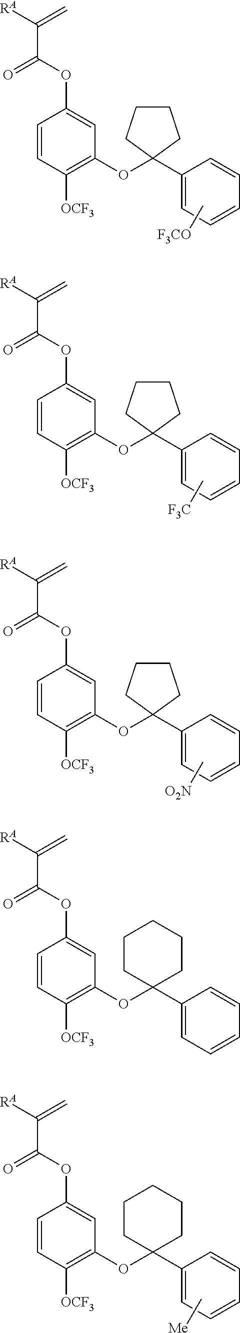

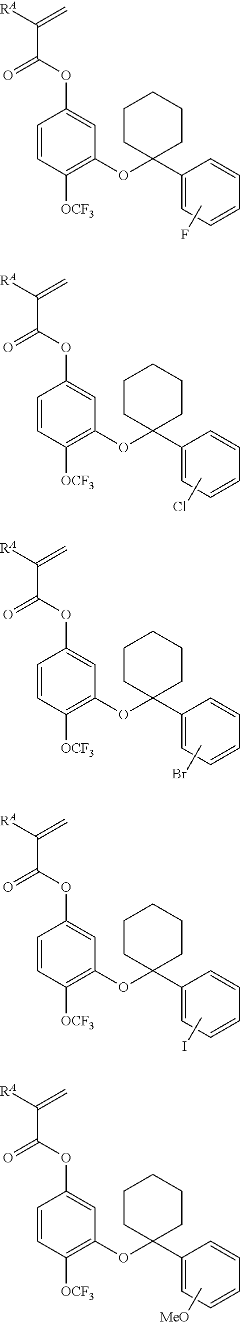

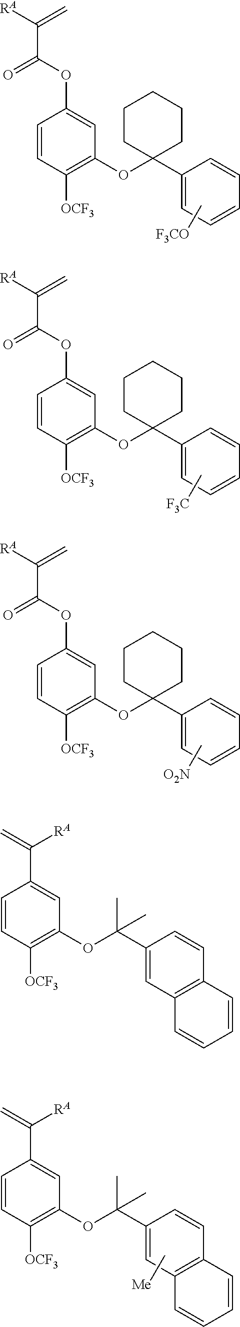

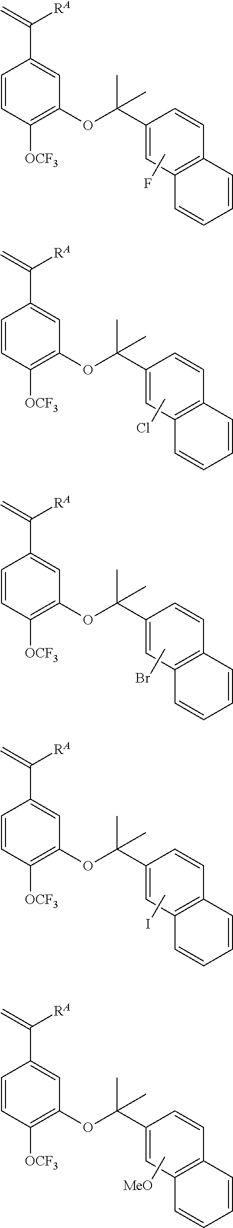

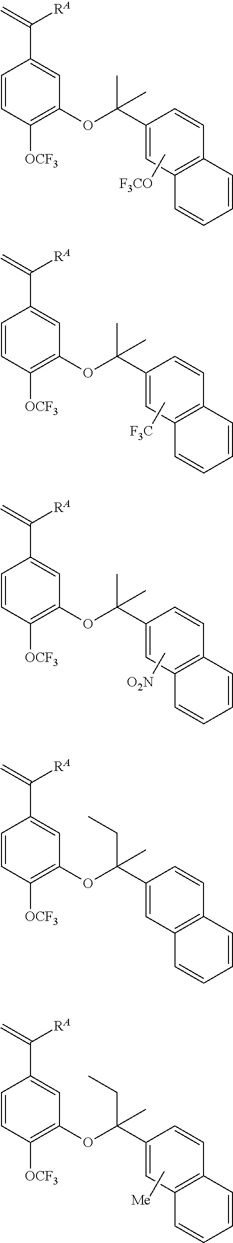

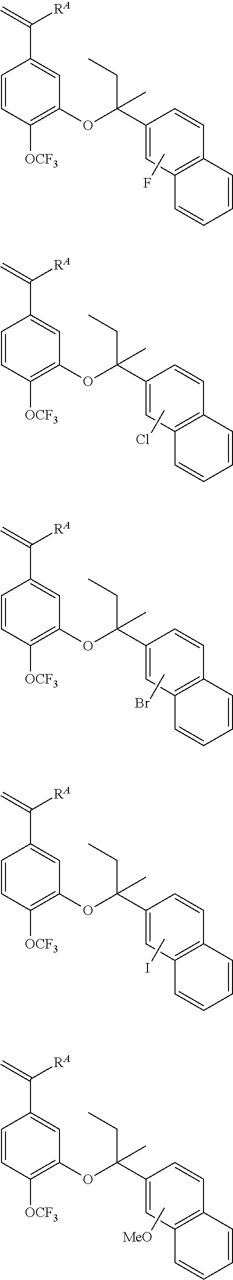

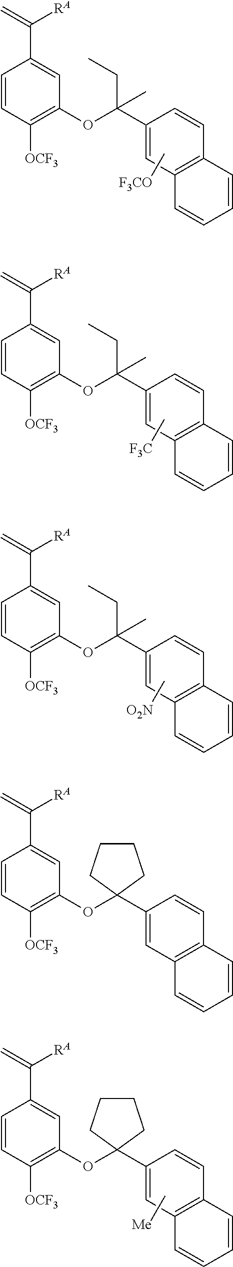

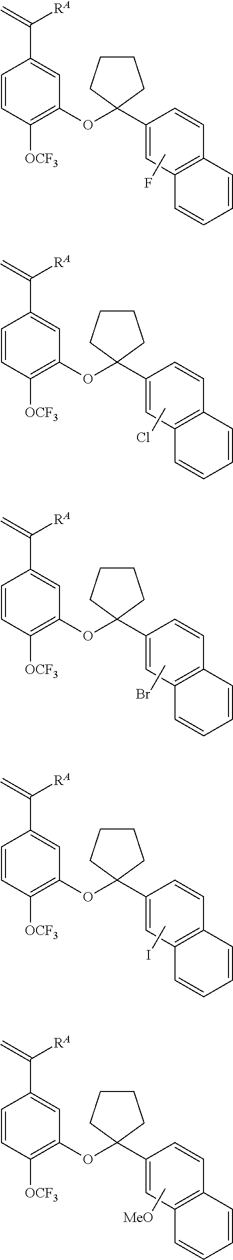

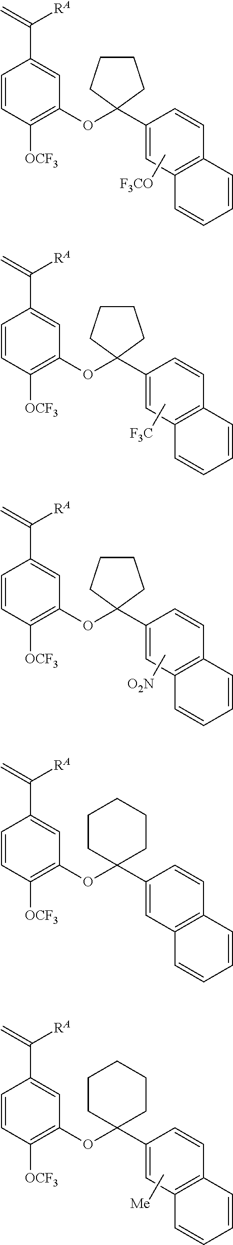

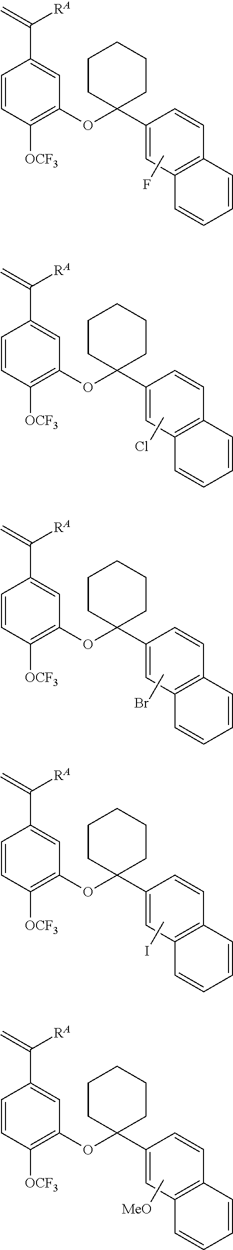

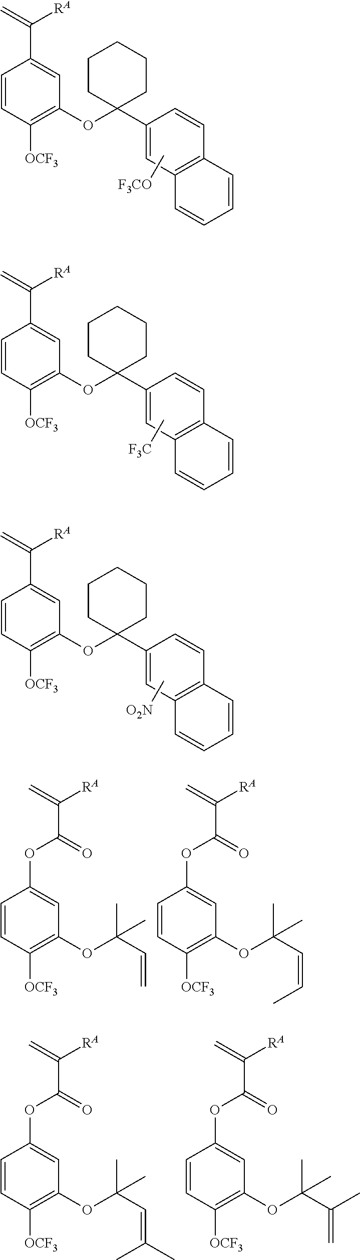

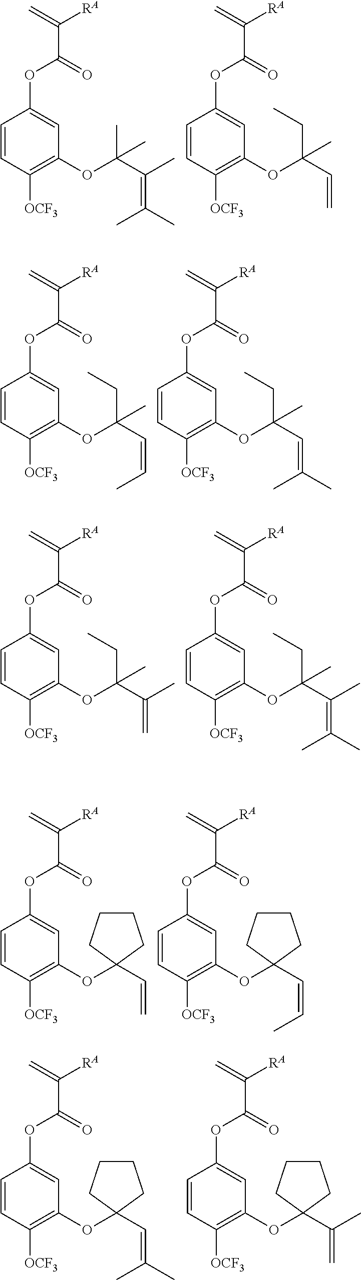

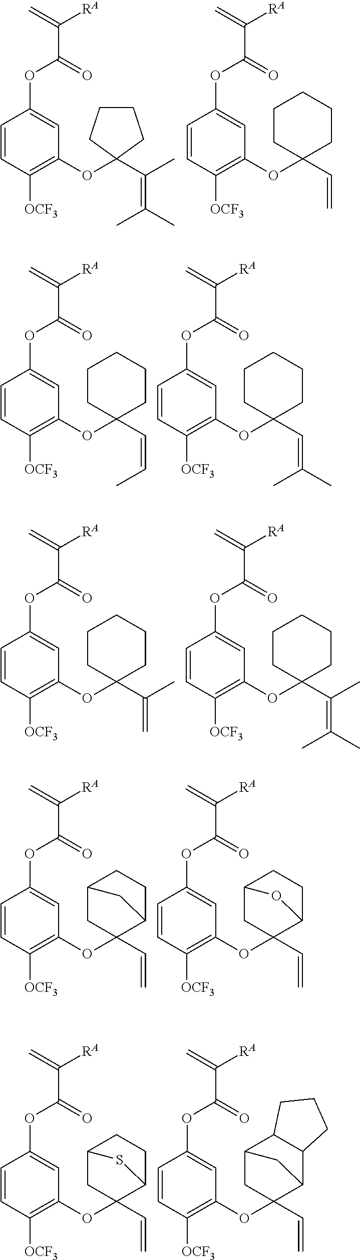

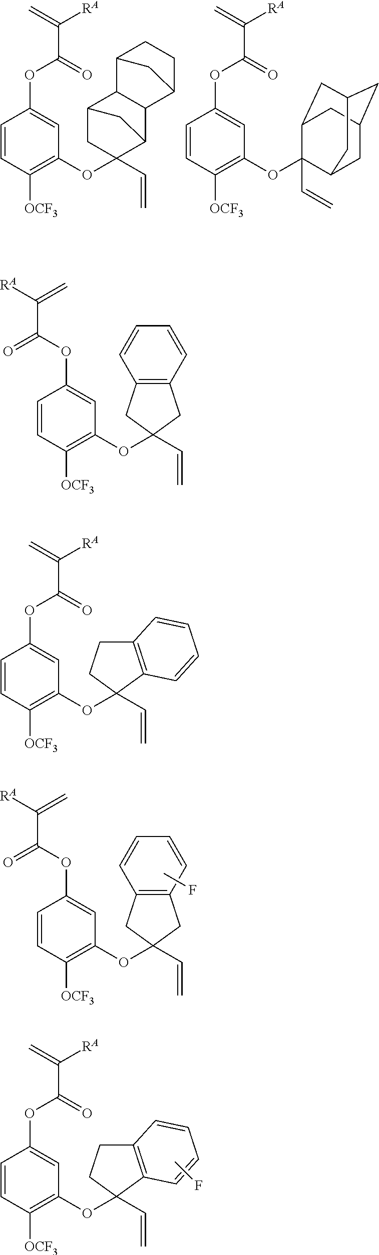

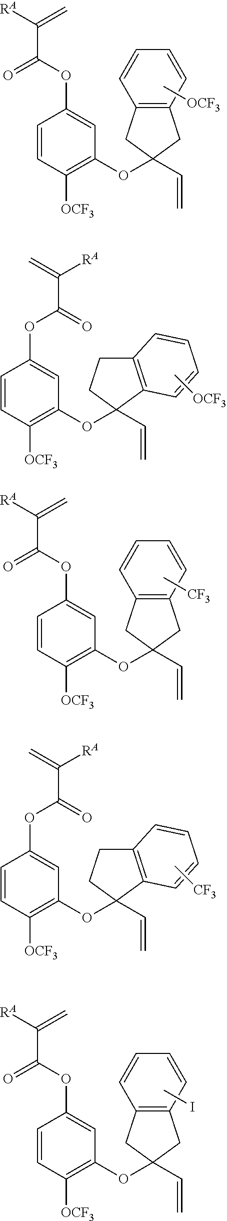

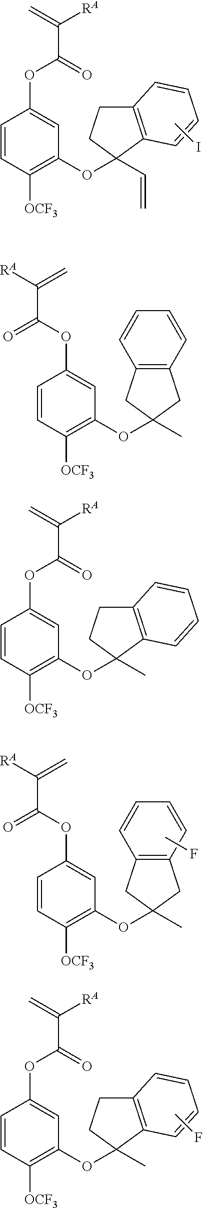

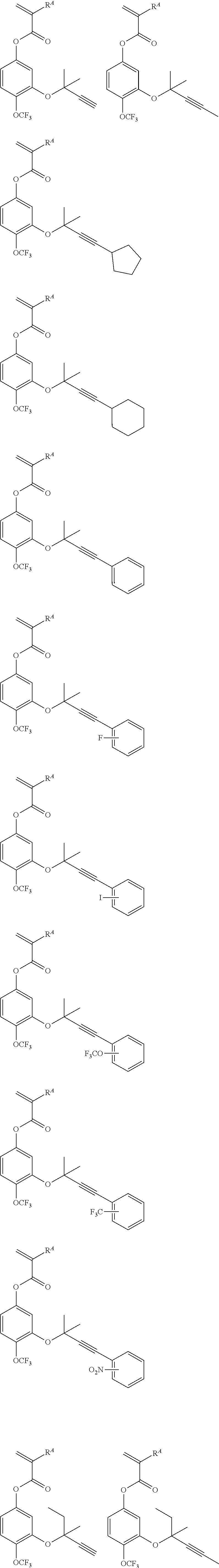

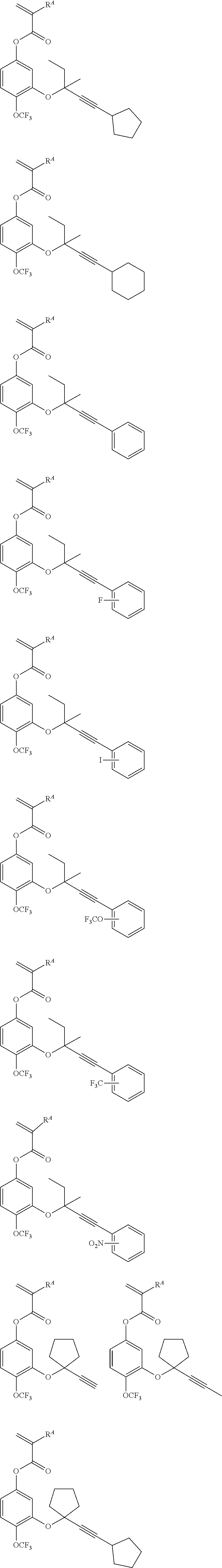

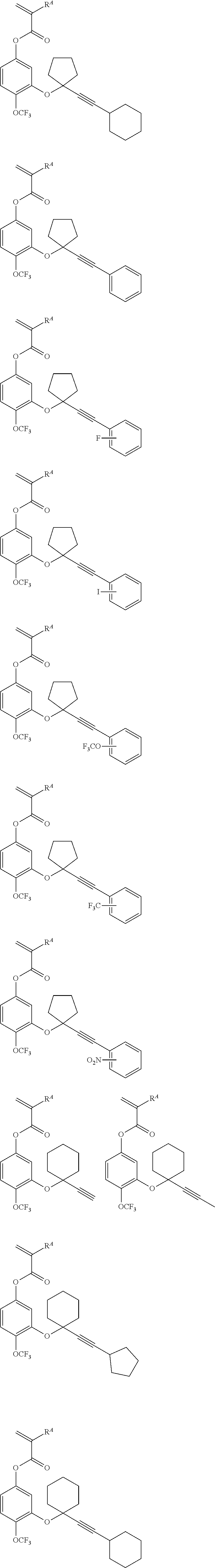

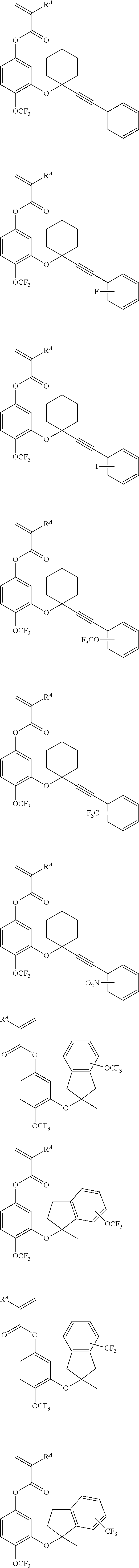

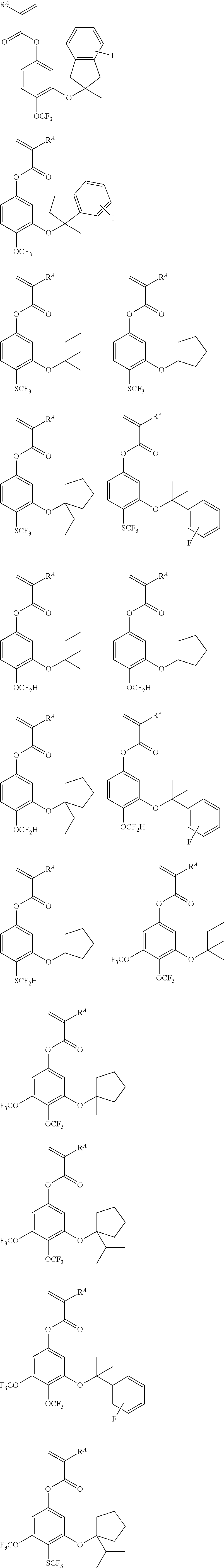

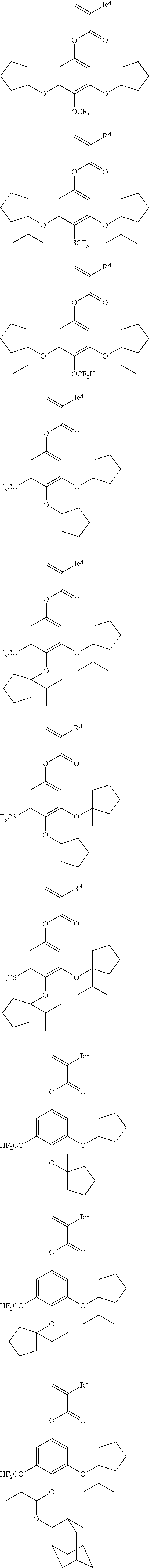

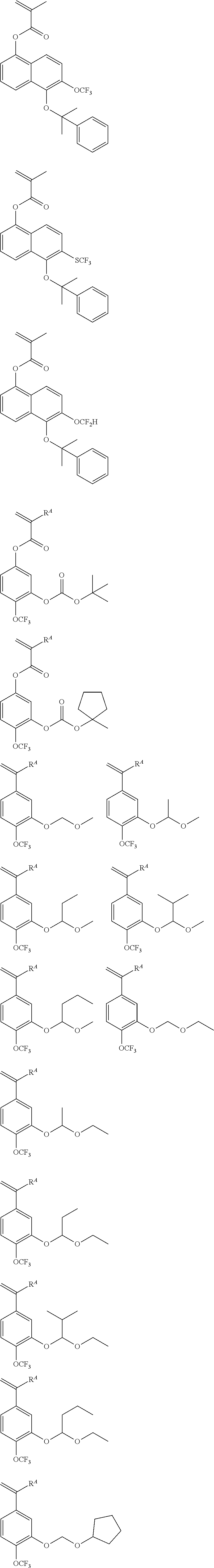

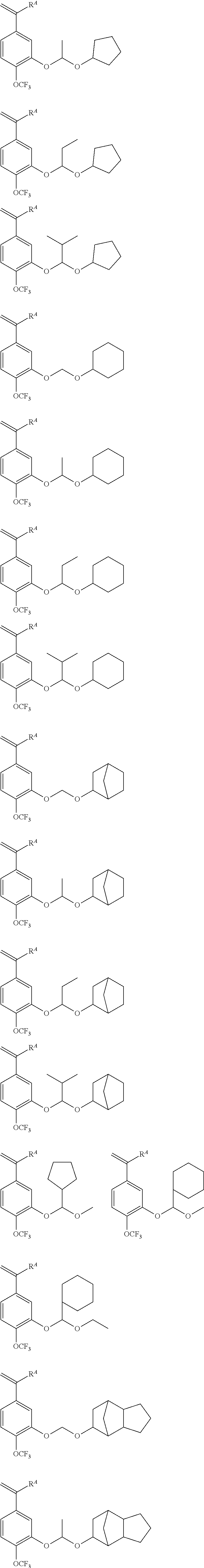

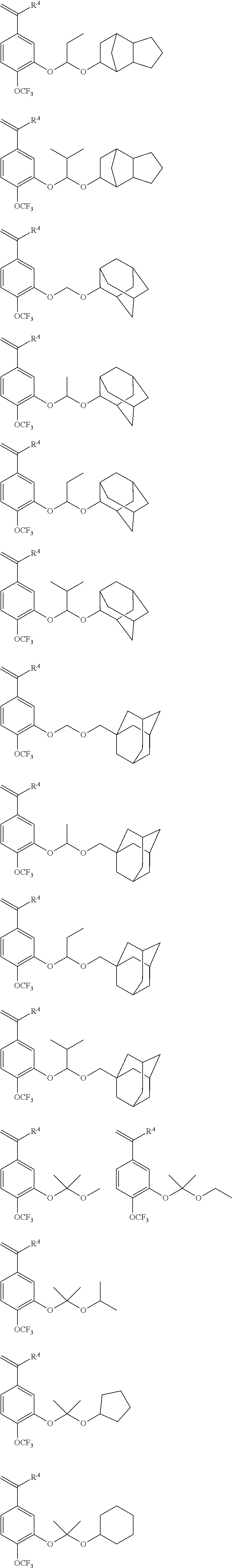

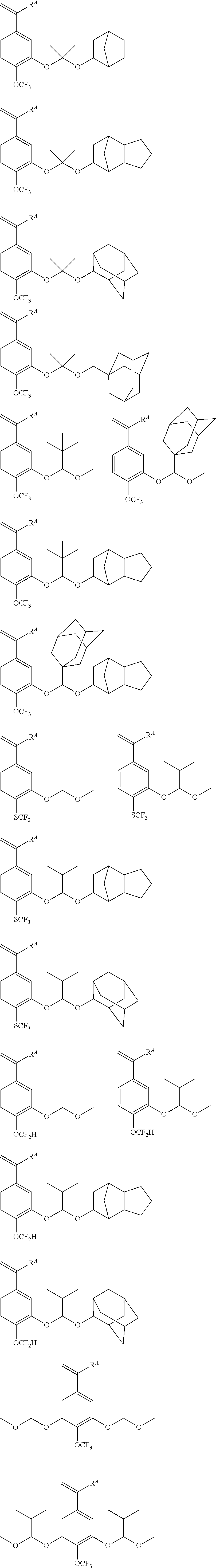

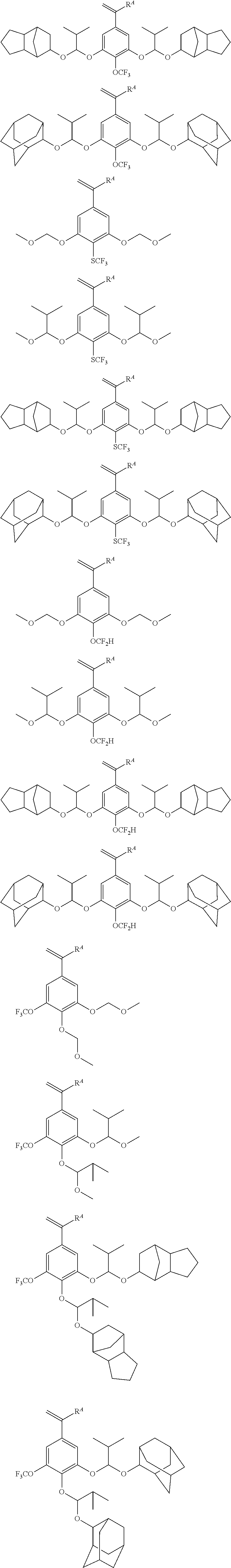

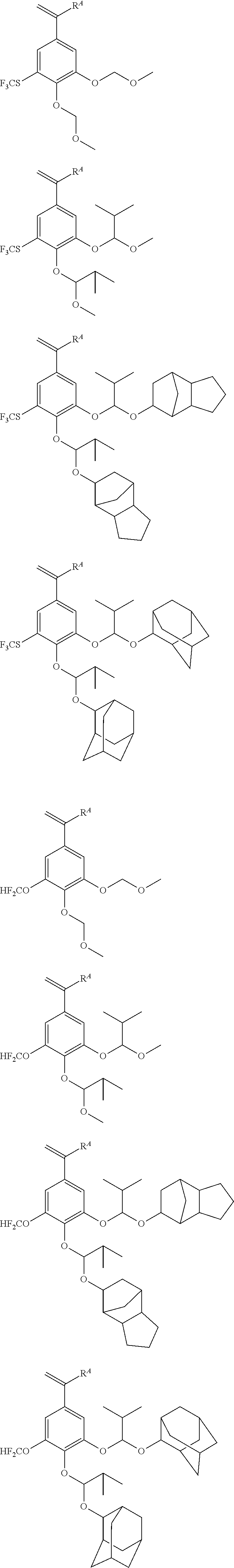

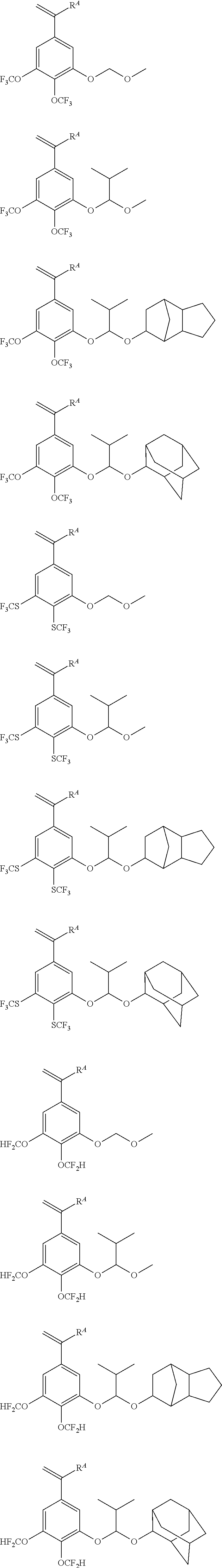

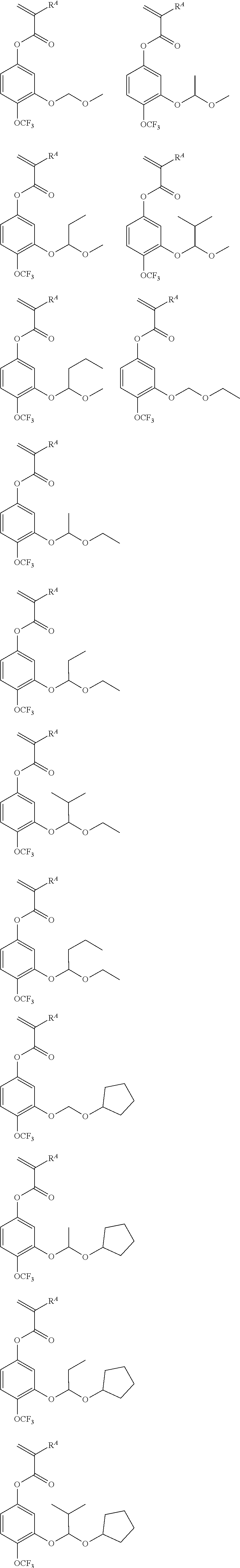

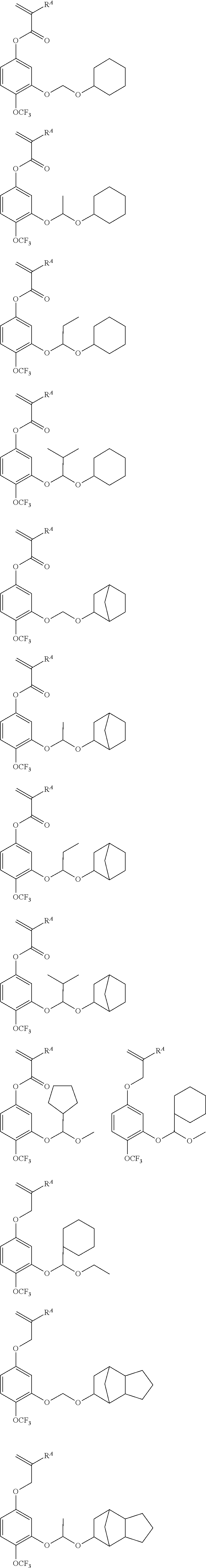

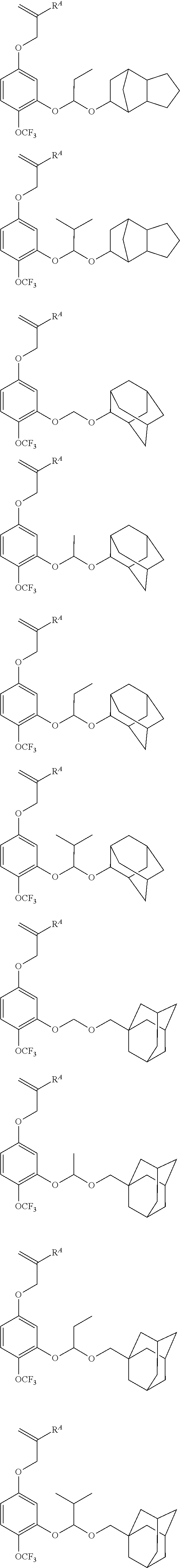

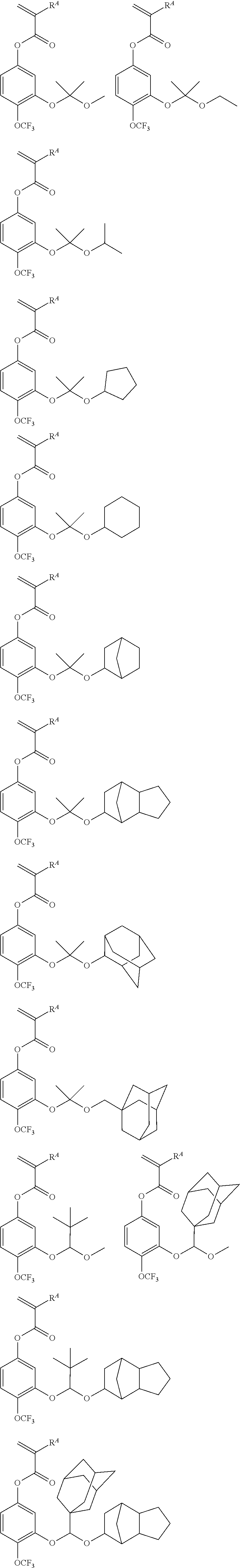

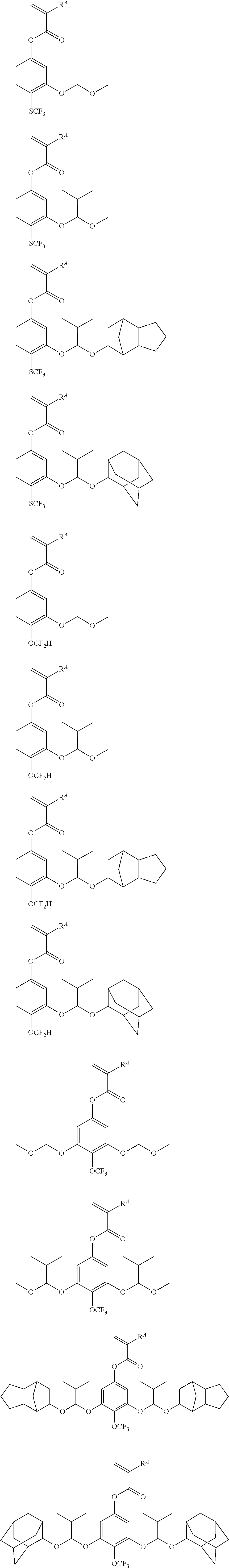

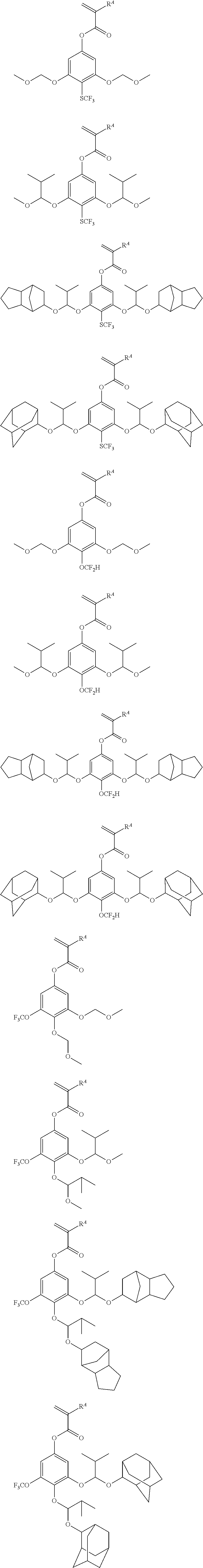

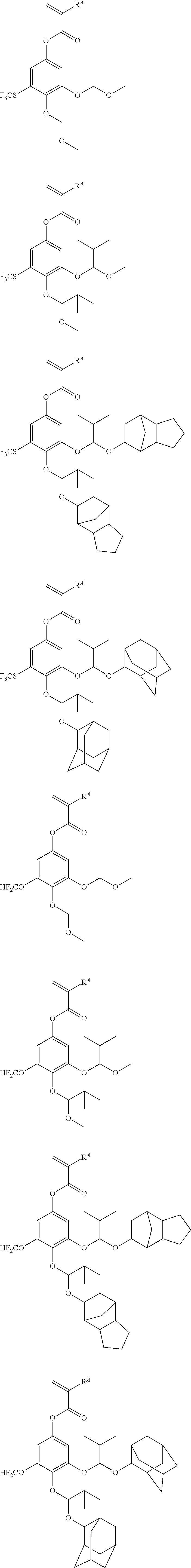

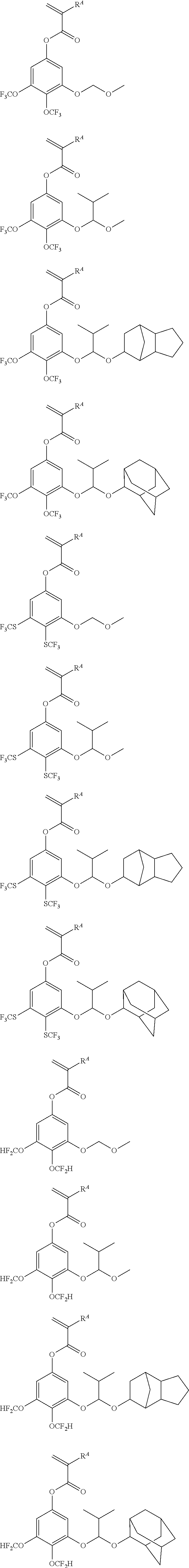

The polymerizable monomer (1) of the present invention can be synthesized by a known method. As an example, a polymerizable monomer (1′) in which Z^{L} is a single bond and X^{L} is an oxygen atom in the formula (1) is described below,

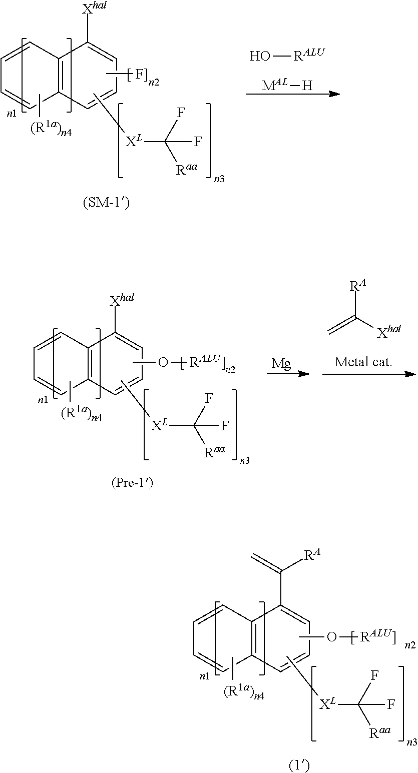

wherein R^{A}, R^{1a}, R^{ALU}, n1, n2, n3, and n4 are as defined above. X^{ha1} is any of a chlorine atom, a bromine atom, or an iodine atom. M^{AL}-H is a hydride of an alkali metal or an alkaline earth metal. Metal cat. is a transition-metal complex catalyst.

The first reaction is a reaction in which a metal alkoxide is prepared from an alcohol of HO—R^{ALU}, and (Pre-1′), which is a precursor of the target product, is obtained by an aromatic nucleophilic substitution reaction with respect to (SM-1′), which is a raw material.

The raw materials (SM-1′) and HO—R^{ALU} can be synthesized according to a known method or can be obtained as a commercially-available product. The hydride of an alkali metal or alkaline earth metal represented by M^{AL}-H is suspended in a solvent such as THF, and HO—R^{ALU} is added dropwise thereto to prepare a metal alkoxide. In order to efficiently prepare the metal alkoxide, it is preferable to appropriately heat the internal temperature of the solvent from 50° C. to about the boiling point of the solvent. After the metal alkoxide is prepared, the raw material (SM-1′) is added, and the reaction can be performed by heating or the like as necessary. From the viewpoint of yield, it is preferable to monitor the reaction by gas chromatography (GC) or silica gel thin layer chromatography (TLC) to complete the reaction. The reaction time is usually about 12 to 24 hours. The (Pre-1′), which is a precursor of the target product, can be obtained from the reaction mixture by ordinary aqueous work-up, and, if necessary, the precursor can be purified by a standard method such as distillation, chromatography, recrystallization, and the like.

The second reaction is a reaction in which a Grignard reagent is prepared from (Pre-1′), which is a precursor of the target product, and subjected to a cross coupling reaction with a vinyl halide compound in the presence of a transition metal complex catalyst to obtain the target product (1′).

The preparation of the Grignard reagent from the precursor of the target product (Pre-1′) can be performed by a known method. After the Grignard reagent is prepared, the reaction system is cooled, and a transition metal complex catalyst is added. Preferable examples of the transition metal complex catalyst to be used include those having palladium, nickel, platinum, cobalt, rhodium, iridium, iron, ruthenium, copper or the like as a central metal and containing various amine ligands, phosphine ligands or N-heterocyclic carbene ligands. Thereafter, a vinyl halide compound diluted with a reaction solvent is added dropwise. In order to improve the reaction conversion rate, the reaction may be performed by heating or the like, as necessary. From the viewpoint of yield, it is preferable to monitor the reaction by gas chromatography (GC) or silica gel thin layer chromatography (TLC) to complete the reaction. The reaction time is usually about 0.5 to 3 hours. The target product (1′) can be obtained from the reaction mixture by ordinary aqueous work-up, and, if necessary, the product can be purified by a standard method such as distillation, chromatography, recrystallization, and the like.

Should be noted that the above-described production method is merely an example, and the production method of the polymerizable monomer of the present invention is not limited thereto.

The structural feature of the polymerizable monomer of the present invention is such that it has an acid-labile group bonded to a hydroxyl group on an aromatic ring and a fluorine-containing alkoxy group, and that these groups are bonded to carbon atoms adjacent to each other. The acid-labile group in a portion subjected to exposure undergoes a deprotection reaction by the generated acid, thereby generating an aromatic hydroxyl group. This increases the contrast between the exposed portion and the unexposed portion. Further, the adjacent fluorine-containing alkoxy group improves the resist-solvent solubility of the base polymer itself obtained by copolymerization, and also improves the acidity of the aromatic hydroxyl group generated in the exposed portion by virtue of the electron-withdrawing property thereof. When the resist film is developed by an alkaline developer after the exposure, the affinity between the generated aromatic hydroxyl group and the alkaline developer is improved; thus, the exposed portion is effectively removed by the developer. In addition, it is assumed that, compared with a simple carboxyl group, the aromatic hydroxyl group adjacent to the fluorine-containing alkoxy group does not introduce an alkaline developer to an unexposed portion due to the water repellent effect of the plurality of fluorine atoms, thereby exhibiting an effect of reducing swelling caused by an alkaline developer. As a result, the collapse of the resist pattern generated in the unexposed portion is suppressed. By the synergistic effects of these, when the polymerizable monomer of the present invention is used, it is possible to form a pattern having a high dissolution contrast, being excellent in LWR of a line pattern and excellent in CDU of a hole pattern, and exhibiting resistance to pattern collapse; therefore, the polymerizable monomer of the present invention is suitable as a positive resist material.

### [Resist Composition]

The resist composition, in particular, the chemically-amplified resist composition of the present invention includes:
(A) a base resin formed from a polymer compound, in particular, a copolymerized polymer compound, containing a repeating unit obtained from the polymerizable monomer represented by the formula (1); and
(B) an organic solvent.

As necessary, the resist composition of the present invention further includes:
(C) a quencher; and
(D) an acid generator, such as a photoacid generator.

As necessary, the resist composition of the present invention still further includes:

(E) a surfactant which is insoluble or substantially insoluble in water and soluble in an alkaline developer, and/or a surfactant which is insoluble or substantially insoluble in water and an alkaline developer.

As necessary, the resist composition of the present invention yet further includes:

(F) other components.

### [Polymer Compound]

The polymerizable monomer can be suitably used as a constituent unit of the base polymer in the resist composition. The polymer compound of the present invention contains a repeating unit obtained from the polymerizable monomer of the present invention, and the repeating unit includes the repeating unit (hereinafter, also referred to as a repeating unit “A”) represented by the following general Formula (Ia),

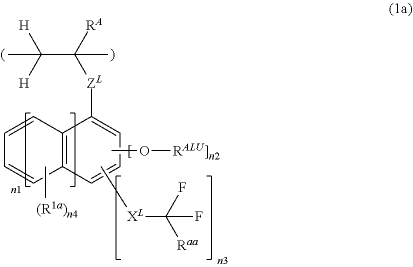

wherein R^{A}, Z^{L}, X^{L}, R^{1a}, R^{ALU}, R^{aa}, n1, n2, n3, and n4 are as defined above.

It is preferable that the polymer compound of the present invention further contain the repeating unit represented by the following general formula (1-Aa),

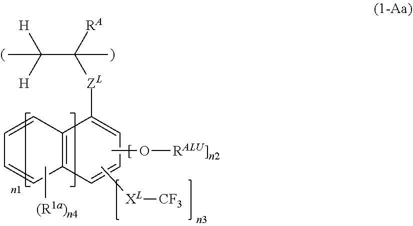

wherein R^{A}, Z^{L}, X^{L}, R^{1a}, n1, n2, n3, and n4 are as defined above.

Further, the R^{ALU} is preferably represented by the following formula (AL-1) or (AL-2) together with the adjacent oxygen atom,

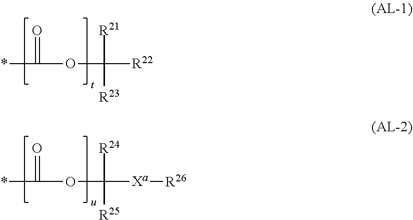

wherein R²¹, R²², R²³ t, R²⁴, R²⁵, R²⁶, X^{a}, and u are as defined above.

Regarding the repeating unit “A”, two or more different types may be copolymerized as a constituent unit of the base polymer.

The polymer compound of the present invention preferably contains the repeating unit (hereinafter, also referred to as a repeating unit “a1”) represented by the following general formula (a1), or the repeating unit represented by the following general formula (a2) (hereinafter, also referred to as a repeating unit “a2”)

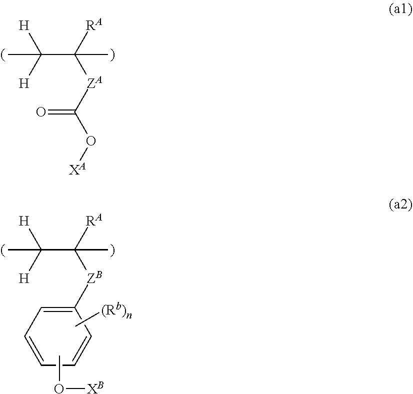

wherein R^{A} independently represents a hydrogen atom, a fluorine atom, a methyl group, or a trifluoromethyl group; Z^{A} is a single bond, an alkoxy group optionally substituted with a halogen atom, a phenylene group or a naphthylene group optionally substituted with a halogen atom, or (backbone) —C(═O)—O—Z^{A1}—; Z^{A1} is a heteroatom, an alkoxy group having 1 to 10 carbon atoms which may contain a fluorine atom, a linear, branched or cyclic alkanediyl group having 1 to 20 carbon atoms which may contain a hydroxy group, an ether bond, an ester bond, a lactone ring, a thioether bond, a sulfonyl group, or a sulfonamide structure, or a phenylene group or a naphthylene group; Z^{B} is a single bond or (backbone) —C(═O)—O—Z^{B1}—; Z^{B1} is a single bond or an alkanediyl group having 1 to 10 carbon atoms which may contain an ester and/or an ether bond; X^{A} and X^{B} independently represent an acid-labile group; R^{b} represents a halogen atom, a nitro group, or a linear, branched or cyclic monovalent hydrocarbon group having 1 to 20 carbon atoms which may contain a heteroatom; n is an integer of 0 to 4.

In the formulae (a1) and (a2), R^{A} independently represents a hydrogen atom, a fluorine atom, a methyl group, or a trifluoromethyl group. Z^{A} is preferably a single bond, (backbone) —C(═O)—O—Z^{A1}—, or an alkoxy group having 1 to 10 carbon atoms which may contain a fluorine atom, or a phenylene or naphthylene group which may contain a halogen atom. Z^{A1} is a heteroatom, an alkoxy group having 1 to 10 carbon atoms which may contain a fluorine atom, a linear, branched or cyclic alkanediyl group (aliphatic hydrocarbylene group) having 1 to 20, preferably 1 to 10, carbon atoms which may contain a hydroxy group, an ether bond, an ester bond, a lactone ring, a thioether bond, a sulfonyl group, or a sulfonamide structure, or a phenylene group or a naphthylene group. Z^{B} is preferably a single bond or (backbone) —C(═O)—O—. X^{A} and X^{B} independently represent an acid-labile group.

In the formula (a2), RID represents a halogen atom, a nitro group, or a hydrocarbyl group having 1 to 20 carbon atoms which may contain a heteroatom. The hydrocarbyl group may be any of a saturated, unsaturated, linear, branched, or cyclic hydrocarbyl group. Specific examples thereof include those similar to those exemplified for R^{1a} in the formula (1a). n is an integer of 0 to 4, preferably 0 or 1.

Examples of the acid-labile groups represented by X^{A} and X^{B} in the formulae (a1) and (a2) include those described in JP 2013-80033 A and those described in JP No. 2013-83821 A.

Typical examples of the acid-labile group include groups represented by the following formulae (AL-1)′ to (AL-3)′,

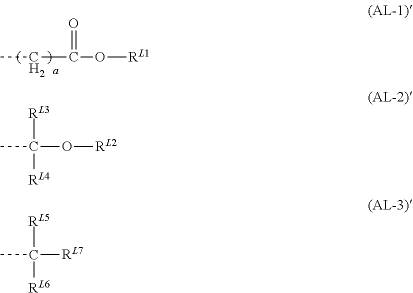

wherein the dashed line represents a bonding arm.

In the formulae (AL-1)′ and (AL-2)′, R^{L1} and R^{L2} independently represent a saturated hydrocarbyl group having 1 to 40 carbon atoms which may contain a heteroatom, such as an oxygen atom, a sulfur atom, a nitrogen atom and a fluorine atom. The saturated hydrocarbyl group may be linear, branched, or a cyclic saturated hydrocarbyl group. The saturated hydrocarbyl group preferably has 1 to 20 carbon atoms.

In the formula (AL-1)′, is an integer of 0 to 10, preferably 1 to 5.

In the formula (AL-2)′, R^{L3} and R^{L4} independently represent a hydrogen atom or a saturated hydrocarbyl group having 1 to 20 carbon atoms which may contain a heteroatom, such as an oxygen atom, a sulfur atom, a nitrogen atom and a fluorine atom. The hydrocarbyl group may be linear, branched, or a cyclic hydrocarbyl group. Any two of R^{L2}, R^{L3} and R^{L4} may be bond to each other to form a ring having 3 to 20 carbon atoms with the carbon atom or carbon and oxygen atoms to which they are bonded. The ring is preferably a ring having 4 to 16 carbon atoms, in particular, an aliphatic ring.

In the formula (AL-3)′, R^{L5}, R^{L6} and R^{L7} independently represent a saturated hydrocarbyl group having 1 to 20 carbon atoms which may contain a heteroatom, such as an oxygen atom, a sulfur atom, a nitrogen atom and a fluorine atom. The hydrocarbyl group may be linear, branched, or a cyclic hydrocarbyl group. Further, any two of R^{L5}, R^{L6} and R^{L7} may be bond to each other to form a ring having 3 to 20 carbon atoms with the carbon atom to which they are bonded. The ring is preferably a ring having 4 to 16 carbon atoms, in particular, an aliphatic ring.

Examples of the repeating unit “a1” include, but are not limited thereto. In the following formulae, R^{A} and X^{A} are as defined above.

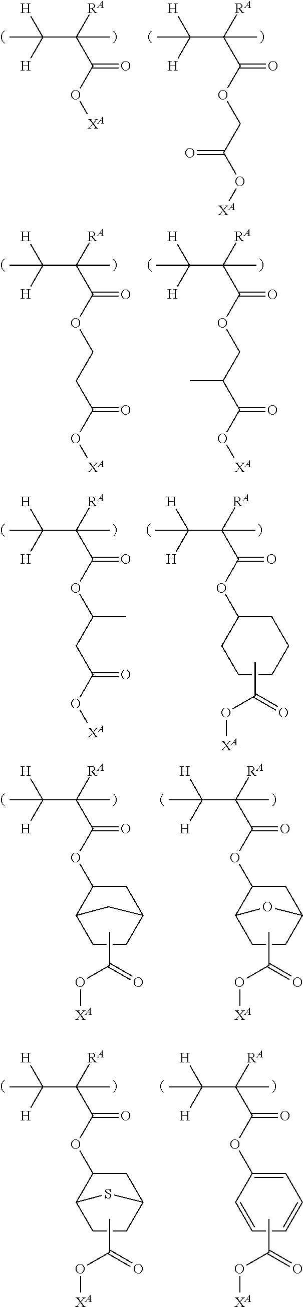

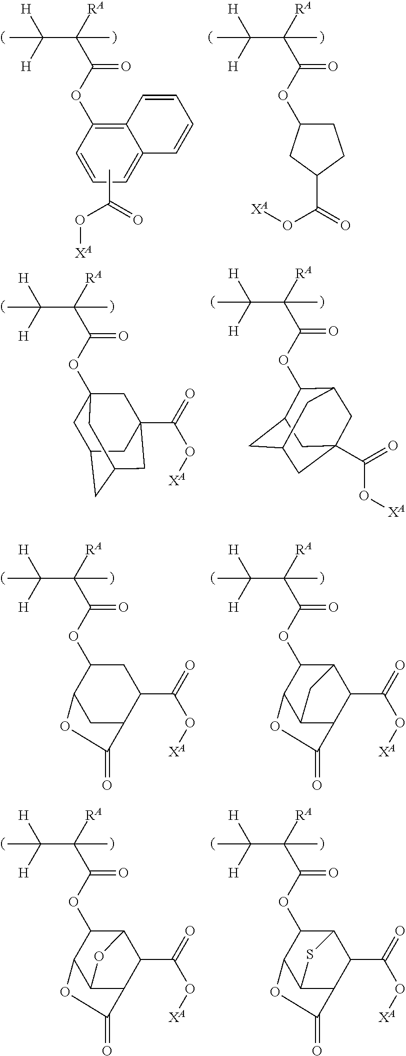

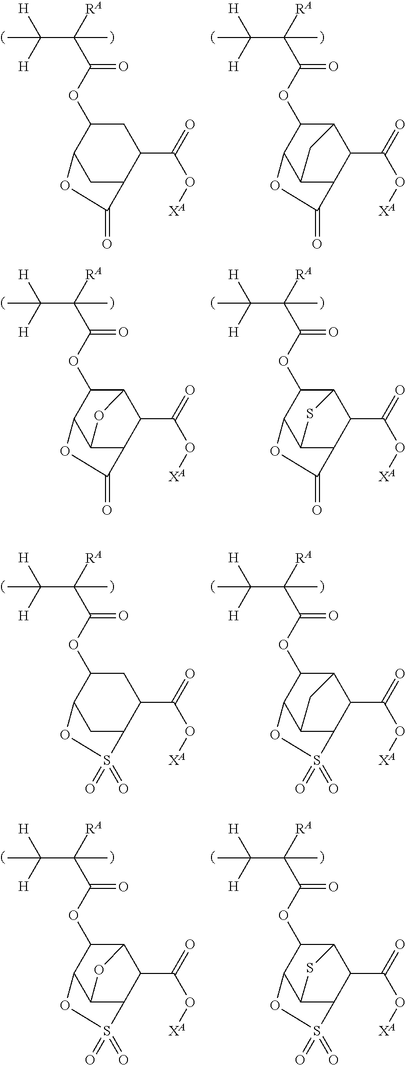

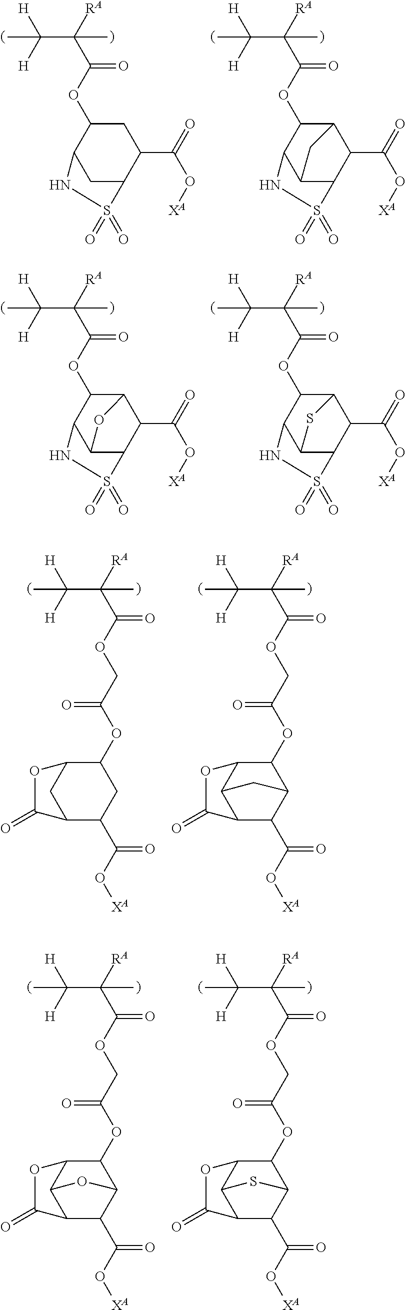

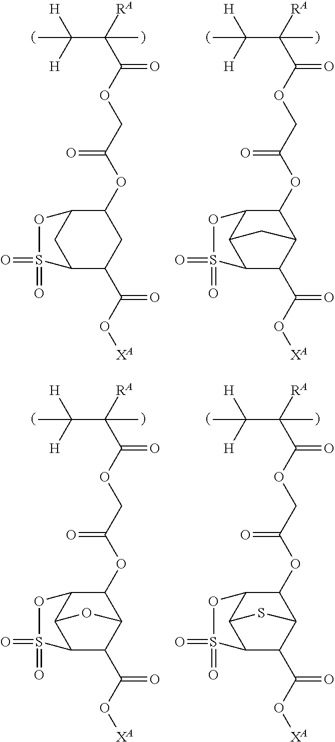

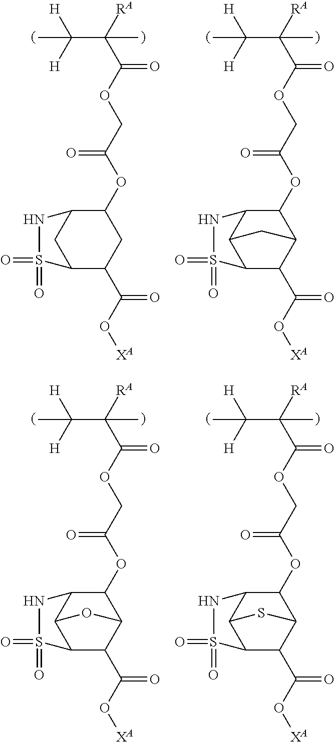

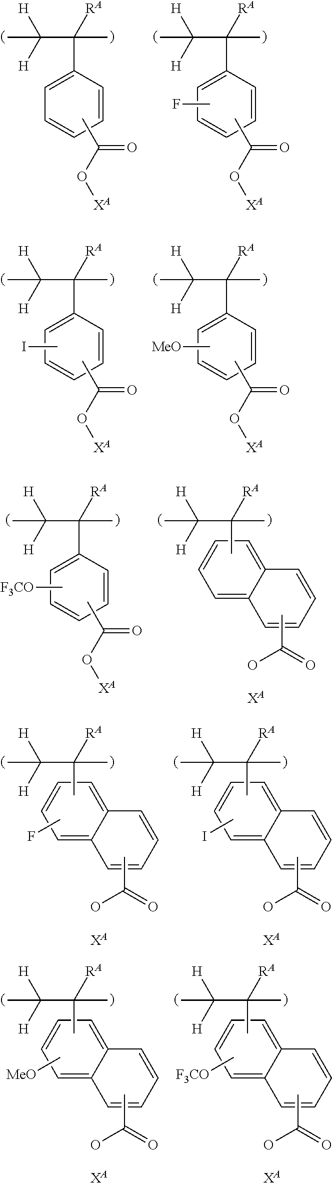

Examples of the repeating unit “a2” include, but are not limited thereto. In the following formula, R^{A} and X^{B} are as defined above.

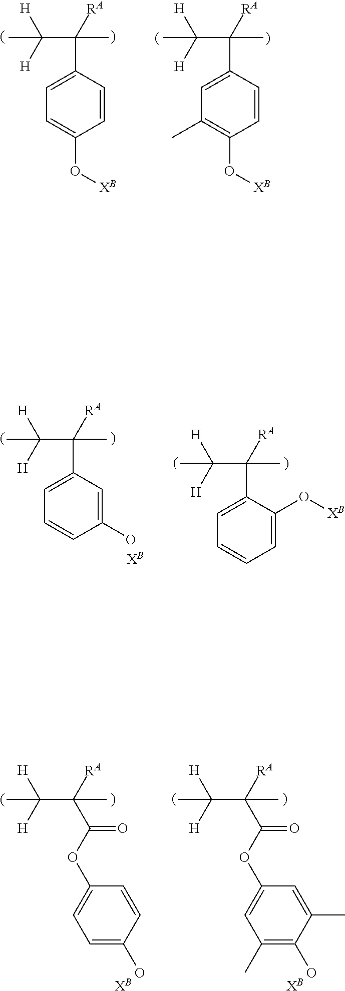

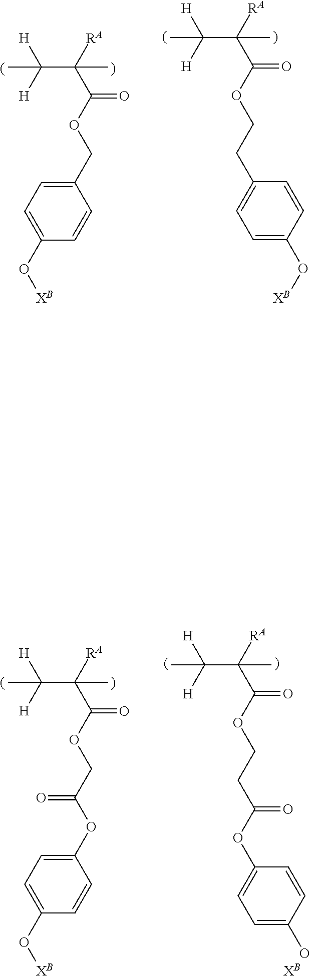

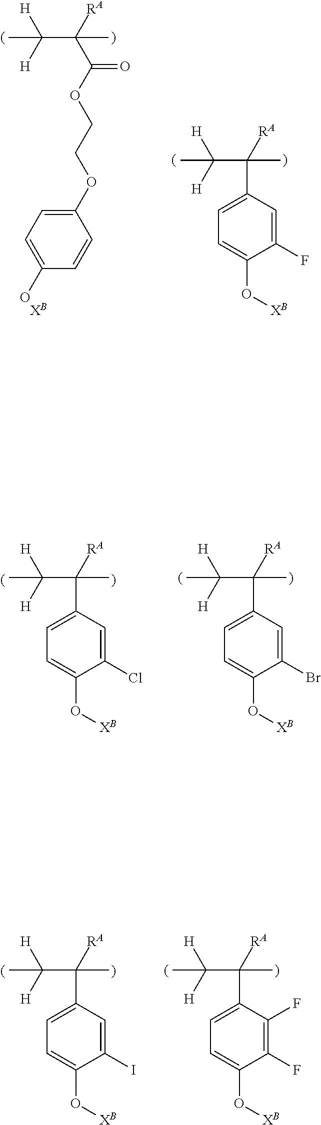

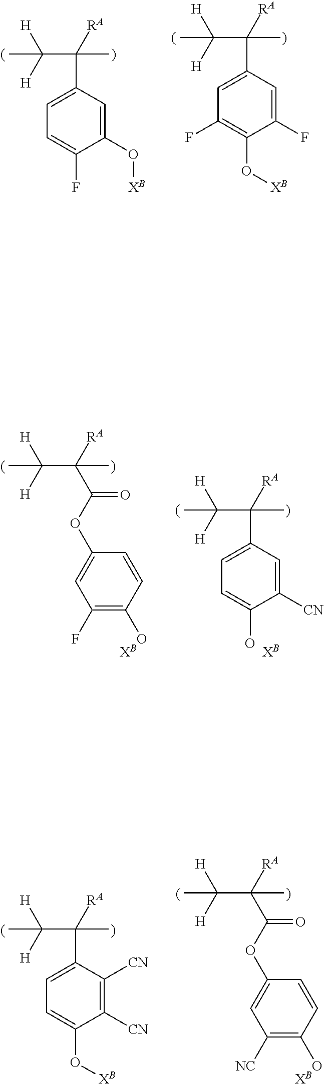

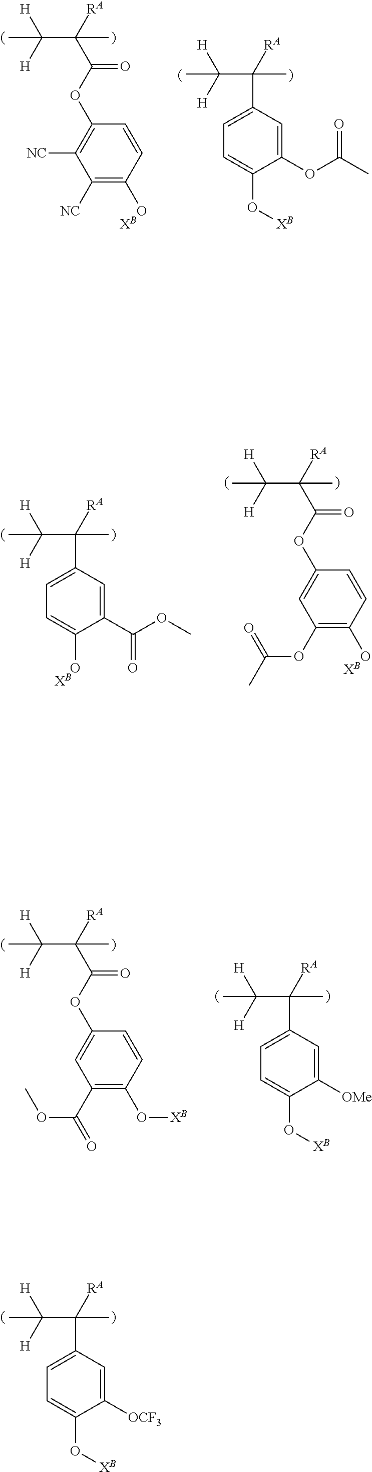

It is preferable that the polymer compound further contain the repeating unit (hereinafter, also referred to as a repeating unit “b1”) represented by the following formula (b1), or the repeating unit (hereinafter, also referred to as a repeating unit “b2”) represented by the following formula (b2),

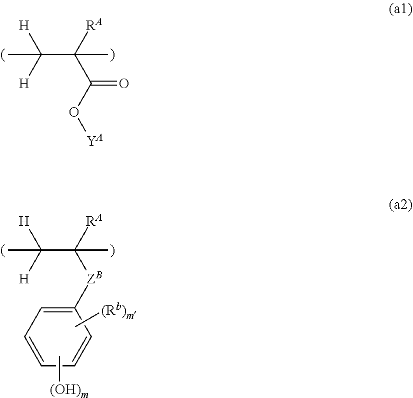

wherein R^{A} independently represents a hydrogen atom, a fluorine atom, a methyl group, or a trifluoromethyl group; YA is a hydrogen atom, or a polar group including at least one or more structures selected from the group consisting of a hydroxy group, a cyano group, a carbonyl group, a carboxy group, an ether bond, an ester bond, a sulfonic acid ester bond, a sulfonic acid amide bond, a carbonate bond, a lactone ring, a sultone ring, a sulfur atom, and a carboxylic acid anhydride; Z^{B} is a single bond or (backbone) —C(═O)—O—Z^{B1}—; Z^{B1} is a single bond or an alkanediyl group having 1 to 10 carbon atoms which may contain an ester and/or an ether bond; R^{b} represents a halogen atom, a nitro group, or a linear, branched or cyclic monovalent hydrocarbon group having 1 to 20 carbon atoms which may contain a heteroatom; m is an integer of 1 to 4; and m′ is an integer of 0 to 4.

In the formulae (b1) and (b2), R^{A}, R^{b} and Z^{B} are as defined above. YA is preferably a hydrogen atom, or a polar group containing at least one or more structures selected from the group consisting of a hydroxy group, a cyano group, a carbonyl group, a carboxy group, an ether bond, an ester bond, a sulfonic acid ester bond, a sulfonic acid amide bond, a carbonate bond, a lactone ring, a sultone ring, a sulfur atom, and a carboxylic acid anhydride. m is an integer of 1 to 4. m′ is an integer of 0 to 4.

YA may be a hydrogen atom, or may be a polar group comprising at least one or more structures selected from the group consisting of a hydroxy group other than phenolic hydroxy groups, a cyano group, a carbonyl group, a carboxy group, an ether bond, an ester bond, a sulfonic acid ester bond, a carbonate bond, a lactone ring, a sultone ring, and a carboxylic acid anhydride.

Examples of the repeating unit “b1” include, but are not limited to, those shown below. In the following formulae, R^{A} is as defined above.

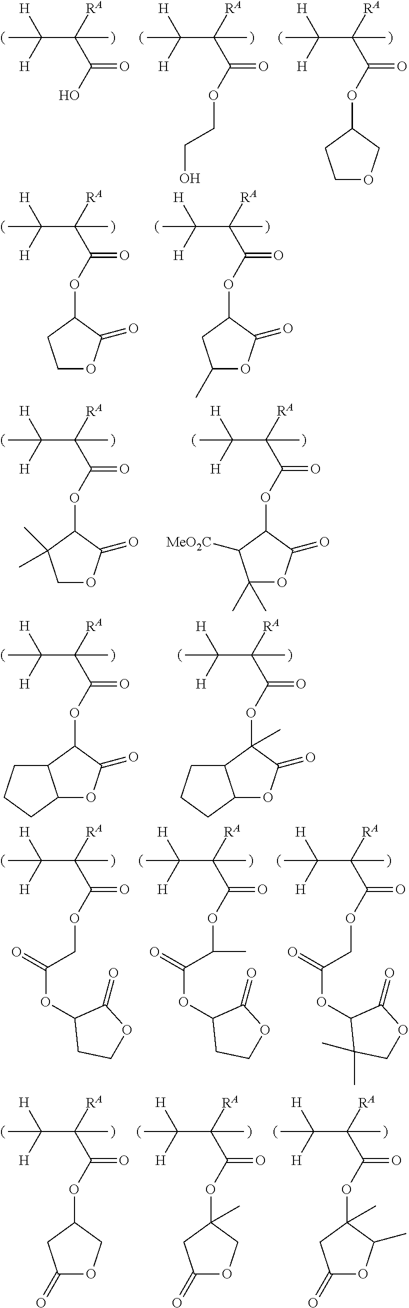

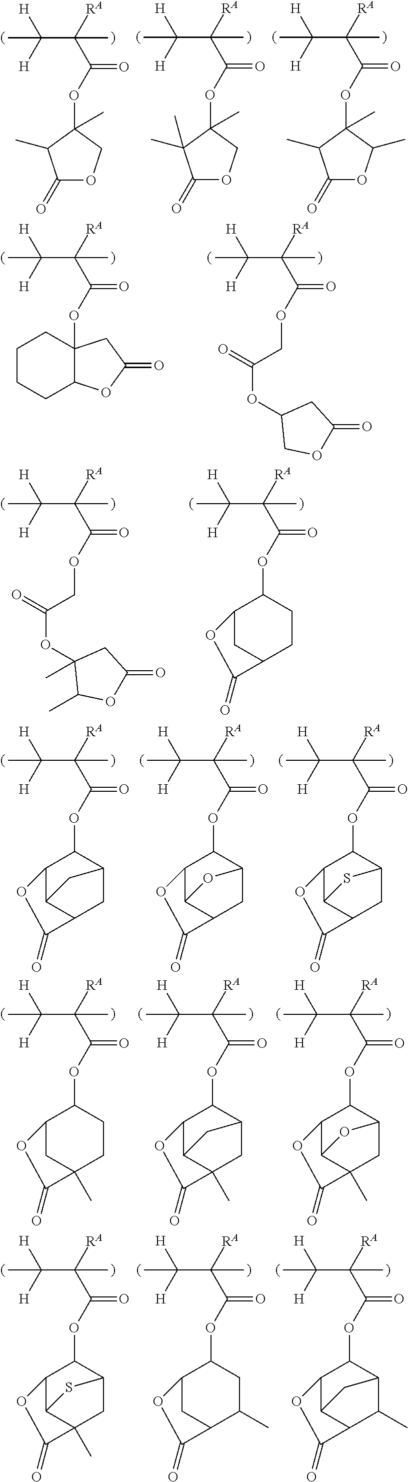

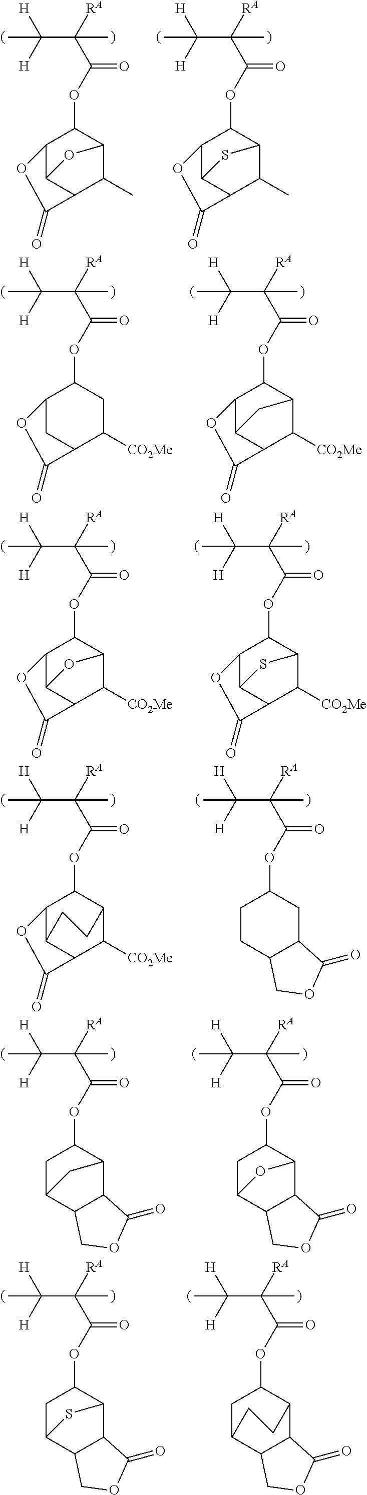

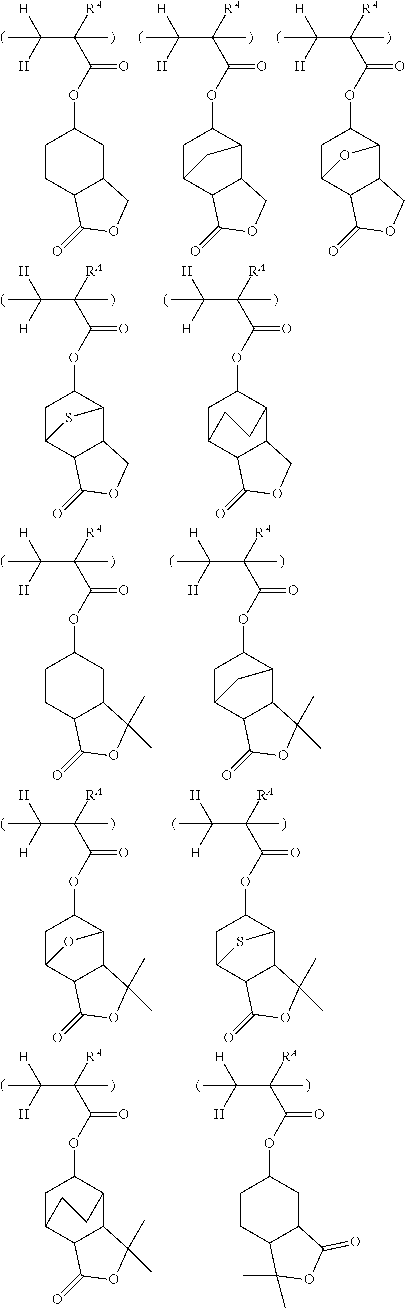

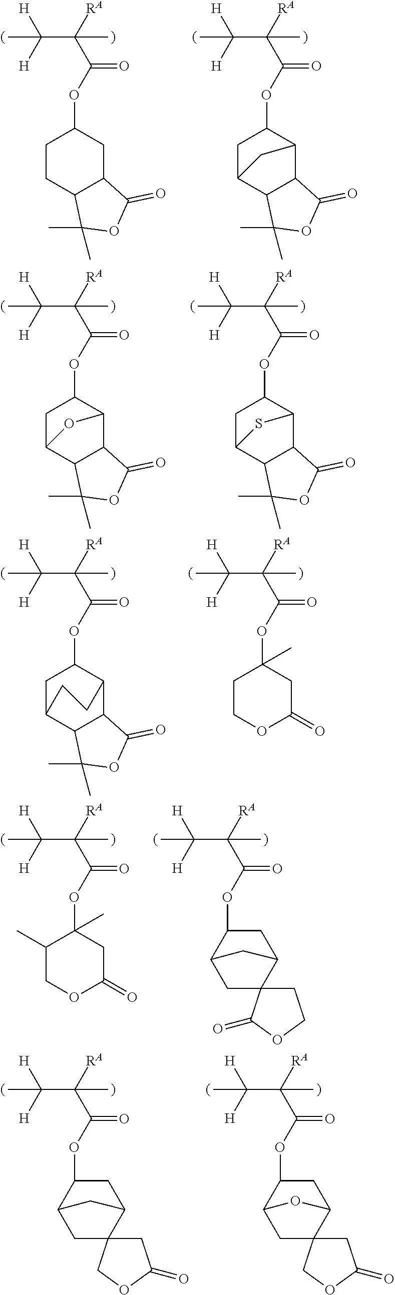

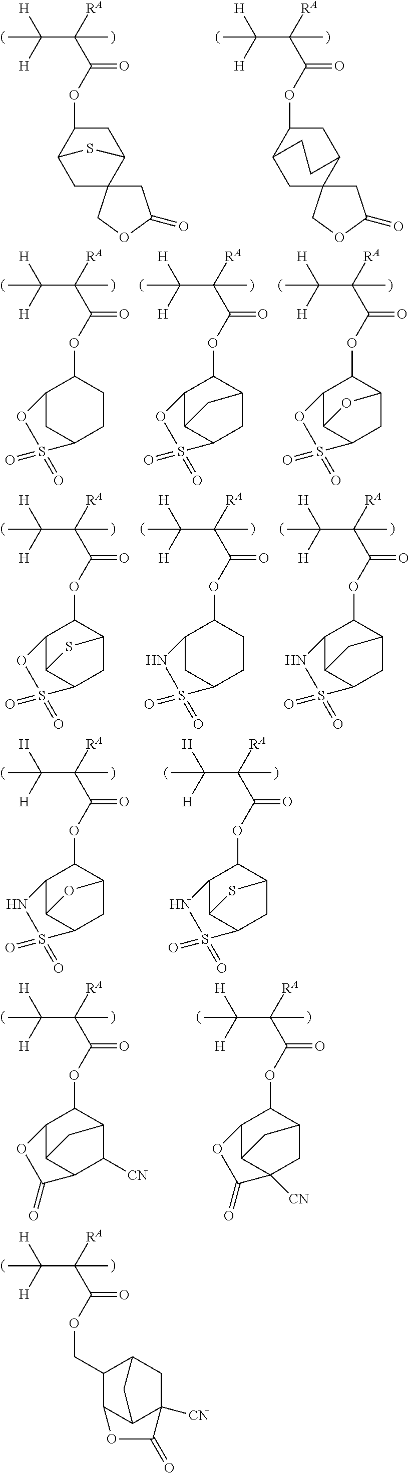

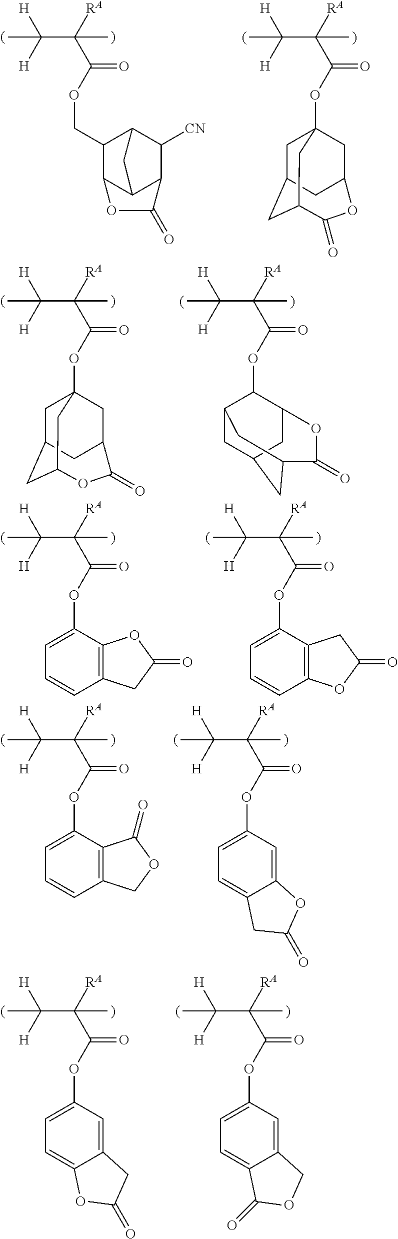

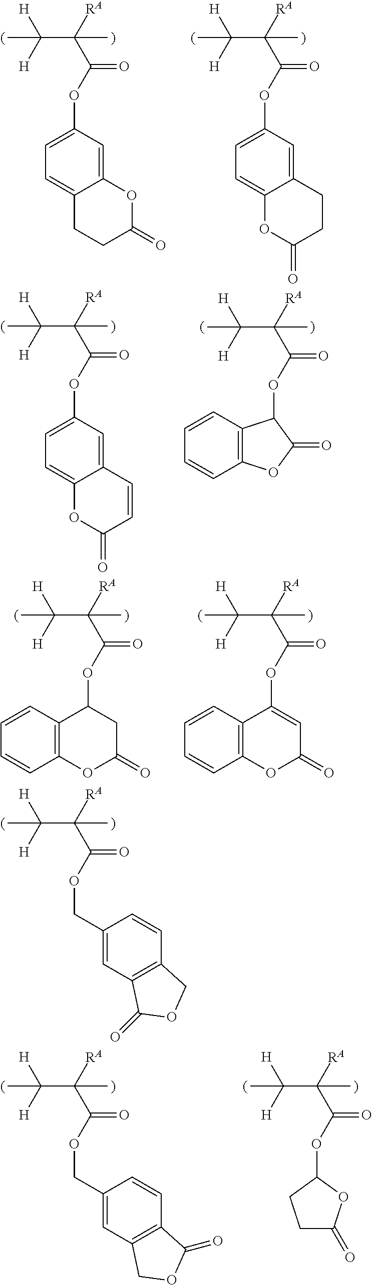

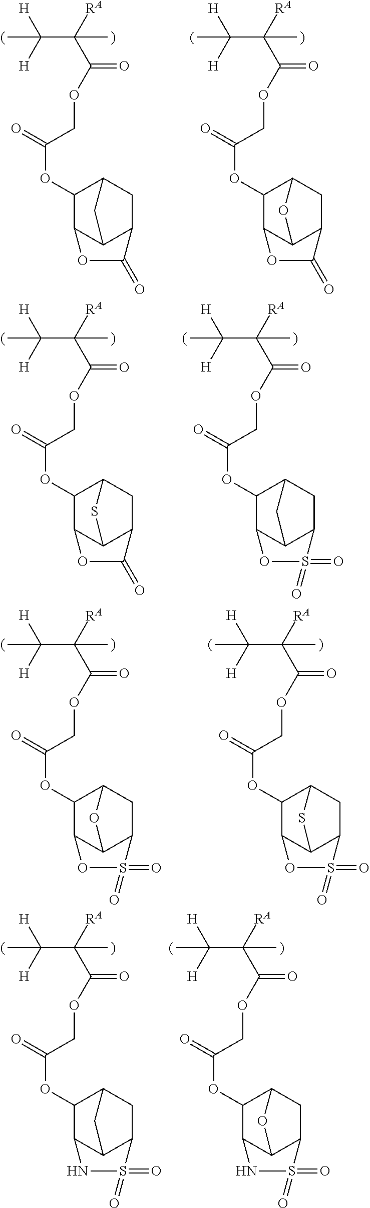

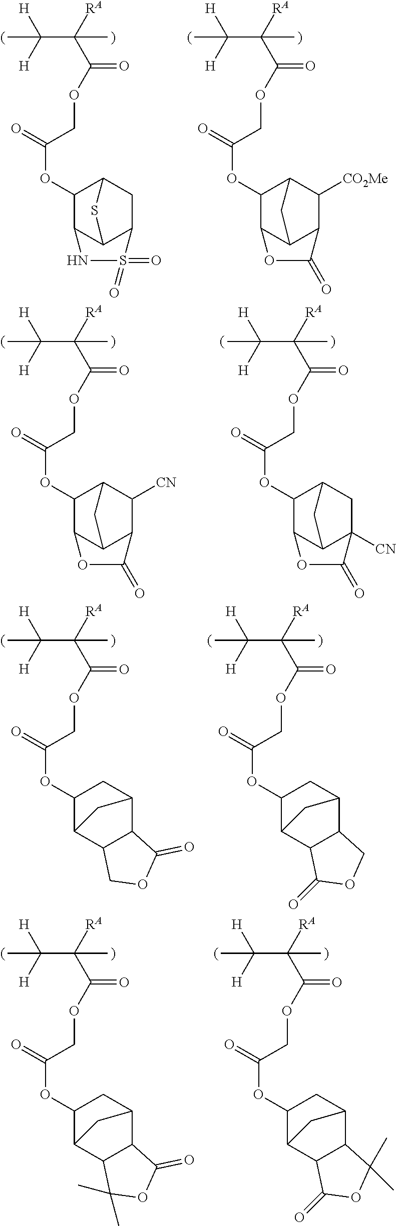

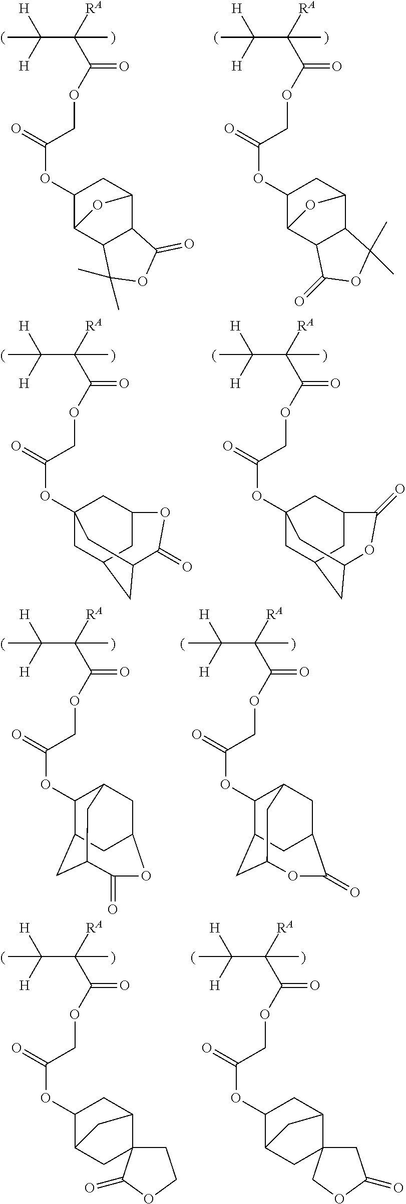

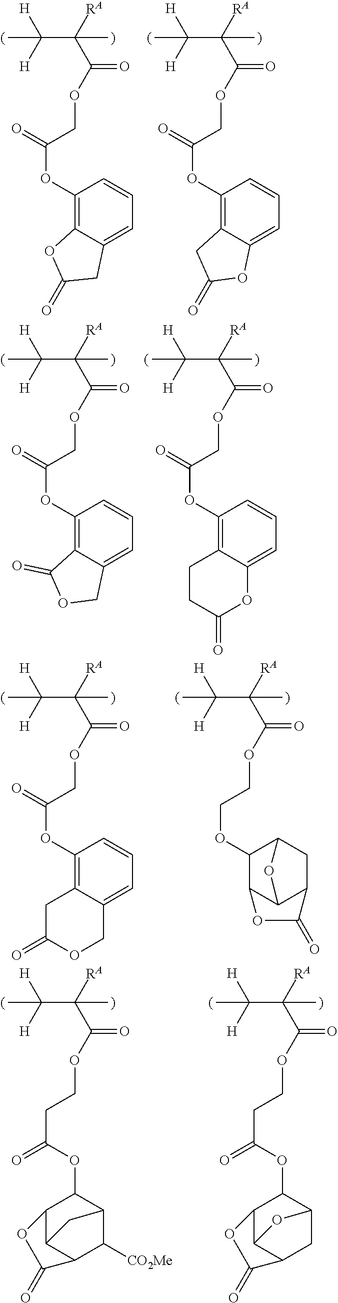

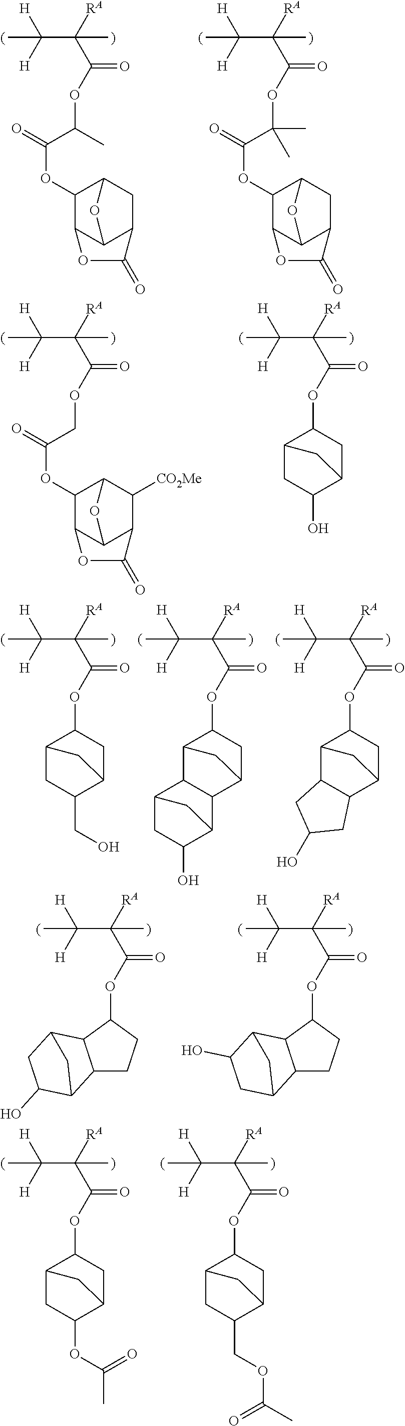

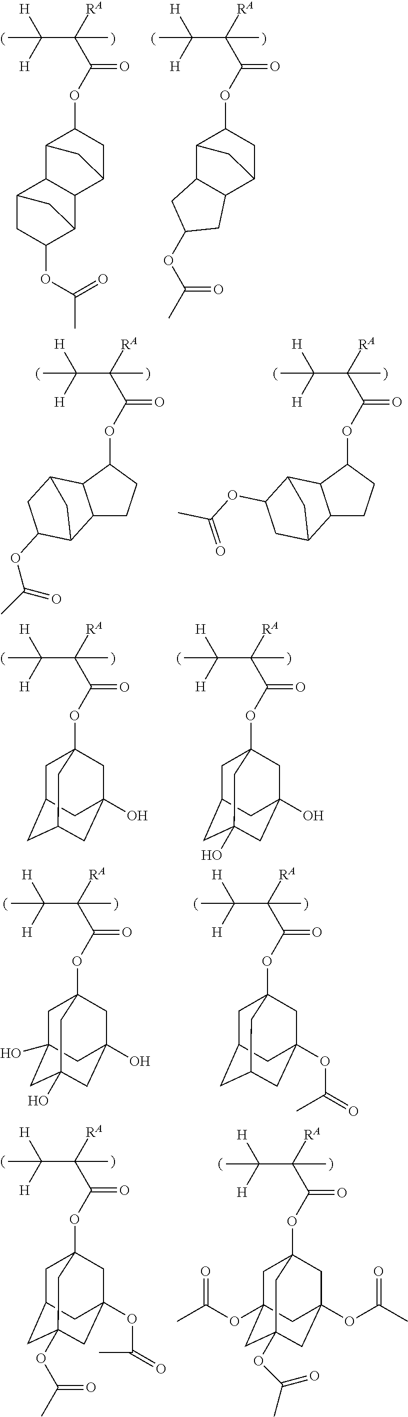

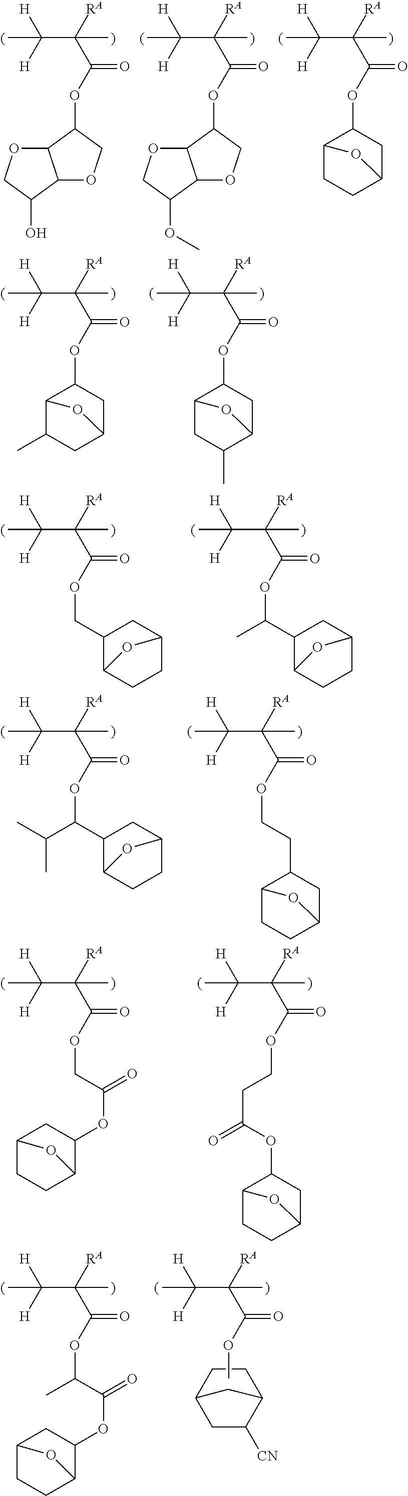

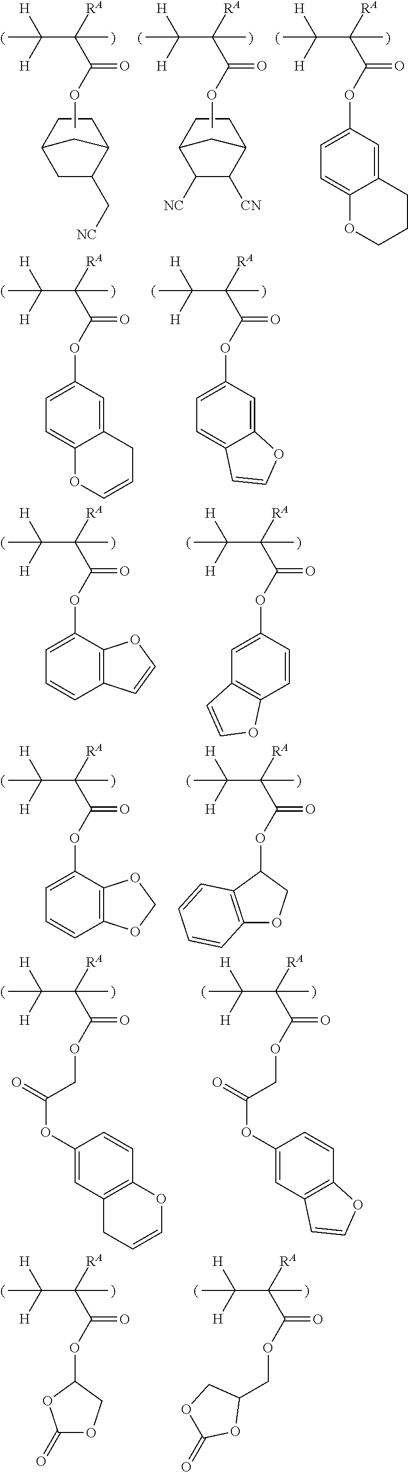

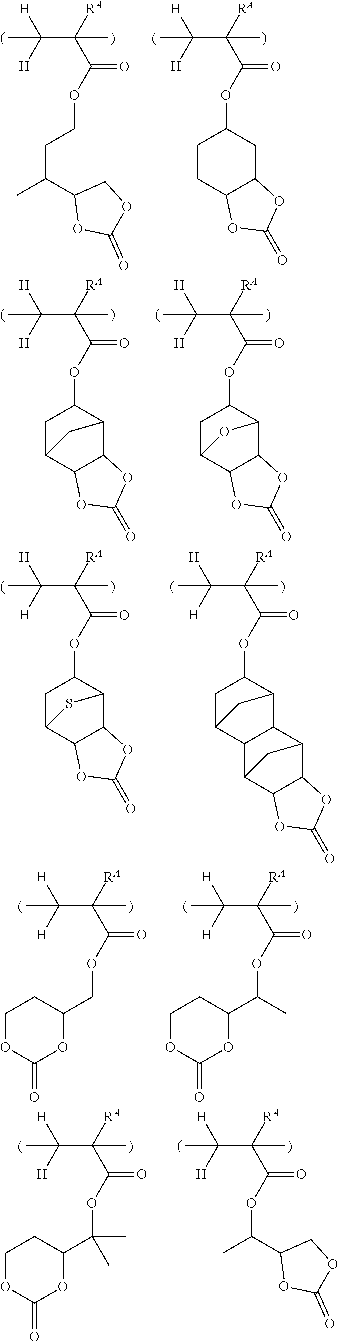

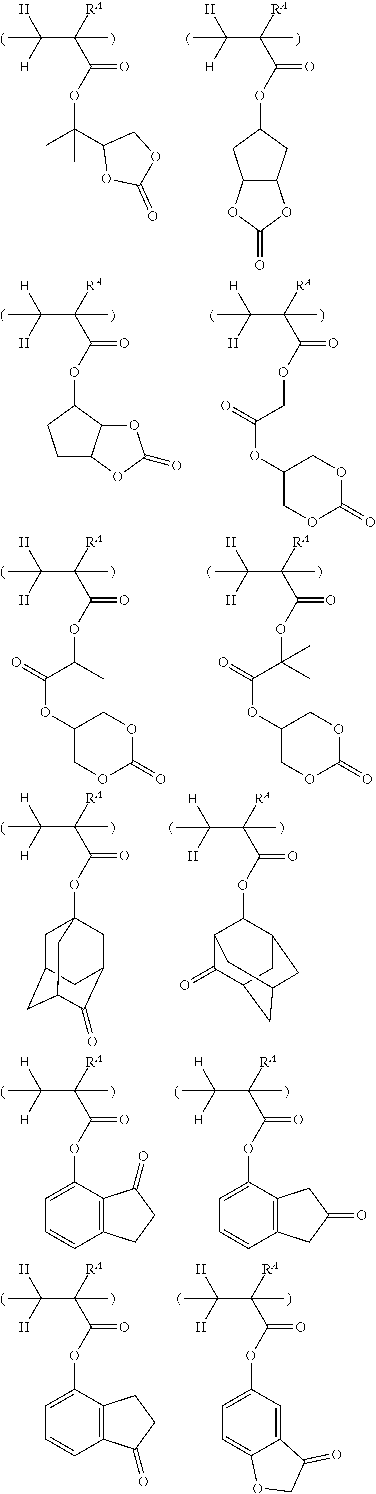

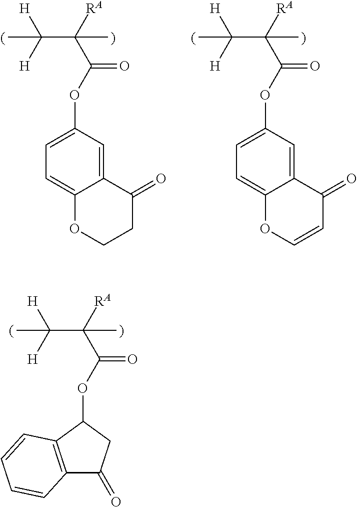

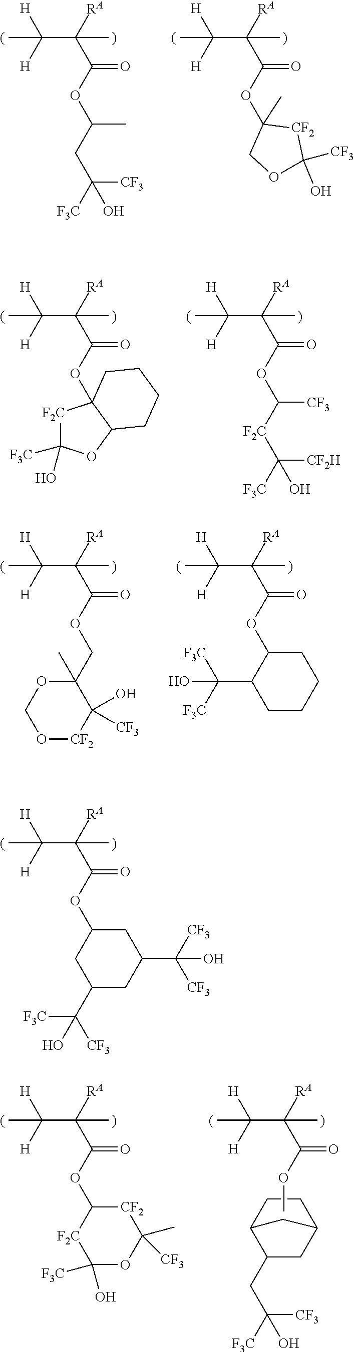

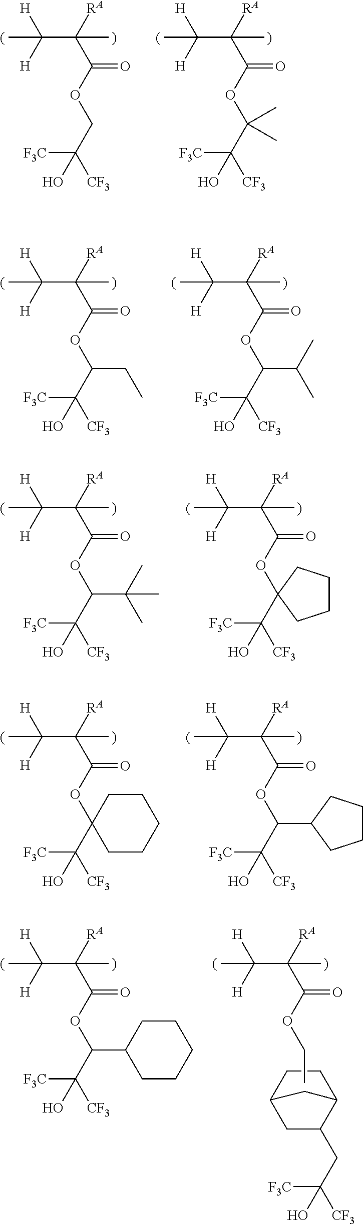

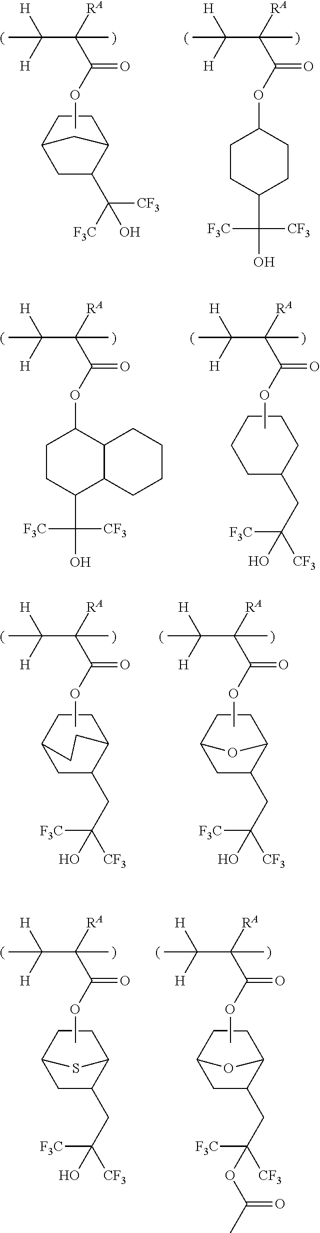

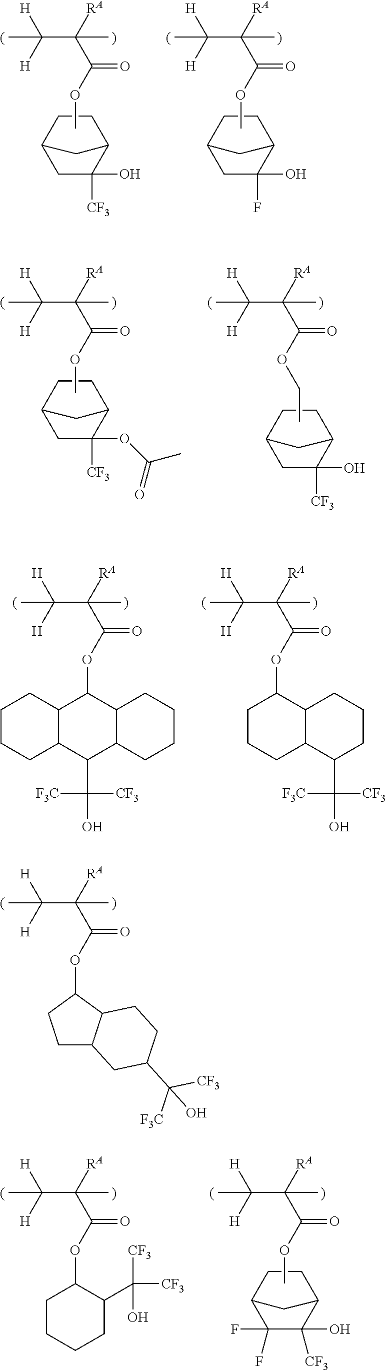

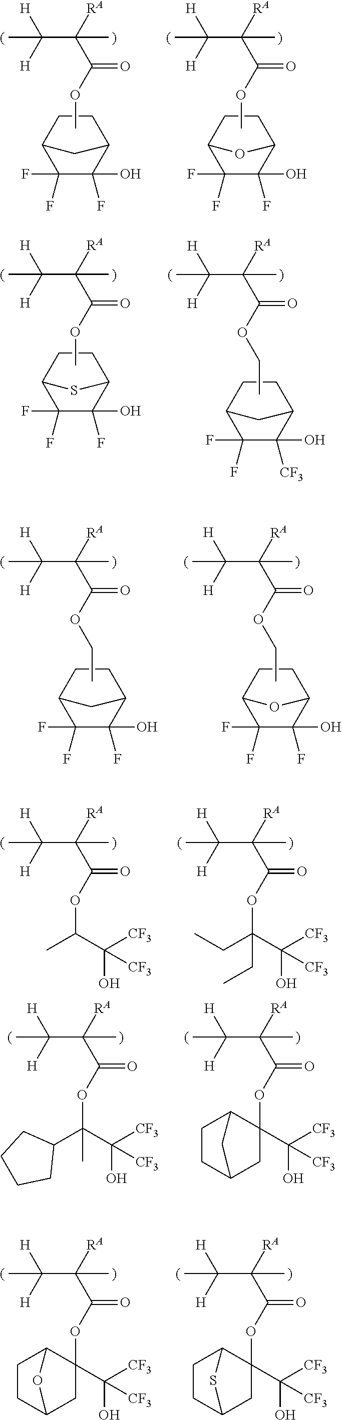

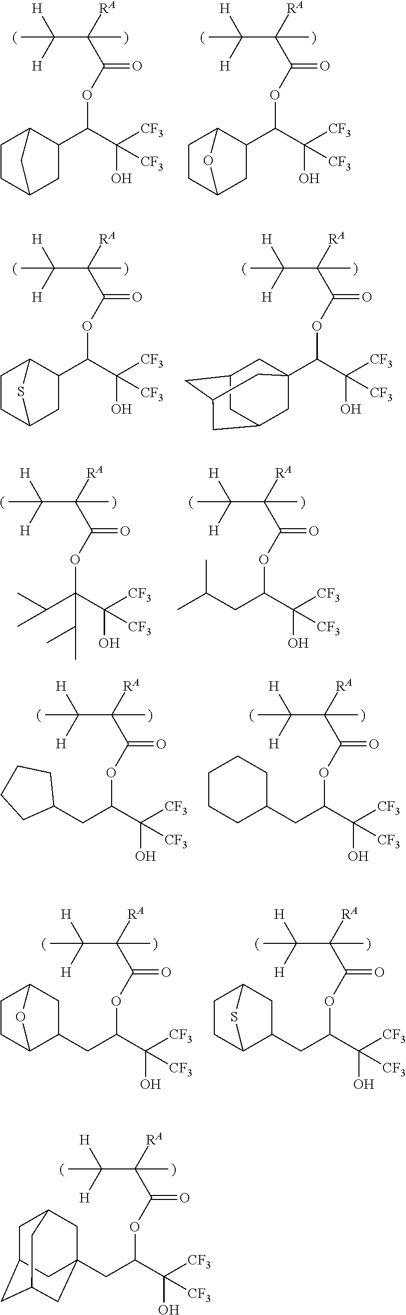

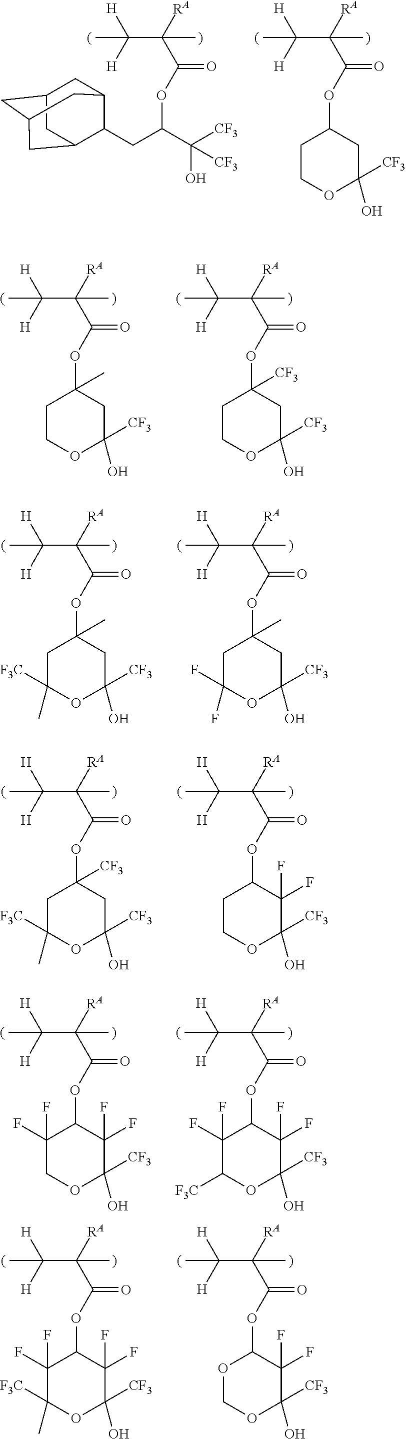

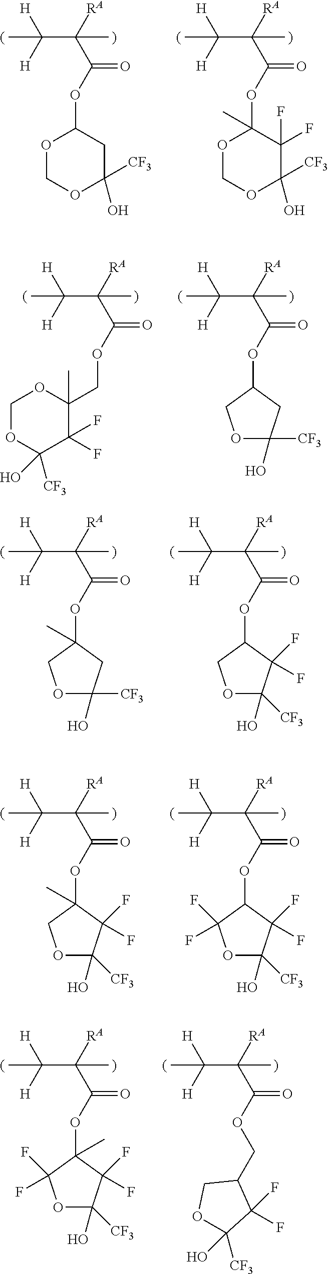

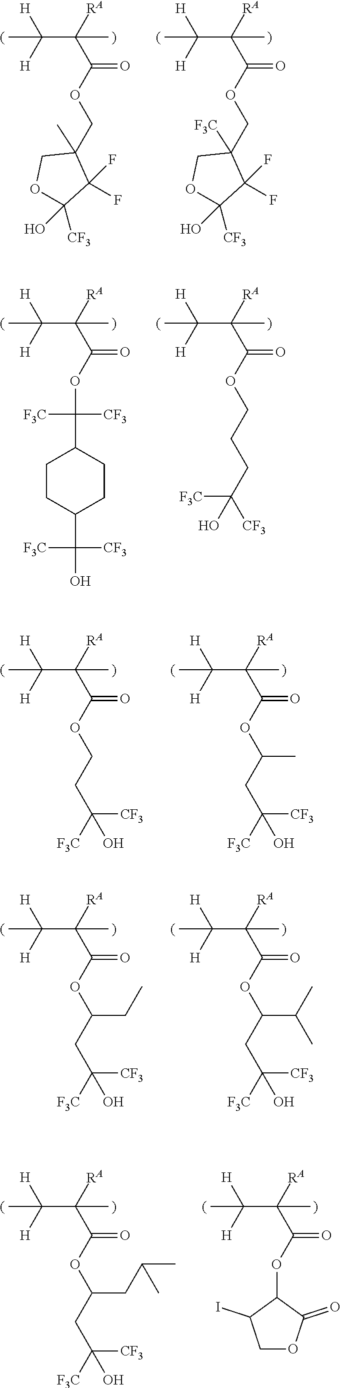

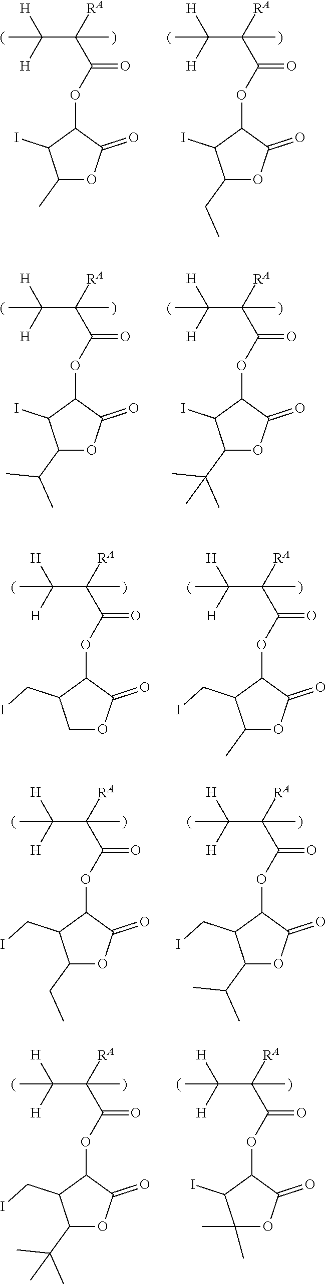

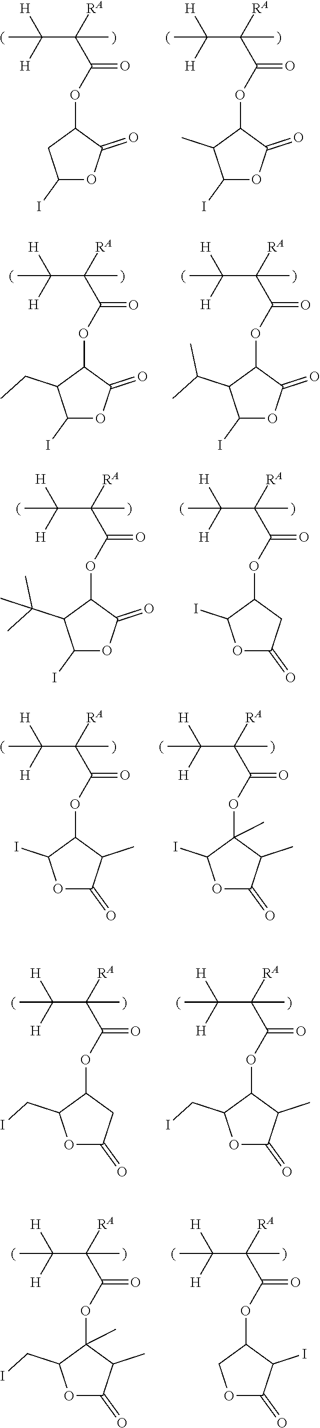

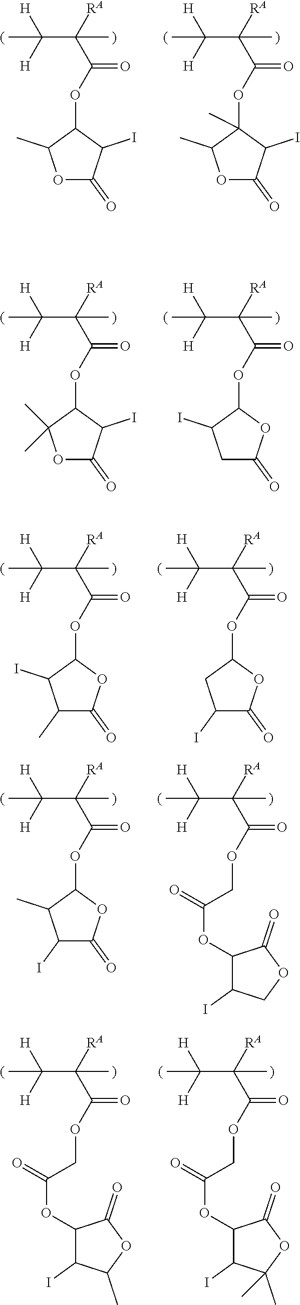

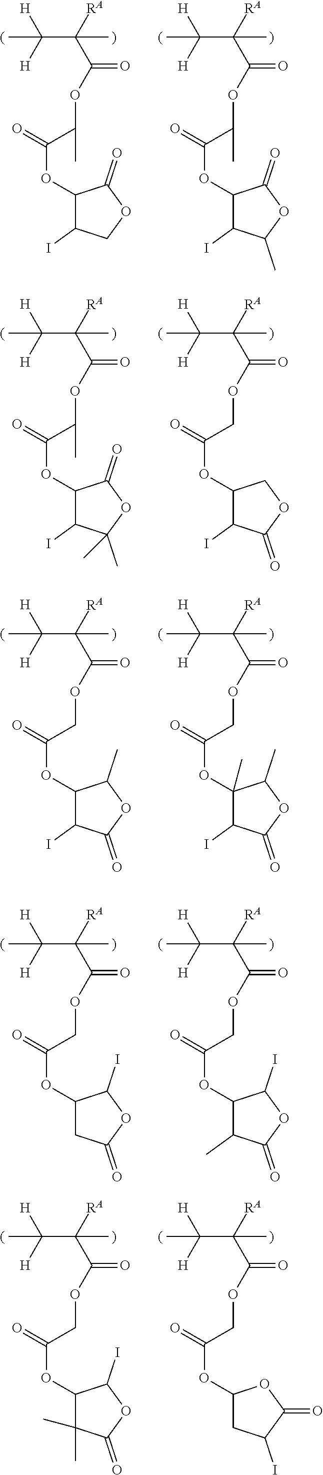

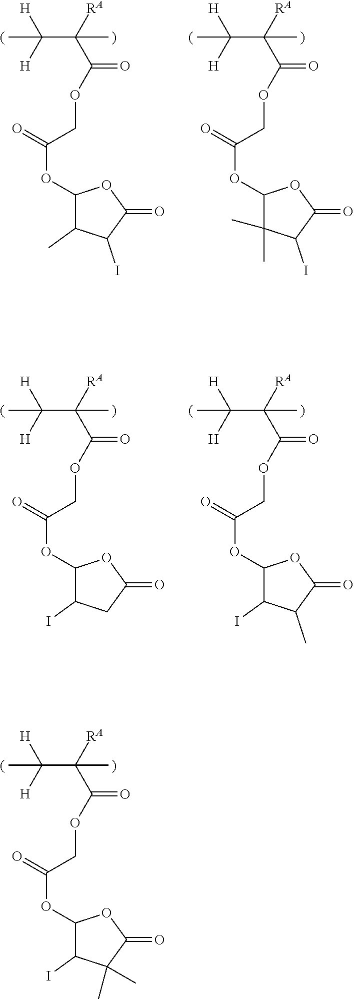

Examples of the repeating unit “b2” include, but are not limited thereto. In the following formulae, R^{A} is as defined above.

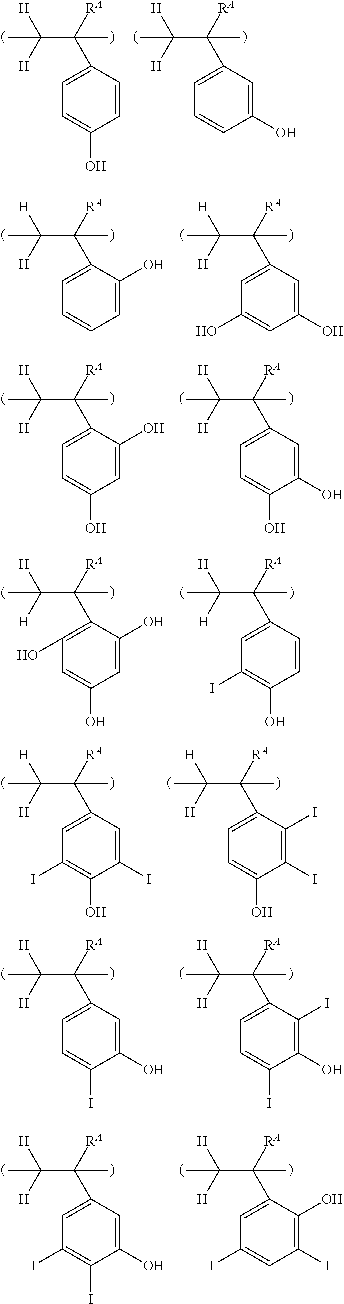

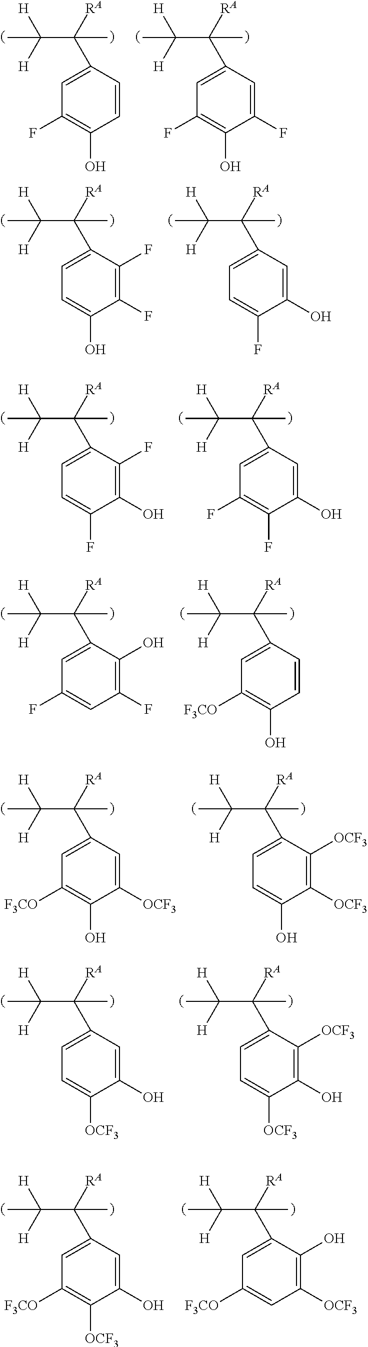

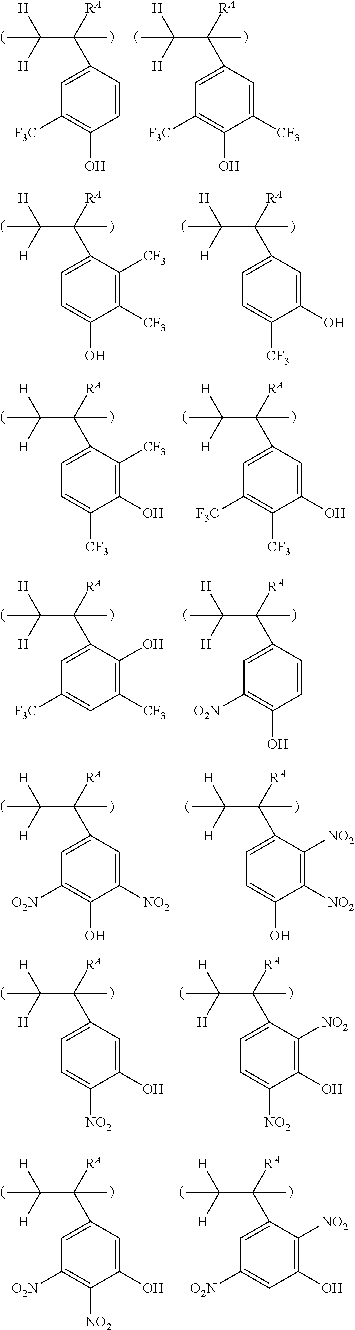

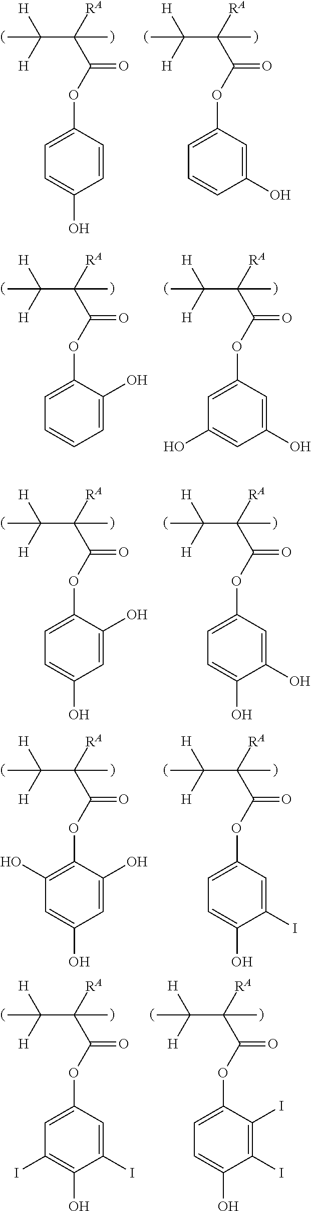

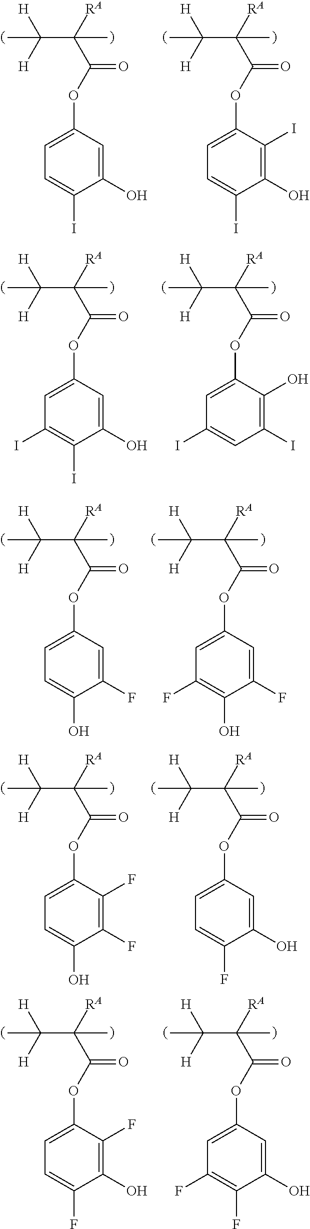

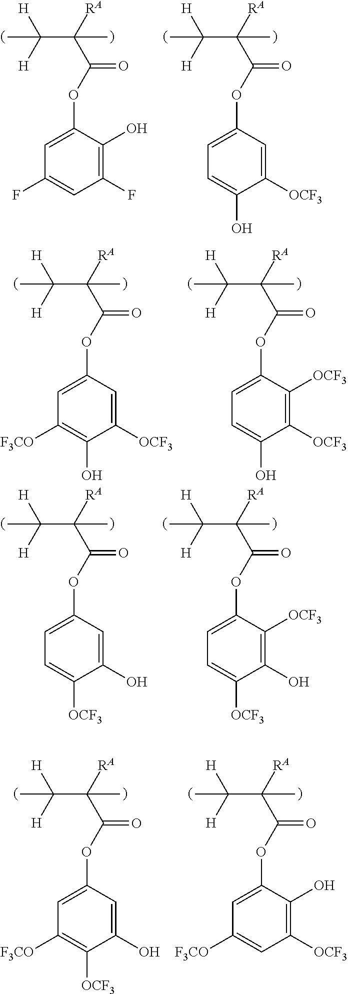

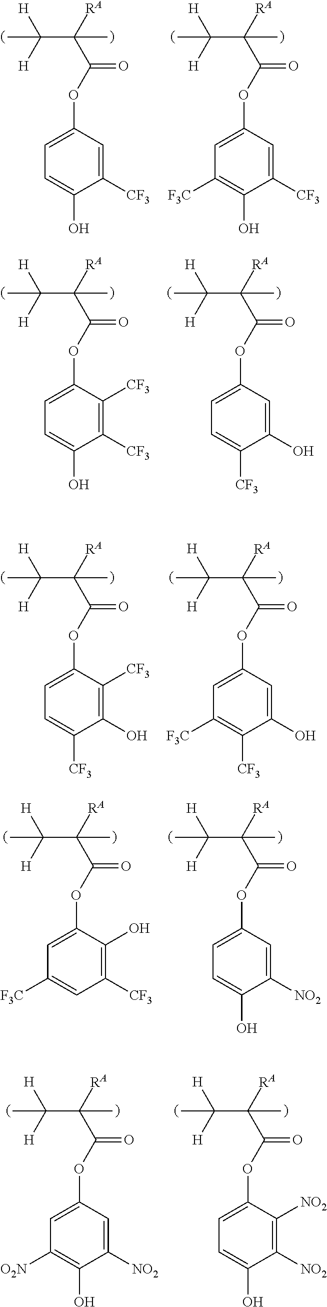

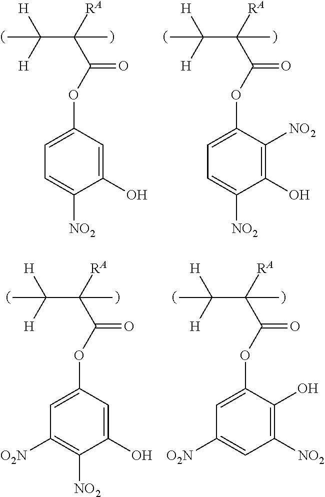

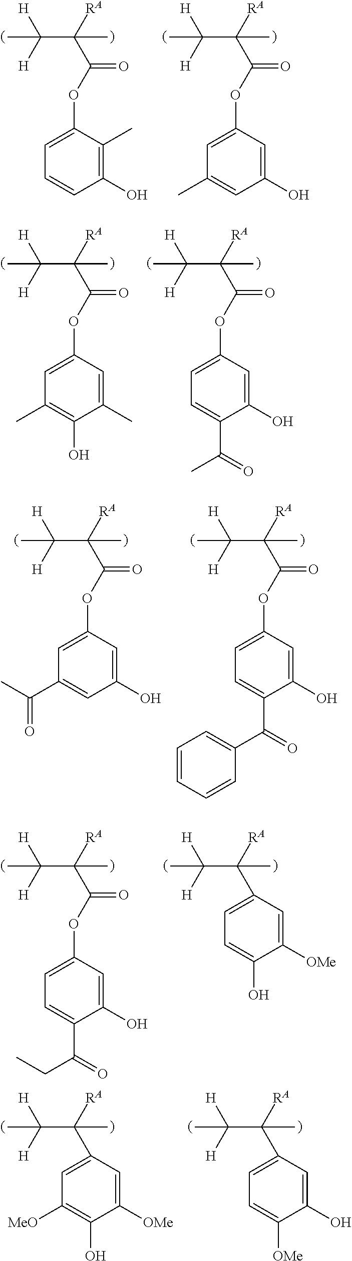

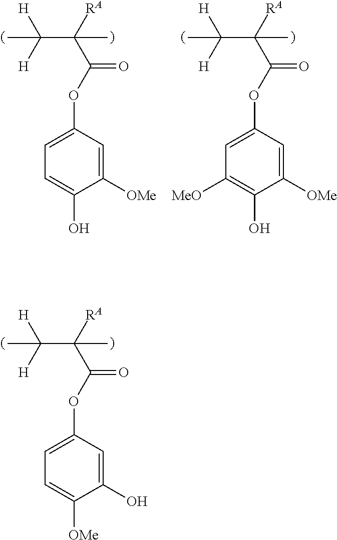

As the repeating unit “b1” or “b2”, units having a lactone ring as the polar group are particularly preferred in the ArF lithography and units having a phenol moiety are preferred in the KrF, EB and EUV lithography.

The polymer compound may further contain any of the repeating units (hereinafter, also referred to as repeating units “c1” to “c4”) represented by the following formulae (C1) to (C4),

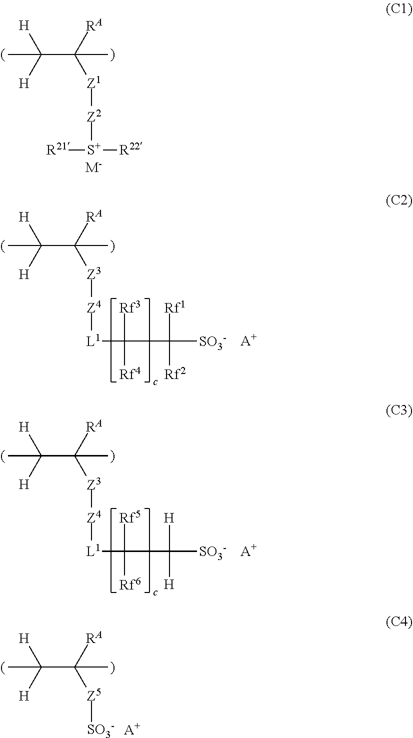

wherein R^{A} independently represents a hydrogen atom, a fluorine atom, a methyl group, or a trifluoromethyl group; Z¹ is a single bond or a phenylene group; Z² is a single bond, *—C(═O)—O—Z²¹—, *—C(═O)—NH—Z²¹—, or *—O—Z²¹—; Z²¹ is an aliphatic hydrocarbylene group having 1 to 12 carbon atoms, a phenylene group, or a divalent group obtained by combining these groups, and may contain a carbonyl group, an ester bond, an ether bond or a hydroxy group; Z³ is a single bond, a phenylene group, a naphthylene group, or *—C(═O)—O—Z³¹—; Z³¹ is a single bond, an aliphatic hydrocarbylene group having 1 to 14 carbon atoms which may contain a hydroxy group, an ether bond, an ester bond, or a lactone ring, or a phenylene group or a naphthylene group; Z⁴ is a single bond, a methylene group, or *—Z⁴¹—C(═O)—O—; Z⁴¹ represents a hydrocarbylene group having 1 to 20 carbon atoms which may contain an ether bond, an ester bond, or a heteroatom; Z⁵ is a single bond, a methylene group, an ethylene group, a phenylene group, a fluorinated phenylene group, a phenylene group substituted with a trifluoromethyl group, *—C(═O)—O—Z⁵¹—, *—C(═O)—N(H)—Z⁵¹—, or *—O—Z⁵¹—; Z⁵¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond or a hydroxy group; and * represents a bonding arm to a carbon atom in the backbone; R^{21′} and R^{22′} independently represent a hydrocarbyl group having 1 to 20 carbon atoms which may contain a heteroatom; R^{21′} and R^{22′} may also be bonded to each other to form a ring with a sulfur atom to which R^{21′} and R^{22′} are bonded; L¹ is a single bond, an ether bond, an ester bond, a carbonyl group, a sulfonic acid ester bond, a carbonate bond or a carbamate bond; Rf¹ and Rf² independently represent a fluorine atom or a fluorinated alkyl group having 1 to 6 carbon atoms; Rf³ and Rf⁴ independently represent a hydrogen atom, a fluorine atom, or a fluorinated alkyl group having 1 to 6 carbon atoms; Rf⁵ and Rf⁶ independently represent a hydrogen atom, a fluorine atom, or a fluorinated alkyl group having 1 to 6 carbon atoms, provided that not all of Rf⁵ and Rf⁶ are hydrogen atoms at the same time; M⁻ is a non-nucleophilic counter ion; A⁺ is an onium cation; and c is an integer of 0 to 3.

In the formulae (C1) to (C4), R^{A} is as defined above. Z¹ is a single bond or a phenylene group; Z² is a single bond, —C(═O)—O—Z²¹—, —C(═O)—NH—Z²¹—, or —O—Z²¹—; Z²¹ is an aliphatic hydrocarbylene group having 1 to 12, preferably 1 to 6 carbon atoms, a phenylene group, or a divalent group obtained by combining these groups, and may contain a carbonyl group, an ester bond, an ether bond or a hydroxy group; Z³ is a single bond, a phenylene group, a naphthylene group, or (backbone) —C(═O)—O—Z³¹—; Z³¹ is an aliphatic hydrocarbylene group having 1 to 14 carbon atoms which may contain a hydroxy group, an ether bond, an ester bond, or a lactone ring, or a phenylene group or a naphthylene group; Z⁴ is a single bond, a methylene group, or —Z⁴¹—C(═O)—O—; Z⁴¹ represents a hydrocarbylene group having 1 to 20 carbon atoms which may contain an ether bond, an ester bond, or a heteroatom; Z⁵ is a single bond, a methylene group, an ethylene group, a phenylene group substituted with a trifluoromethyl group, a phenylene group, a fluorinated phenylene group, —C(═O)—O—Z⁵¹—, —C(═O)—N(H)—Z⁵¹—, or —O—Z³¹—; Z⁵¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond or a hydroxy group.

Specific examples of the structures represented by Z²¹, Z³¹ and Z⁵¹ include any of linear, branched and cyclic structures, such as those exemplified in the description of Z^{A} in the formula (a1).

The hydrocarbylene group represented by Z⁴¹ may be any of a saturated, unsaturated, linear, branched, or cyclic hydrocarbyl group. Examples thereof include, but are not limited thereto,

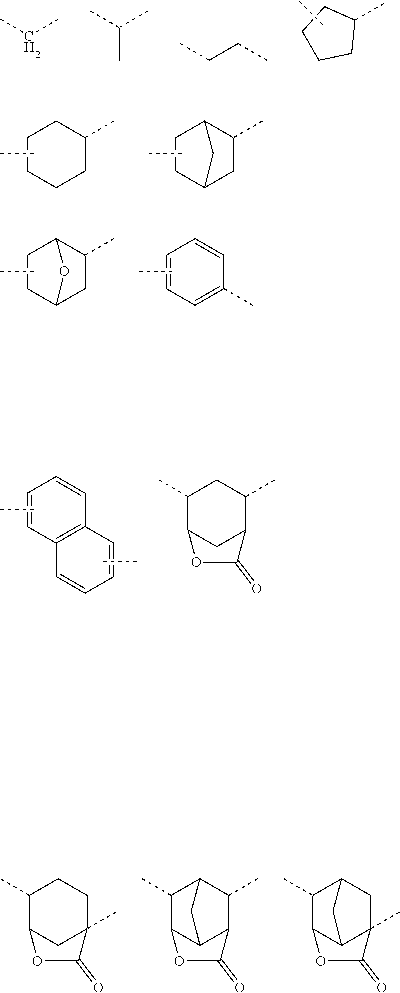

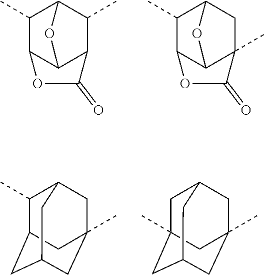

wherein the dashed line represents a bonding arm.

In the formula (C1), R^{21′} and R^{22′} independently represent a hydrocarbyl group having 1 to 20 carbon atoms which may contain a heteroatom. The hydrocarbyl group represented by R^{21′} and R^{22′} may be any of a saturated, unsaturated, linear, branched, or cyclic hydrocarbyl group. Examples thereof include alkyl groups such as a methyl group, an ethyl group, a n-propyl group, an isopropyl group, a n-butyl group, and a tert-butyl group; cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups such as a vinyl group, an allyl group, a propenyl group, a butenyl group, and a hexenyl group; cyclic unsaturated hydrocarbyl groups such as a cyclohexenyl group; aryl groups such as a phenyl group, a naphthyl group, and a thienyl group; aralkyl groups such as a benzyl group, a 1-phenylethyl group, and a 2-phenylethyl group; and groups obtained by combining these groups. Among them, aryl groups are preferable. Also included are the foregoing hydrocarbyl groups in which at least one hydrogen atom is optionally substituted with a group containing a heteroatom such as an oxygen atom, a sulfur atom, and a nitrogen atom; further optionally, the carbon atoms of these groups may be intervened by a group containing a heteroatom such as an oxygen atom, a sulfur atom and a nitrogen atom, so that the groups may contain a hydroxy group, a cyano group, a carbonyl group, an ether bond, an ester bond, a sulfonic acid ester bond, a carbonate bond, a lactone ring, a sultone ring, a carboxylic acid anhydride, a haloalkyl group or the like.

R^{21′} and R^{22′} may also be bonded to each other to form a ring with a sulfur atom to which R^{21′} and R^{22′} are bonded. Examples thereof include those represented by the following formulae.

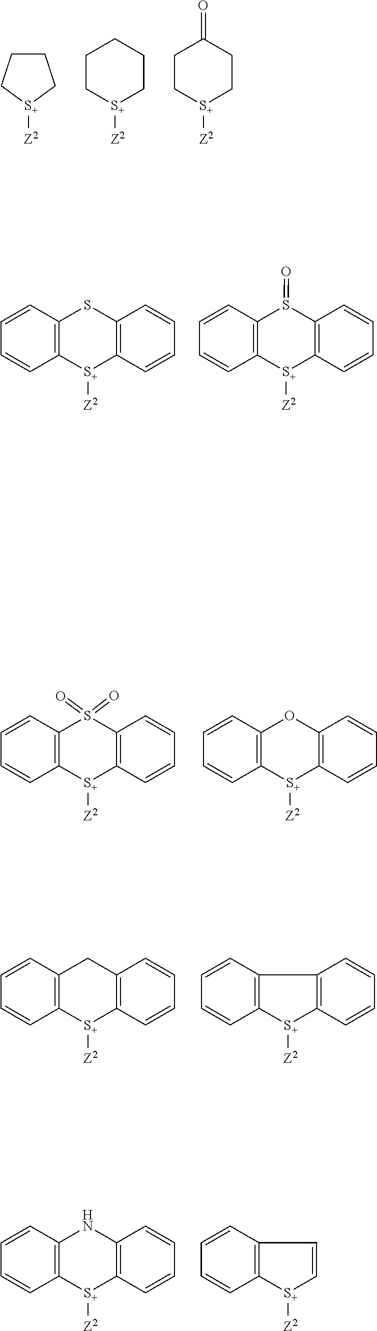

Examples of the cations in the repeating unit “c1” include, but are not limited thereto. In the following formulae, R^{A} is as defined above.

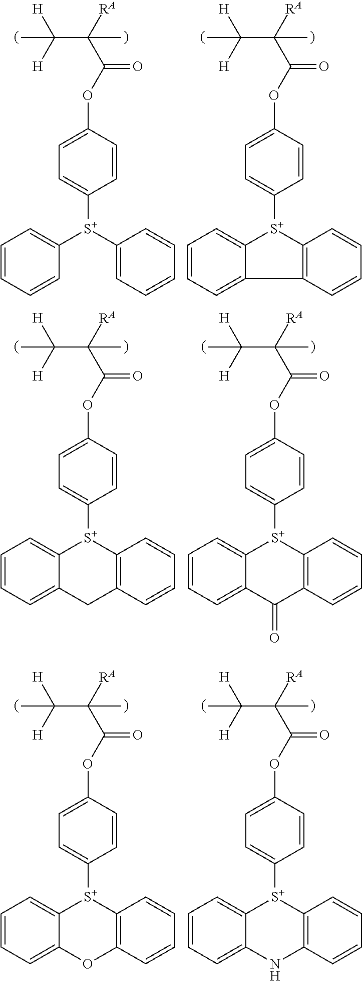

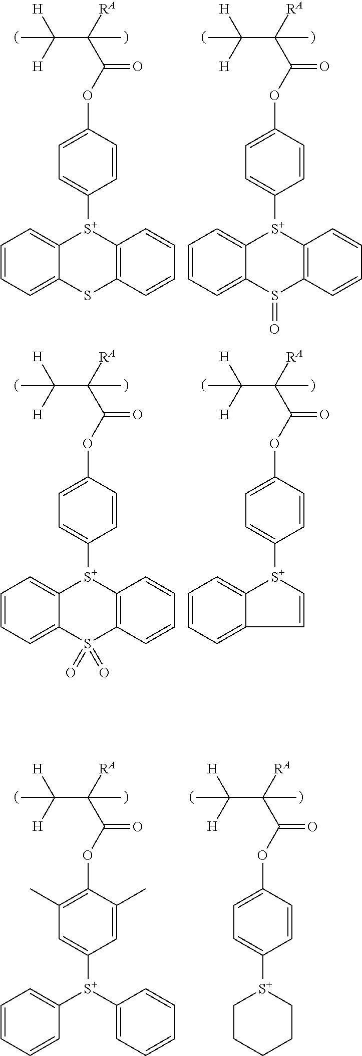

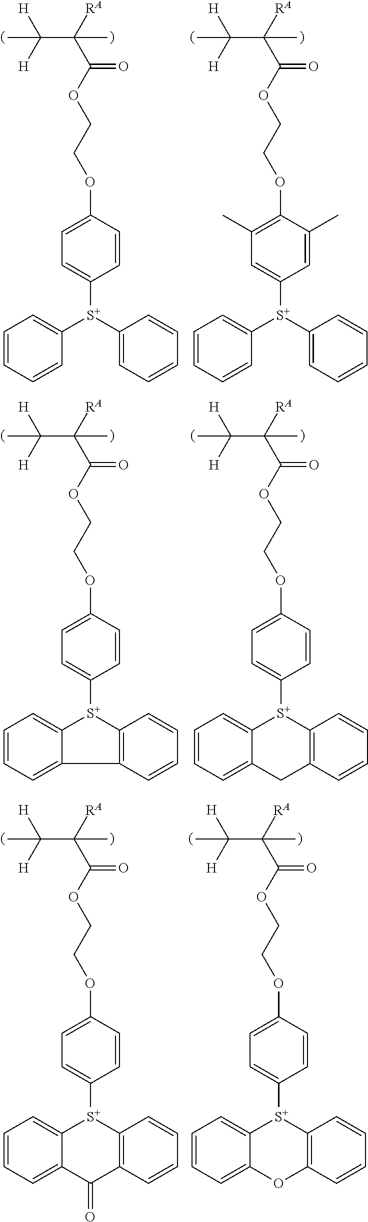

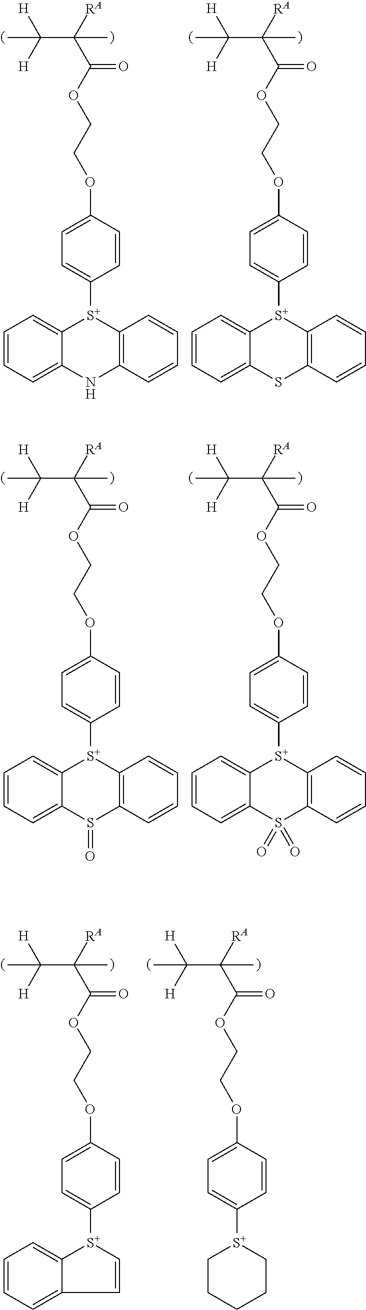

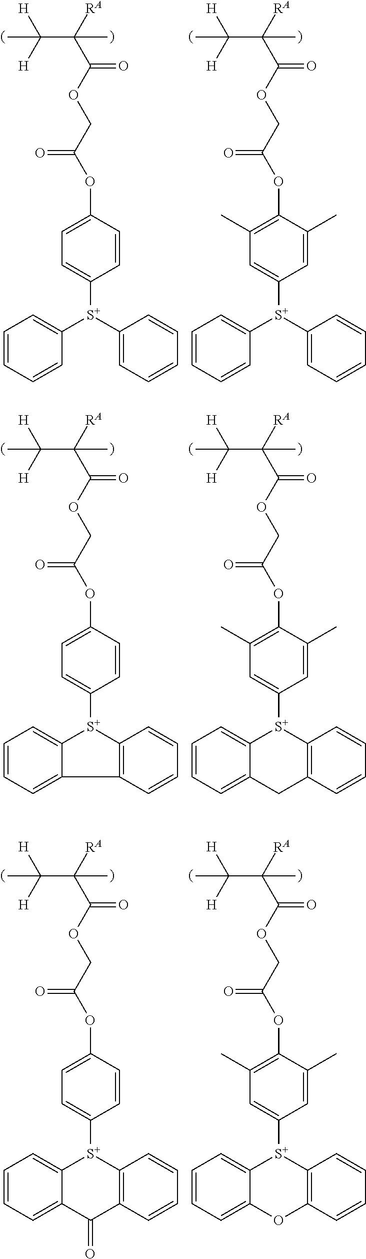

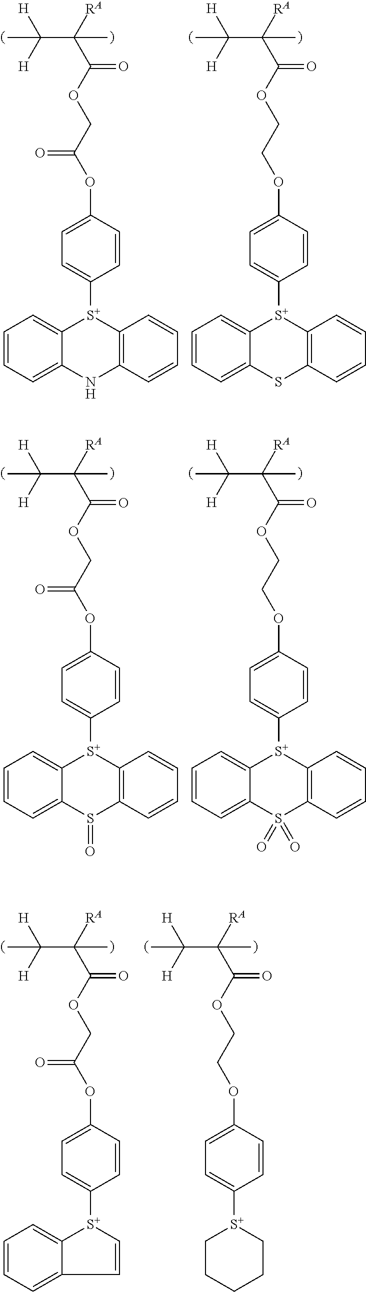

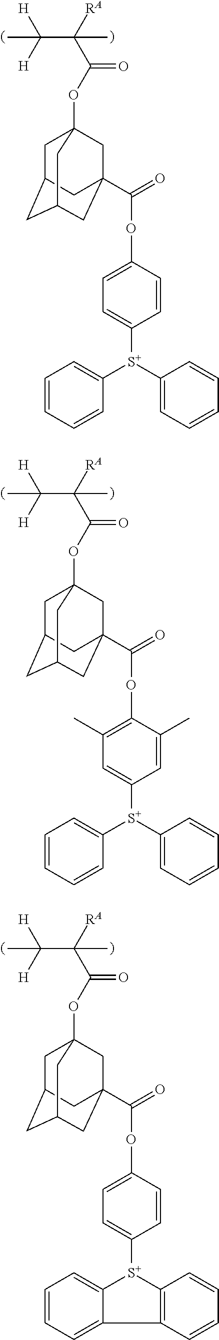

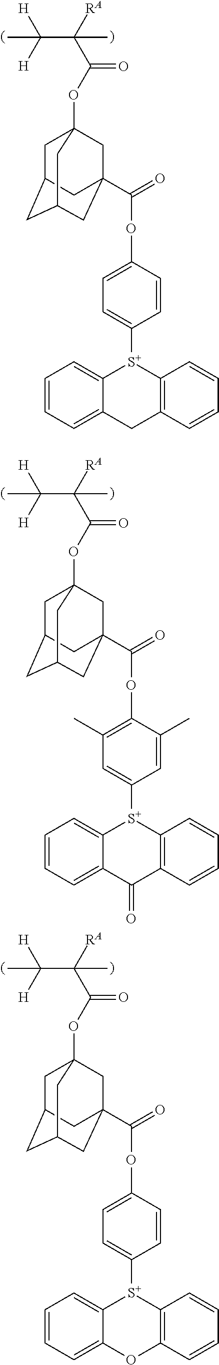

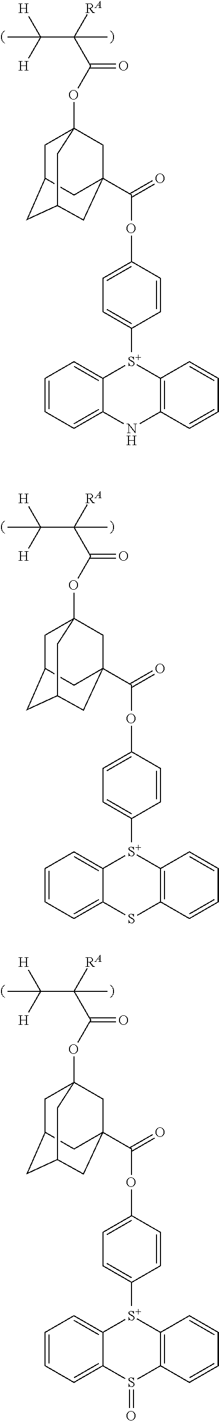

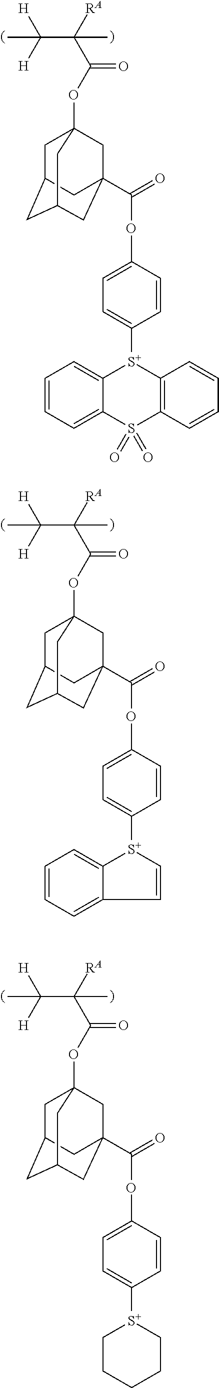

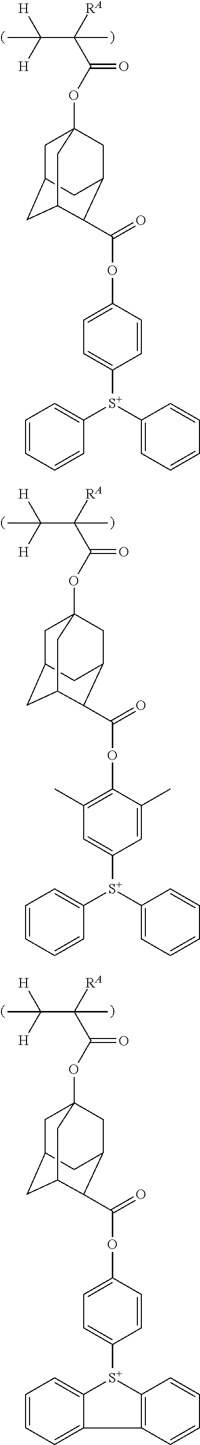

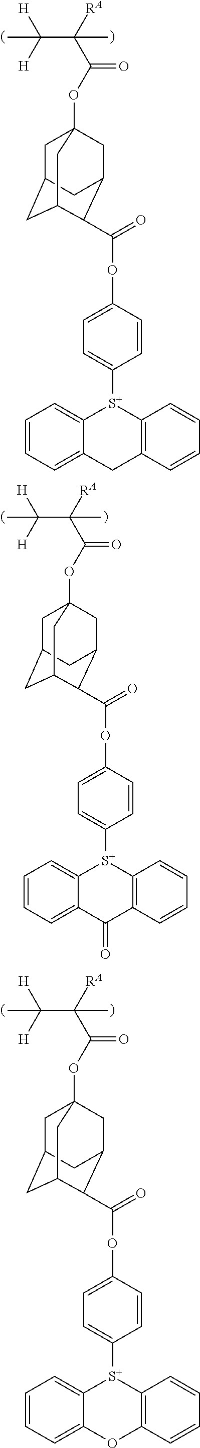

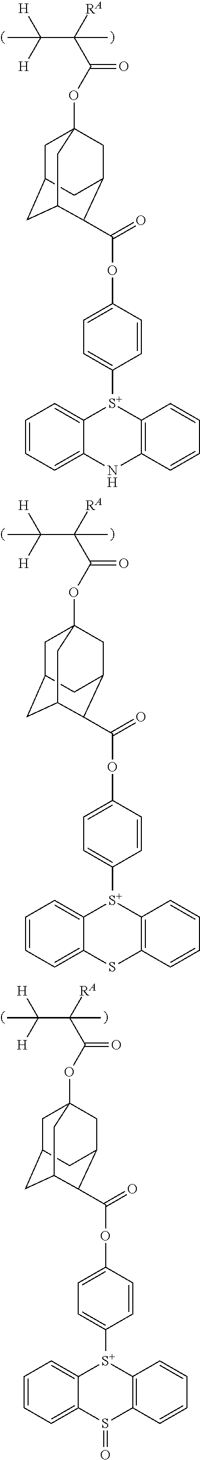

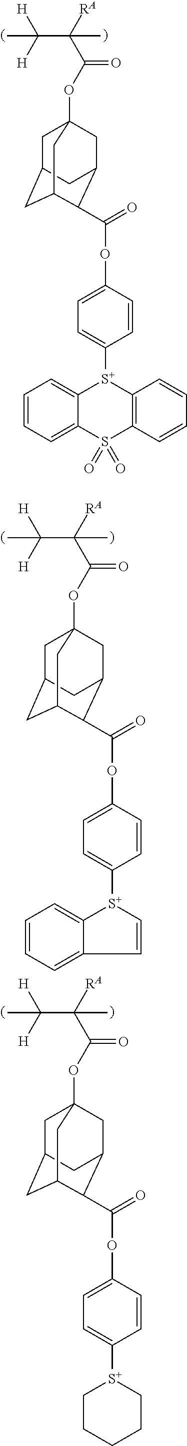

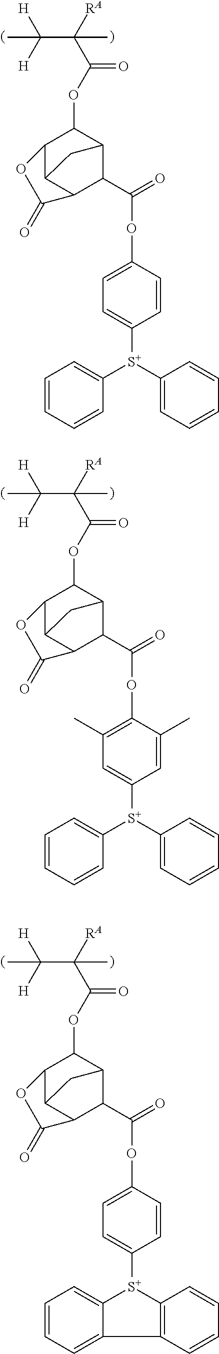

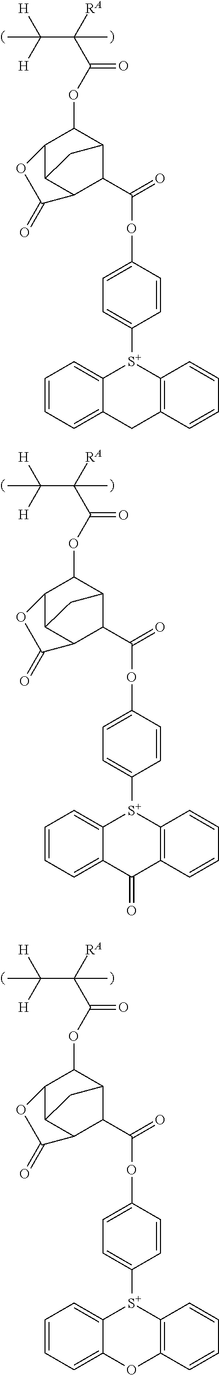

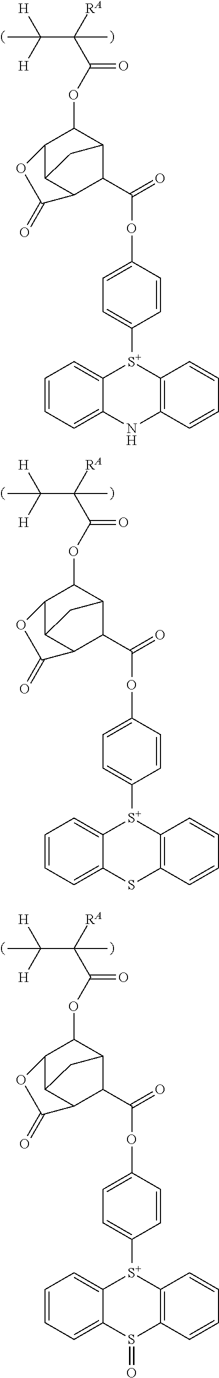

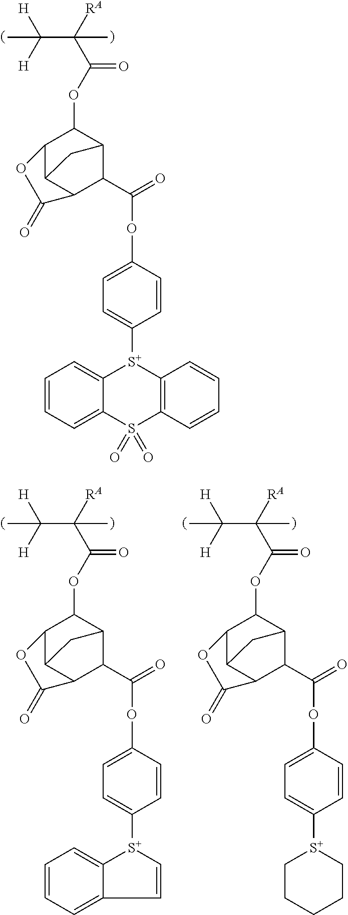

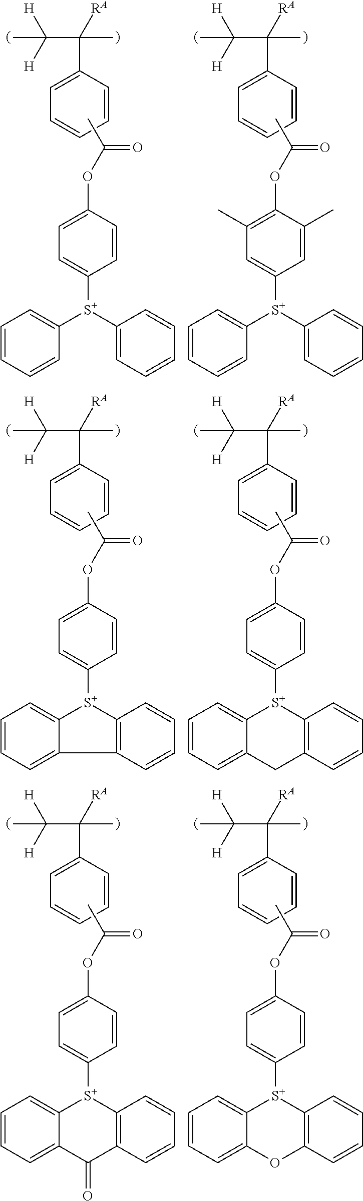

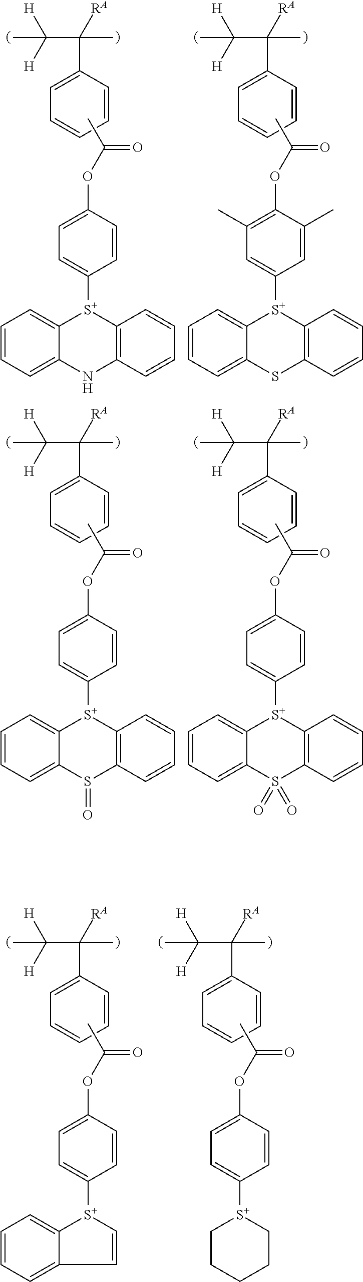

M⁻ in the formula (C1) is a non-nucleophilic counter ion. Examples of the non-nucleophilic counter ion include those listed in paragraphs [0236] to [0290] described later.

In the formula (C2), L¹ is a single bond, an ether bond, an ester bond, a carbonyl group, a sulfonic acid ester bond, a carbonate bond or a carbamate bond. Among these, an ether bond, an ester bond, and a carbonyl group are preferable in view of the synthesis, and an ester bond and a carbonyl group are more preferable.

In the formula (C2), Rf¹ and Rf² independently represent a fluorine atom or a fluorinated alkyl group having 1 to 6 carbon atoms. Among them, Rf¹ and Rf² are each preferably a fluorine atom in order to increase the acid intensity of the generated acid. Rf³ and Rf⁴ independently represent a hydrogen atom, a fluorine atom, or a fluorinated alkyl group having 1 to 6 carbon atoms. Among these, at least one of Rf³ and Rf⁴ is preferably a trifluoromethyl group in order to improve the solvent solubility.

In the formula (C2), c is an integer of 0 to 3, and preferably 1.

Examples of the anions in the repeating unit represented by the formula (C2) include, but are not limited thereto. In the following formulae, R^{A} is as defined above.

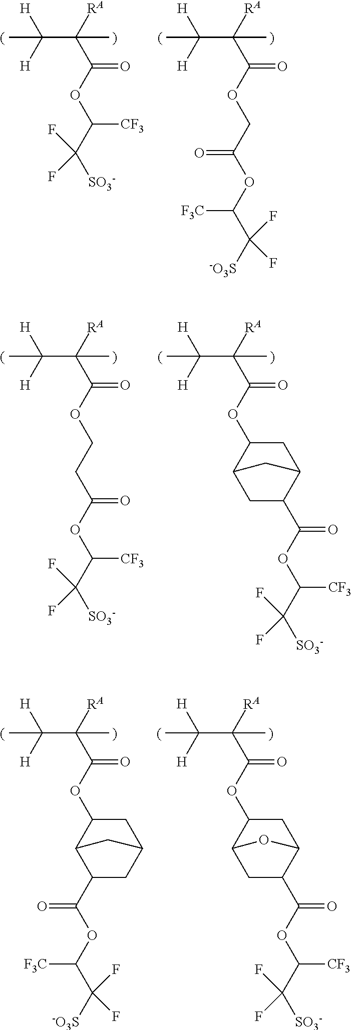

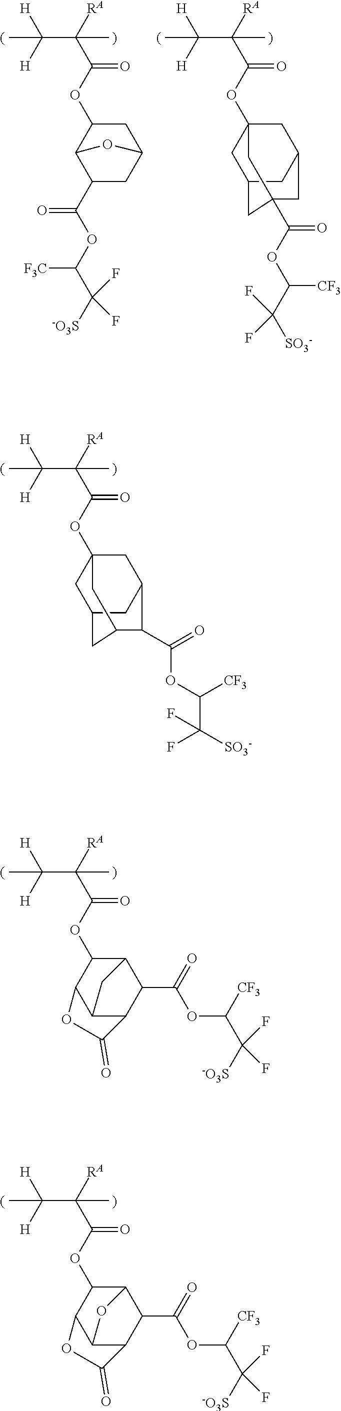

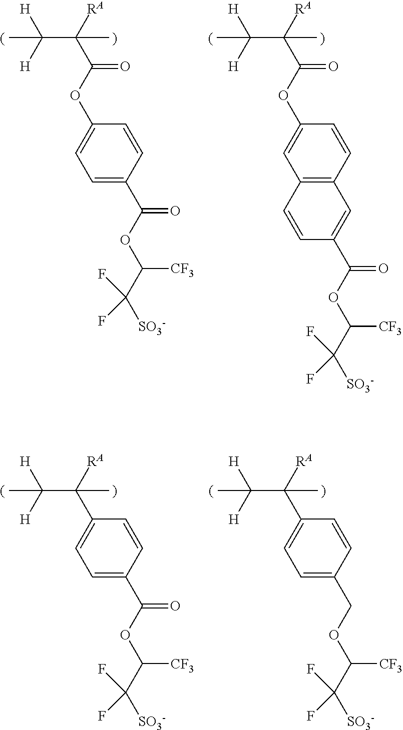

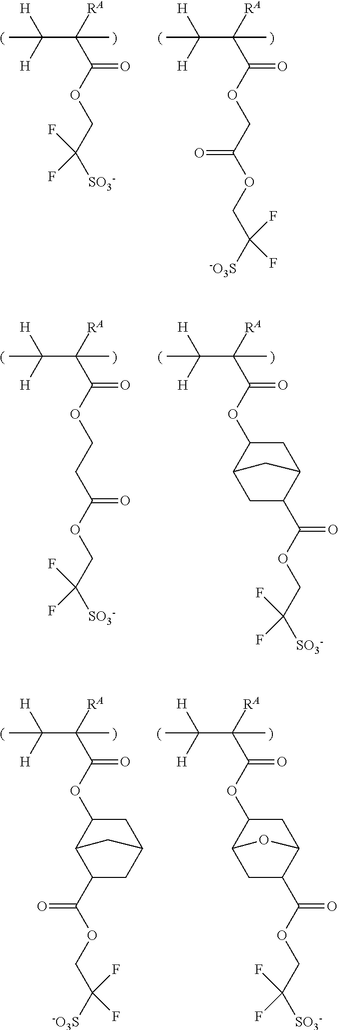

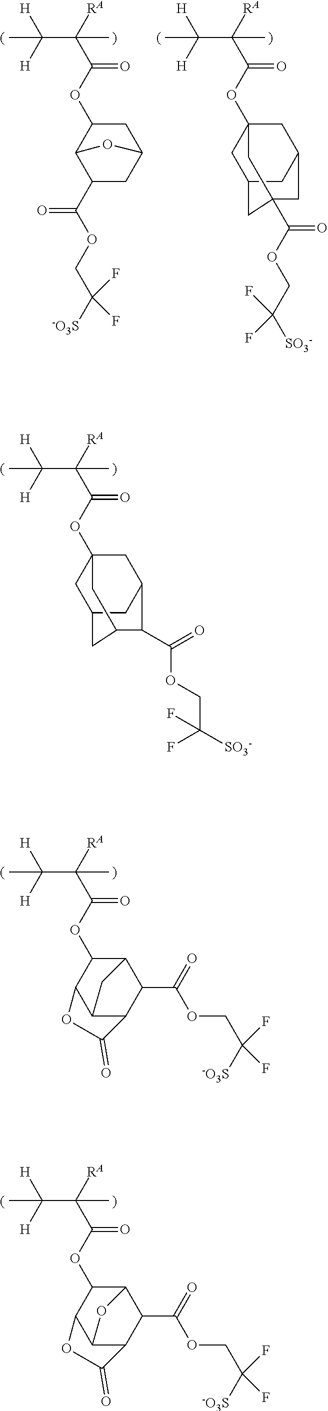

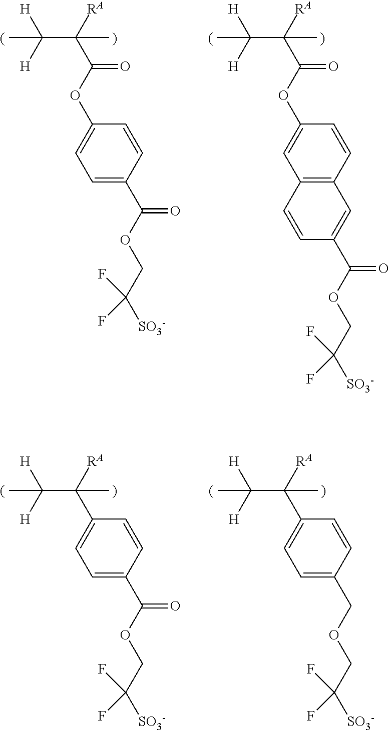

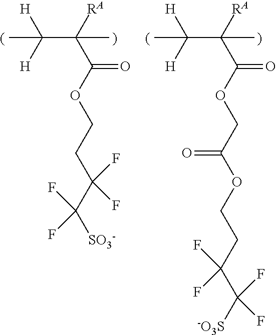

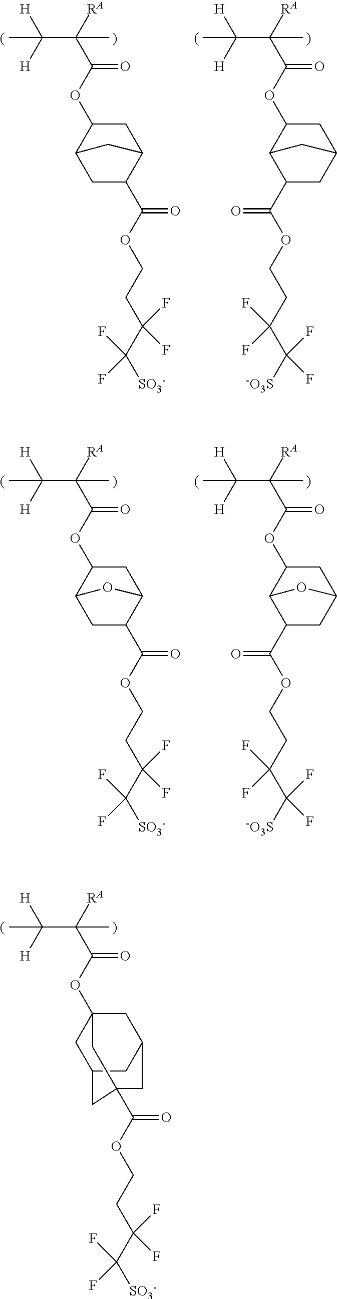

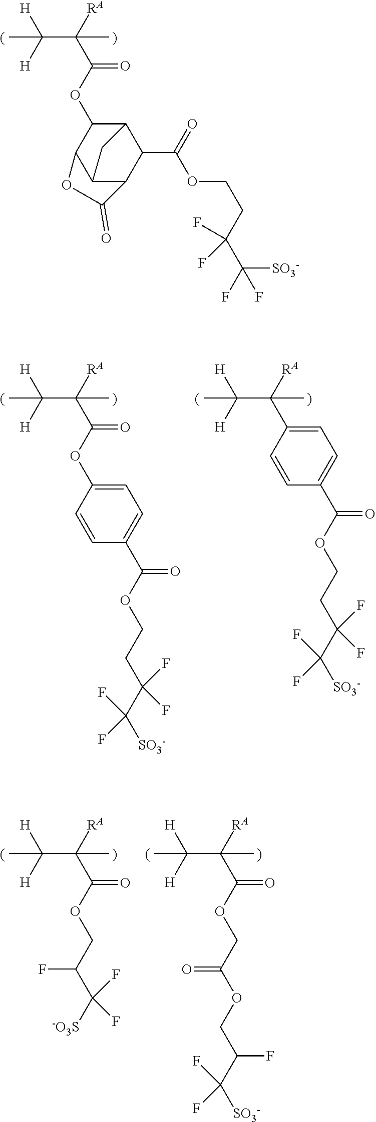

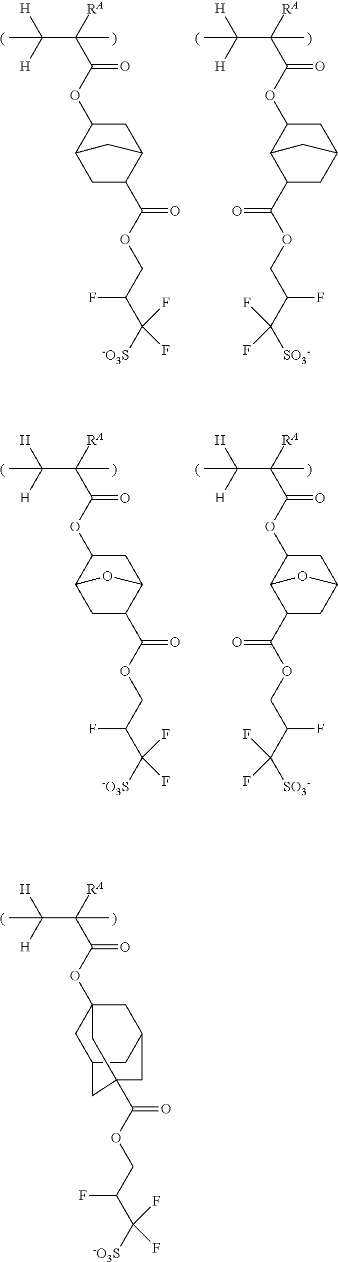

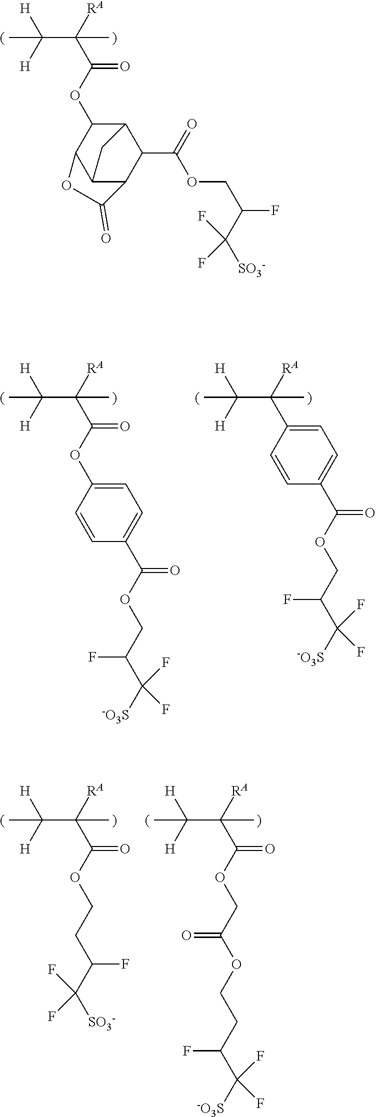

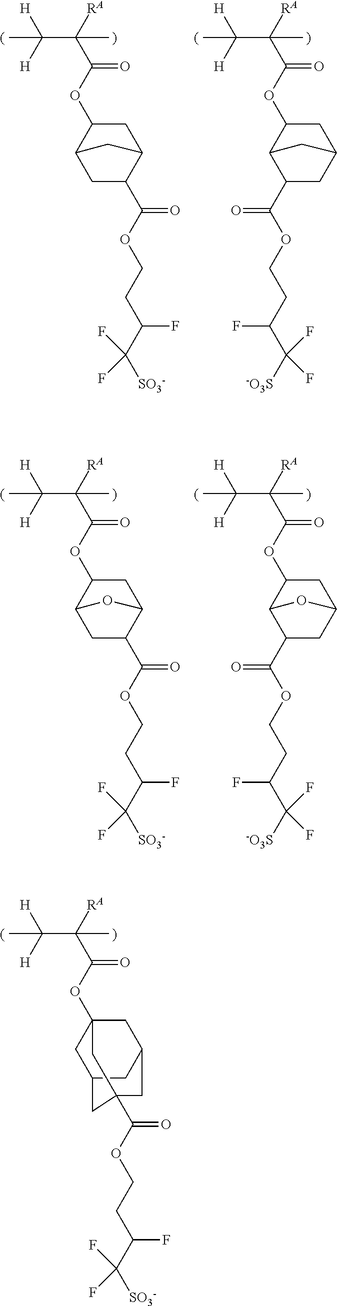

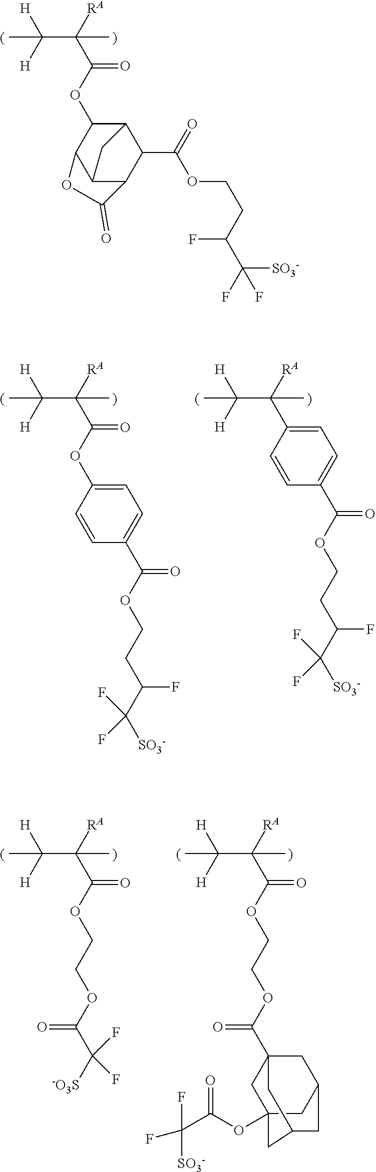

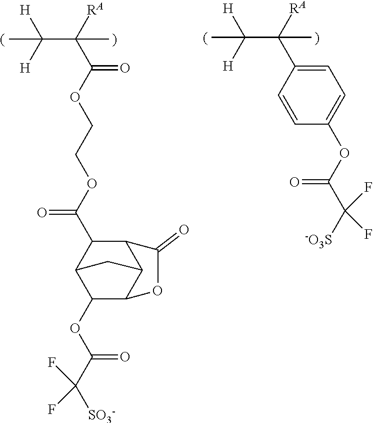

In the formula (c3), L¹ is a single bond, an ether bond, an ester bond, a carbonyl group, a sulfonic acid ester bond, a carbonate bond or a carbamate bond. Among these, an ether bond, an ester bond, and a carbonyl group are preferable in view of the synthesis, and an ester bond and a carbonyl group are more preferable.

In the formula (c3), Rf⁵ and Rf⁶ independently represent a hydrogen atom, a fluorine atom, or a fluorinated alkyl group having 1 to 6 carbon atoms. Among these, at least one of Rf⁵ and Rf⁶ is preferably a trifluoromethyl group in order to improve the solvent solubility.

In the formula (c3), c is an integer of 0 to 3, and preferably 1.

Examples of the anions in the repeating unit represented by the formula (c3) include, but are not limited thereto. In the following formulae, R^{A} is as defined above.

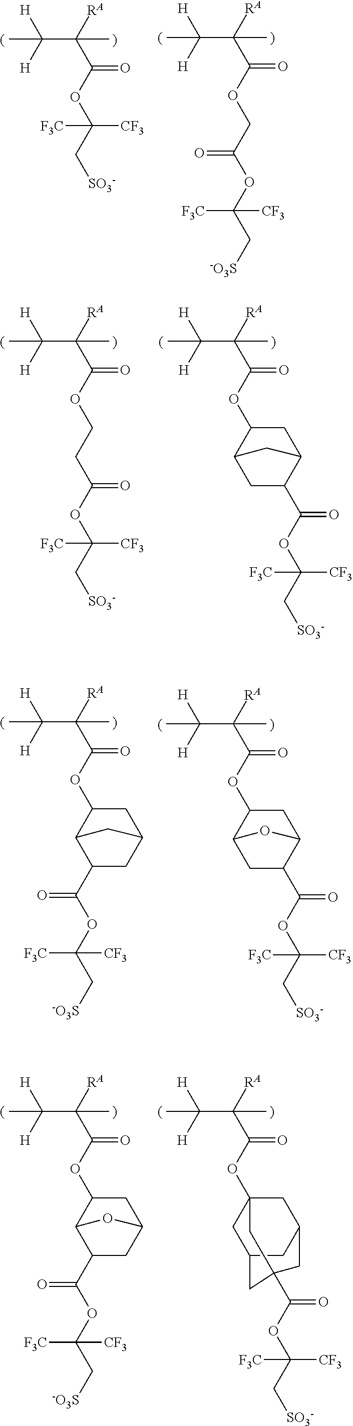

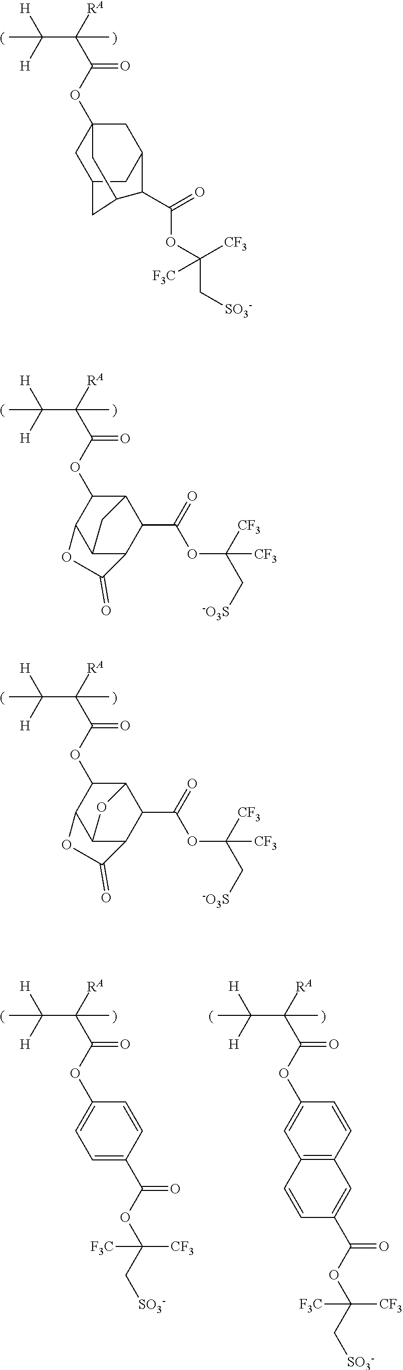

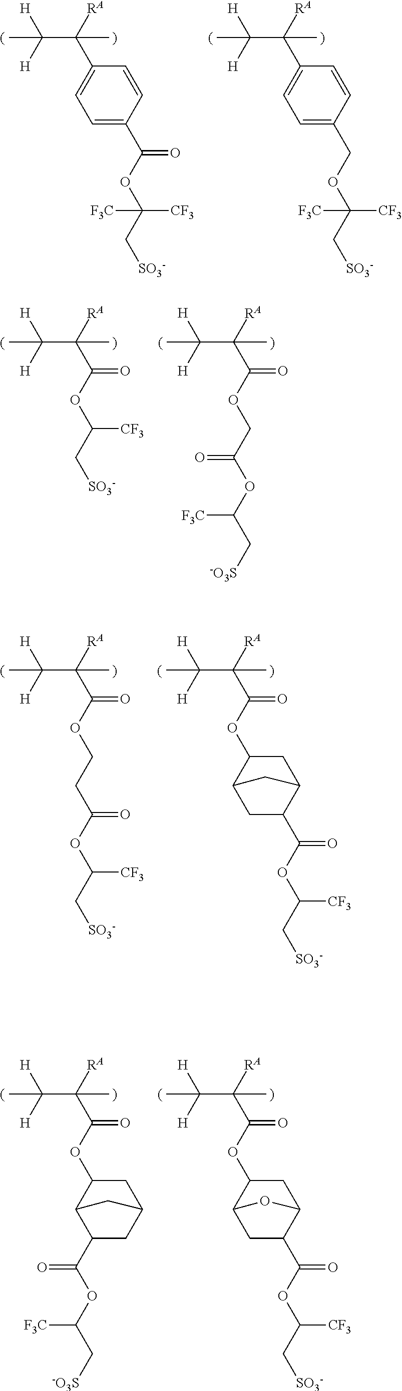

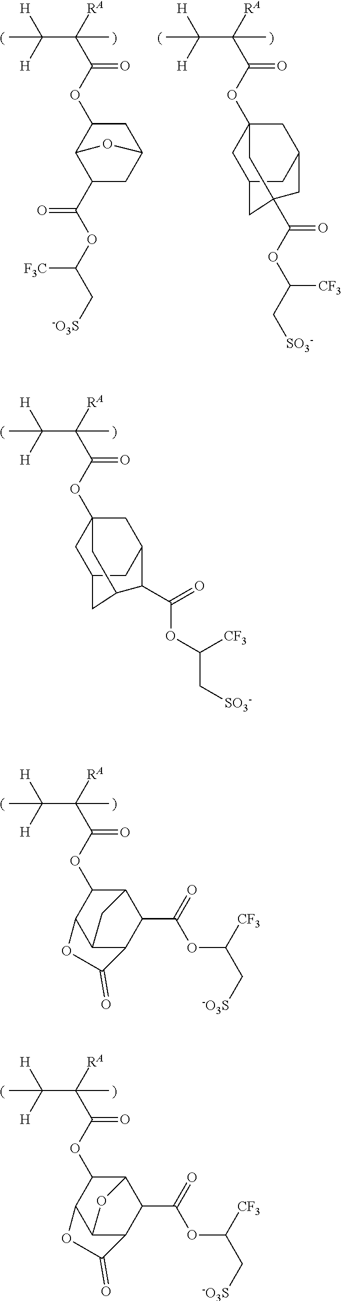

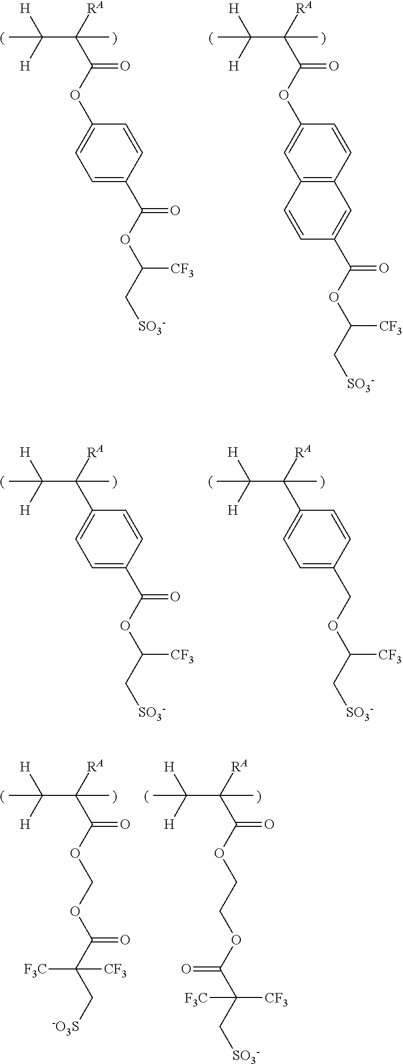

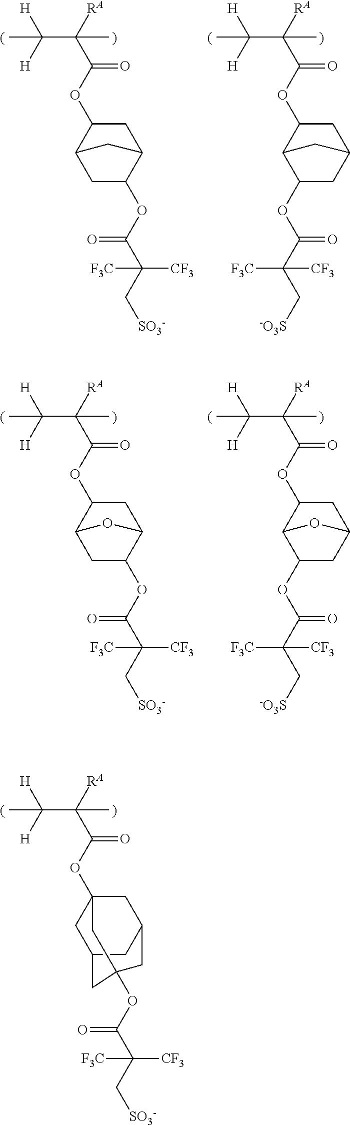

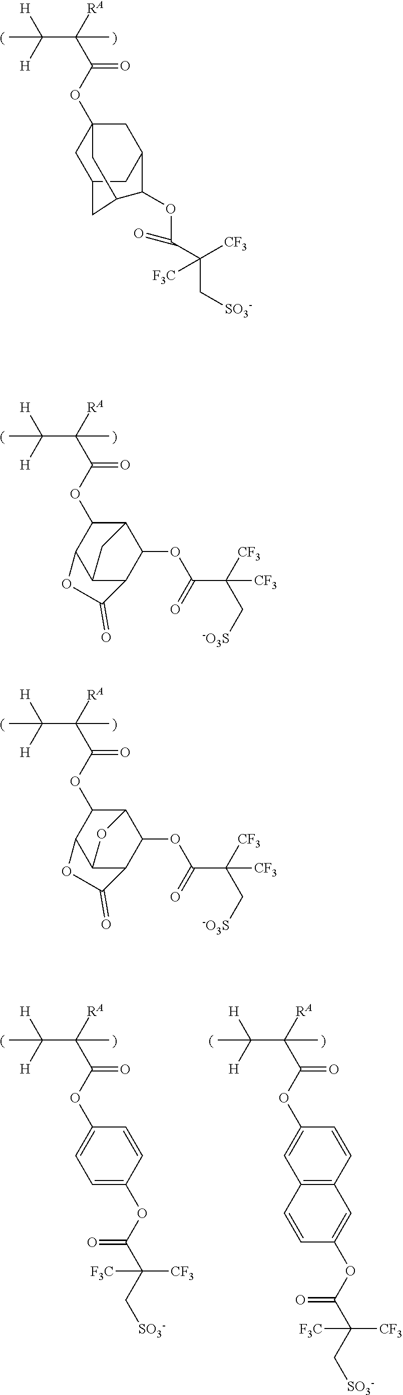

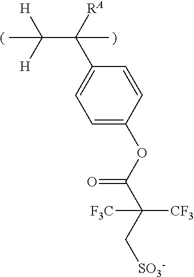

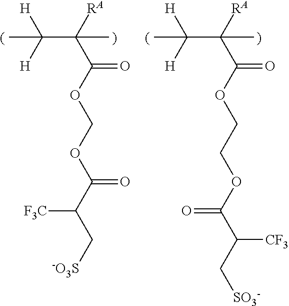

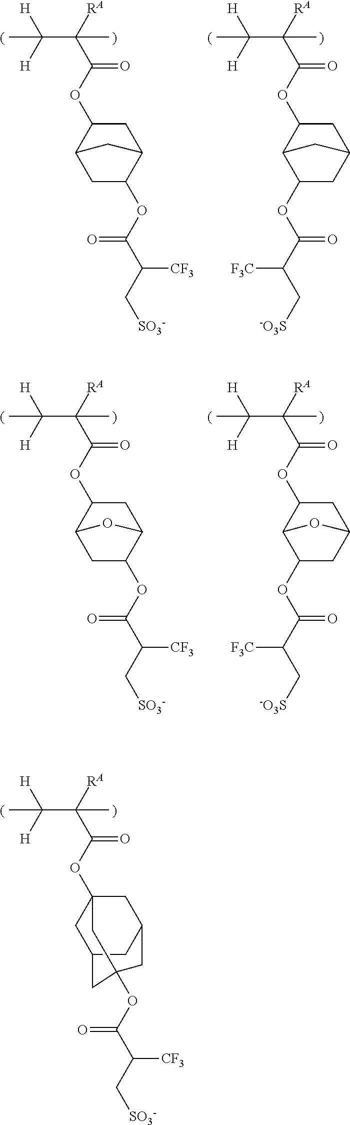

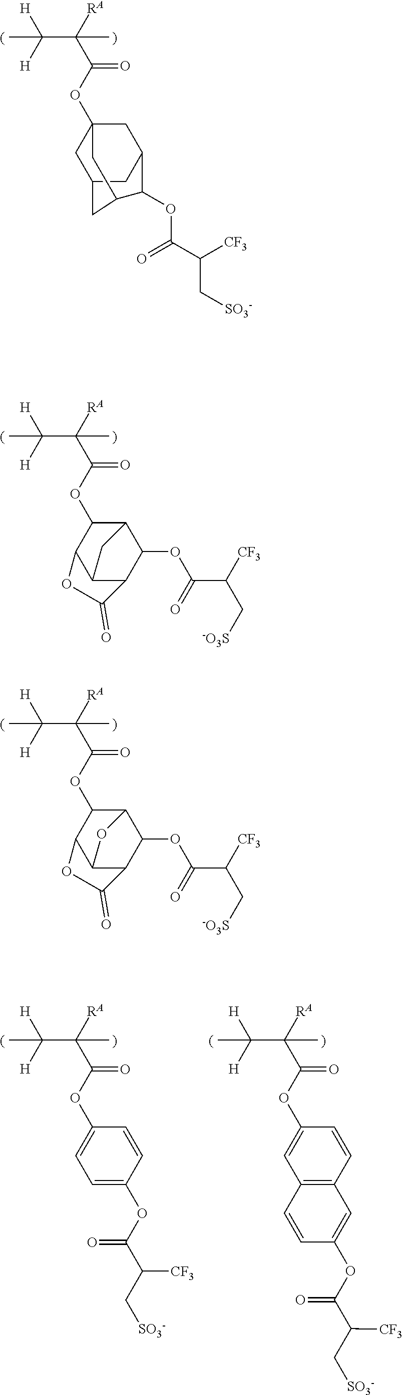

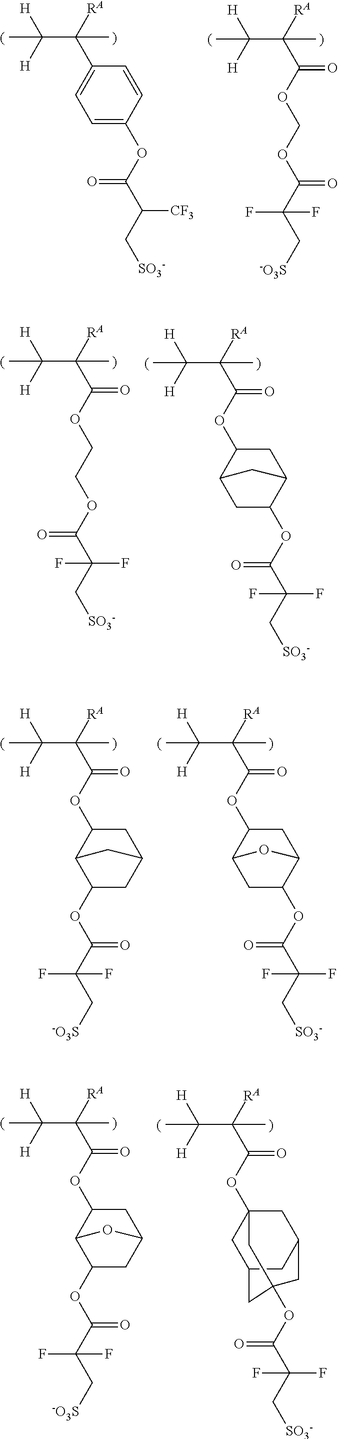

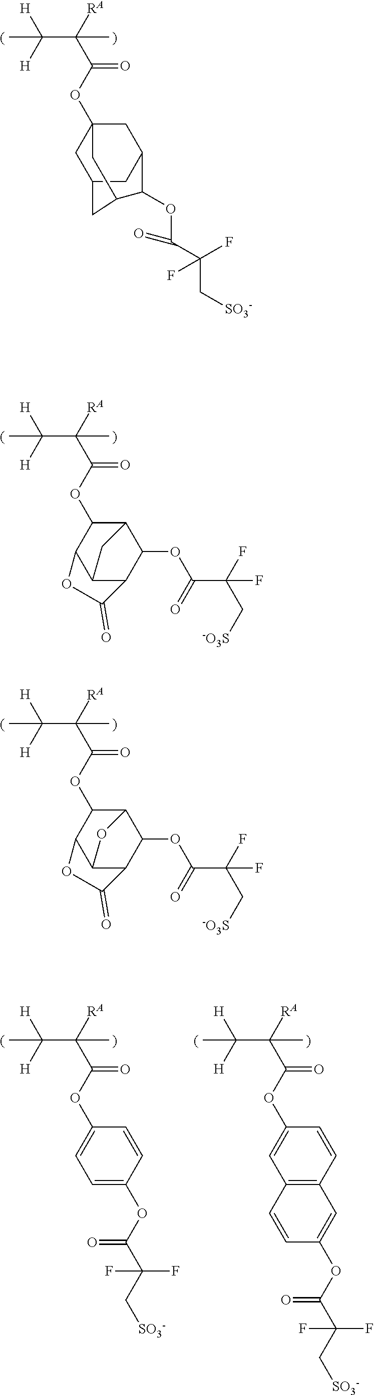

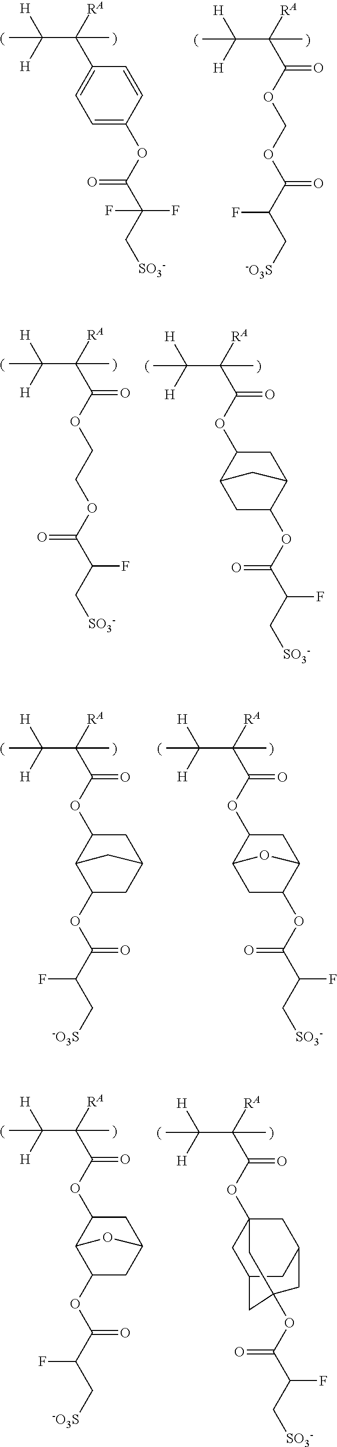

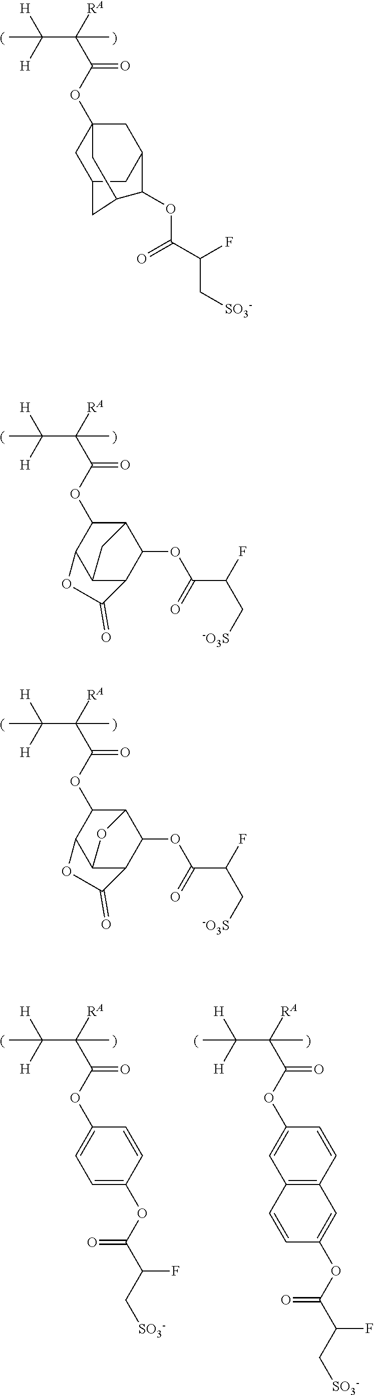

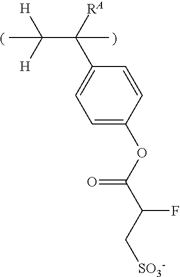

Examples of the anions in the repeating unit represented by the formula (C4) include, but are not limited thereto. In the following formulae, R^{A} is as defined above.

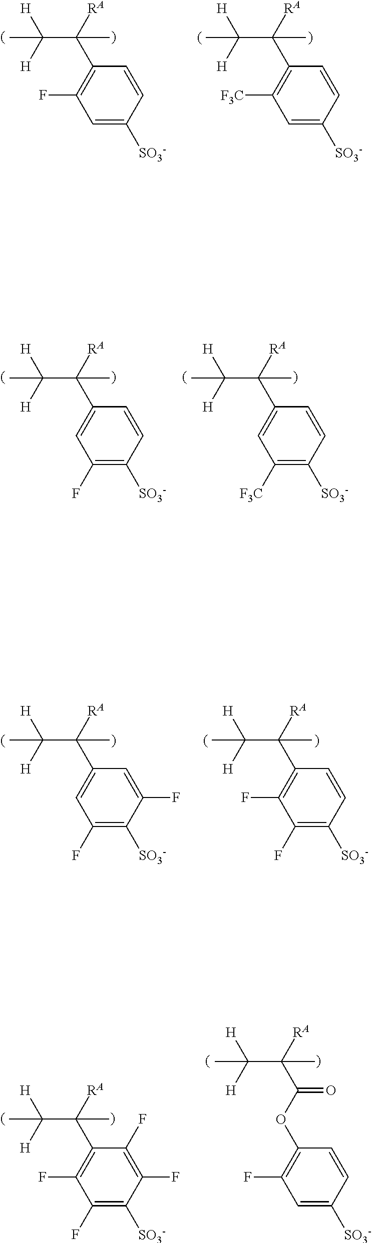

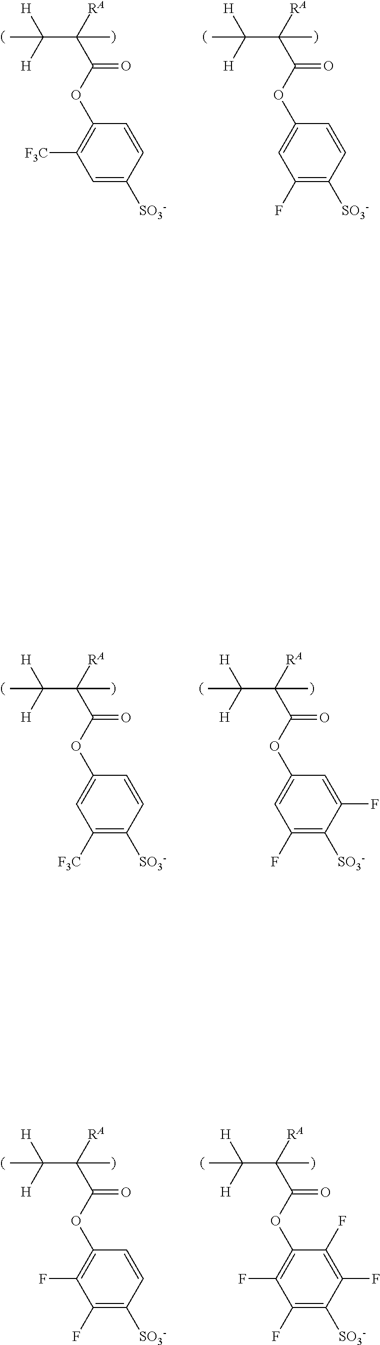

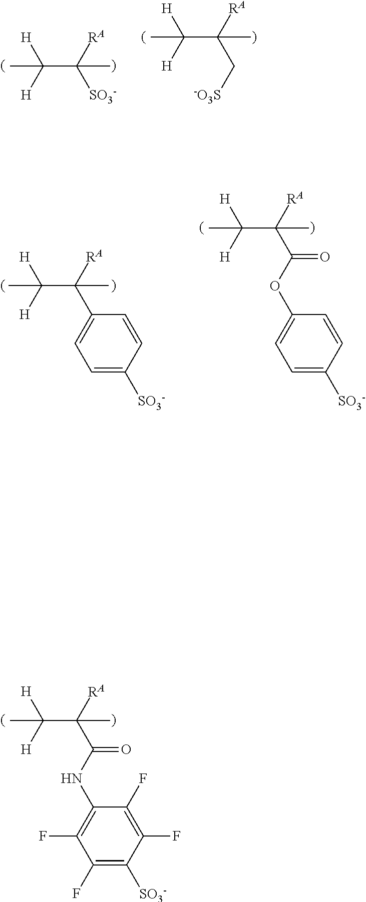

In the formulae (C2) to (C4), A⁺ represents an onium cation. Examples of the onium cation include an ammonium cation, a sulfonium cation, and an iodonium cation. Among these, a sulfonium cation and an iodonium cation are preferable, and a sulfonium cation represented by the general formula (cation-1) and an iodonium cation represented by the general formula (cation-2) shown below are more preferable.

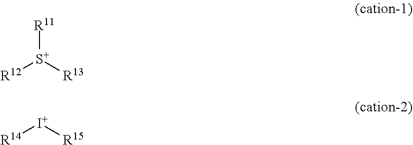

It is preferable that in the formulae (cation-1) and (cation-2), R¹¹ to R¹⁵ independently represent a hydrocarbyl group having 1 to 30 carbon atoms which may contain a heteroatom. The hydrocarbyl group may be any of a saturated, unsaturated, linear, branched, or cyclic hydrocarbyl group. Specific examples thereof include alkyl groups such as a methyl group, an ethyl group, a n-propyl group, an isopropyl group, a n-butyl group, and a tert-butyl group; cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups such as a vinyl group, an allyl group, a propenyl group, a butenyl group, and a hexenyl group; cyclic unsaturated hydrocarbyl groups such as a cyclohexenyl group; aryl groups such as a phenyl group, a naphthyl group, and a thienyl group; aralkyl groups such as a benzyl group, a 1-phenylethyl group, and a 2-phenylethyl group; groups obtained by combining these groups; and the like. Among them, aryl groups are preferable. Also included are the foregoing hydrocarbyl groups in which at least one hydrogen atom is optionally substituted with a group containing a heteroatom such as an oxygen atom, a sulfur atom, and a nitrogen atom; further optionally, the carbon atoms of these groups may be intervened by a group containing a heteroatom such as an oxygen atom, a sulfur atom and a nitrogen atom, so that the groups may contain a hydroxy group, a cyano group, a carbonyl group, an ether bond, an ester bond, a sulfonic acid ester bond, a carbonate bond, a lactone ring, a sultone ring, a carboxylic acid anhydride, a haloalkyl group or the like.

Further, R¹¹ and R¹² may also be bonded to each other to form a ring with a sulfur atom to which R¹¹ and R¹² are bonded. Examples of the sulfonium cation represented by the formula (cation-1) include those represented by the following formulae,

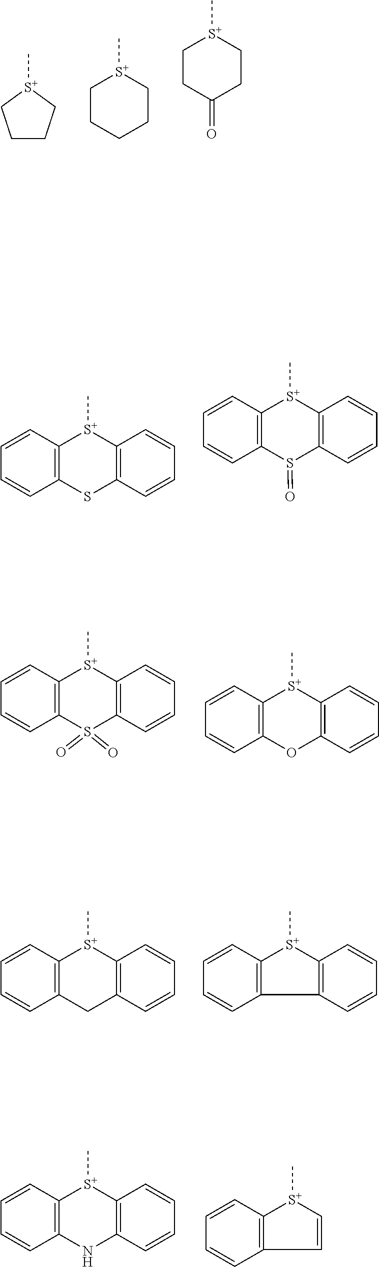

wherein the dashed line represents a bonding arm to R¹³.

Examples of the sulfonium cation represented by the formula (cation-1) include, but are not limited thereto.

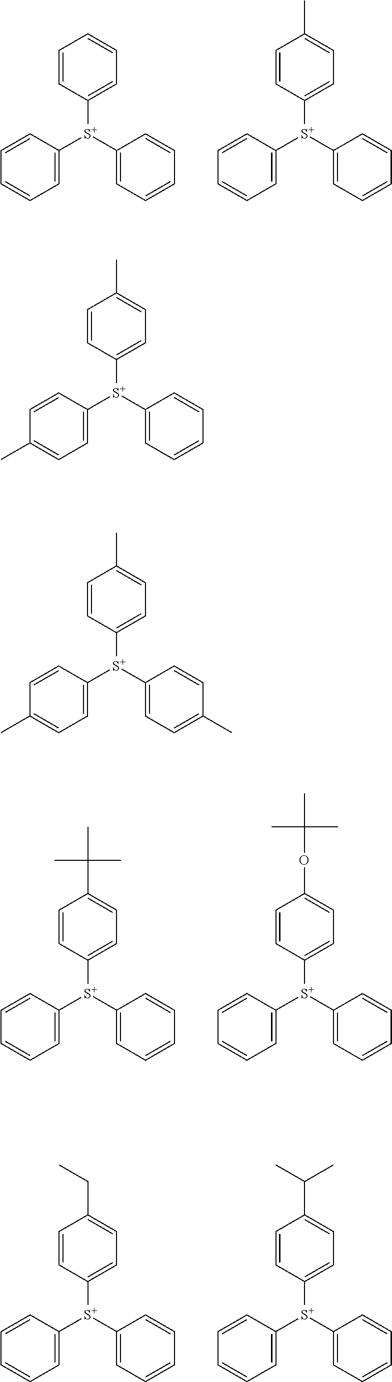

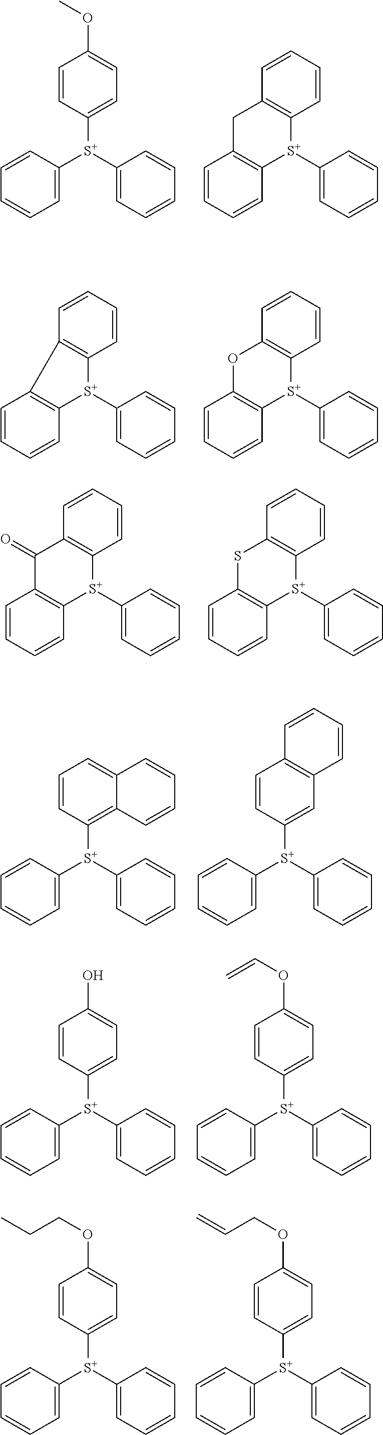

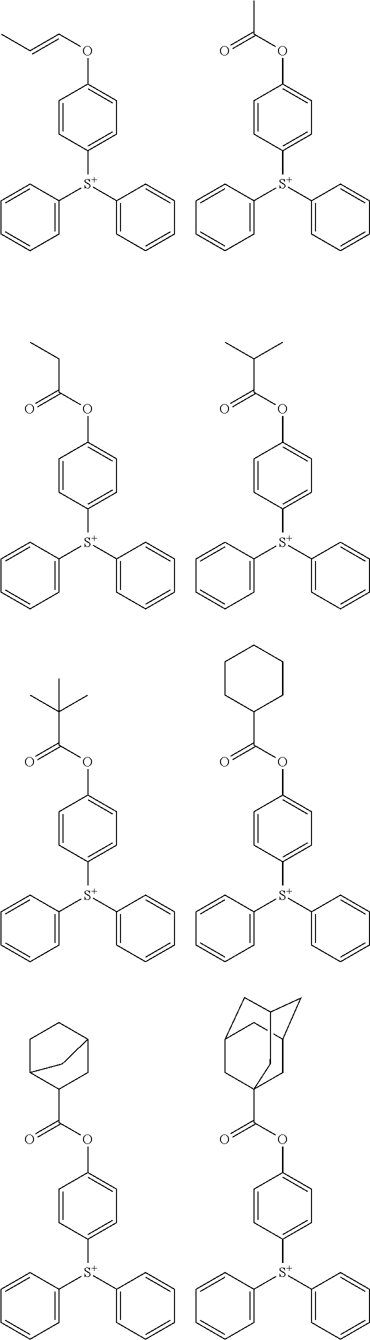

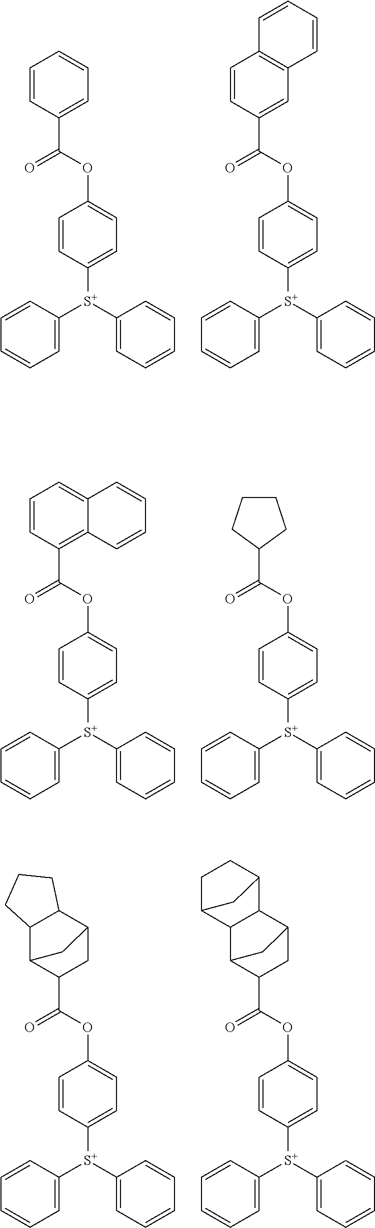 [SMILES: O=C(Oc1ccc([S+](c2ccccc2)c2ccccc2)cc1)c1ccccc1 ; O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21]

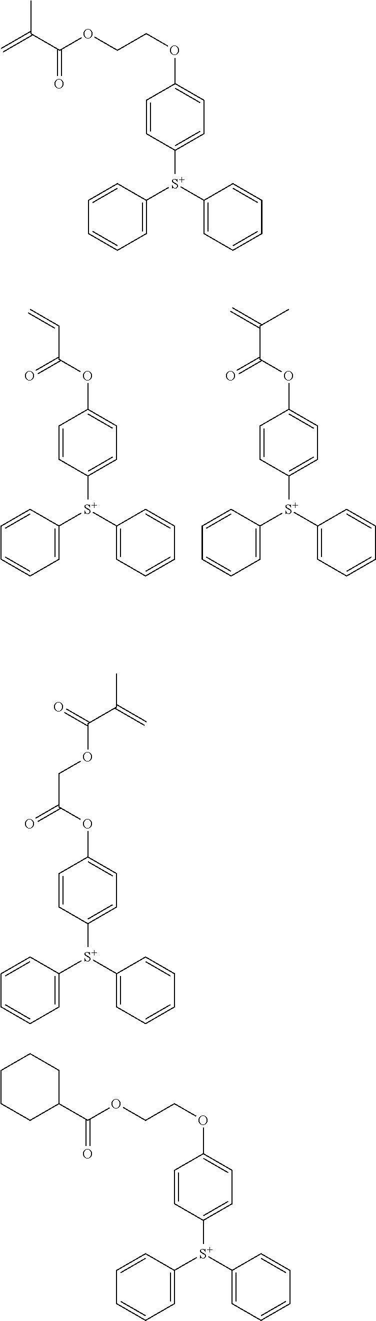 [SMILES: C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1]

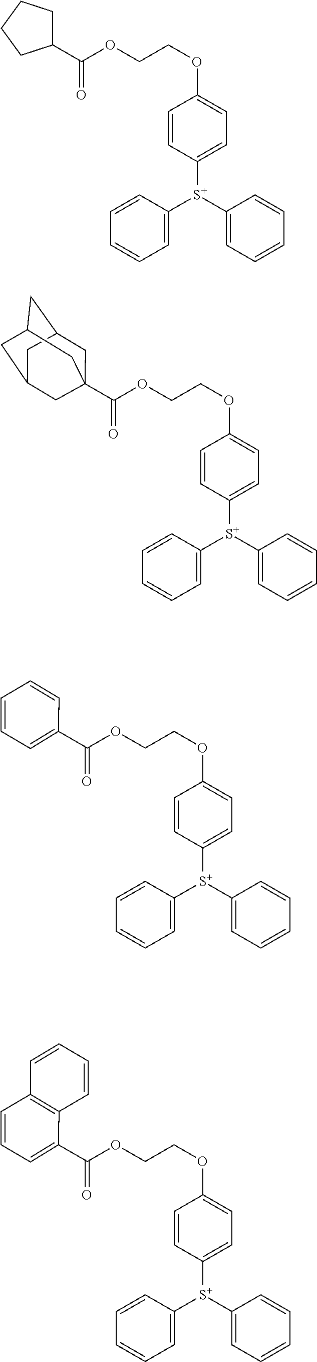 [SMILES: O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12]

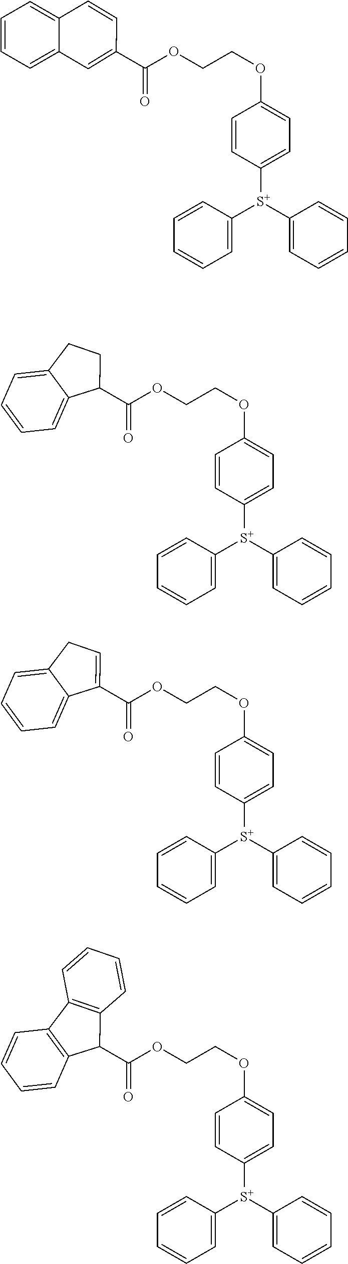 [SMILES: O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21]

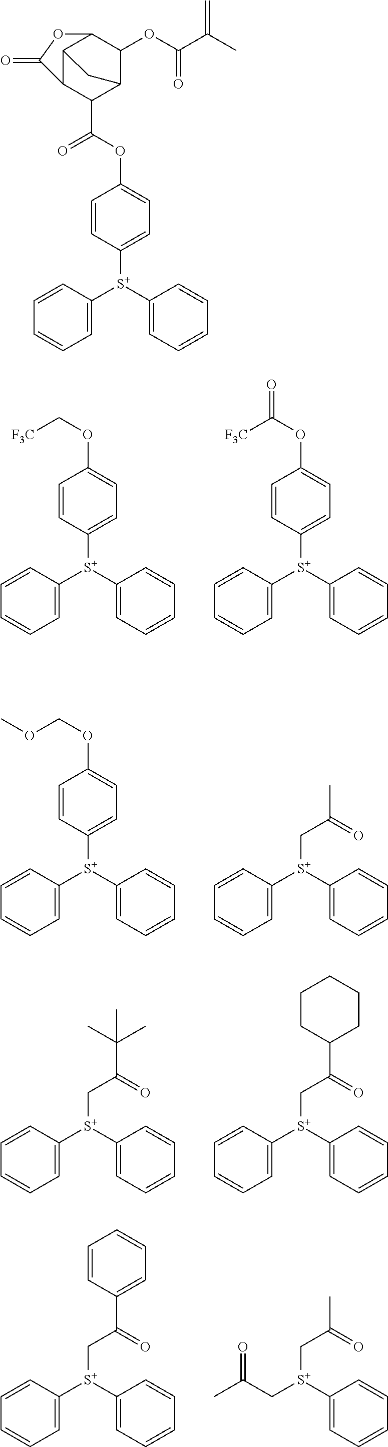

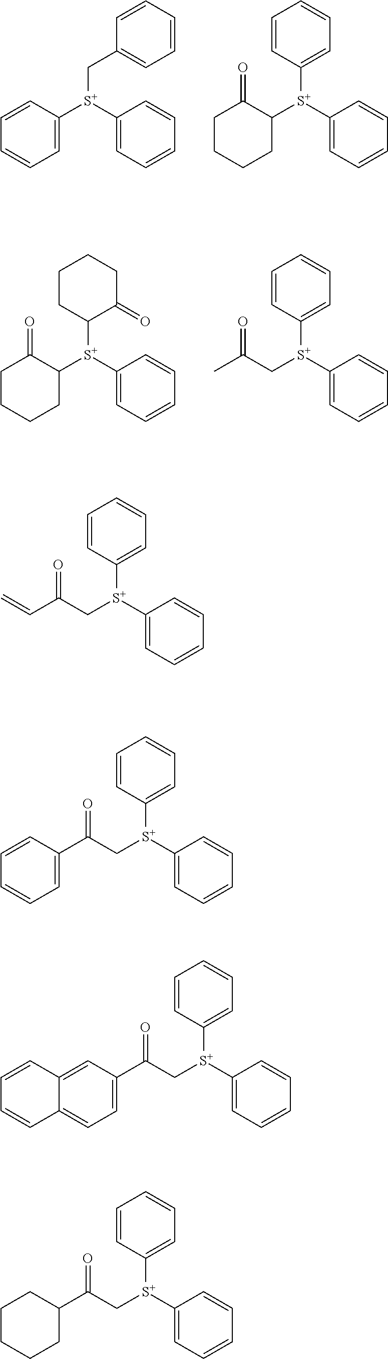

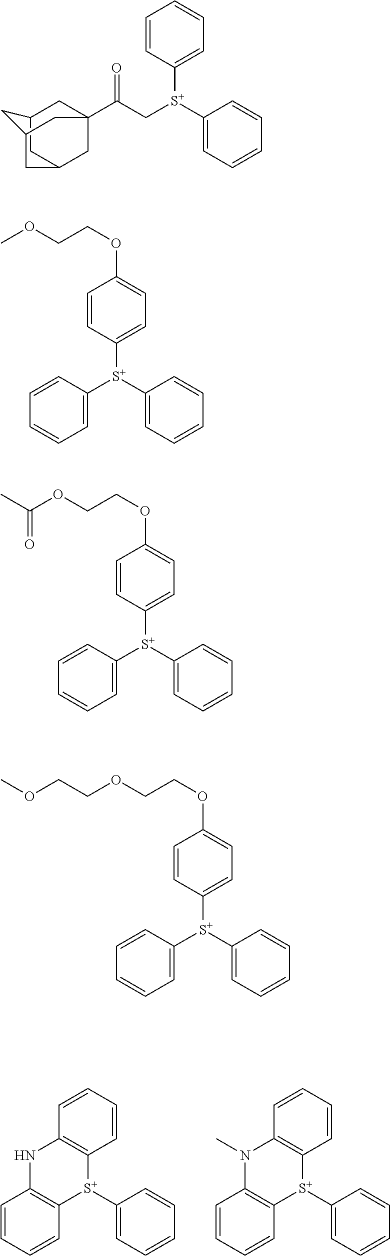 [SMILES: O=C(C[S+](c1ccccc1)c1ccccc1)C12CC3CC(CC(C3)C1)C2 ; COCCOc1ccc([S+](c2ccccc2)c2ccccc2)cc1 ; CC(=O)OCCOc1ccc([S+](c2ccccc2)c2ccccc2)cc1 ; COCCOCCOc1ccc([S+](c2ccccc2)c2ccccc2)cc1 ; c1ccc([S+]2c3ccccc3Nc3ccccc32)cc1 ; CN1c2ccccc2[S+](c2ccccc2)c2ccccc21]

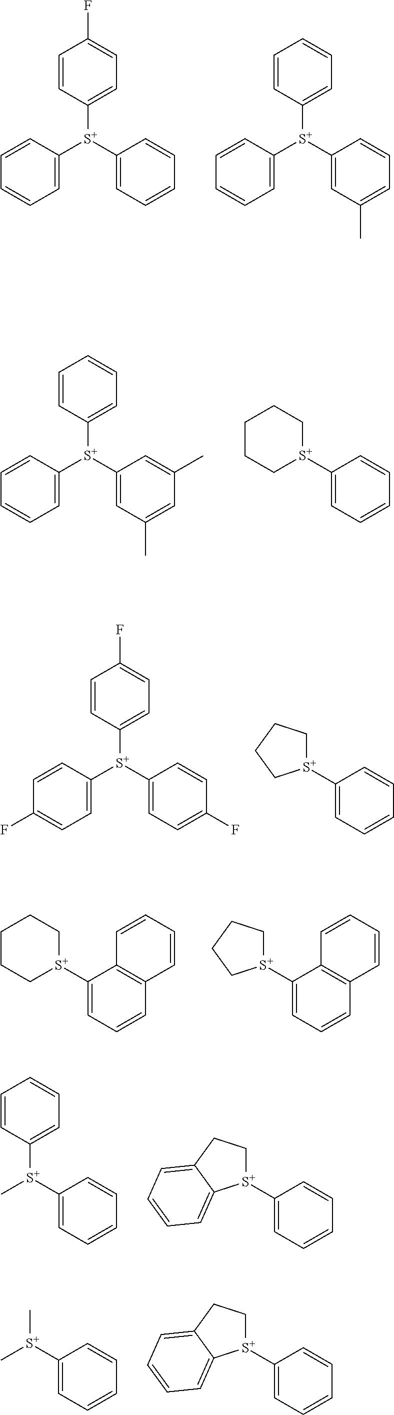

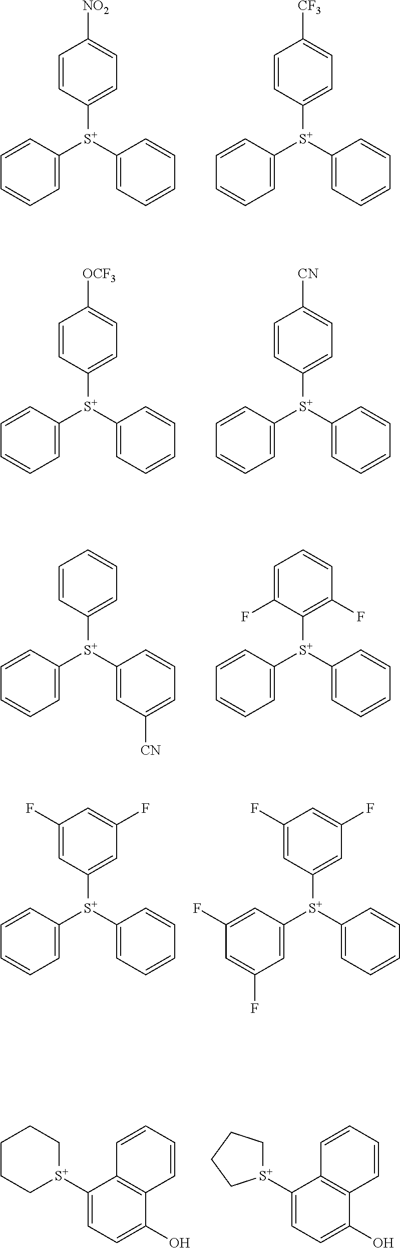

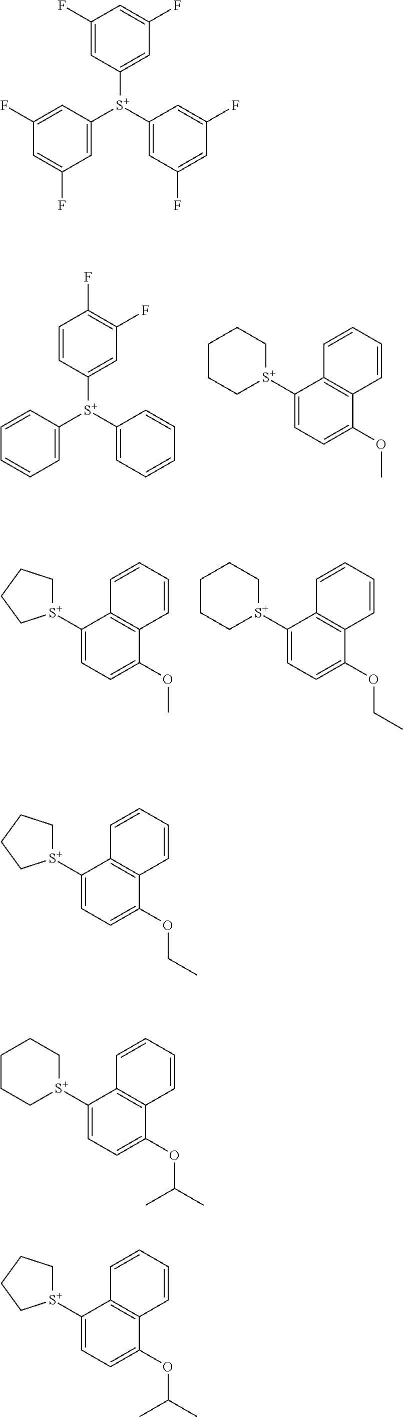

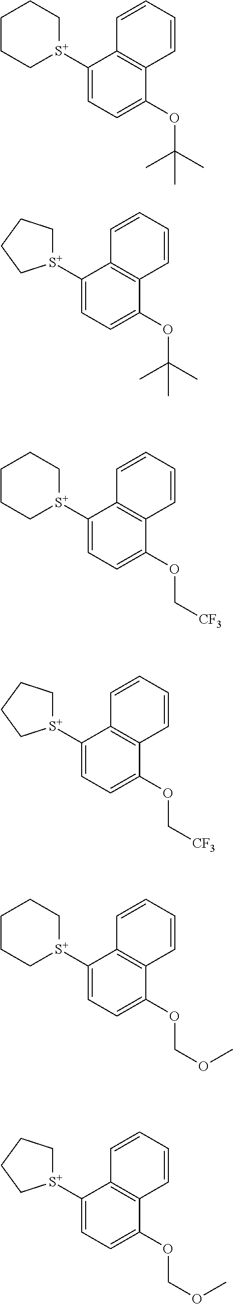 [SMILES: CC(C)(C)Oc1ccc([S+]2CCCCC2)c2ccccc12 ; CC(C)(C)Oc1ccc([S+]2CCCC2)c2ccccc12 ; FC(F)(F)COc1ccc([S+]2CCCCC2)c2ccccc12 ; FC(F)(F)COc1ccc([S+]2CCCC2)c2ccccc12 ; COCOc1ccc([S+]2CCCCC2)c2ccccc12 ; COCOc1ccc([S+]2CCCC2)c2ccccc12]

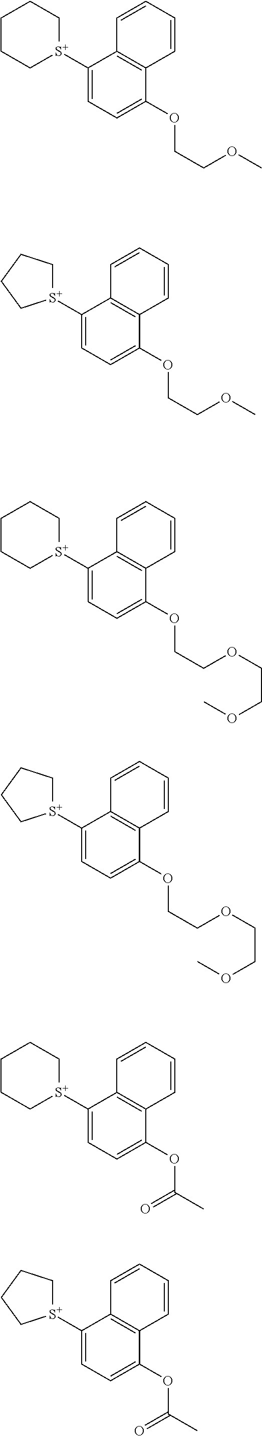 [SMILES: COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12 ; CC(=O)Oc1ccc([S+]2CCCCC2)c2ccccc12 ; CC(=O)Oc1ccc([S+]2CCCC2)c2ccccc12]

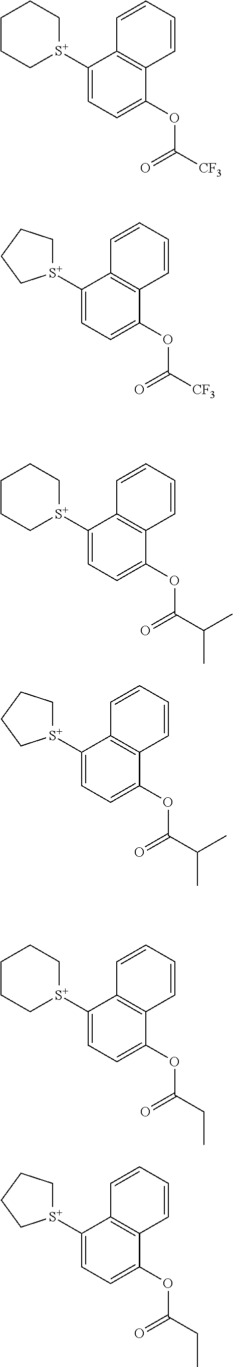 [SMILES: O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12 ; CC(C)C(=O)Oc1ccc([S+]2CCCC2)c2ccccc12 ; CCC(=O)Oc1ccc([S+]2CCCCC2)c2ccccc12 ; CCC(=O)Oc1ccc([S+]2CCCC2)c2ccccc12]

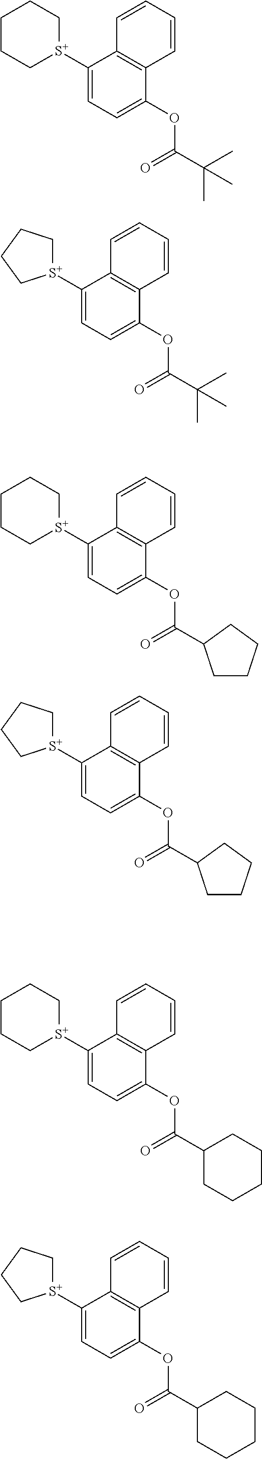 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

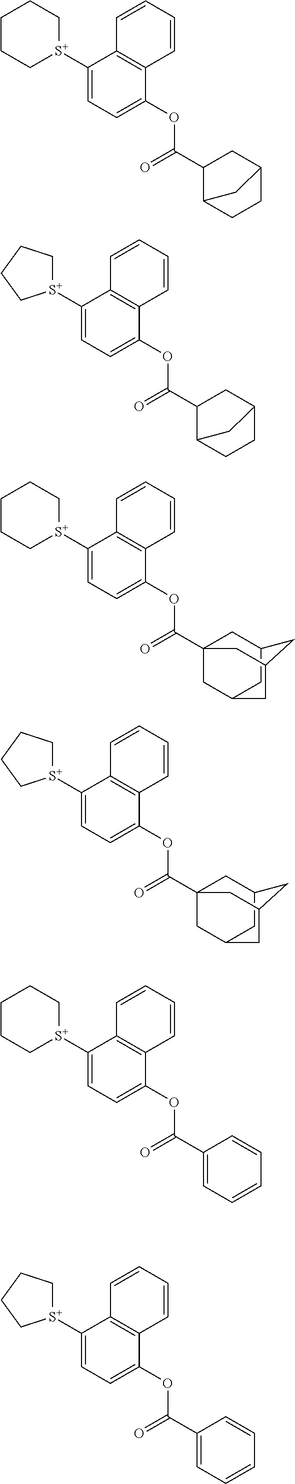 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; O=C(Oc1ccc([S+]2CCCCC2)c2ccccc12)c1ccccc1 ; O=C(Oc1ccc([S+]2CCCC2)c2ccccc12)c1ccccc1]

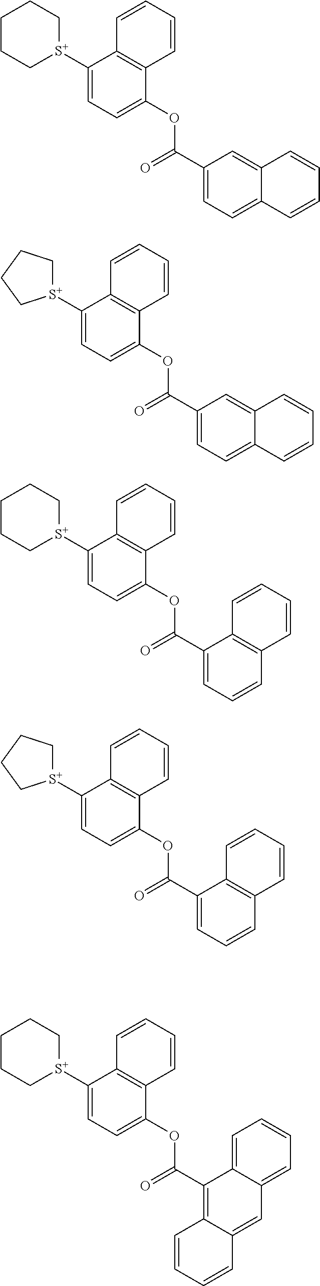 [SMILES: O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12]

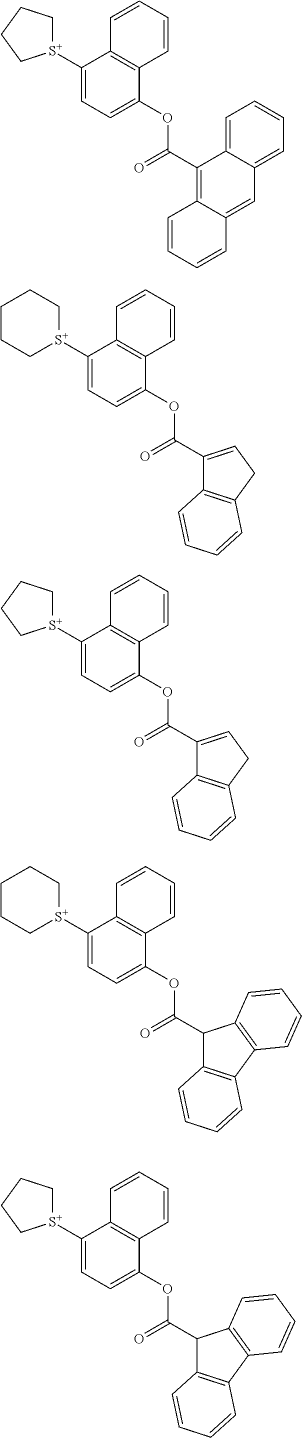 [SMILES: O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21 ; O=C(Oc1ccc([S+]2CCCC2)c2ccccc12)C1c2ccccc2-c2ccccc21]

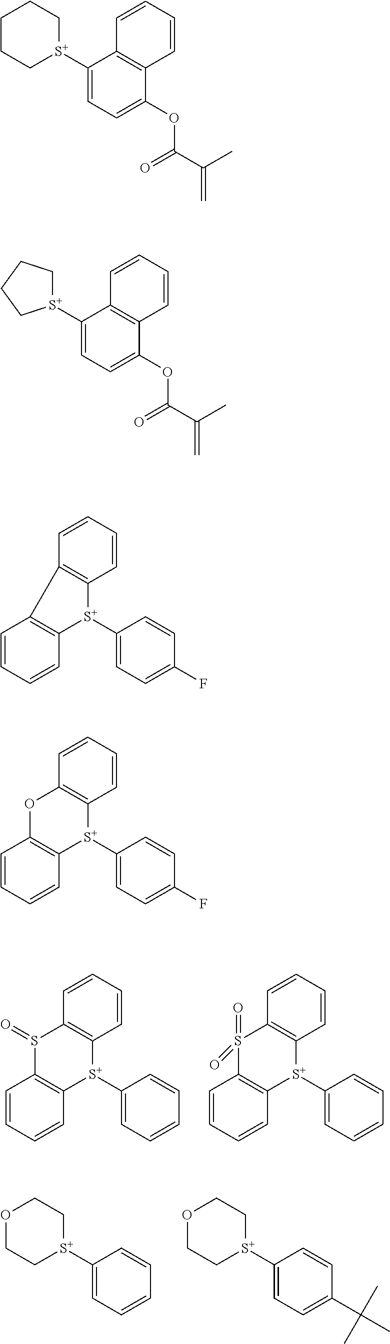

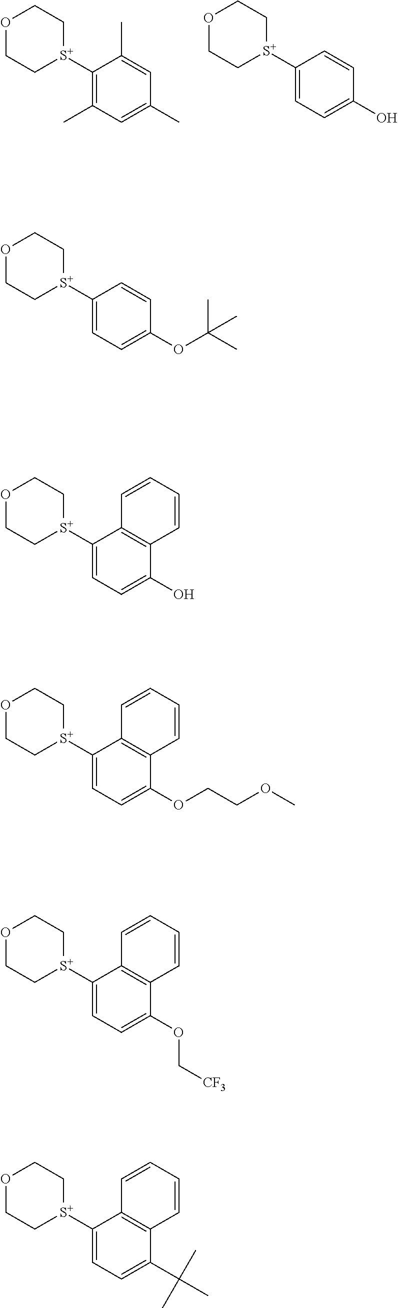

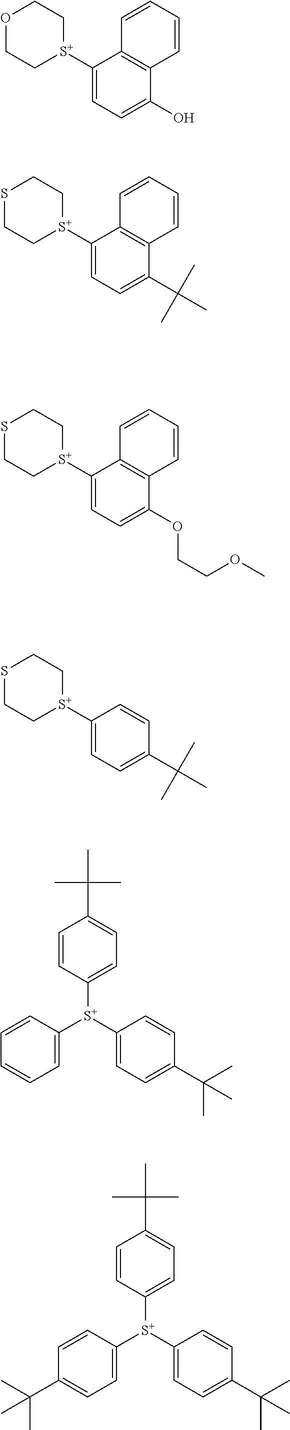

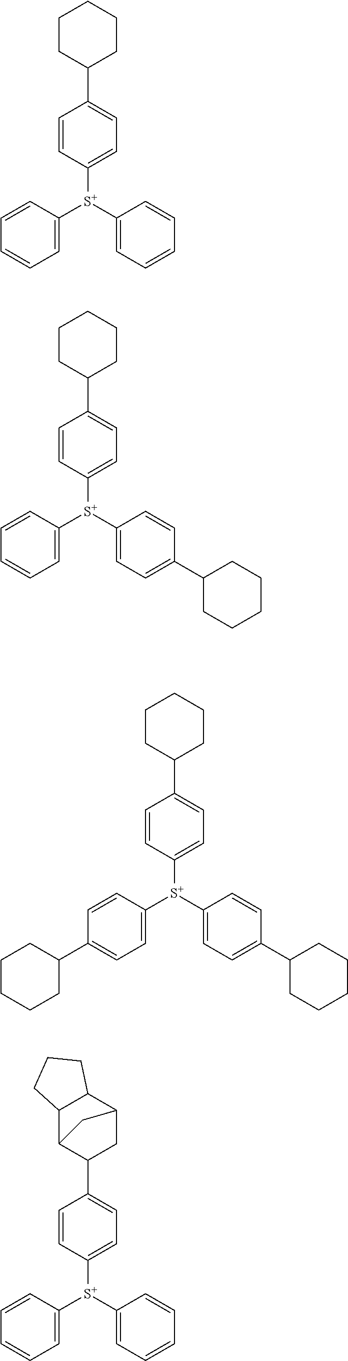 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1]

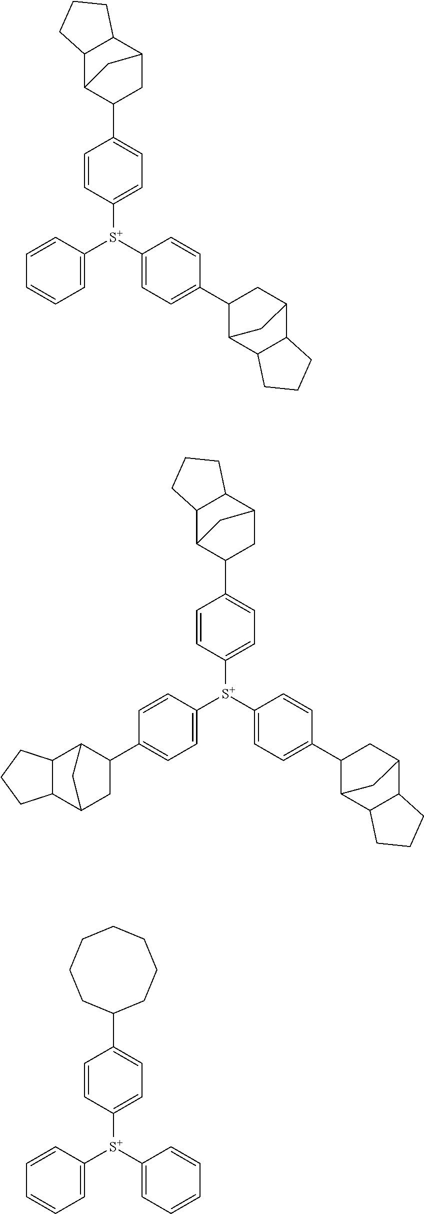 [SMILES: c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

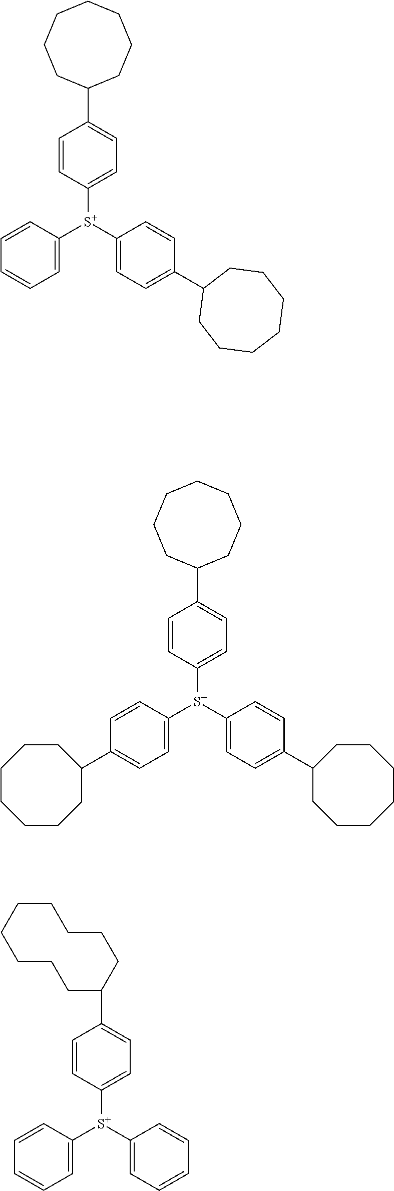 [SMILES: c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

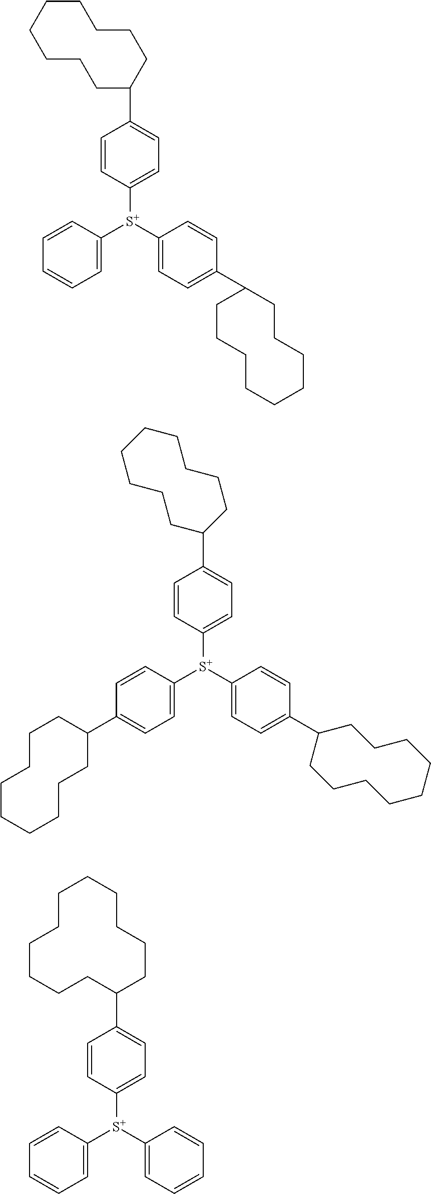 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

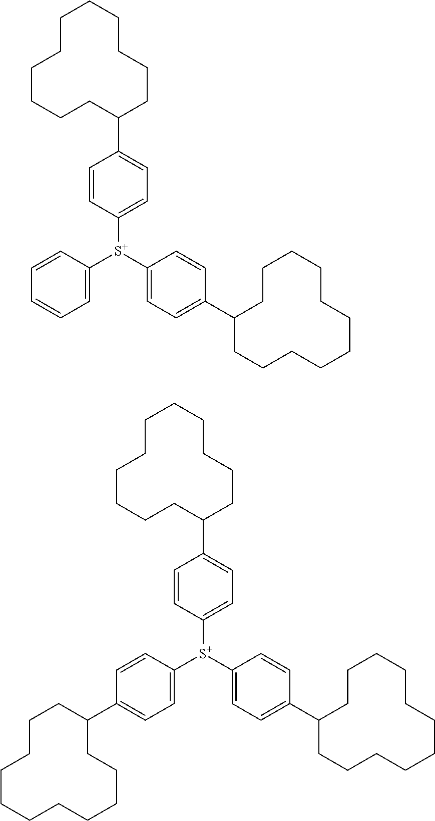 [SMILES: c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

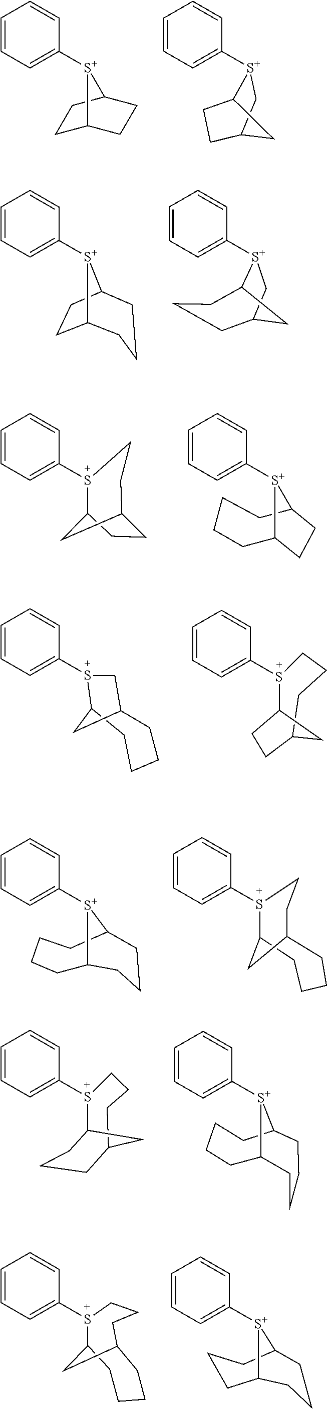

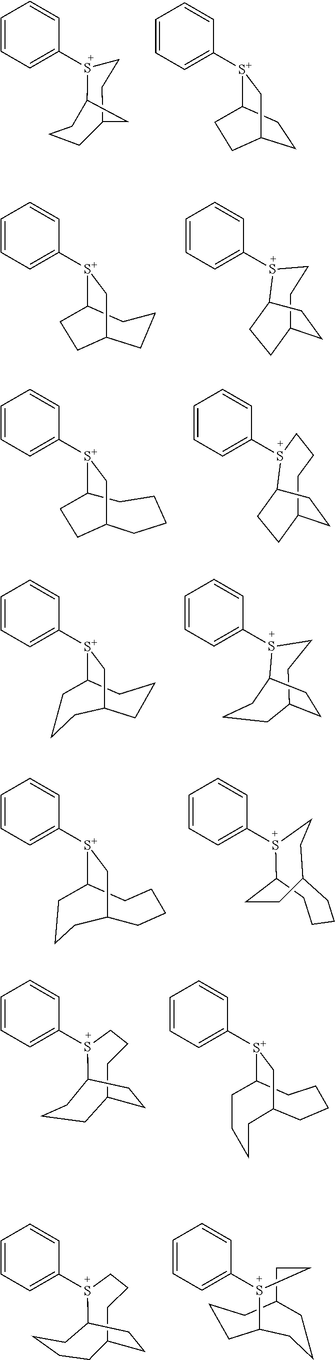

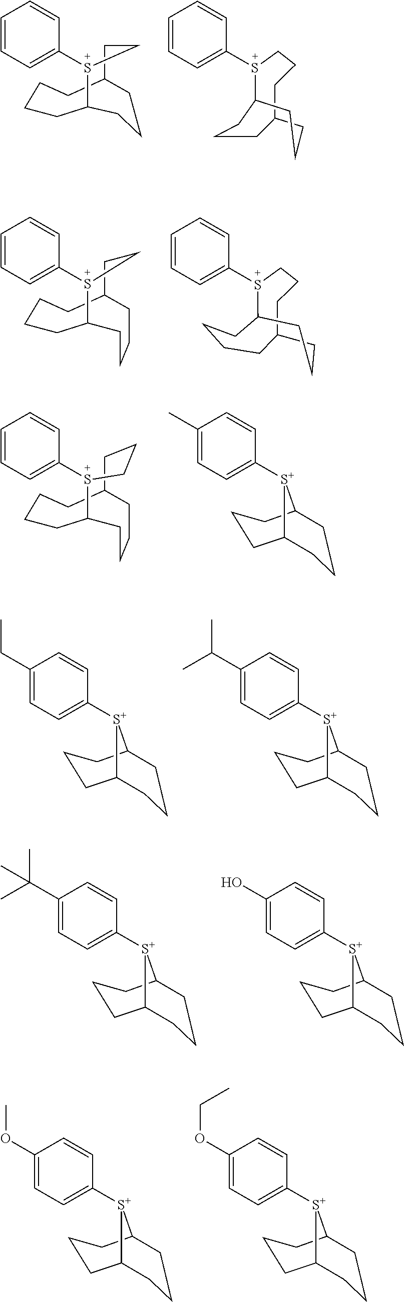

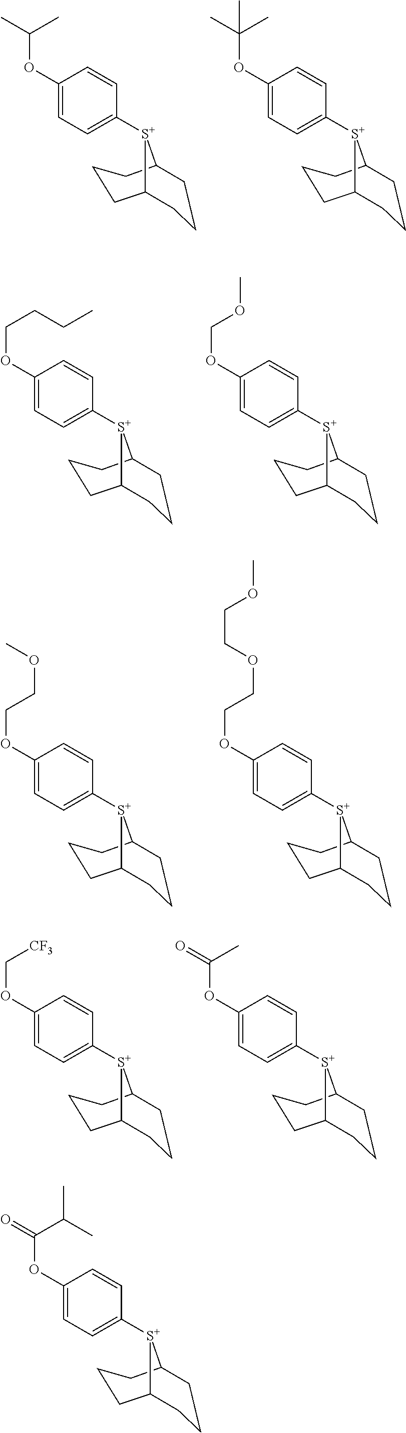

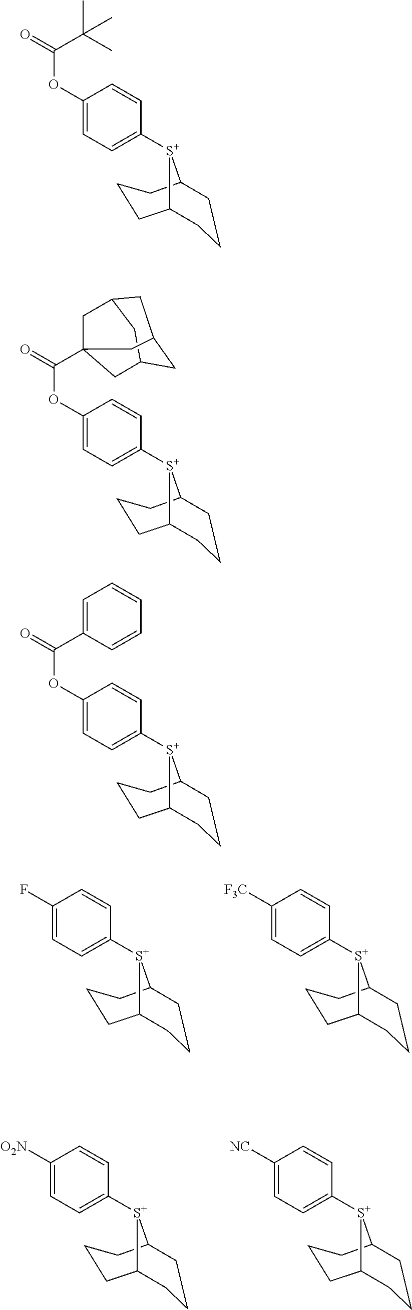

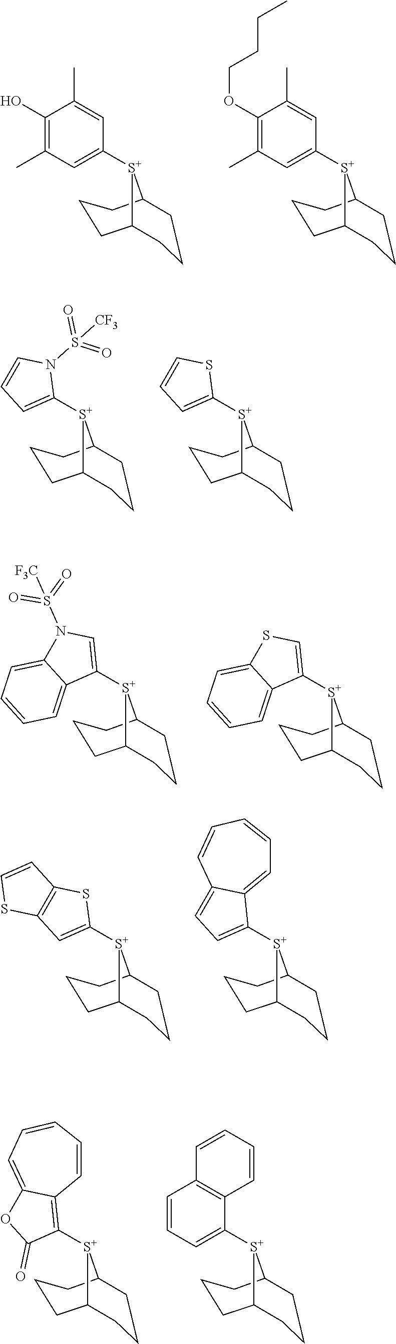

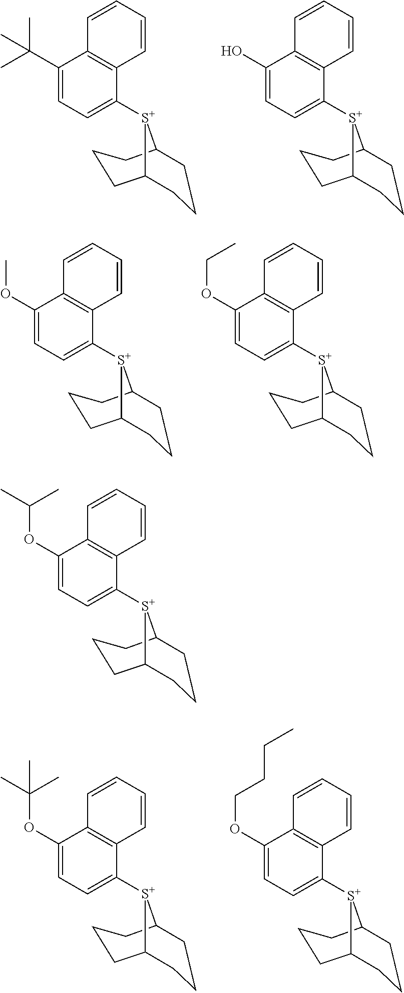

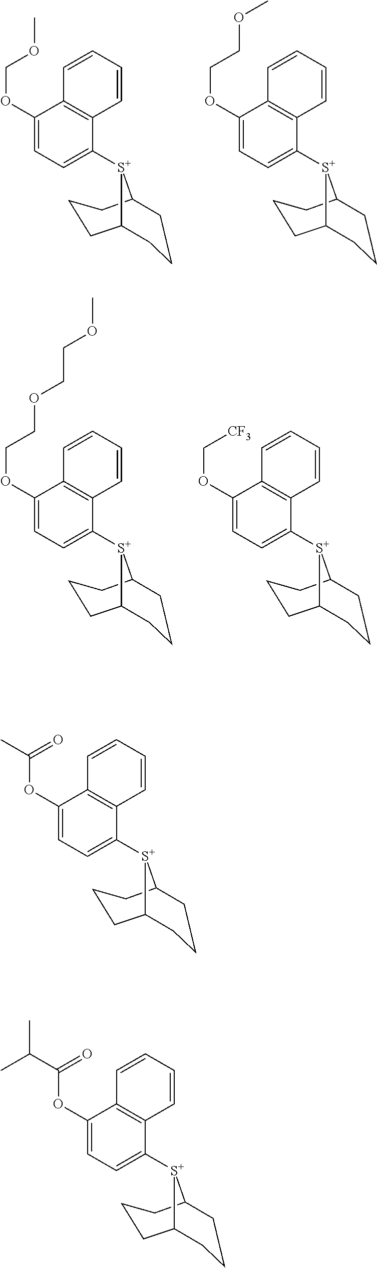 [SMILES: COCOc1ccc([S+]2C3CCCC2CCC3)c2ccccc12 ; COCCOc1ccc([S+]2C3CCCC2CCC3)c2ccccc12 ; COCCOCCOc1ccc([S+]2C3CCCC2CCC3)c2ccccc12 ; FC(F)(F)COc1ccc([S+]2C3CCCC2CCC3)c2ccccc12 ; CC(=O)Oc1ccc([S+]2C3CCCC2CCC3)c2ccccc12 ; CC(C)C(=O)Oc1ccc([S+]2C3CCCC2CCC3)c2ccccc12]

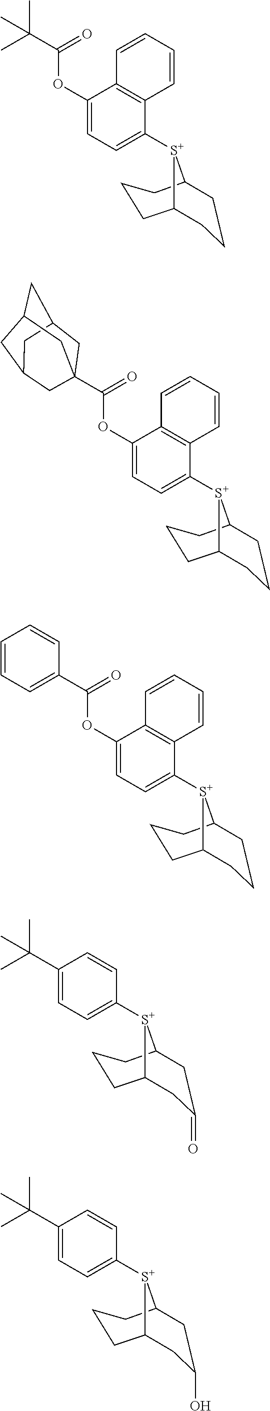 [SMILES: CC(C)(C)C(=O)Oc1ccc([S+]2C3CCCC2CCC3)c2ccccc12 ; O=C(Oc1ccc([S+]2C3CCCC2CCC3)c2ccccc12)C12CC3CC(CC(C3)C1)C2 ; O=C(Oc1ccc([S+]2C3CCCC2CCC3)c2ccccc12)c1ccccc1 ; CC(C)(C)c1ccc([S+]2C3CCCC2CC(=O)C3)cc1 ; CC(C)(C)c1ccc([S+]2C3CCCC2CC(O)C3)cc1]

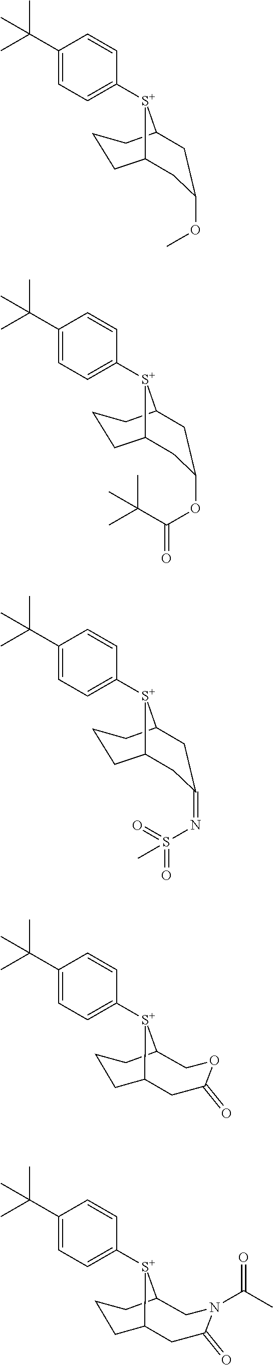 [SMILES: COC1CC2CCCC(C1)[S+]2c1ccc(C(C)(C)C)cc1 ; CC(C)(C)C(=O)OC1CC2CCCC(C1)[S+]2c1ccc(C(C)(C)C)cc1 ; CC(C)(C)c1ccc([S+]2C3CCCC2CC(=NS(C)(=O)=O)C3)cc1 ; CC(C)(C)c1ccc([S+]2C3CCCC2CC(=O)OC3)cc1 ; CC(=O)N1CC2CCCC(CC1=O)[S+]2c1ccc(C(C)(C)C)cc1]

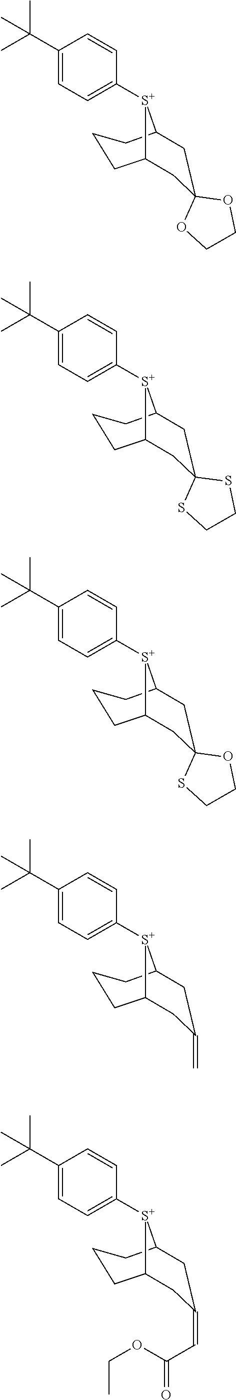

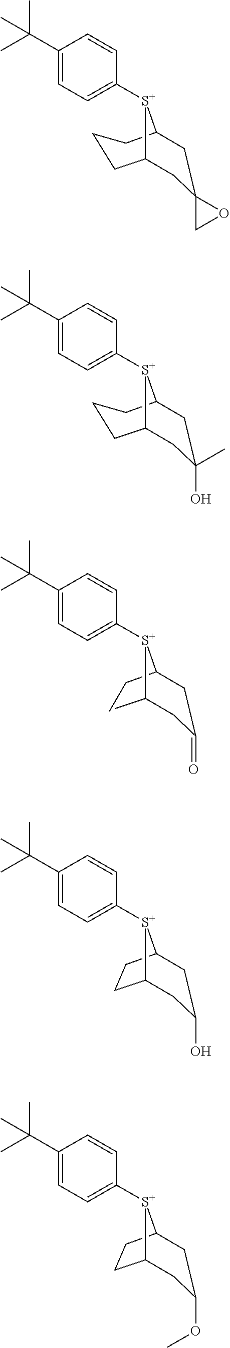

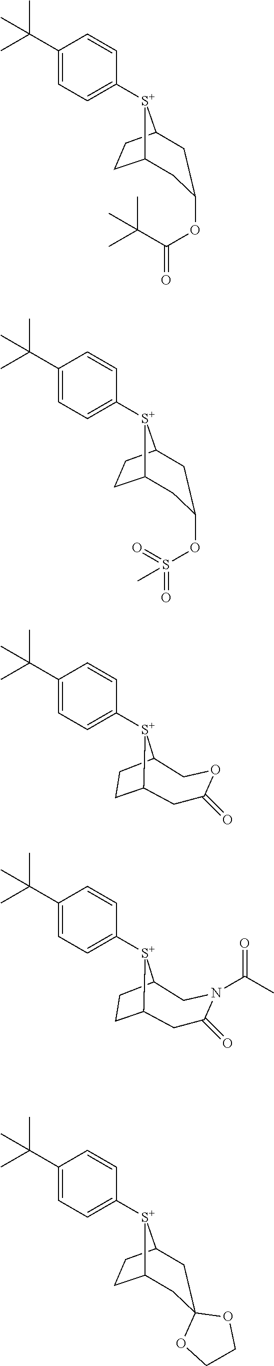

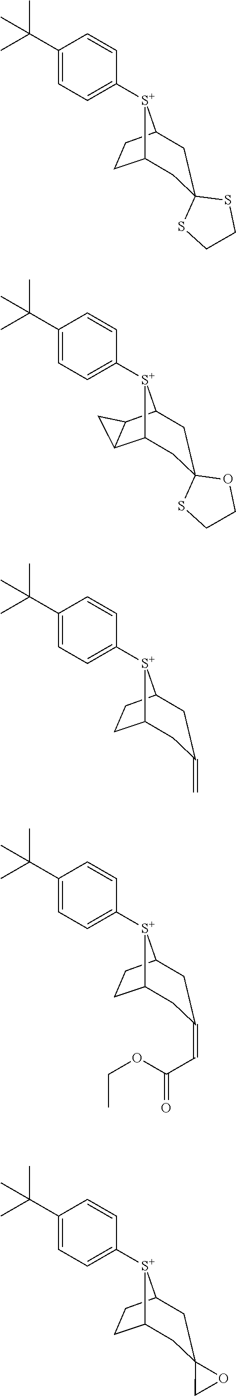

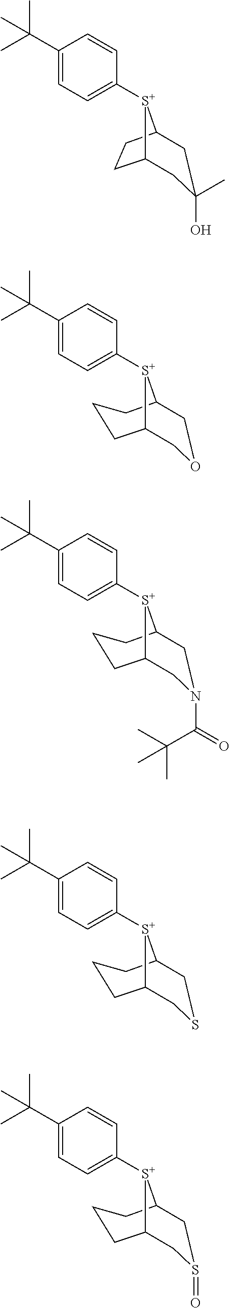

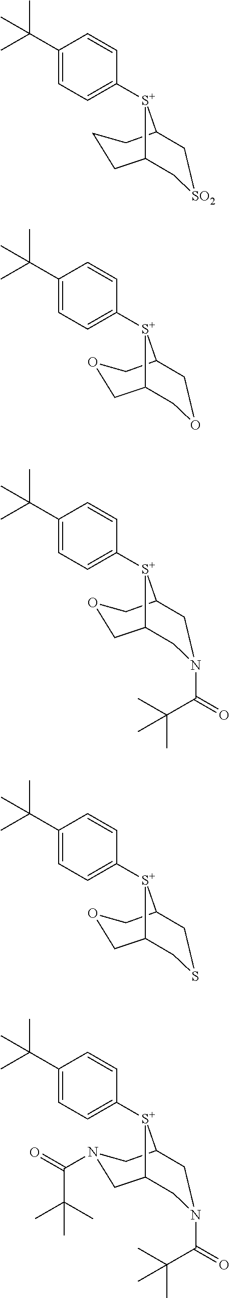 [SMILES: CC(C)(C)c1ccc([S+]2C3CCCC2CS(=O)(=O)C3)cc1 ; CC(C)(C)c1ccc([S+]2C3COCC2COC3)cc1 ; CC(C)(C)C(=O)N1CC2COCC(C1)[S+]2c1ccc(C(C)(C)C)cc1 ; CC(C)(C)c1ccc([S+]2C3COCC2CSC3)cc1 ; CC(C)(C)C(=O)N1CC2CN(C(=O)C(C)(C)C)CC(C1)[S+]2c1ccc(C(C)(C)C)cc1]

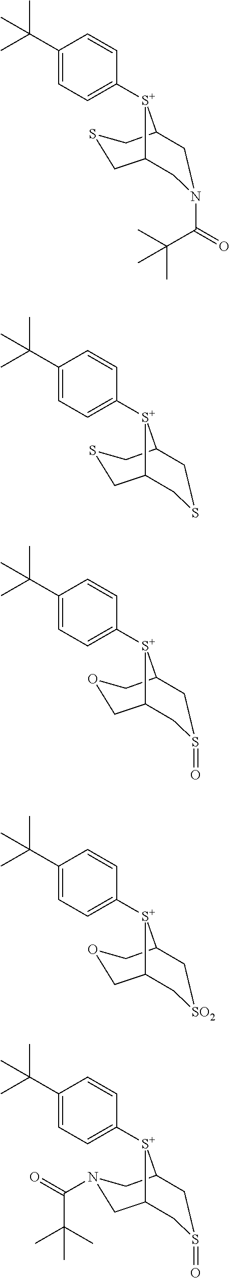 [SMILES: CC(C)(C)C(=O)N1CC2CSCC(C1)[S+]2c1ccc(C(C)(C)C)cc1 ; CC(C)(C)c1ccc([S+]2C3CSCC2CSC3)cc1 ; CC(C)(C)c1ccc([S+]2C3COCC2CS(=O)C3)cc1 ; CC(C)(C)c1ccc([S+]2C3COCC2CS(=O)(=O)C3)cc1 ; CC(C)(C)C(=O)N1CC2CS(=O)CC(C1)[S+]2c1ccc(C(C)(C)C)cc1]

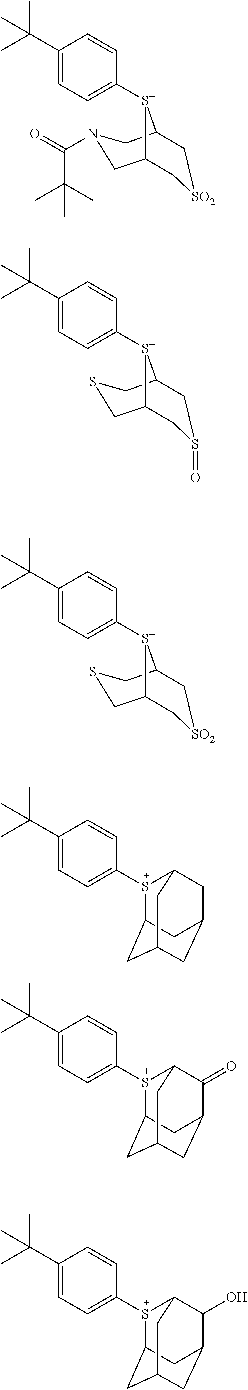

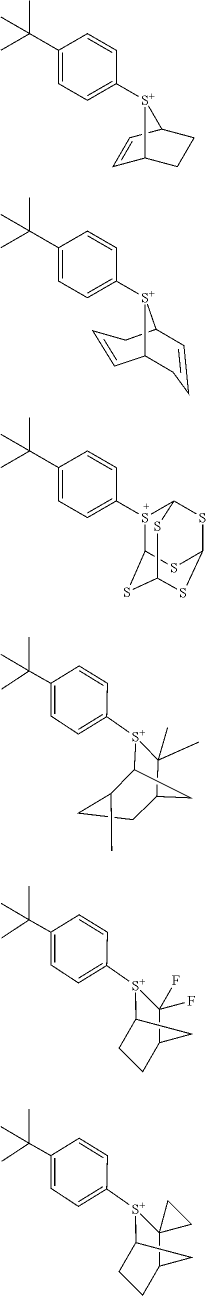

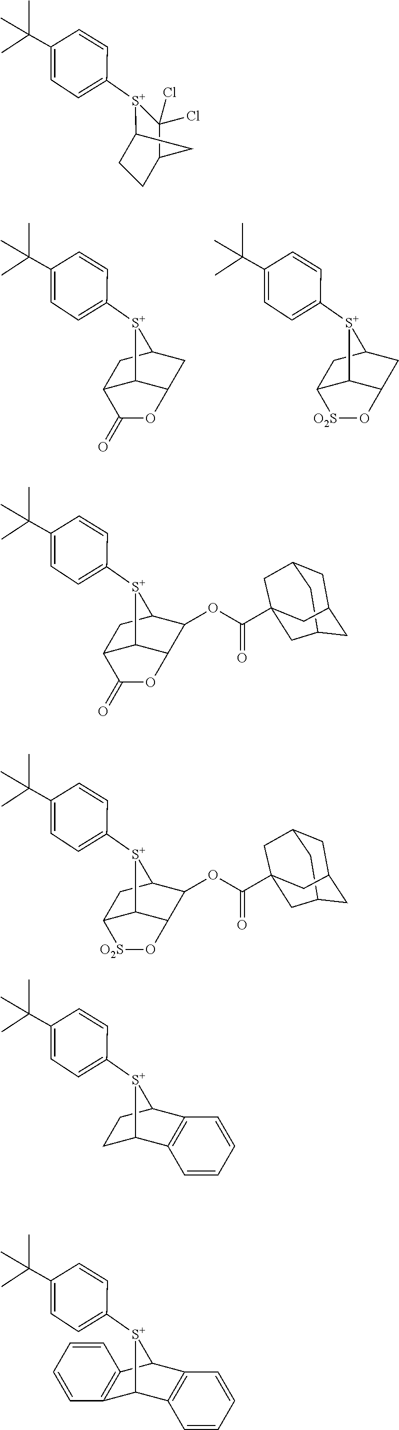

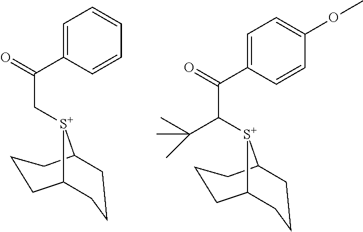 [SMILES: O=C(C[S+]1C2CCCC1CCC2)c1ccccc1 ; COc1ccc(C(=O)C([S+]2C3CCCC2CCC3)C(C)(C)C)cc1]

Examples of the iodonium cation represented by the formula (cation-2) include, but are not limited thereto.

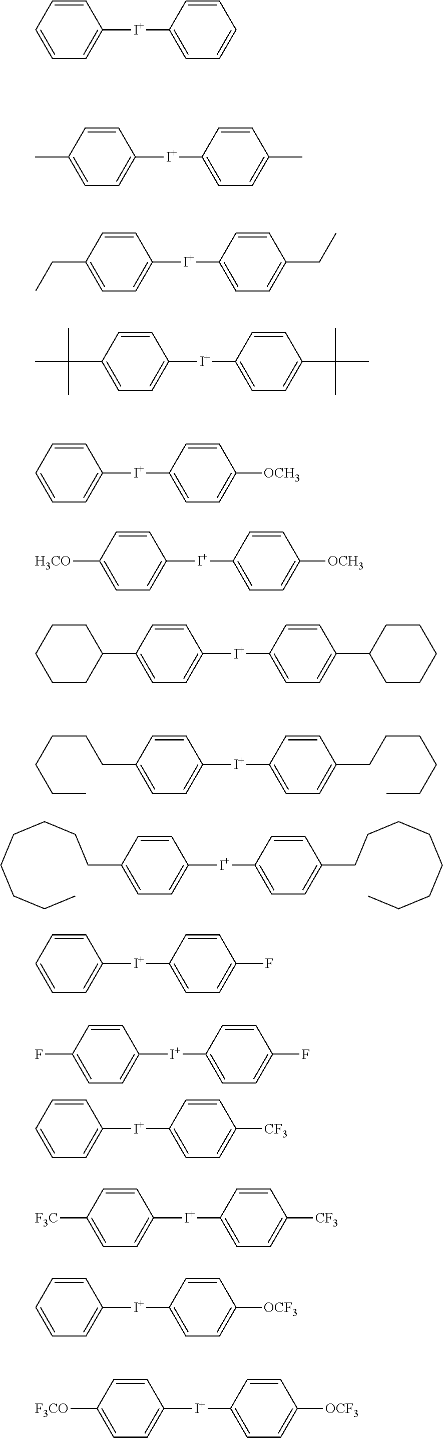

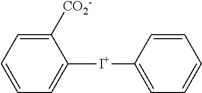

Specific examples of the structures of the repeating units represented by the formulae (C1) to (C4) include any combination of the anion and the cation described above.

The repeating unit C is preferably the repeating unit “c2”, “c3” or “c4” in view of controlling acid diffusion, more preferably the repeating unit “c2” or “c4” in view of the acid intensity of the generated acid, and still more preferably the repeating unit “c2” in view of the solvent solubility.

The polymer compound may further contain a repeating unit (hereinafter may also be referred to as a repeating unit “d”) having a structure in which the hydroxy group is protected by an acid-labile group. The repeating unit “d” is not particularly limited insofar as the unit includes one or two or more structures in which the hydroxy group is protected and the protecting group is to be decomposed under the action of acid to generate the hydroxy group. The repeating unit represented by the following formula (d1) is preferable.

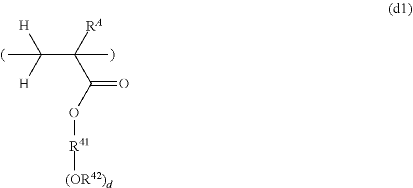

In the formula (d1), R^{A} is as defined above. R⁴¹ is a (d+1)-valent hydrocarbon group having 1 to 30 carbon atoms which may contain a heteroatom. R⁴² is an acid-labile group, and d is an integer of 1 to 4.

In the formula (d1), the acid-labile group represented by R⁴² is not particularly limited insofar as it is to be deprotected under the action of acid to generate a hydroxy group. The structure of R⁴² is not particularly limited; however, it is preferably an acetal structure, a ketal structure, an alkoxycarbonyl group, an alkoxymethyl group represented by the following formula (d2), or the like, and particularly preferably an alkoxymethyl group represented by the following formula (d2),

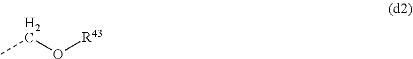

wherein the dashed line represents a bonding arm; and R⁴³ is a hydrocarbyl group having 1 to 15 carbon atoms.

Specific examples of the acid-labile group represented by R⁴², the alkoxymethyl group represented by the formula (d2) and the repeating unit “d” are the same as those exemplified in the description of the repeating unit “d” in JP 2020-111564 A.

The polymer compound may further contain a repeating unit “e” derived from indene, benzofuran, benzothiophene, acenaphthylene, chromone, coumarin, and norbornadiene, or derivatives thereof. Examples of the monomer to give the repeating unit “e” include, but are not limited to, those listed below.

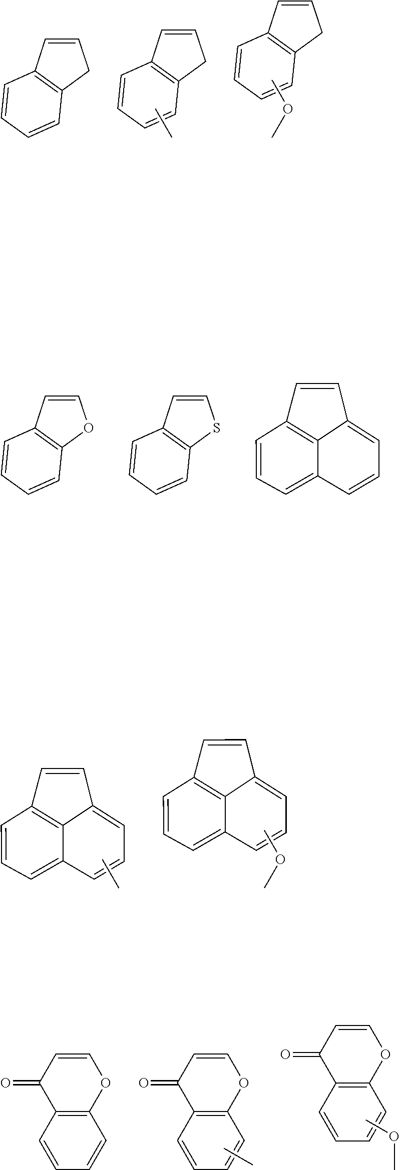

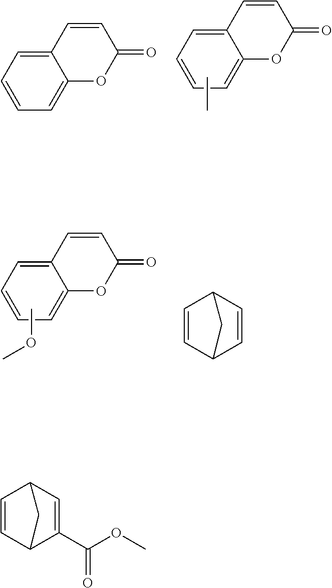

The polymer compound may further contain a repeating unit “f” derived from indane, vinylpyridine or vinylcarbazole.

In the polymer of the present invention, the content ratio of the repeating units “A”, “a1”, “a2”, “b1”, “b2”, “c1” to “c4”, “d”, “e” and “f” is preferably 0<A≤0.8, 0≤a1≤ 0.8, 0≤a2≤ 0.8, 0≤ b1≤ 0.6, 0≤b2≤ 0.6, 0≤c1≤ 0.4, 0≤c2≤0.4, 0≤ c3≤ 0.4, 0≤c4≤0.4, 0≤d≤0.5, 0≤e≤0.3 and 0≤f≤ 0.3, and more preferably 0<A≤ 0.7, 0≤ a1≤ 0.7, 0≤a2≤0.7, 0≤b1≤0.5, 0≤ b2≤ 0.5, 0≤ c1≤ 0.3, 0≤ c2<<0.3, 0≤ c3≤ 0.3, 0≤ c4≤ 0.3, 0≤ d≤0.3, 0≤ e≤0.3 and 0≤ f≤ 0.3.

The weight-average molecular weight (Mw) of the polymer is preferably 1,000 to 500,000, and more preferably 3,000 to 100,000. The above range of Mw ensures satisfactory etching resistance and a dissolution rate difference before and after the exposure, thus eliminating the risk of a decrease in resolution. In the present invention, Mw is a polystyrene-based value determined by gel permeation chromatography (GPC) using tetrahydrofuran (THF) or N,N-dimethylformamide (DMF) as an eluent.

In addition, the influences of the molecular weight distribution (Mw/Mn) of the polymer tend to become stronger as the pattern rule becomes finer. Therefore, the polymer preferably has a narrow distribution, i.e., Mw/Mn of 1.0 to 2.0, in order to obtain a resist composition suitably used for fine pattern size. When the molecular weight distribution is within the above range, the amount of a polymer having a low molecular weight or a high molecular weight is small, and there is no possibility that foreign matters are observed on the pattern or the shape of the pattern is deteriorated after the exposure.

The polymer may be synthesized, for example, by performing polymerization in which monomers giving the repeating unit described above is heated in an organic solvent while adding a radical polymerization initiator.

Examples of the organic solvent used for the polymerization include toluene, benzene, THF, diethyl ether, dioxane, cyclohexane, cyclopentane, methyl ethyl ketone (MEK), propylene glycol monomethyl ether acetate (PGMEA), γ-butyrolactone (GBL), and the like. Examples of the polymerization initiator include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, lauroyl peroxide, and the like. The addition amount of these initiators is preferably 0.01 to 25 mol % relative to the total of the monomers to be polymerized. The reaction temperature is preferably 50 to 150° C., and more preferably 60 to 100° C. The reaction time is preferably 2 to 24 hours, and more preferably 2 to 12 hours in view of production efficiency.

The polymerization initiator may be added to the monomer solution and then the mixture may be supplied to the reaction vessel; or an initiator solution may be prepared separately from the monomer solution and each may be independently supplied to the reaction vessel. In view of quality control, it is preferable that the monomer solution and the initiator solution being independently prepared and added dropwise, because there is a possibility that a radical generated from the initiator during the wait time promotes the polymerization reaction to thereby produce an ultra-high molecular weight polymer. The acid-labile group that has been introduced into the monomer may be used as it is, or protection or partial protection thereof after the polymerization is also possible. In order to adjust the molecular weight, a known chain transfer agent such as dodecyl mercaptan or 2-mercaptoethanol may also be used. In this case, the addition amount of these chain transfer agents is preferably 0.01 to 20 mol % relative to the total of the monomers to be polymerized.

In the case of a monomer having a hydroxy group, the hydroxy group may be substituted with an acetal group susceptible to deprotection by an acid, such as an ethoxyethoxy group, upon the polymerization, and deprotection may be performed with weak acid and water after the polymerization. Alternatively, the hydroxy group may be substituted with an acetyl group, a formyl group, a pivaloyl group, and the like, and the polymerization may be followed by alkaline hydrolysis.

In the case of copolymerizing hydroxystyrene or hydroxyvinylnaphthalene, hydroxystyrene or hydroxyvinylnaphthalene and other monomers may be polymerized by heating in an organic solvent in the presence of a radical polymerization initiator. Alternatively, acetoxystyrene or acetoxyvinylnaphthalene may be used, and after the polymerization, the acetoxy group may be deprotected by alkaline hydrolysis to obtain polyhydroxystyrene or hydroxypolyvinylnaphthalene.

As the base to be used in the alkaline hydrolysis, aqueous ammonia, triethylamine, and the like may be used. Further, the reaction temperature is preferably 20 to 100° C., more preferably 0 to 60° C. The reaction time is preferably 0.2 to 100 hours, more preferably 0.5 to 20 hours.

The amount of each monomer in the monomer solution may be appropriately set so as to achieve, for example, the above-described preferable content ratio of the repeating unit.

Regarding the polymer obtained by the above production method, the reaction solution obtained by the polymerization reaction may be used as a final product; alternatively, a powder obtained through a purification step such as a reprecipitation method in which a polymerization solution is added to a poor solvent to obtain a powder may be used as a final product. However, in view of working efficiency and quality stabilization, a polymer solution obtained by dissolving the powder obtained by the purification step in a solvent is preferably used as a final product.

Examples of the solvent to be used herein include ketones such as cyclohexanone and methyl-2-n-pentylketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol-mono-tert-butylether acetate; lactones such as GBL; alcohols such as diacetone alcohol (DAA); high-boiling-point alcohol-based solvents such as diethylene glycol, propylene glycol, glycerin, 1,4-butanediol, and 1,3-butanediol; mixed solvents thereof, and the like, which are described in paragraphs [0144] to [0145] in JP 2008-111103 A.

The concentration of the polymer in the polymer solution is preferably 0.01 to 30 mass %, and more preferably 0.1 to 20 mass %.

The reaction solution and the polymer solution are preferably filtered through a filter. By performing filtration with a filter, it is possible to remove foreign substances and gels which may cause defects; thus, it is effective in terms of quality stabilization.

Examples of the material of the filter used in the filtration include fluorocarbon-based materials, cellulose-based materials, nylon-based materials, polyester-based materials, and hydrocarbon-based materials. In the filtration step of the resist composition, a filter formed of a fluorocarbon-based material called Teflon (registered trademark), a hydrocarbon-based filter formed of polyethylene, polypropylene or the like, or a nylon filter is preferable. The pore size of the filter can be appropriately selected according to the target cleanness; however, the pore size is preferably 100 nm or less, and more preferably 20 nm or less. One of these filters may be used alone, or a plurality of filters may be used in combination. In the filtration method, the solution may be passed through only once; however, it is more preferable to circulate the solution and perform filtration multiple times. The filtration step may be performed in any order and any number of times in the production process of the polymer; however, it is preferable to filter the reaction solution, the polymer solution, or both, after the polymerization reaction.

Only one kind of the polymer may be used alone or two or more polymers having different composition ratios, Mw, and/or Mw/Mn may be used in combination. In addition to the polymers described above, the polymer compound of the present invention may contain a hydrogenated ring-opening metathesis polymer, such as that described in JP 2003-66612 A.

[(B) Organic Solvent]

Any organic solvents may be used as the organic solvent as the component (B) insofar as the components described above and below are soluble therein. Examples of the organic solvent include ketones such as cyclopentanone, cyclohexanone, and methyl-2-n-pentylketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol-mono-tert-butylether acetate; lactones such as GBL; mixed solvents thereof; and the like.

Among these organic solvents, it is preferable to use 1-ethoxy-2-propanol, PGMEA, cyclohexanone, GBL, DAA, and mixed solvents thereof, which are particularly superior in solubility for the polymer compound as the component (A).

The amount of the organic solvent to be used is preferably 200 to 5,000 parts by mass, more preferably 400 to 3,500 parts by mass, per 80 parts by mass of the polymer compound (A). Only one kind of the organic solvent (B) may be used alone or two or more kinds thereof may be mixed.

### [(C) Quencher]

Examples of the quencher (C) include onium salts represented by the following general formula (1)′ or (2)′.

R^{q1}—SO₃⁻Mq⁺ (1)′

R^{q2}—CO₂⁻Mq⁺ (2)′

In the general formula (1)′ above, R^{q1} is a hydrogen atom or a hydrocarbyl group having 1 to 40 carbon atoms which may contain a heteroatom, excluding a group in which the hydrogen atom bonded to the carbon atom at α-position of the sulfo group is substituted with a fluorine atom or a fluoroalkyl group. In the general formula (2)′ above, R^{q2} is a hydrogen atom or a hydrocarbyl group having 1 to 40 carbon atoms which may contain a heteroatom.

Examples of the hydrocarbyl group represented by R^{q1} include alkyl groups such as a methyl group, an ethyl group, a propyl group, an isopropyl group, a n-butyl group, a sec-butyl group, a tert-butyl group, a n-pentyl group, a tert-pentyl group, a n-hexyl group, a n-octyl group, a 2-ethylhexyl group, a n-nonyl group, and a n-decyl group; cyclic saturated hydrocarbyl groups such as a cyclopentyl group, a cyclohexyl group, a cyclopentyl methyl group, a cyclopentyl ethyl group, a cyclopentyl butyl group, a cyclohexyl methyl group, a cyclohexyl ethyl group, a cyclohexyl butyl group, a norbornyl group, a tricyclo [5.2.1.0^{2,6}] decanyl group, and an adamantyl group; aryl groups such as a phenyl group, a naphthyl group, and an anthracenyl group; and the like. In these groups, some or all of the hydrogen atoms are optionally substituted with a group containing a heteroatom such as an oxygen atom, a sulfur atom, a nitrogen atom and a halogen atom, or some carbon atoms are optionally substituted with a group containing a heteroatom such as an oxygen atom, a sulfur atom, and a nitrogen atom, so that the groups may contain a hydroxy group, a cyano group, a carbonyl group, an ether bond, an ester bond, a sulfonic acid ester bond, a carbonate bond, a lactone ring, a sultone ring, a carboxylic acid anhydride, a haloalkyl group or the like.

Examples of the hydrocarbyl group represented by R^{q2} include, in addition to those exemplified for R^{q1}, fluorinated alkyl groups such as a trifluoromethyl group or a trifluoroethyl group; and fluorinated aryl groups such as a pentafluorophenyl group and a 4-trifluoromethylphenyl group.

Examples of the anion of the onium salt represented by the general formula (1)′ include, but are not limited thereto.

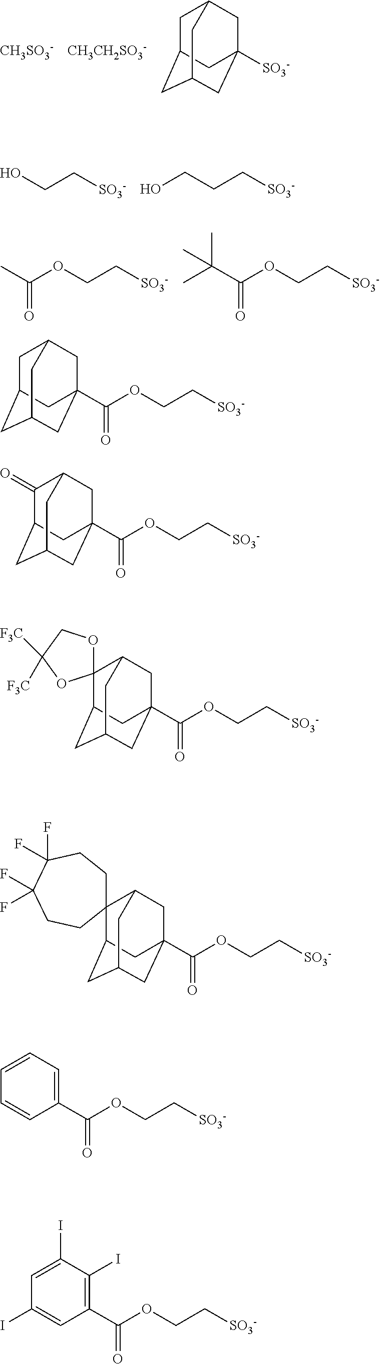

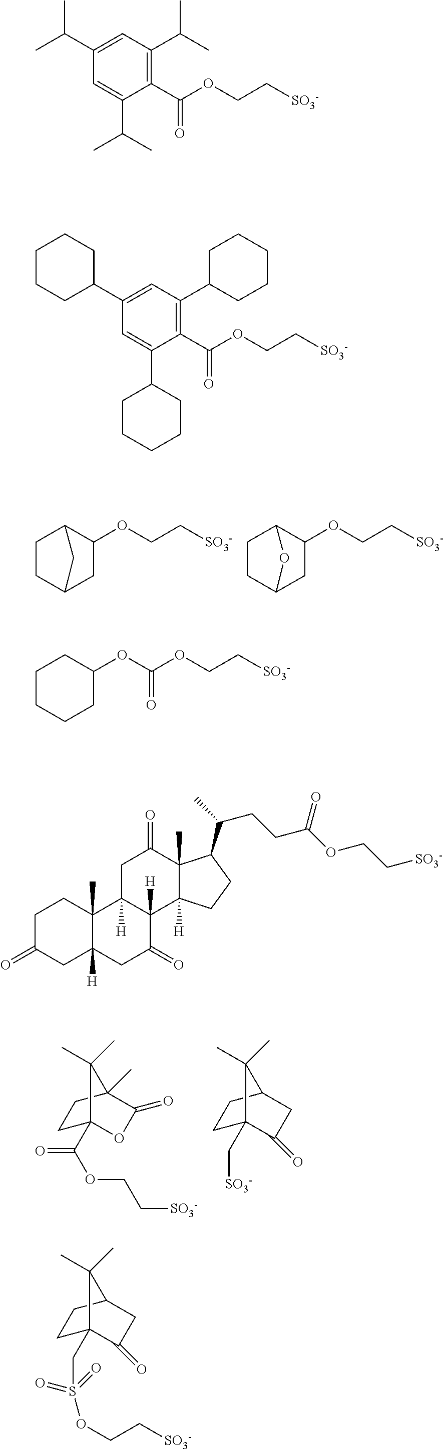

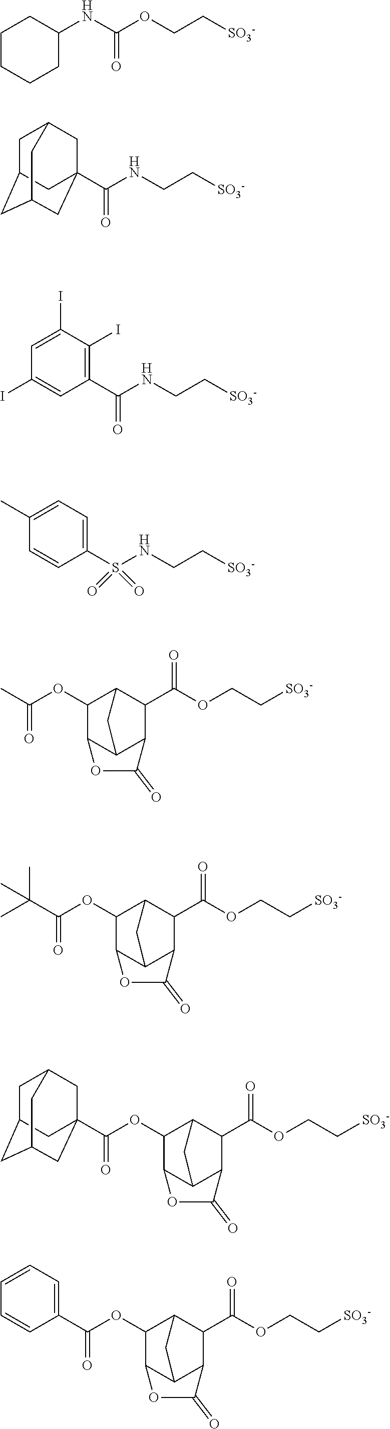

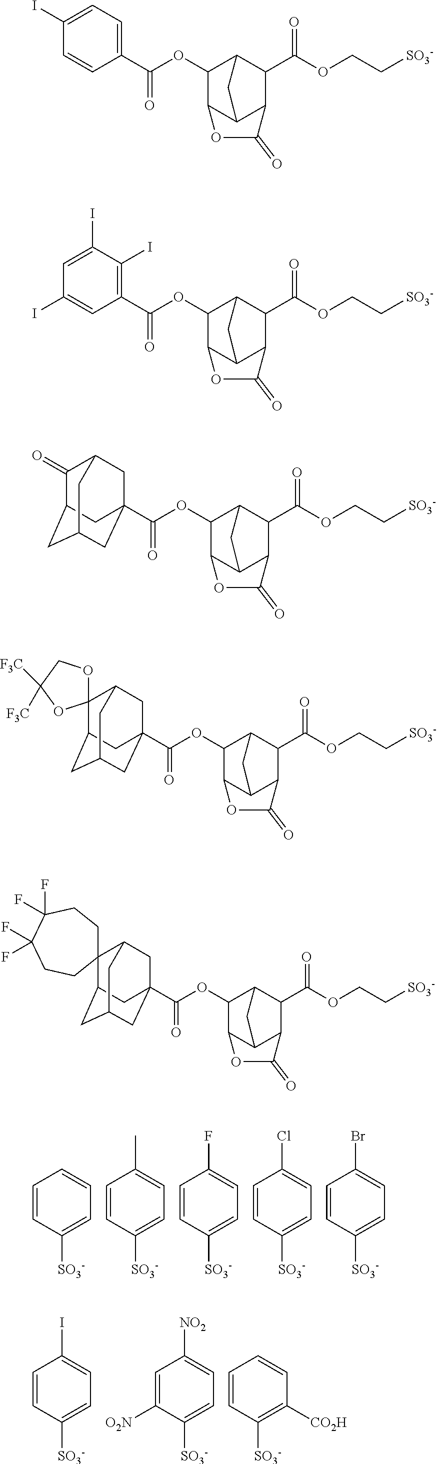

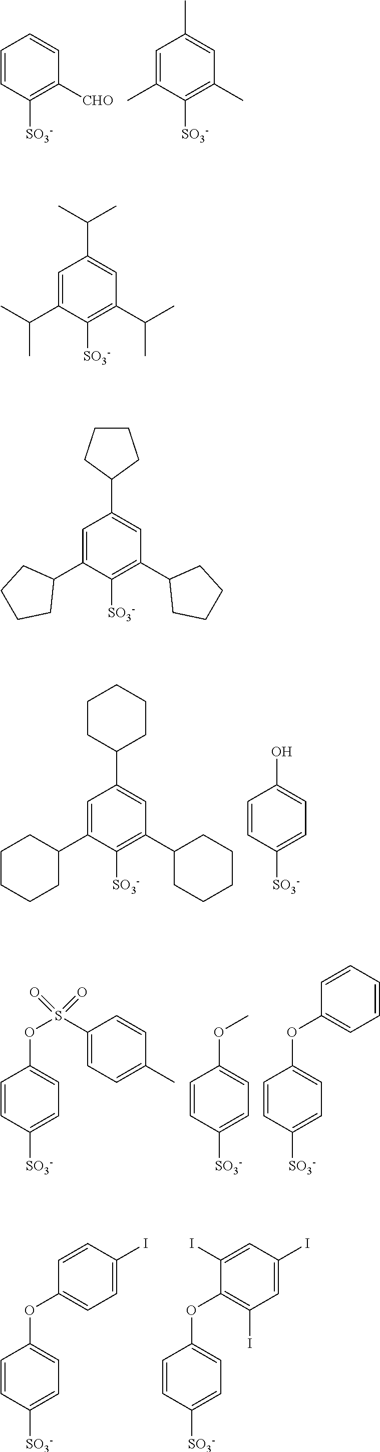

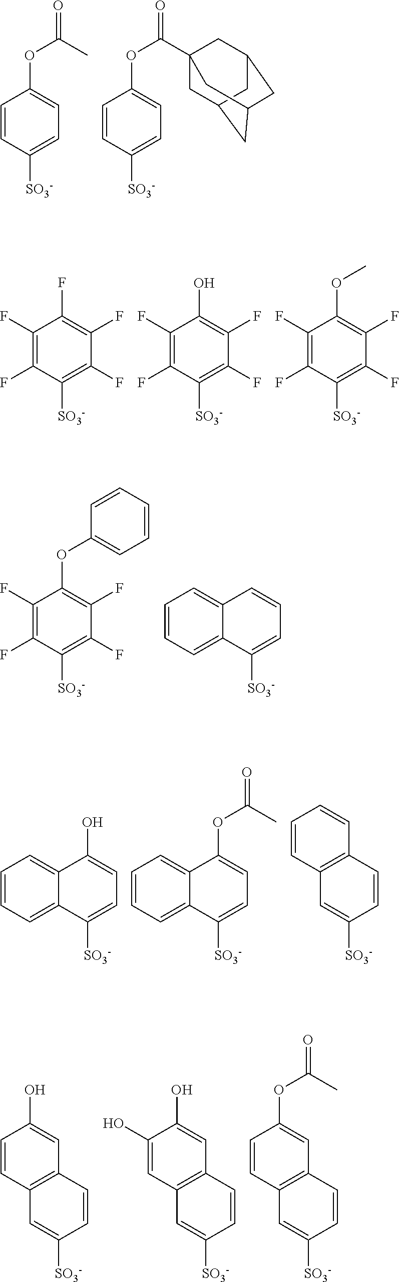

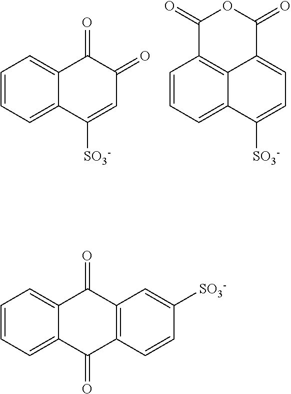

Examples of the anion of the onium salt represented by the general formula (2)′ include, but are not limited thereto.

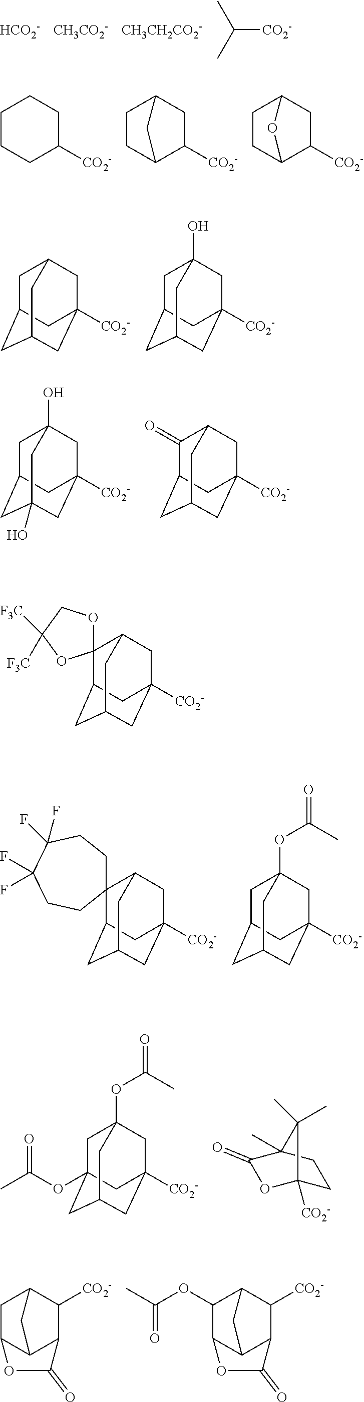

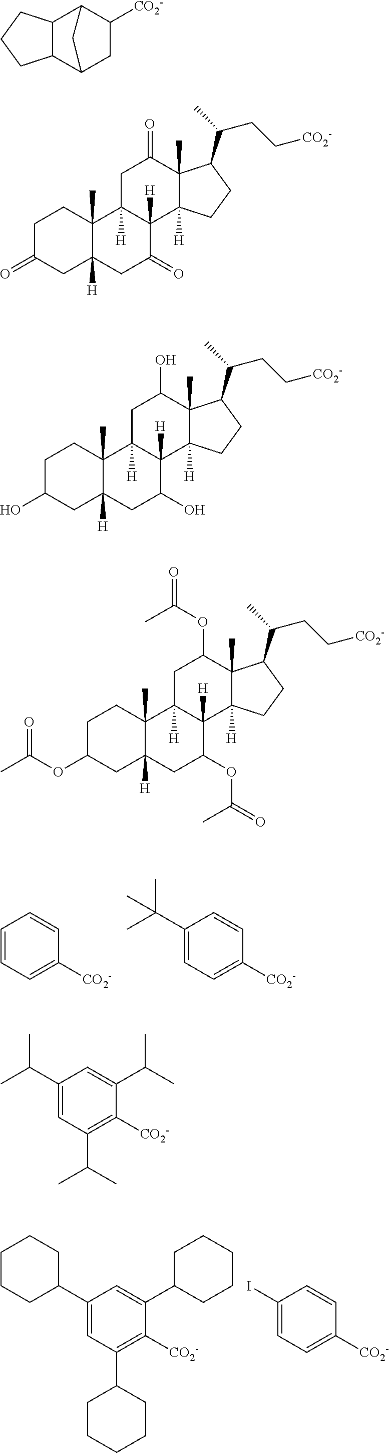

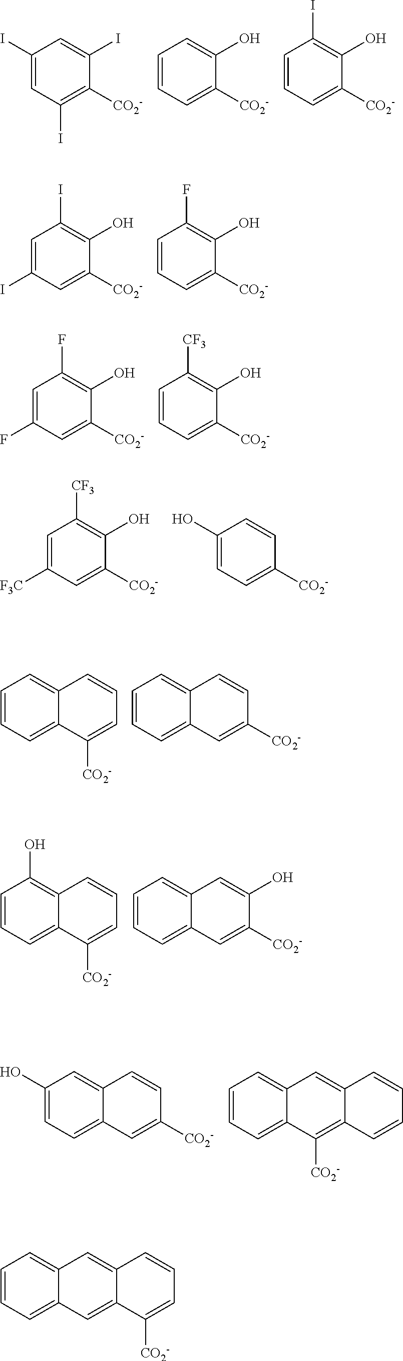

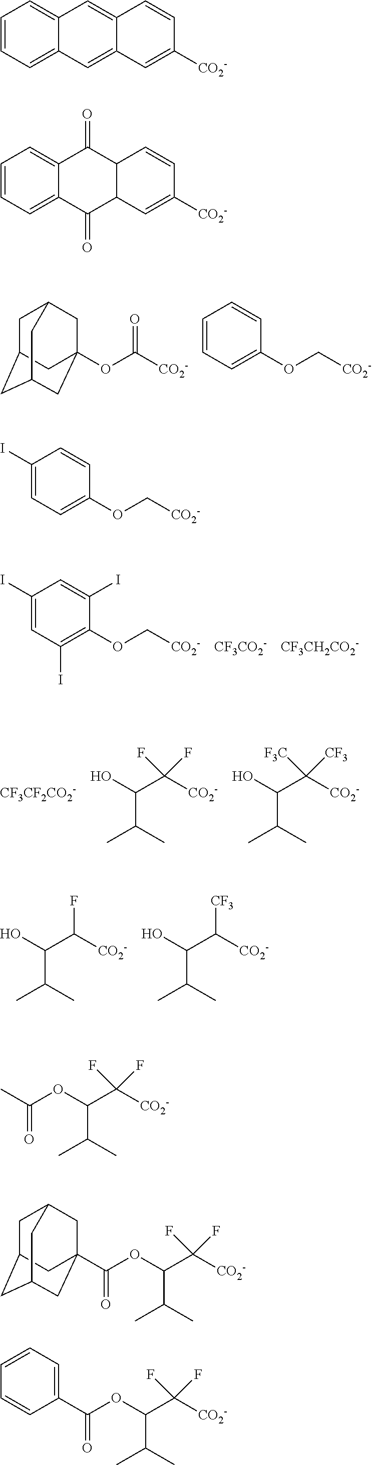

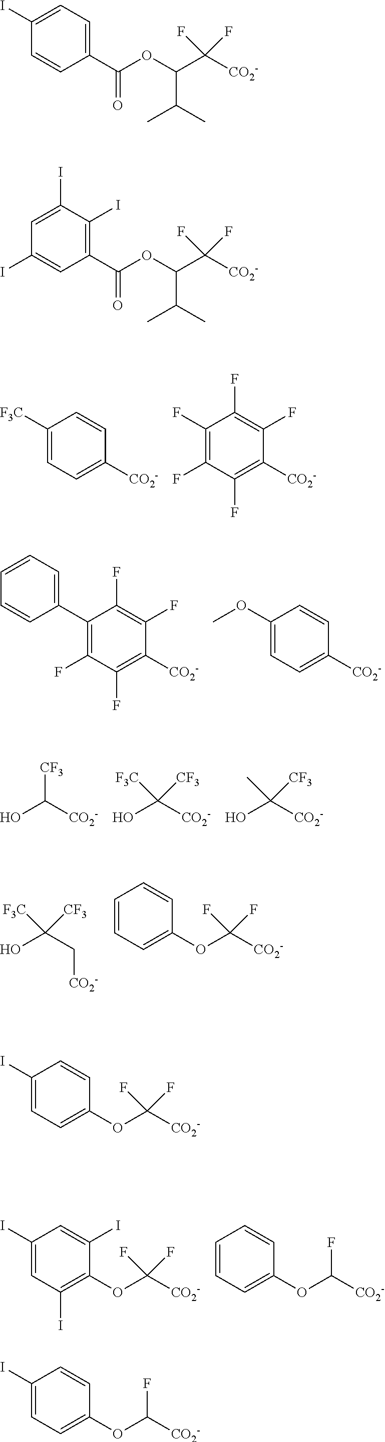

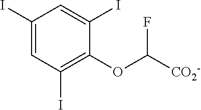

In the general formulae (1)′ and (2)′, Mq⁺ represents an onium cation. The onium cation is preferably an onium cation represented by the following formula (cation-1), (cation-2) or (cation-3).

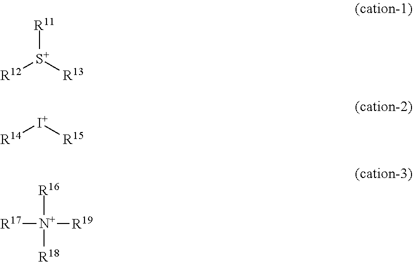

Examples of the formulae (cation-1) and (cation-2) include those similar to A⁺ in the formulae (C2) to (C4) R¹⁶ to R¹⁹ in (cation-3) independently represent a hydrocarbyl group having 1 to 40 carbon atoms which may contain a heteroatom. R¹⁶ and R¹⁷ may also be bonded to each other to form a ring with the nitrogen atom to which they are bonded. Examples of the hydrocarbyl group include the same groups as those exemplified in the description of R¹¹ to R¹⁵ in the formulae (cation-1) and (cation-2).

In the onium cation represented by Mq⁺, examples of the ammonium cation represented by (cation-3) include, but are not limited thereto.

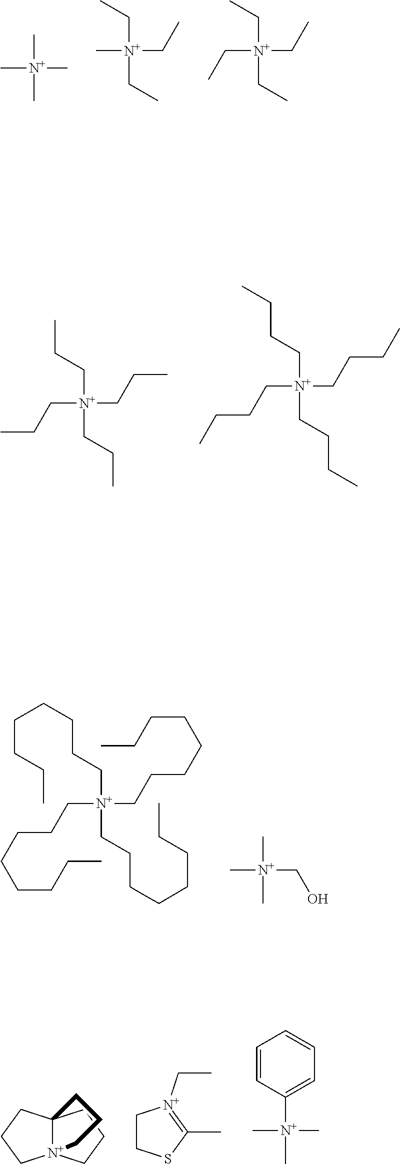

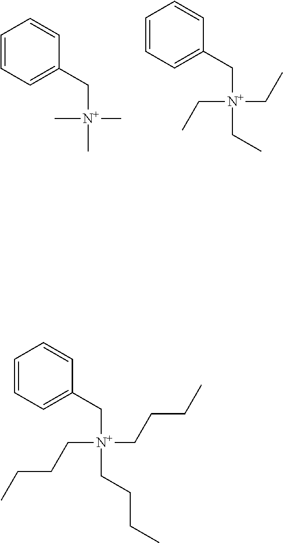 [SMILES: C[N+](C)(C)Cc1ccccc1 ; CC[N+](CC)(CC)Cc1ccccc1 ; CCCC[N+](CCCC)(CCCC)Cc1ccccc1]

Specific examples of the onium salt represented by the general formula (1)′ or (2)′ include arbitrary combinations of anions and cations exemplified above. These onium salts are easily prepared by an ion exchange reaction using known organic chemistry techniques. The ion exchange reaction may be performed by referring to, for example, JP 2007-145797 A.

The onium salt represented by the general formula (1)′ or (2)′ functions as a quencher in the chemically-amplified resist composition of the present invention. This is because the counter anion of the onium salt is a conjugated base of a weak acid. As used herein, the weak acid indicates an acidity insufficient to deprotect the acid-labile group in the acid-labile group-containing unit used for the base polymer.

The onium salt represented by the general formula (1)′ or (2)′ functions as a quencher when it is used in combination with an onium salt-type photoacid generator having a conjugated base of a strong acid, such as a sulfonic acid with its α-position fluorinated, as the counter anion. That is, when a mixture of an onium salt generating a strong acid, such as the sulfonic acid with its α-position fluorinated, and an onium salt generating a weak acid, such as a non-fluorinated sulfonic acid and a carboxylic acid, is used, the strong acid generated from the photoacid generator upon exposure to high-energy beam collides with the unreacted onium salt having a weak acid anion, and a salt exchange occurs whereby the weak acid is released, thereby forming an onium salt having a strong acid anion. In this process, the strong acid is exchanged to a weak acid having a lower catalysis, incurring apparent deactivation of the acid, thereby enabling control of acid diffusion.

Further, as the quencher (C), the onium salt having a sulfonium cation and a phenoxide anion moiety within one molecule described in JP 6848776 B, the onium salts having a sulfonium cation and a carboxylate anion moiety within one molecule described in JP 6583136 B and JP 2020-200311 A, and the onium salt having an iodonium cation and a carboxylate anion moiety within one molecule described in JP 6274755 B may also be used.

If the photoacid generator generating a strong acid is an onium salt, an exchange from the strong acid generated upon exposure to high-energy beam to a weak acid as above can take place; however, it is unlikely that the weak acid generated upon exposure to high-energy beam collides with the unreacted onium salt generating a strong acid to induce a salt exchange. This is because an onium cation tends to form an ion pair with a stronger acid anion.

When the onium salt represented by the general formula (1)′ or (2)′ is contained as the onium salt-type quencher (C), the content thereof is preferably 0.1 to 20 parts by mass, more preferably 0.1 to 10 parts by mass, per 80 parts by mass of the polymer compound (A). As long as the amount of the onium salt-type quencher as the component (C) is within the range specified above, a desirable resolution is ensured and a significant decrease in sensitivity can be avoided. Only one kind of the onium salt represented by the general formula (1)′ or (2)′, or a combination of two or more kinds thereof may be used.

The chemically-amplified resist composition of the present invention may further comprise a nitrogen-containing quencher. In the present invention, the nitrogen-containing quencher refers to a material for forming a desired pattern by trapping acids generated by the photoacid generator in the chemically-amplified resist composition to prevent the acids from diffusing into the unexposed portion.

Examples of the nitrogen-containing quencher as the component (C) include primary, secondary, or tertiary amine compounds described in paragraphs [0146] to [0164] of JP 2008-111103 A, in particular, amine compounds having a hydroxy group, an ether bond, an ester bond, a lactone ring, a cyano group, or a sulfonic acid ester bond. Examples also include compounds in which primary or secondary amines are protected with a carbamate group, such as the compounds described in JP 3790649 B.

As the nitrogen-containing quencher, a sulfonic acid sulfonium salt having a nitrogen-containing substituent may be used. This compound functions as a quencher in the unexposed portion, and functions as so-called a photo-degradable base in the exposed portion as it loses the quencher function due to neutralization with the acid generated by itself. By using a photo-degradable base, the contrast between the exposed portion and the unexposed portion can be further enhanced. For the photo-degradable base, reference may be made to JP 2009-109595 A and JP 2012-46501 A, for example.

When the nitrogen-containing quencher as the component (C) is contained, the content thereof is preferably 0.001 to 12 parts by mass, and more preferably 0.01 to 8 parts by mass, per 80 parts by mass of the polymer compound (A). Only one kind of the nitrogen-containing compound, or a combination of two or more kinds thereof may be used.

### [(D) Acid Generator (Photoacid Generator)]

The chemically-amplified resist composition of the present invention may contain, as a component (D), an acid generator (photoacid generator) other than the repeating units “c1” to “c4” of the component (A). The photoacid generator is not particularly limited insofar as it is a compound capable of generating an acid by being exposed to high-energy beam. Examples of other suitable photoacid generators include those represented by the following general formula (3) or (4).

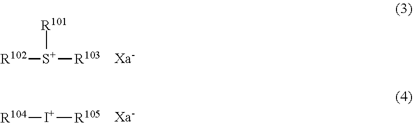

In the general formula (3), R¹⁰¹ to R¹⁰⁵ independently represent a hydrocarbyl group having 1 to 20 carbon atoms which may contain a heteroatom. Further, any two of R¹⁰¹, R¹⁰² and R¹⁰³ may be bonded to each other to form a ring with a sulfur atom to which they are bonded. Examples of the hydrocarbyl group include the same groups as those exemplified in the description of R¹¹ to R¹⁵ in the formulae (cation-1) and (cation-2).

In the general formula (3), examples of the sulfonium cation include similar to those exemplified as the sulfonium cation represented by the formula (cation-1).

In the general formula (4), examples of the iodonium cation include similar to those exemplified as the iodonium cation represented by the formula (cation-2).

In the general formulae (3) and (4), Xa⁻ represents a non-nucleophilic counter ion. Examples of the non-nucleophilic counter ion include halide ions such as chloride ions and bromide ions; fluoroalkyl sulfonate ions such as triflate ions, 1,1,1-trifluoroethane sulfonate ions, and nonafluorobutane sulfonate ions; arylsulfonate ions such as tosylate ions, benzene sulfonate ions, 4-fluorobenzene sulfonate ions, and 1,2,3,4,5-pentafluorobenzene sulfonate ions; alkylsulfonate ions such as mesylate ions and butane sulfonate ions; imide ions such as bis(trifluoromethylsulfonyl)imide ions, bis(perfluoroettylsulfonyl)imide ions, and bis(perfluorobutylsulfonyl)imide ions; methide ions such as tris(trifluoromethylsulfonyl)methide ions and tris(perfluoroethylsulfonyl)methide ions; and the like.

Other examples of the non-nucleophilic counter ion include anions selected from the following general formulae (1A) to (1D).

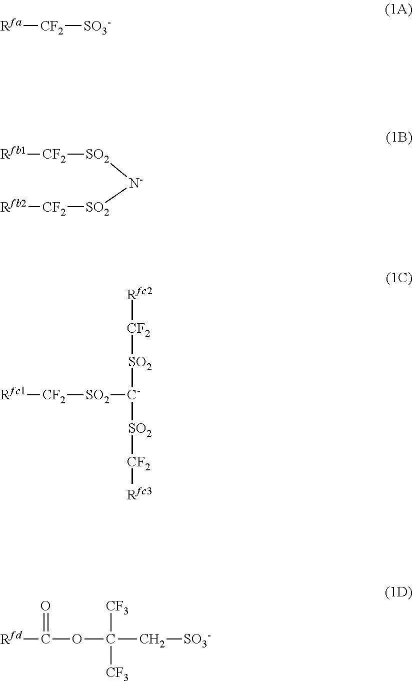

In the general formula (1A), Rf^{a} is a fluorine atom, or a hydrocarbyl group having 1 to 40 carbon atoms which may contain a heteroatom. The hydrocarbyl group may be any of a saturated, unsaturated, linear, branched, or cyclic hydrocarbyl group. Specific examples thereof include those similar to those exemplified as the hydrocarbyl group represented by R^{fa1} in the general formula (1A′) described later.

As the anion represented by the general formula (1A) above, an anion represented by the following formula (1A′) is preferable.

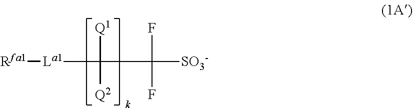

In the formula (1A′), Q¹ and Q² independently represent a hydrogen atom, a fluorine atom, or a fluorinated alkyl group having 1 to 6 carbon atoms; however at least one of them is preferably a trifluoromethyl group to improve the solvent solubility. k is an integer of 0 to 4, particularly preferably 1. R^{fa1} is a hydrocarbyl group having 1 to 50 carbon atoms which may contain a heteroatom. The heteroatom is preferably an oxygen atom, a nitrogen atom, a sulfur atom, a halogen atom, or the like, and more preferably an oxygen atom. In view of obtaining high resolution in the formation of fine patterns, the hydrocarbyl group particularly preferably has 6 to 30 carbon atoms.

In the formula (1A′), the hydrocarbyl group represented by R^{fa1} may be any of a saturated, unsaturated, linear, branched, or cyclic hydrocarbyl group. Specific examples thereof include alkyl groups having 1 to 38 carbon atoms such as a methyl group, an ethyl group, a n-propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosanyl group; cyclic saturated hydrocarbyl groups having 3 to 38 carbon atoms such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; unsaturated aliphatic hydrocarbyl groups having 2 to 38 carbon atoms such as an allyl group, and 3-cyclohexenyl group; aryl groups having 6 to 38 carbon atoms such as a phenyl group, a 1-naphthyl group, a 2-naphthyl group, and a 9-fluorenyl group; aralkyl groups having 7 to 38 carbon atoms such as a benzyl group, and a diphenylmethyl group; groups obtained by combining these groups; and the like.

Further, a part or all of the hydrogen atoms of the hydrocarbyl group may be substituted with a group containing a heteroatom such as an oxygen atom, sulfur atom, nitrogen atom and a halogen atom, and a part of the —CH₂— of the hydrocarbyl group may be substituted with a group containing a heteroatom such as an oxygen atom, sulfur atom, and a nitrogen atom, so that the group may contain a hydroxy group, fluorine atom, chlorine atom, bromine atom, iodine atom, cyano group, nitro group, carbonyl group, ether bond, ester bond, sulfonic acid ester bond, carbonate bond, lactone ring, sultone ring, carboxylic acid anhydride (—C(═O)—O—C(═O)—), haloalkyl group, or the like. Examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 3-oxocyclohexyl group, and the like.

In the formula (1A′), L^{a1} is a single bond, an ether bond, an ester bond, a sulfonic acid ester bond, a carbonate bond, or a carbamate bond; among them, L^{a}1 is preferably an ether bond or an ester bond in view of synthesis, and more preferably an ester bond.

Examples of the anion represented by the formula (1A) include, but are not limited thereto. In the following formulae, Q¹ is as defined above, and Ac is an acetyl group.

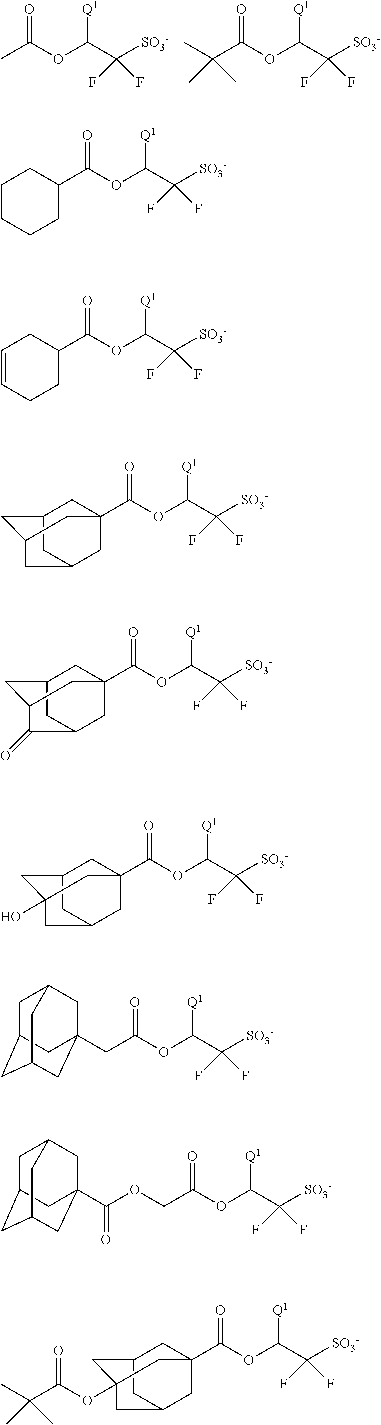

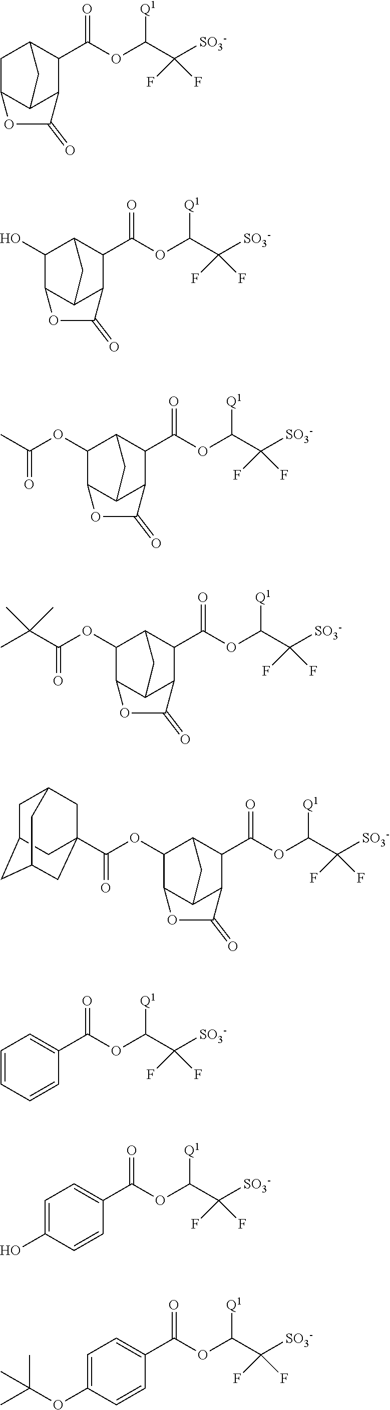

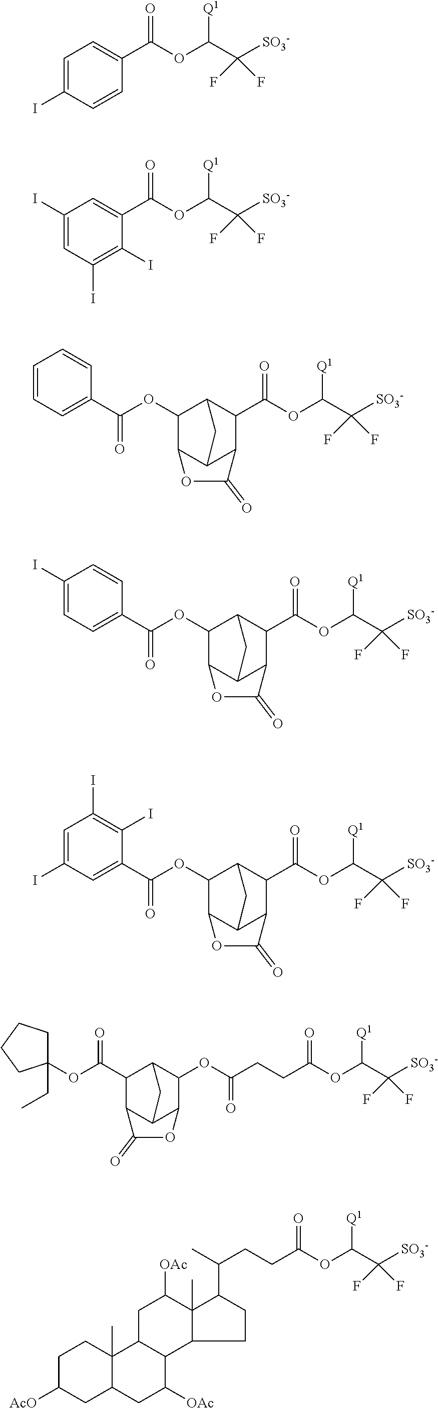

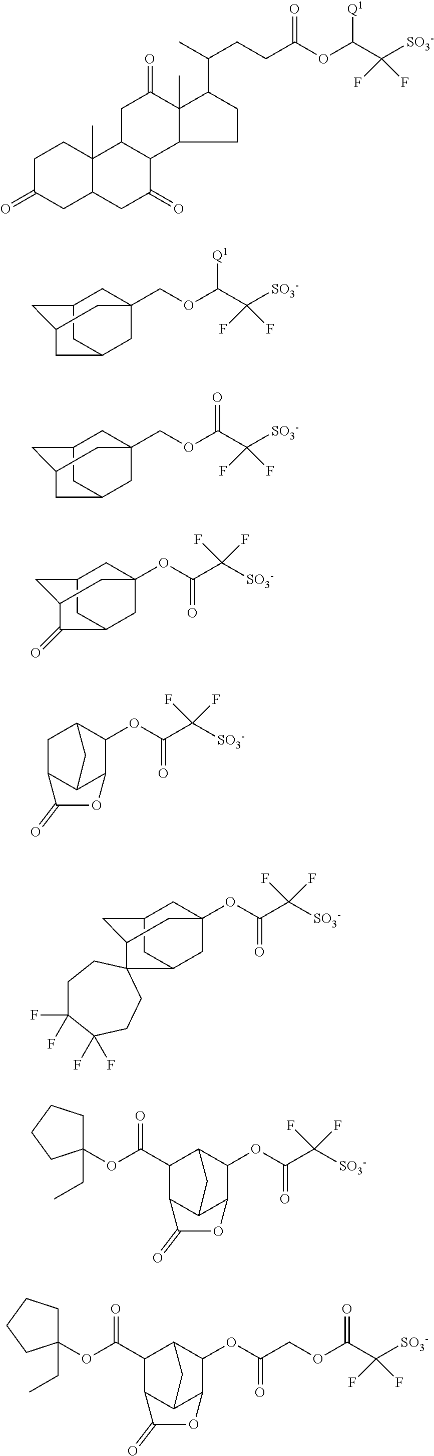

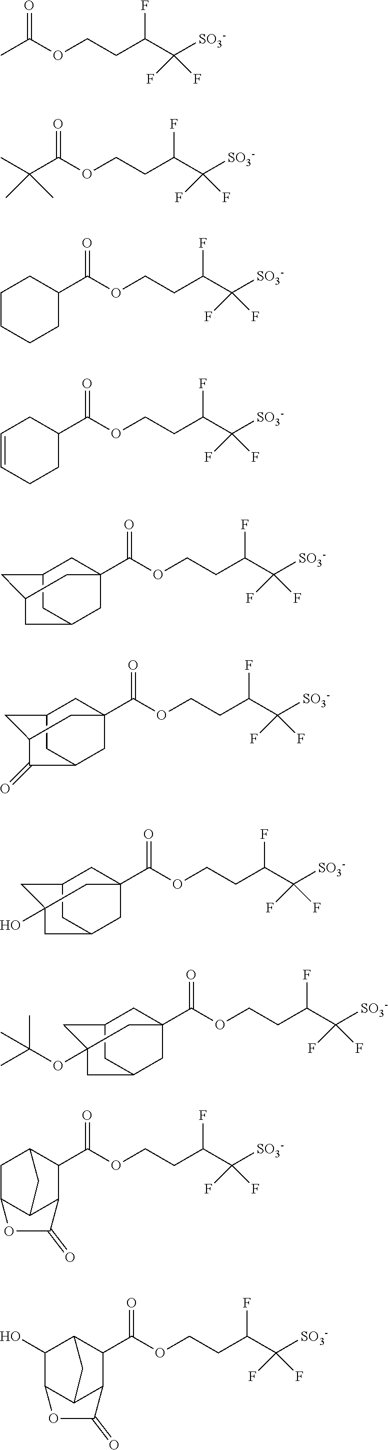

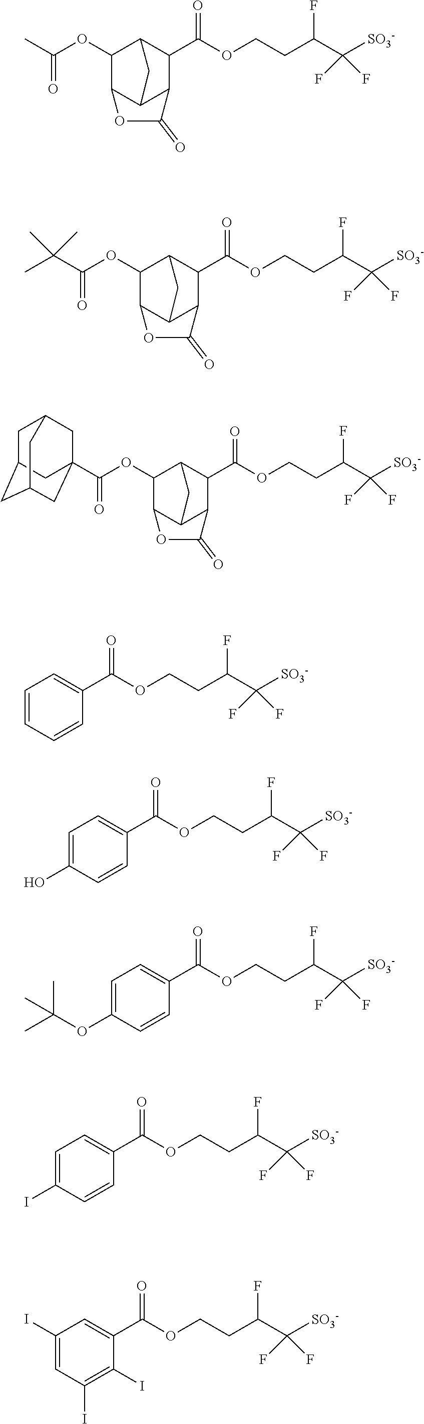

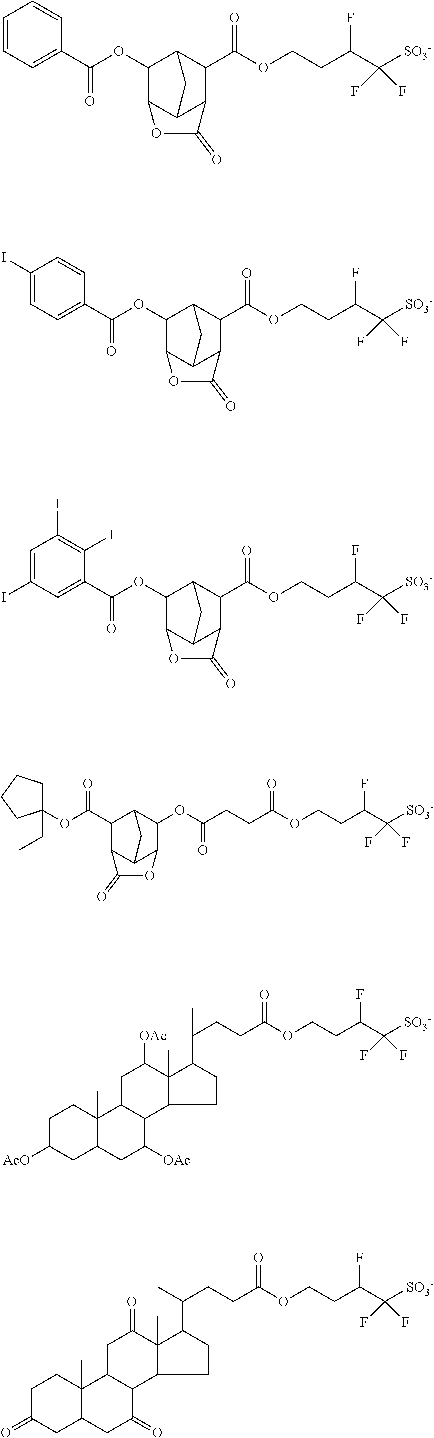

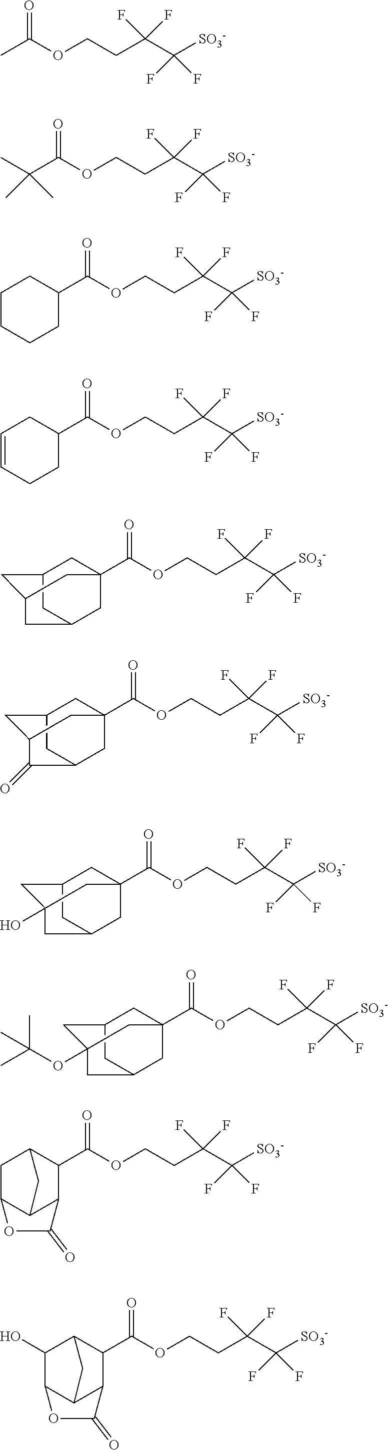

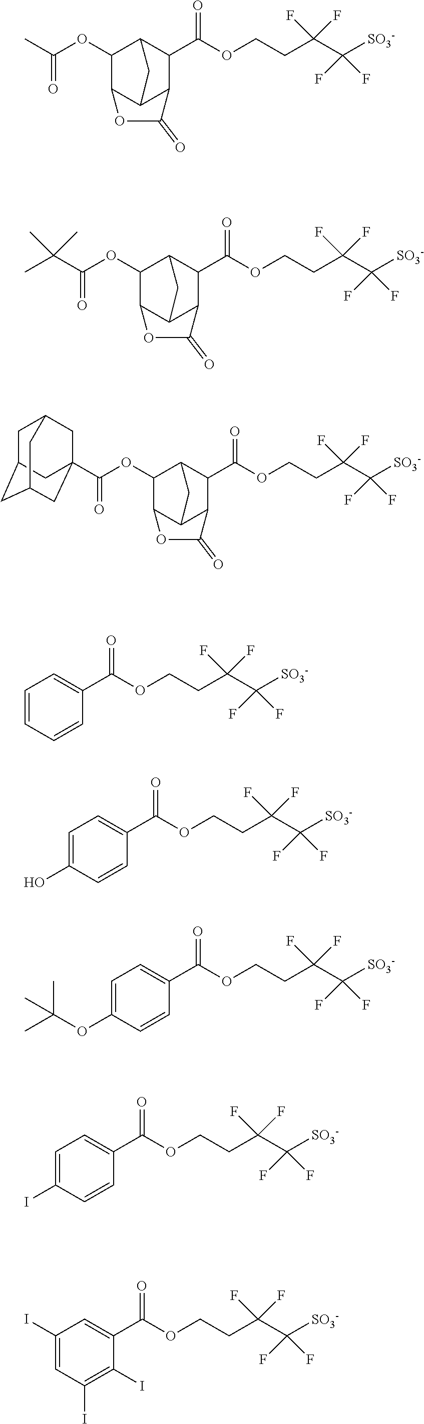

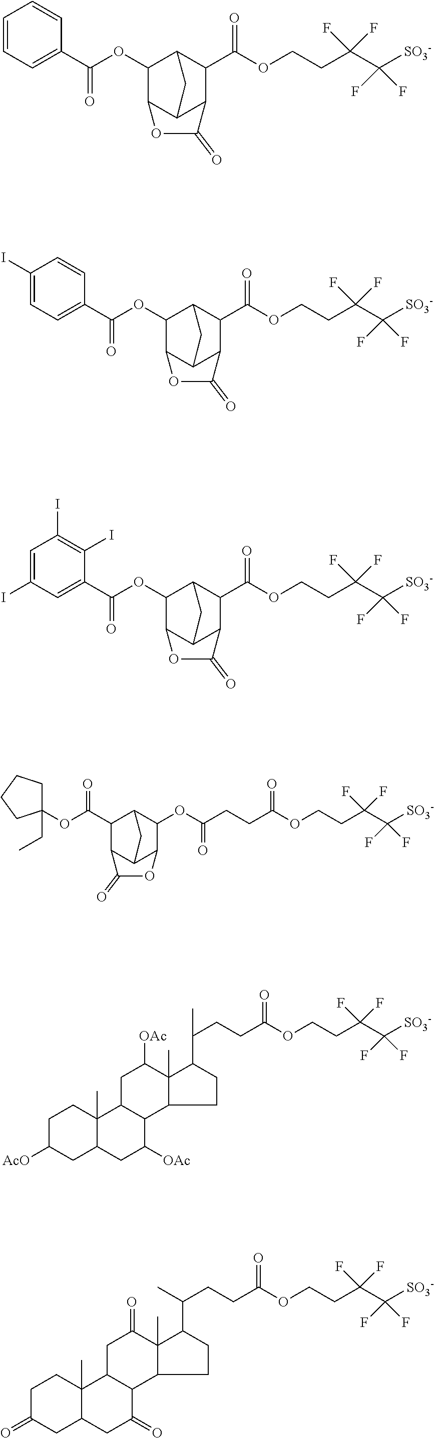

In the formula (1B), R^{fb1} and R^{fb2} independently represent a fluorine atom, or a hydrocarbyl group having 1 to 40 carbon atoms which may contain a heteroatom. The hydrocarbyl group may be any of a saturated, unsaturated, linear, branched, or cyclic hydrocarbyl group. Specific examples thereof include those similar to those exemplified as the hydrocarbyl group represented by R^{fa1} in the formula (1A′). R^{fb1} and R^{fb2} are preferably a fluorine atom or a linear fluorinated alkyl group having 1 to 4 carbon atoms. R^{fb1} and R^{fb2} may be bonded to each other to form a ring together with a group (—CF₂—SO₂—N⁻—SO₂—CF₂—) to which they are bonded, and in this case, a group obtained by bonding R^{fb1} and R^{fb2} to each other is preferably a fluorinated ethylene group or a fluorinated propylene group.

In the formula (1C), R^{fc1}, R^{fc2} and R^{fc3} independently represent a fluorine atom, or a hydrocarbyl group having 1 to 40 carbon atoms which may contain a heteroatom. The hydrocarbyl group may be any of a saturated, unsaturated, linear, branched, or cyclic hydrocarbyl group. Specific examples thereof include those similar to those exemplified as the hydrocarbyl group represented by R^{fa1} in the formula (1A′). R^{fc1}, R^{fc2} and R^{fc3} are preferably a fluorine atom or a linear fluorinated alkyl group having 1 to 4 carbon atoms. R^{fc1} and R^{fc2} may be bonded to each other to form a ring together with a group (—CF₂—SO₂−C⁻—SO₂—CF₂—) to which they are bonded, and in this case, a group obtained by bonding R^{fc1} and R^{fc2} to each other is preferably a fluorinated ethylene group or a fluorinated propylene group.

In the formula (1D), R^{fd} represents a hydrocarbyl group having 1 to 40 carbon atoms which may contain a heteroatom. The hydrocarbyl group may be any of a saturated, unsaturated, linear, branched, or cyclic hydrocarbyl group. Specific examples thereof include those similar to those exemplified as the hydrocarbyl group represented by R^{fa1} in the formula (1A′).

Examples of the anion represented by the formula (1D) include, but are not limited thereto.

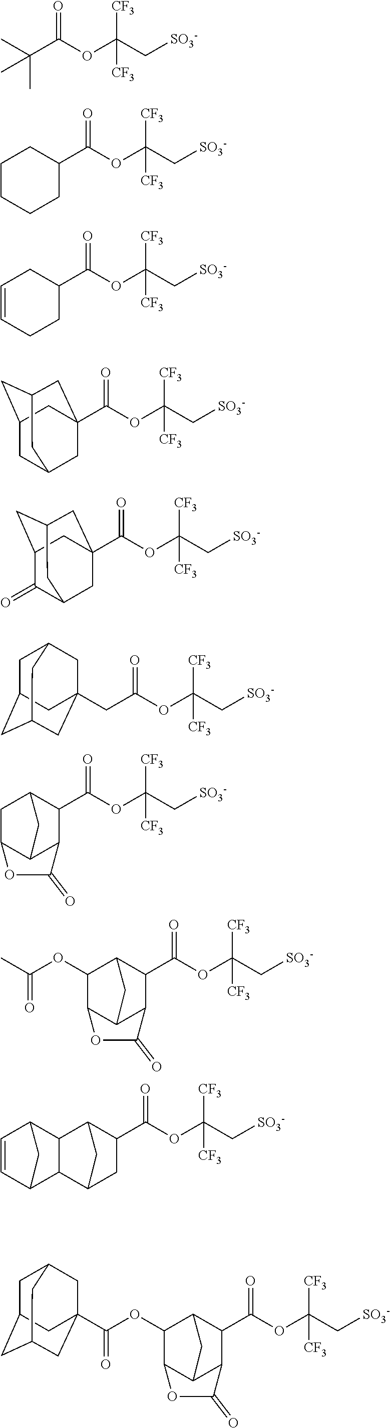

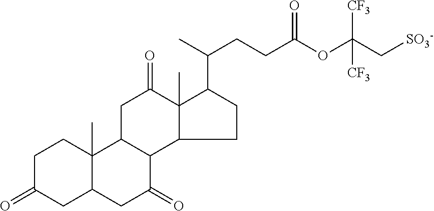

Examples of the non-nucleophilic counter ion further include anions having an aromatic ring substituted with an iodine atom or a bromine atom. Examples of the anion include those represented by the following formula (1E).

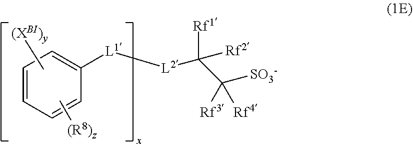

In the formula (1E), x is an integer satisfying 1≤x≤3, y and z are integers satisfying 1≤y≤5, 0≤z≤3, and 1≤y+z≤5, y is preferably an integer satisfying 1≤ y≤ 3, and more preferably 2 or 3, and z is preferably an integer satisfying 0≤z≤2.

In the formula (1E), X^{BI} is an iodine atom or a bromine atom, and when x and/or y is 2 or more, each of X^{BI} may be identical to or different from each other.

In the formula (1E), L^{1′} is a single bond, an ether bond, or an ester bond, or a saturated hydrocarbylene group having 1 to 6 carbon atoms which may contain an ether bond or an ester bond. The saturated hydrocarbylene group may be linear, branched, or a cyclic saturated hydrocarbylene group.

In the formula (1E), when x is 1, L²′ is a single bond or a divalent linkage group having 1 to 20 carbon atoms, and when x is 2 or 3, L^{2′} is a (x+1)-valent linkage group having 1 to 20 carbon atoms which may contain an oxygen atom, a sulfur atom, or a nitrogen atom.

In the formula (1E), R⁸ is a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 10 carbon atoms, a hydrocarbylcarbonyloxy group having 2 to 20 carbon atoms, or a hydrocarbylsulfonyloxy group having 1 to 20 carbon atoms, or —N(R^{8A}) (R^{8B}), —N(R^{8C})—C(═O)—R^{8D} or —N(R^{8C})—C(═O)—O—R^{8D}, which optionally contain a hydroxy group, a carboxyl group, a fluorine atom, a chlorine atom, a bromine atom, or an amino group, or a fluorine atom, a chlorine atom, a bromine atom, a hydroxy group, an amino group, an ether bond, an ester bond or an amide bond. R^{8A} and R^{8B} independently represent a hydrogen atom or a saturated hydrocarbyl group having 1 to 6 carbon atoms. R^{8C} is a hydrogen atom or a saturated hydrocarbyl group having 1 to 6 carbon atoms, and may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{8D} is an aliphatic hydrocarbyl group having 1 to 16 carbon atoms, an aryl group having 6 to 12 carbon atoms, or an aralkyl group having 7 to 15 carbon atoms, which may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be any of a saturated, unsaturated, linear, branched, or cyclic hydrocarbyl group. The hydrocarbyl group, the hydrocarbyloxy group, the hydrocarbylcarbonyl group, the hydrocarbyloxycarbonyl group, the hydrocarbylcarbonyloxy group and the hydrocarbylsulfonyloxy group may be linear, branched or cyclic. When x and/or z is 2 or more, each R⁸ may be identical to or different from each other.

Among these, R⁸ is preferably a hydroxy group, —N(R^{8C})—C(═O)—R^{8D}, —N(R^{8C})—C(═O)—O—R^{8D}, a fluorine atom, a chlorine atom, a bromine atom, a methyl group, a methoxy group, or the like.

In the formula (1E), Rf^{1′} to Rf⁴′ independently represent a hydrogen atom, a fluorine atom, or a trifluoromethyl group, and at least one of them is a fluorine atom or a trifluoromethyl group. Further, Rf^{1′} and Rf²′, taken together, may form a carbonyl group. In particular, both of Rf³′ and Rf⁴′ are preferably a fluorine atom.

Examples of the anion of the onium salt represented by the formula (1E) include, but are not limited thereto. In the following formulae, X^{BI} is as defined above.

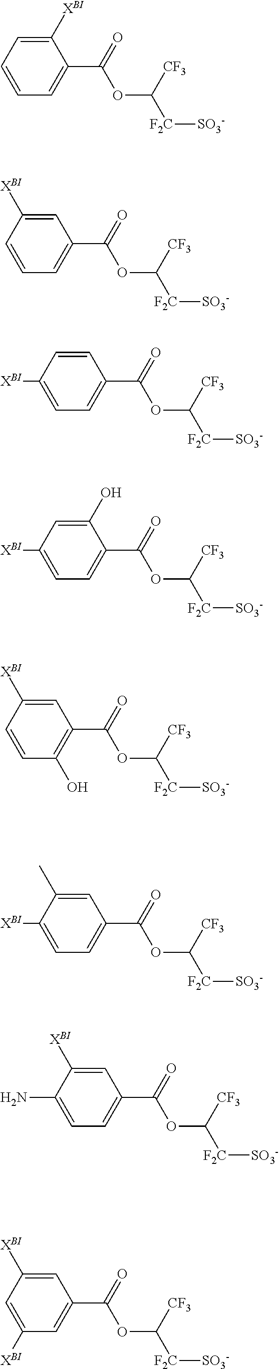

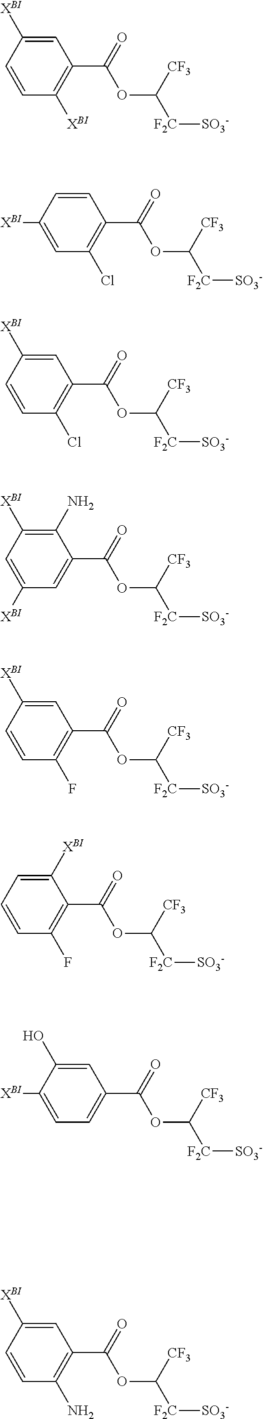

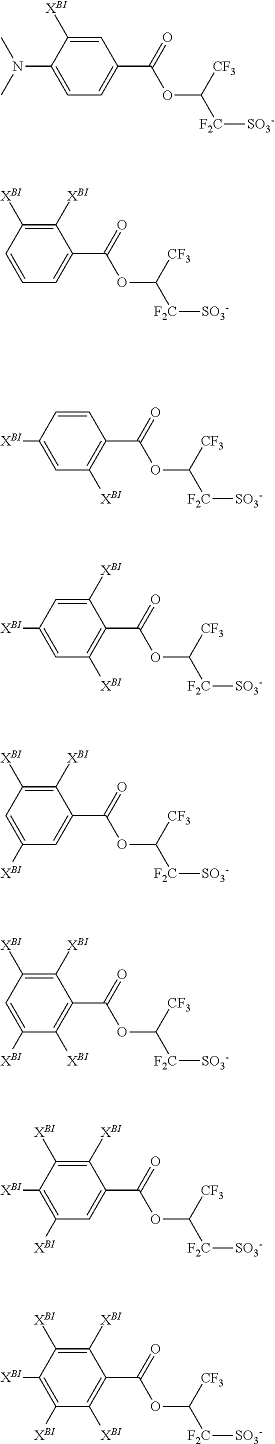

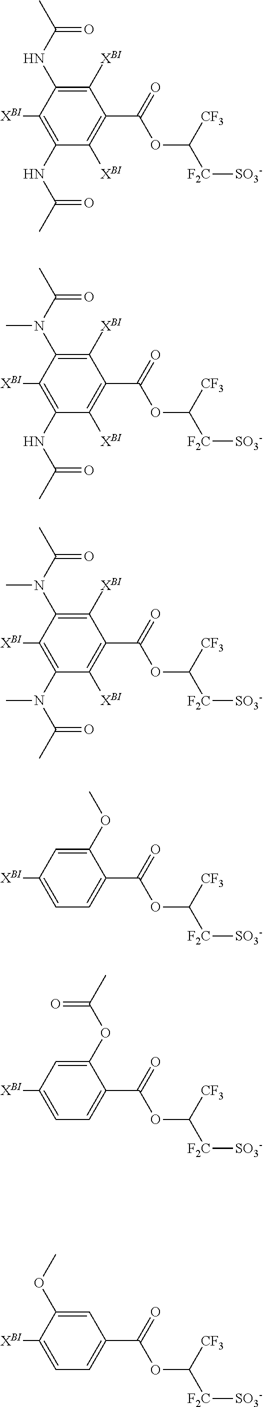

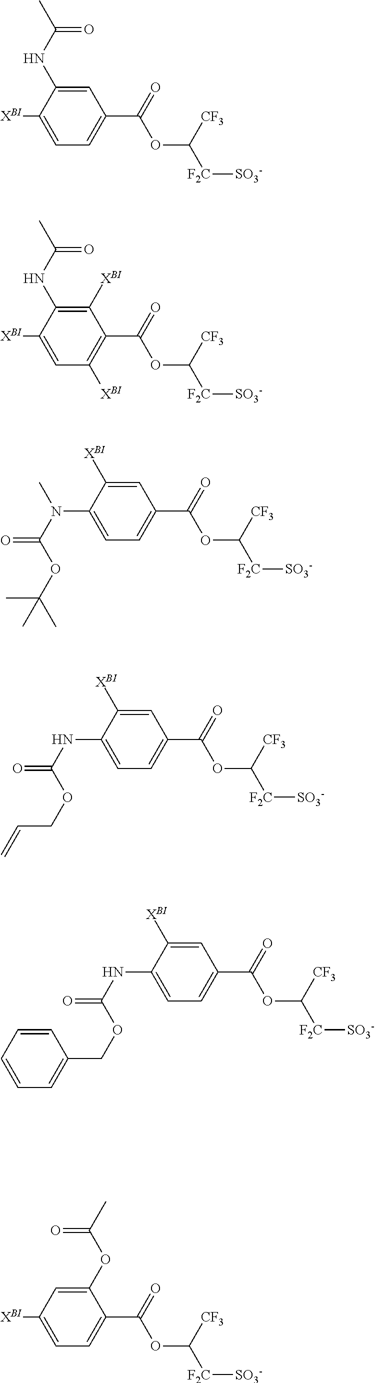

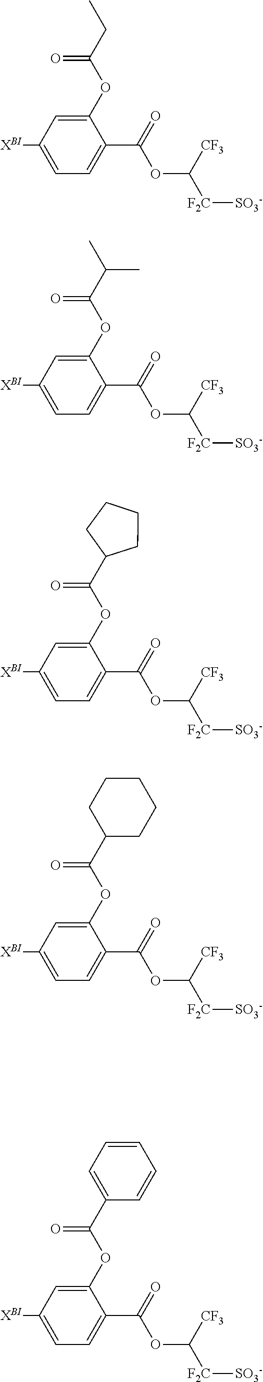

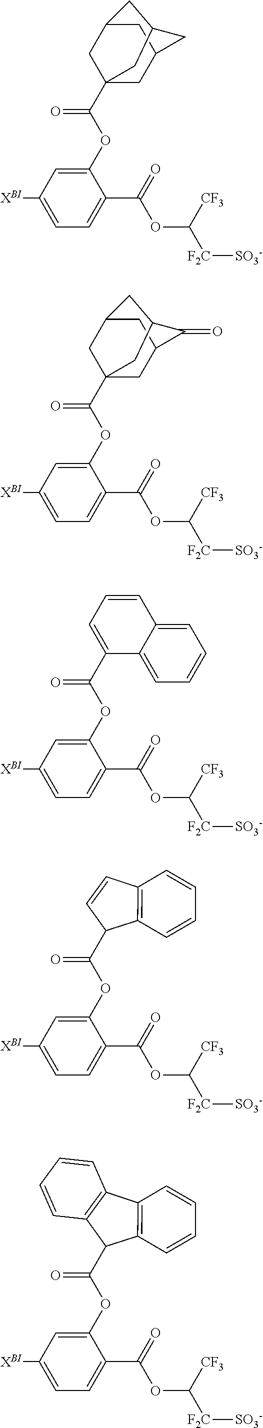

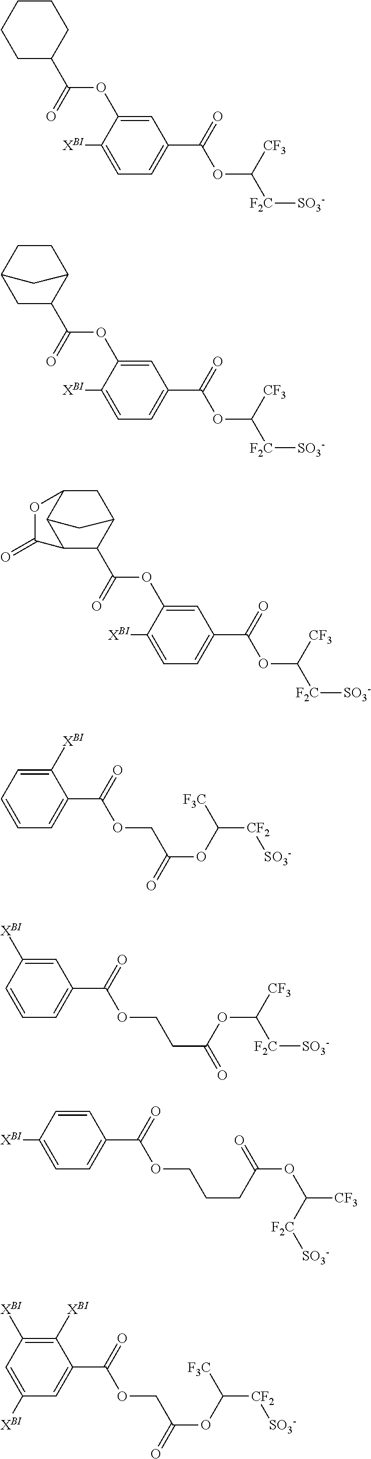

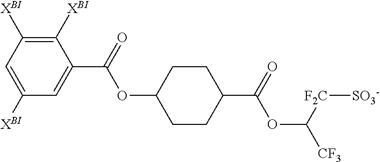

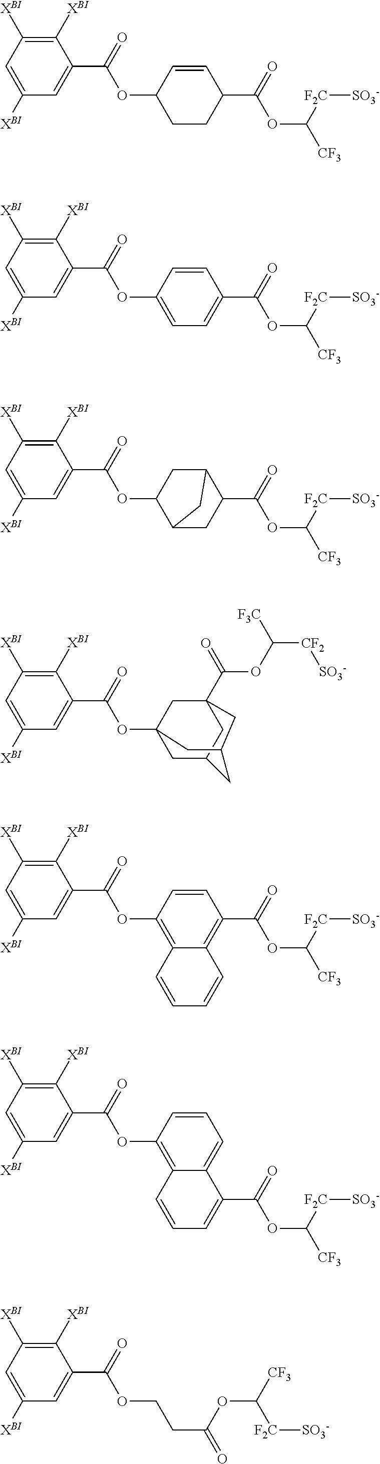

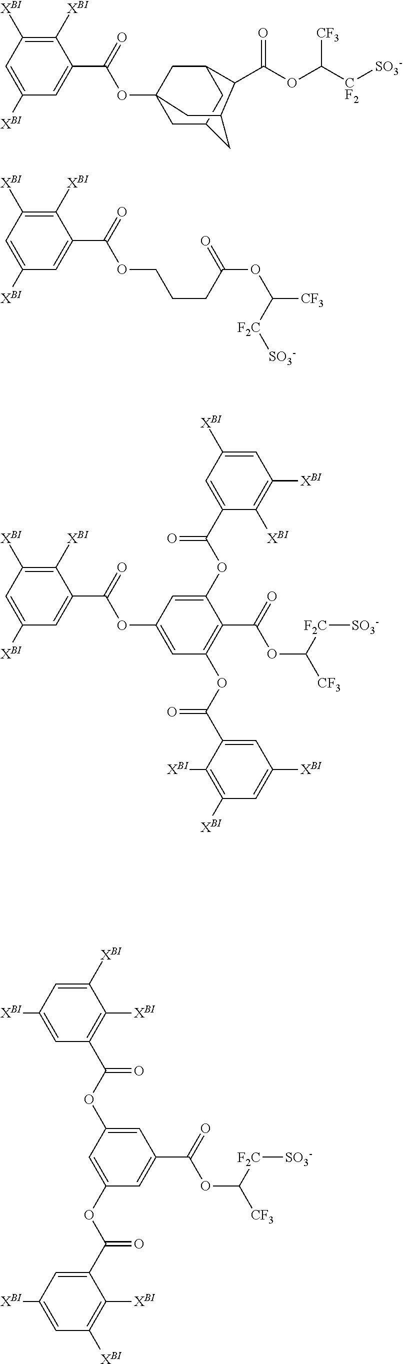

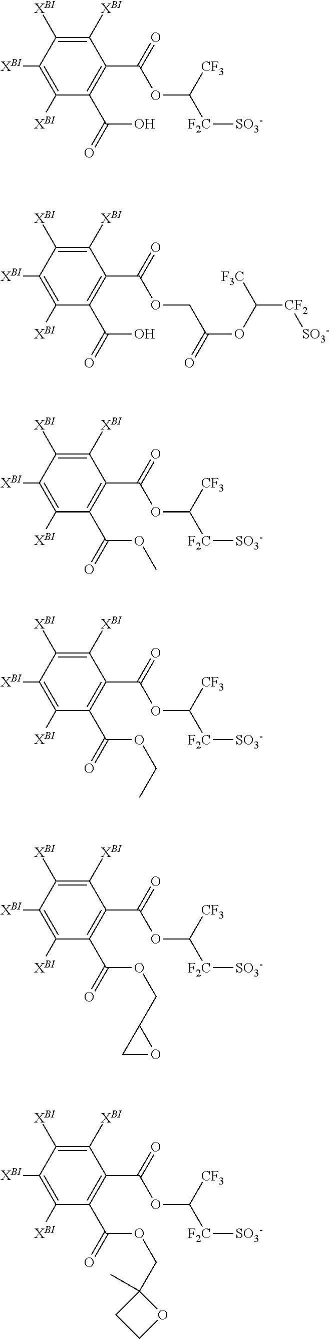

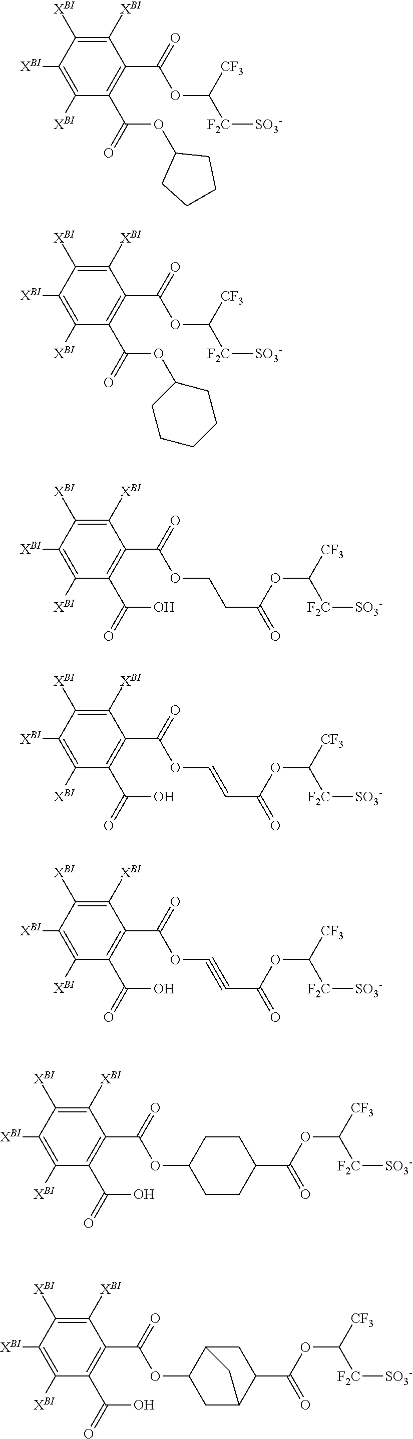

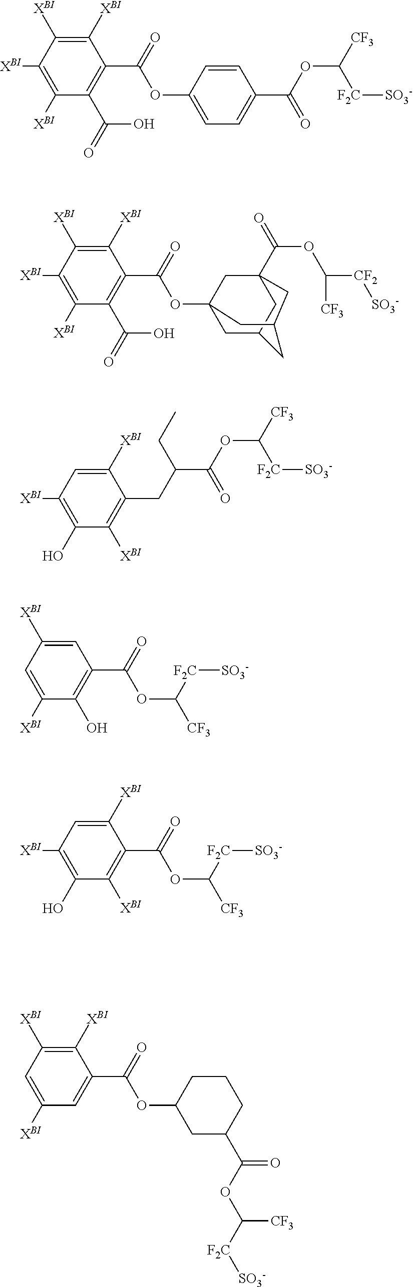

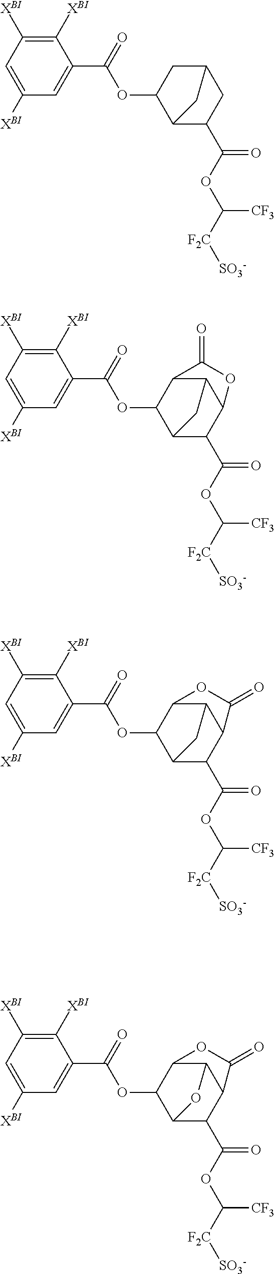

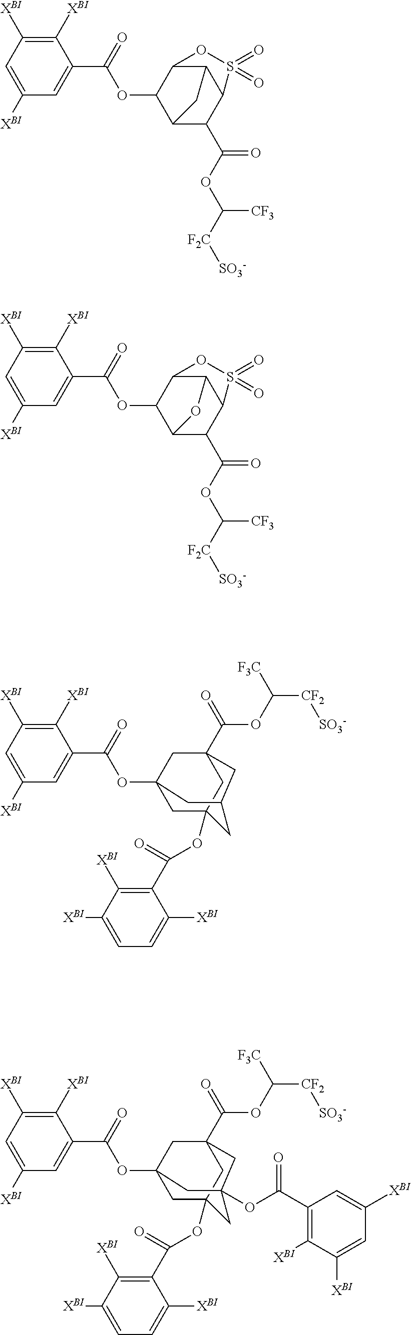

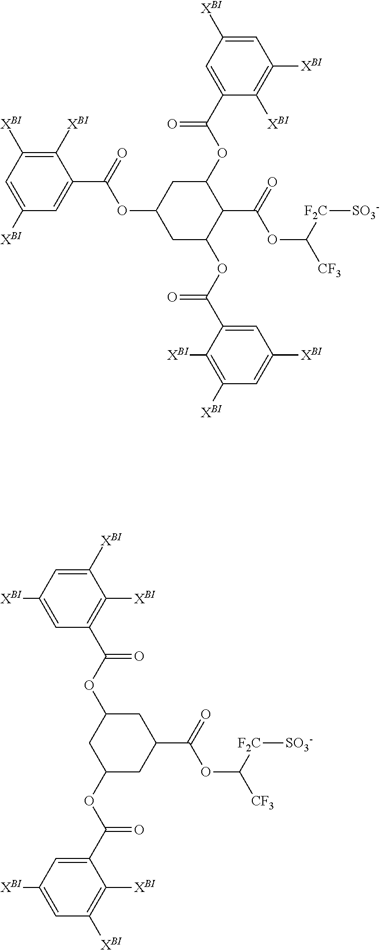

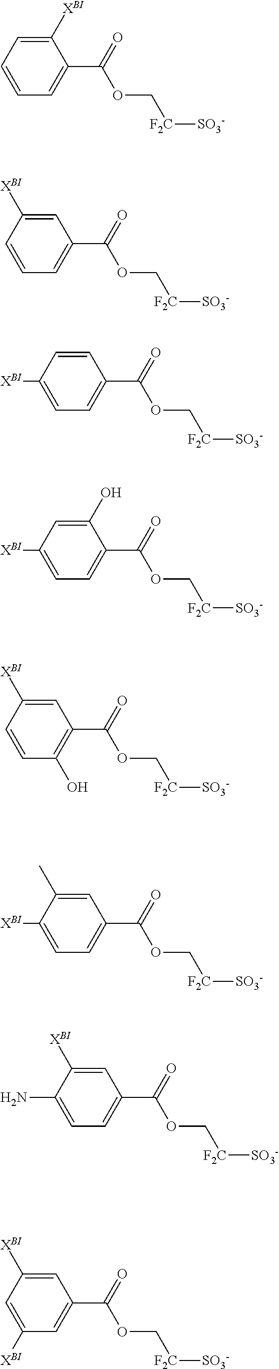

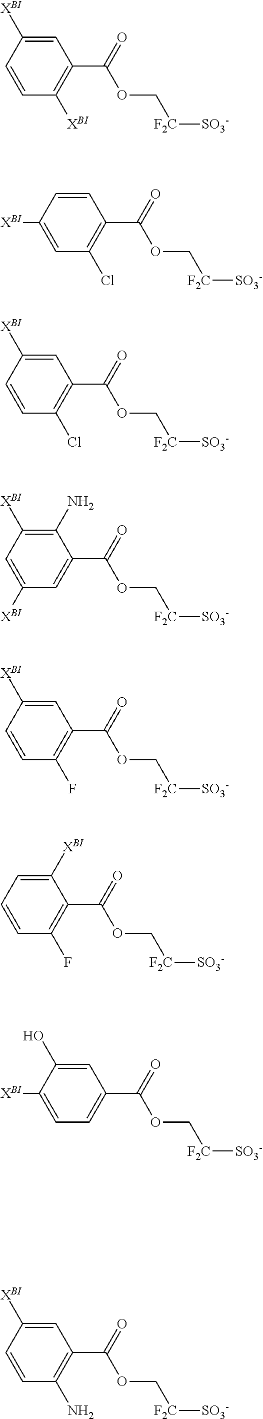

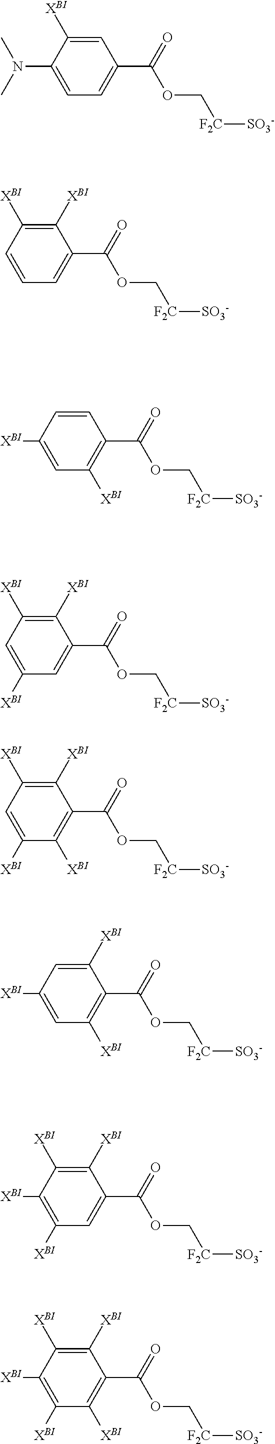

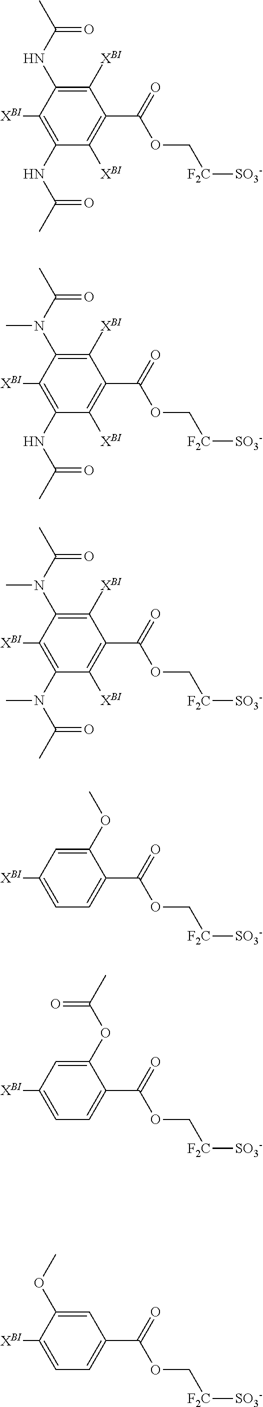

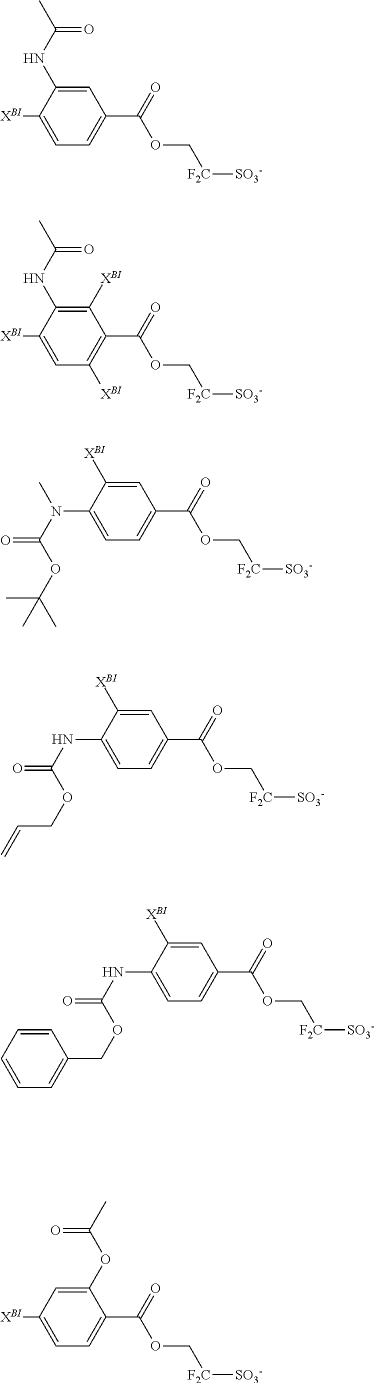

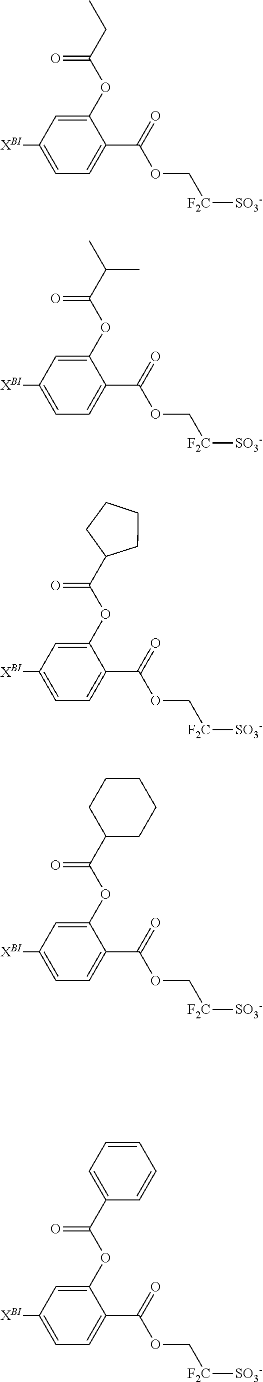

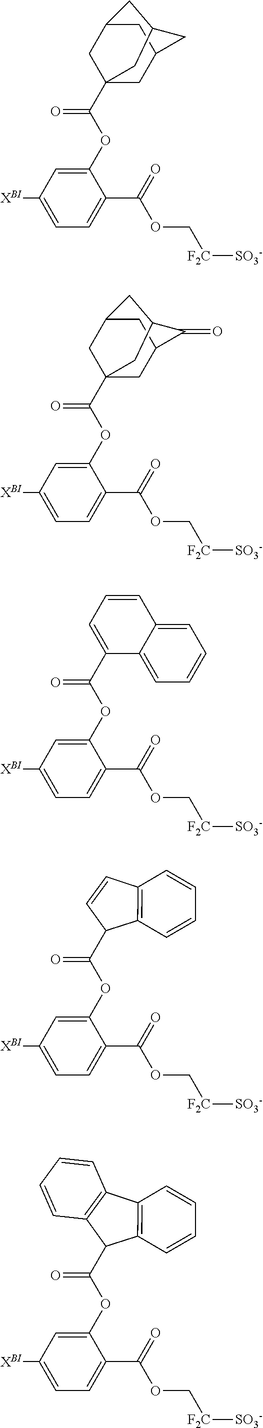

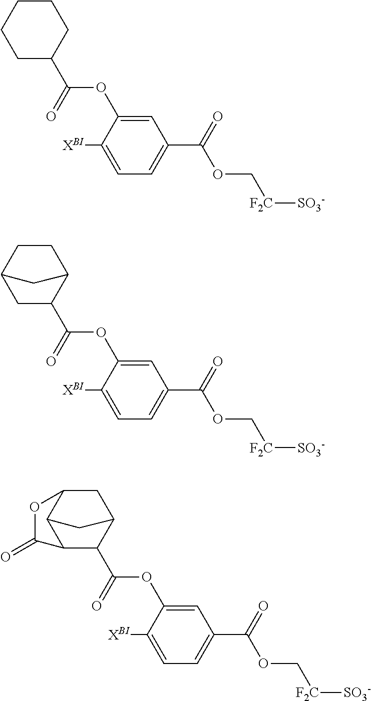

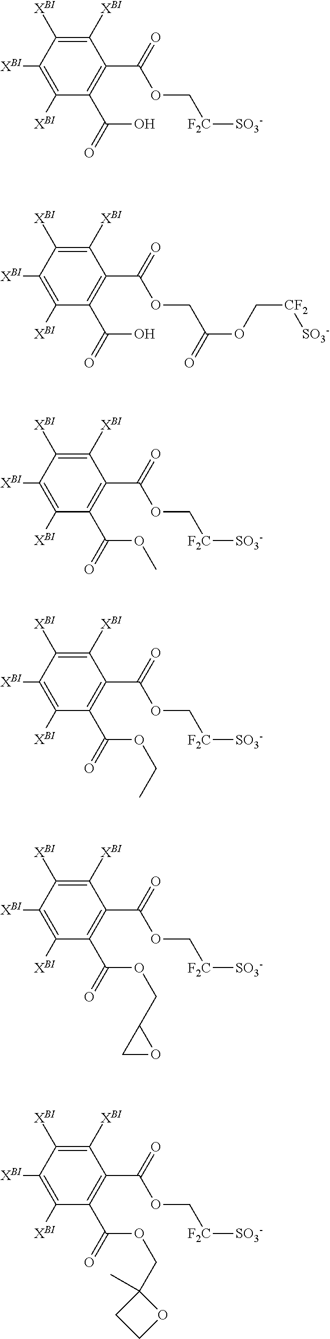

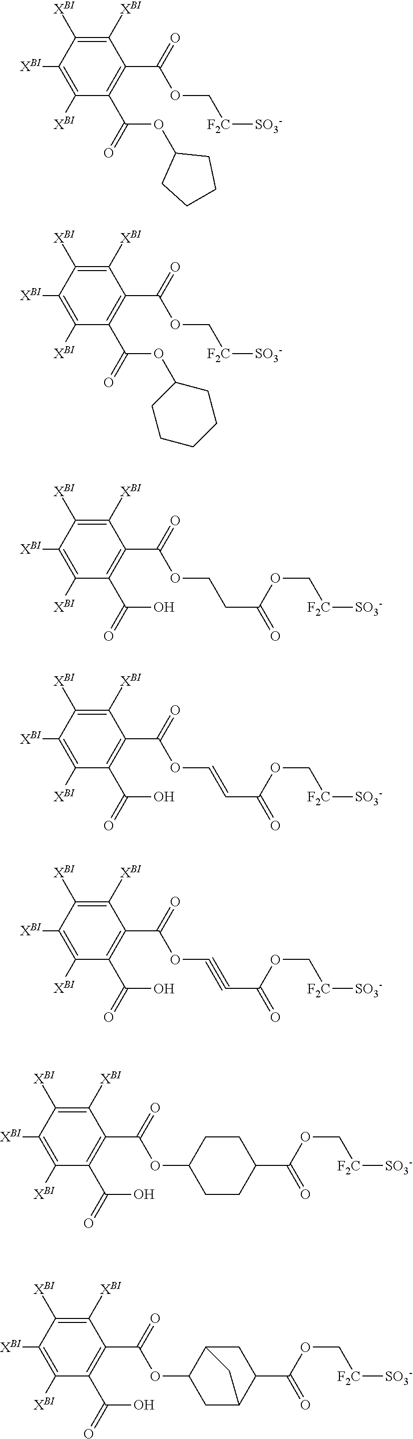

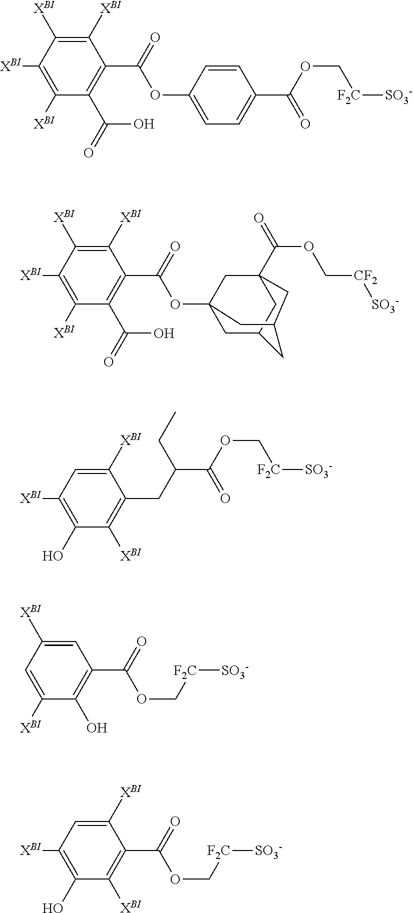

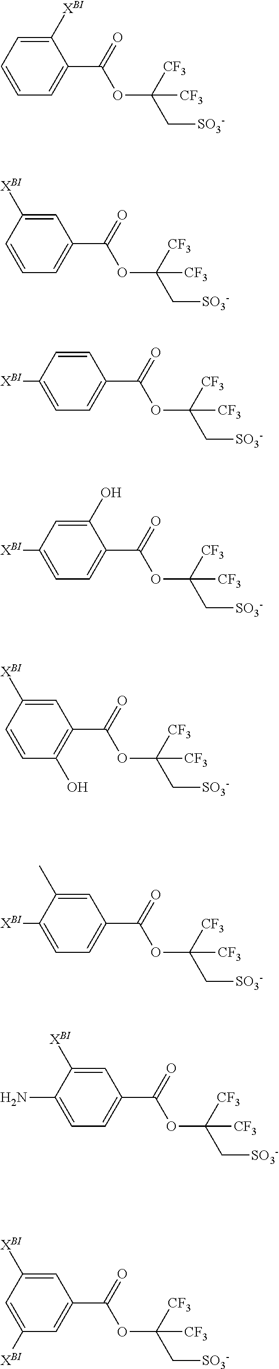

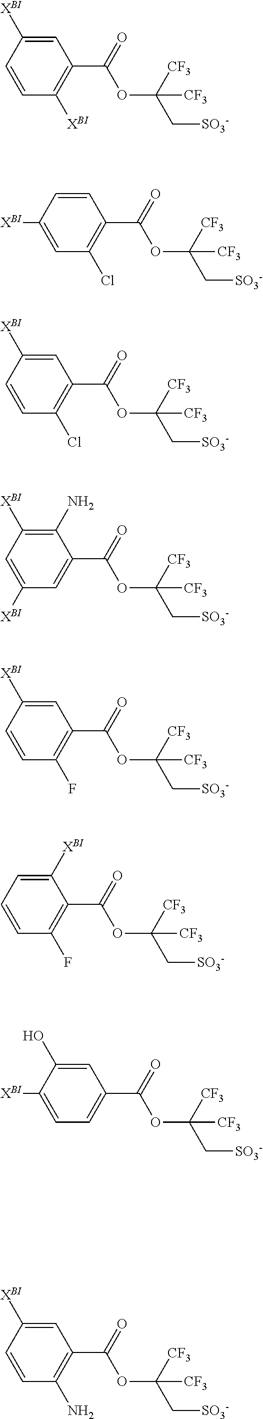

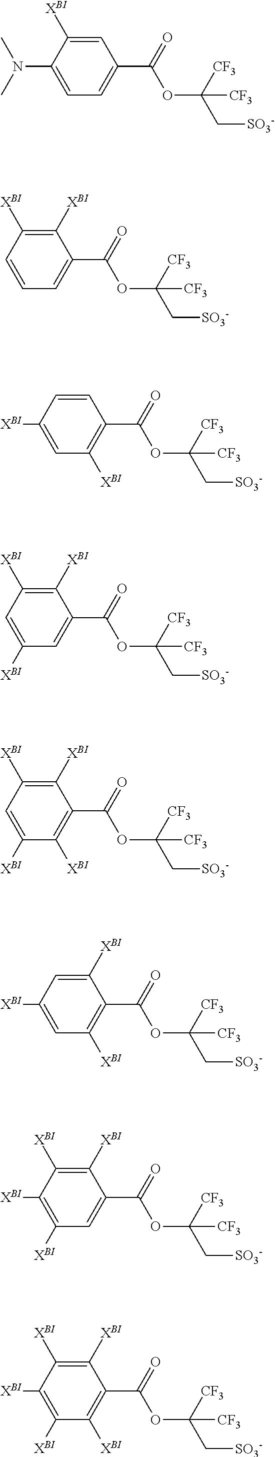

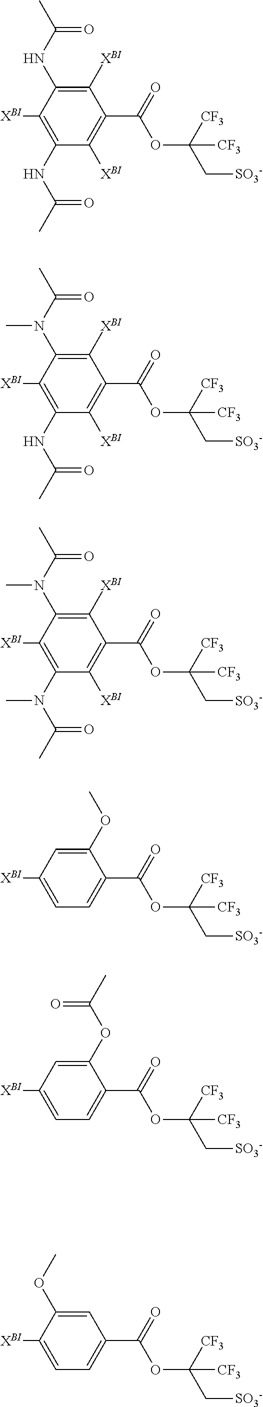

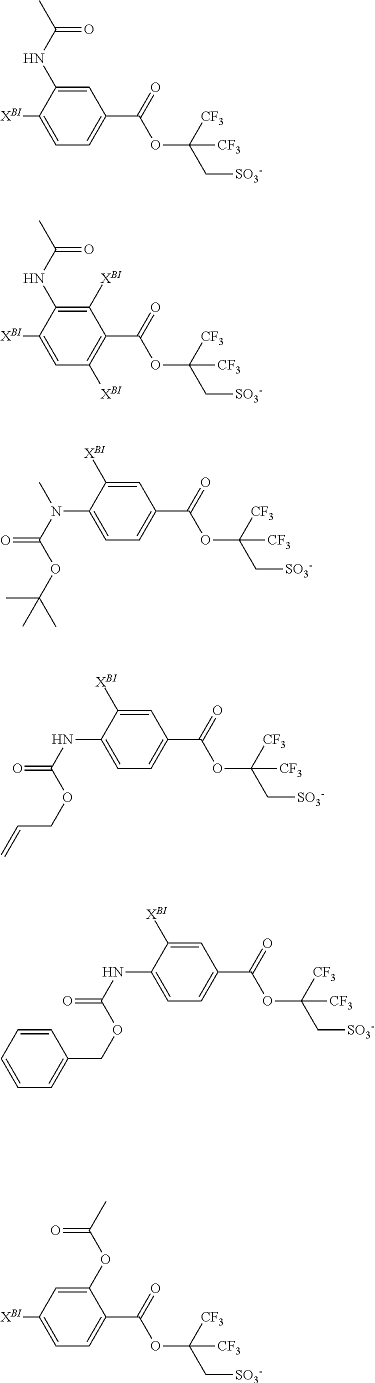

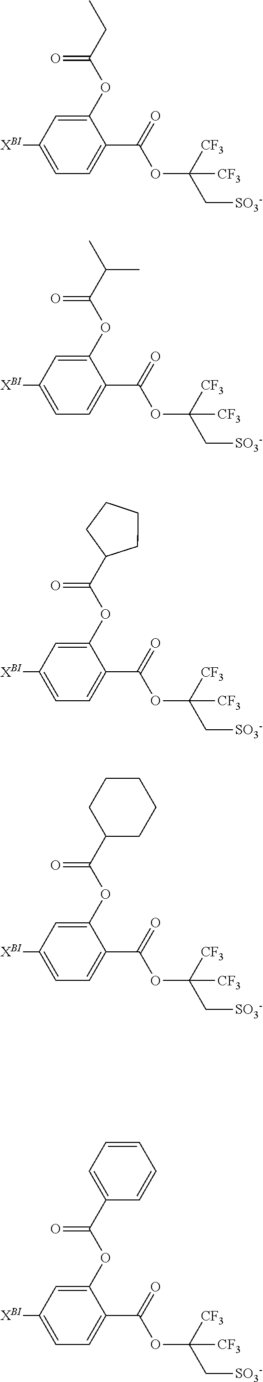

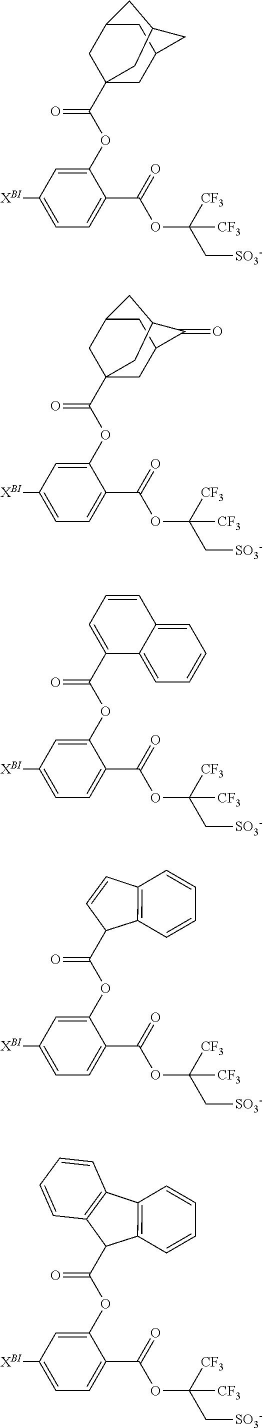

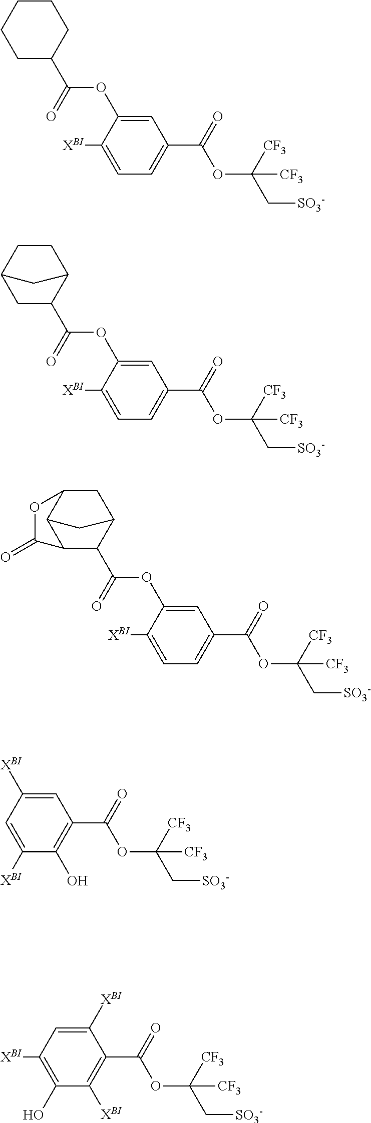

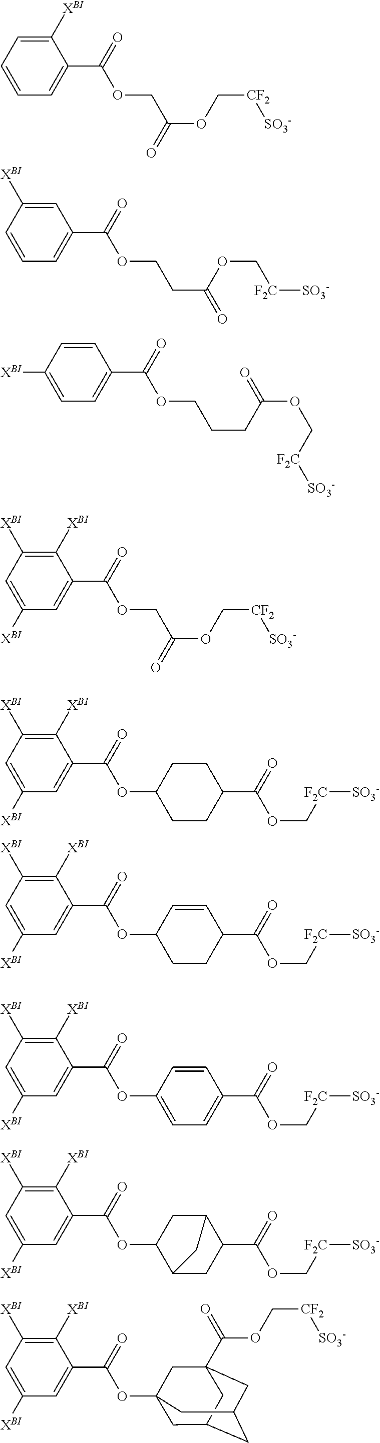

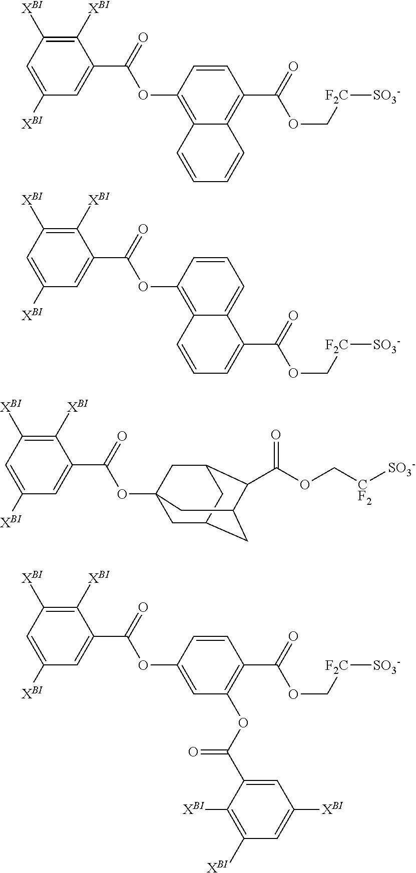

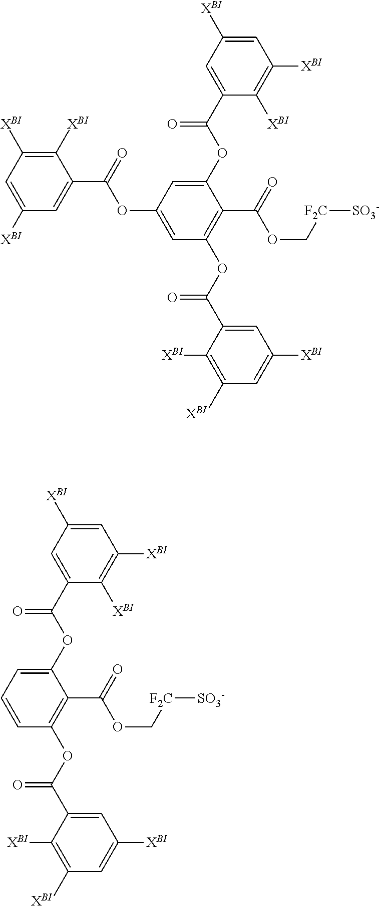

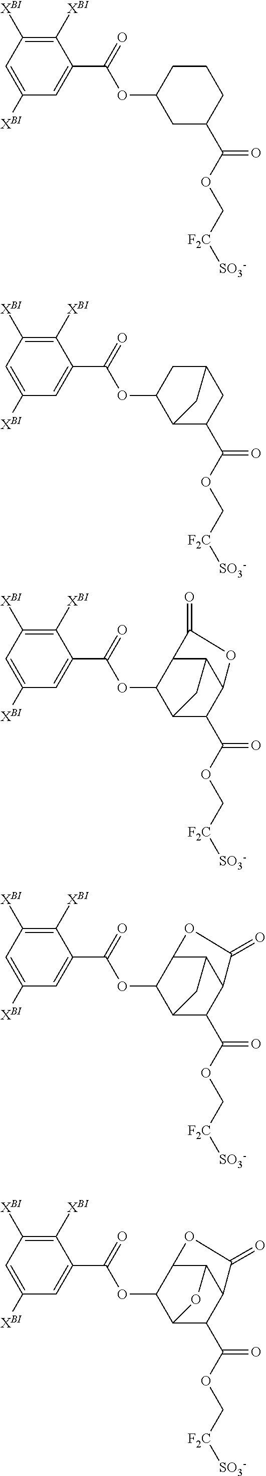

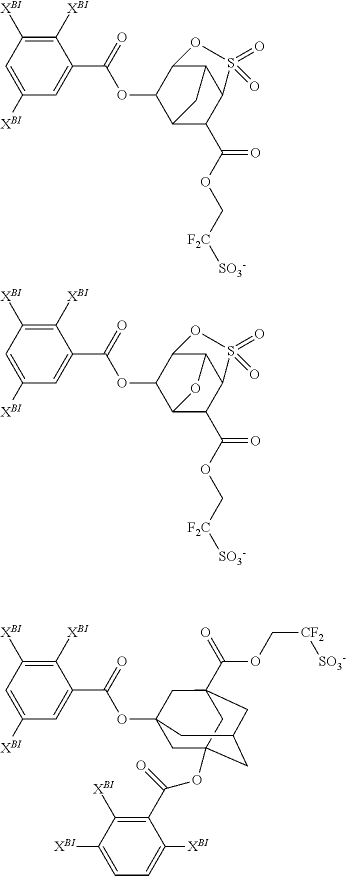

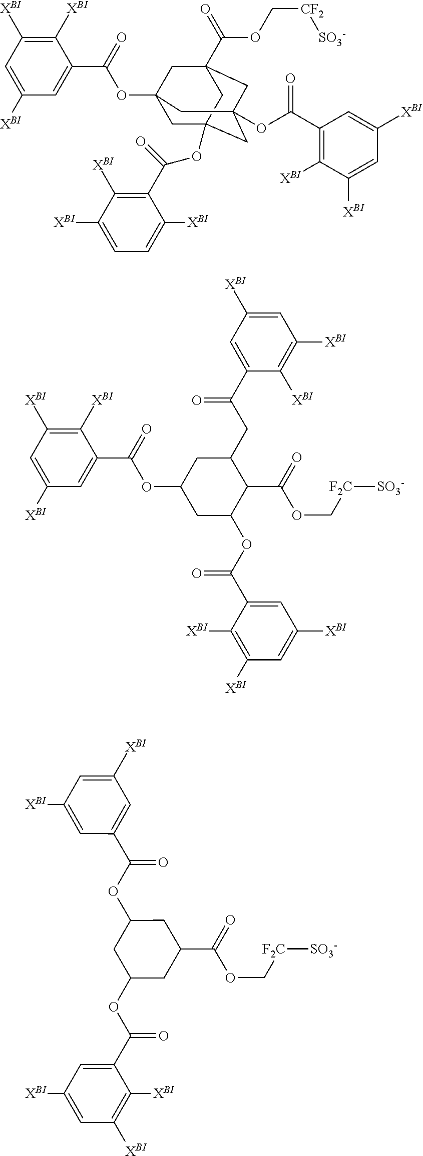

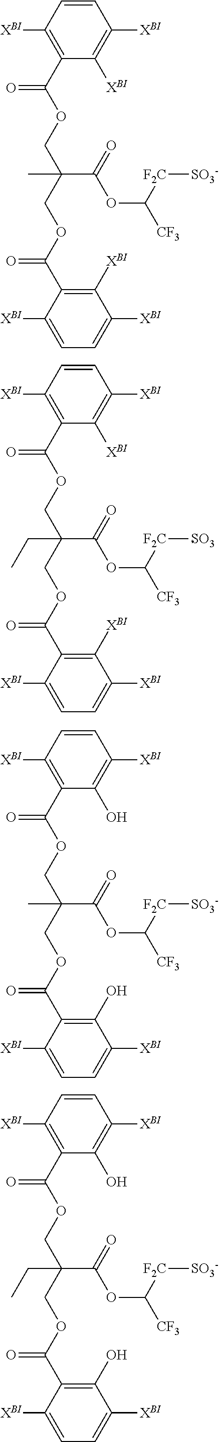

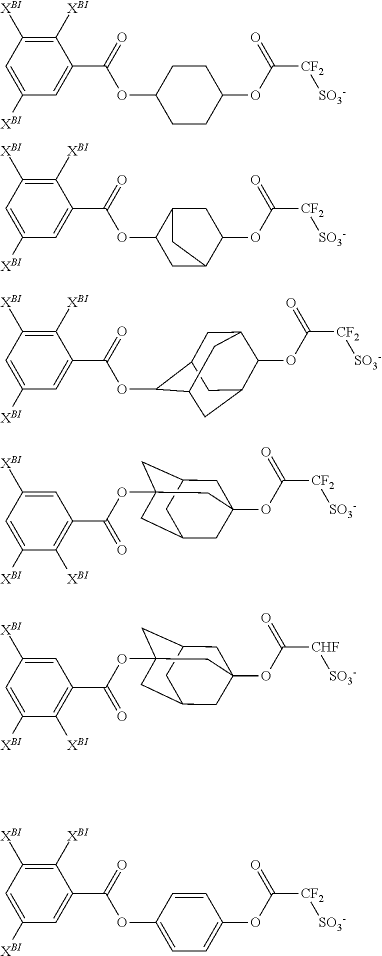

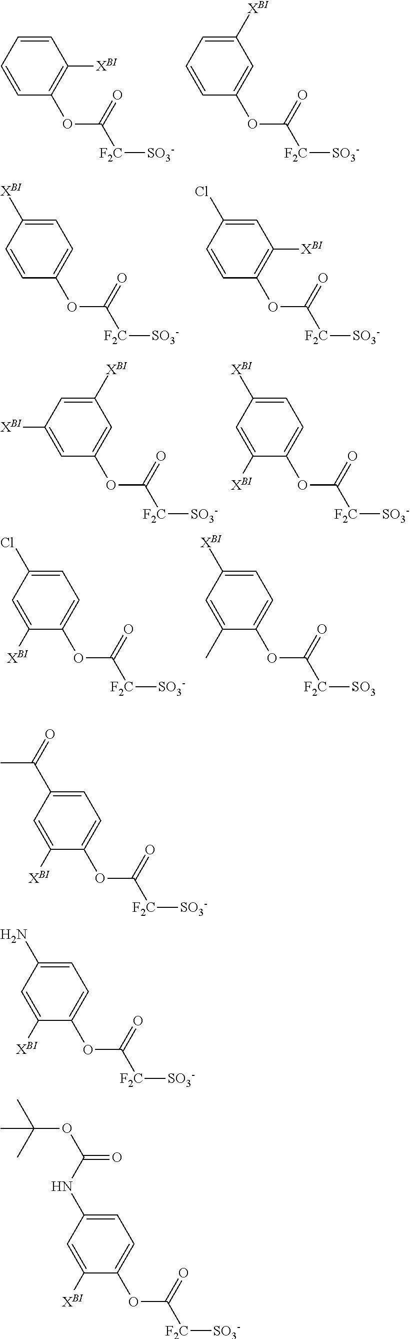

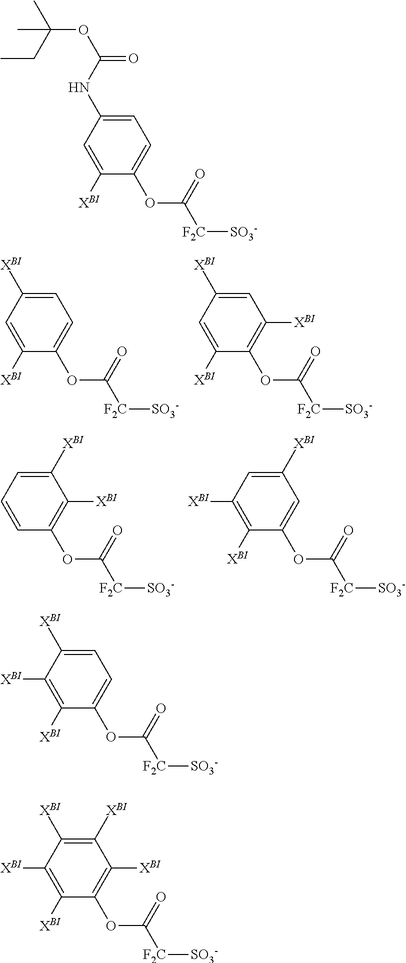

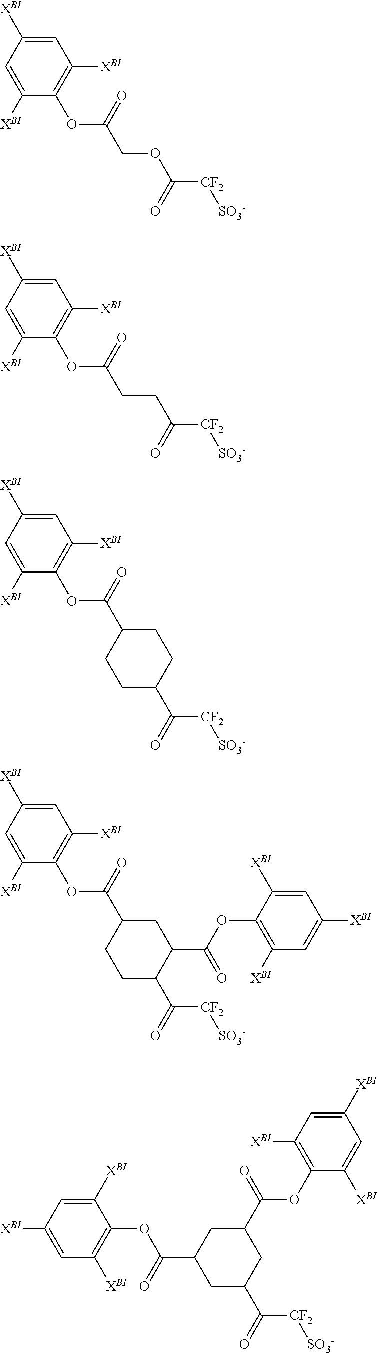

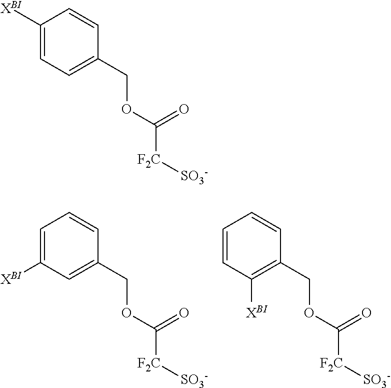

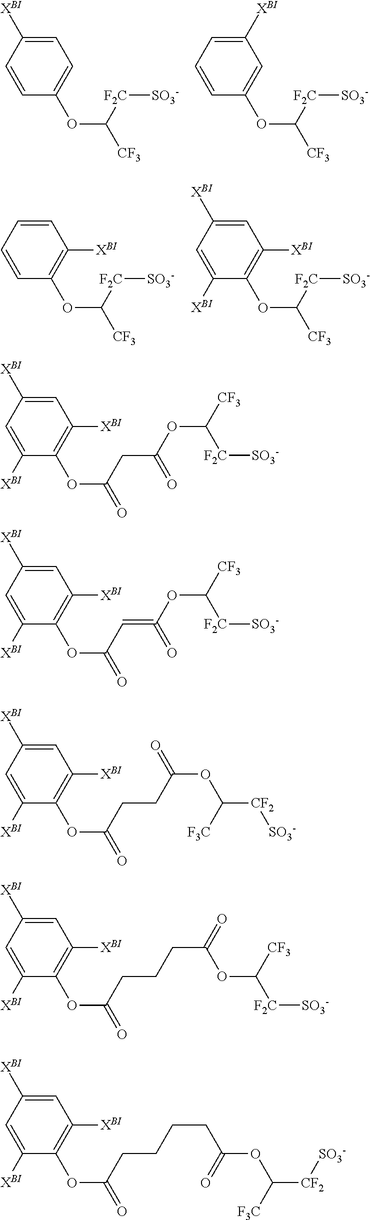

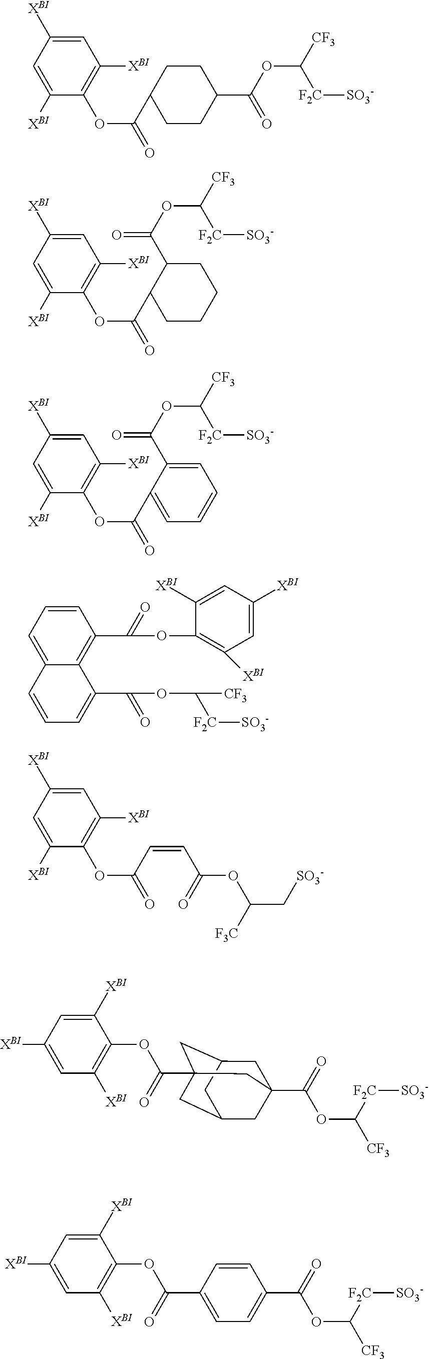

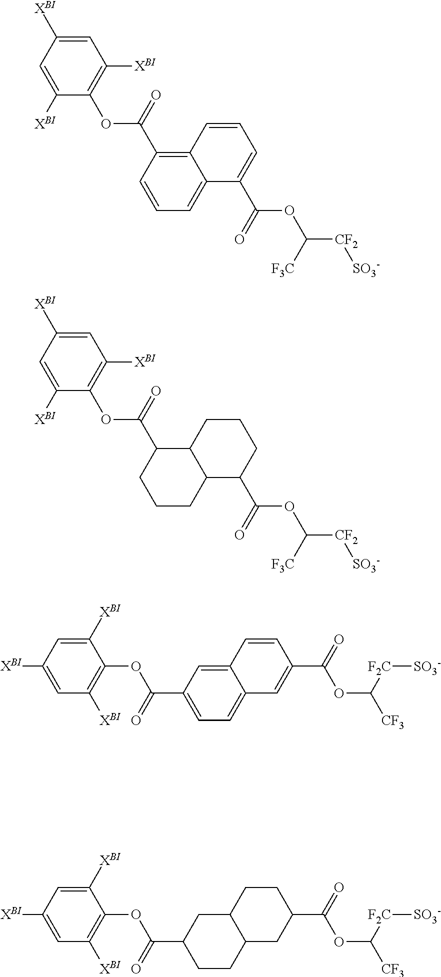

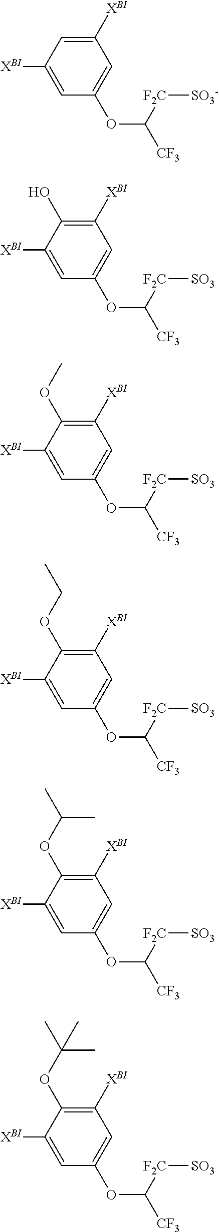

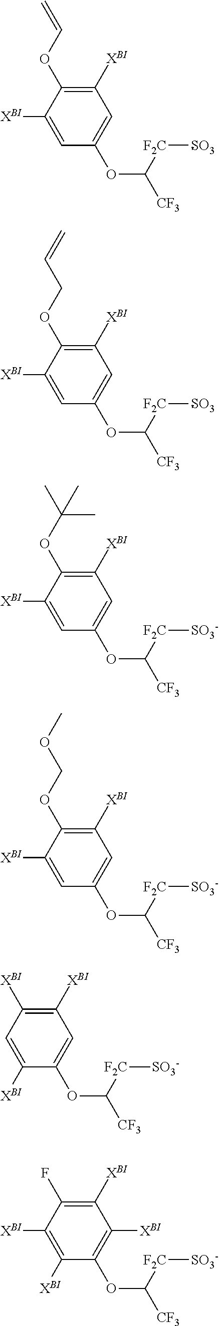

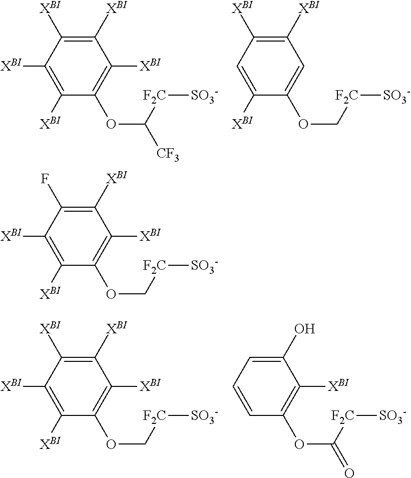

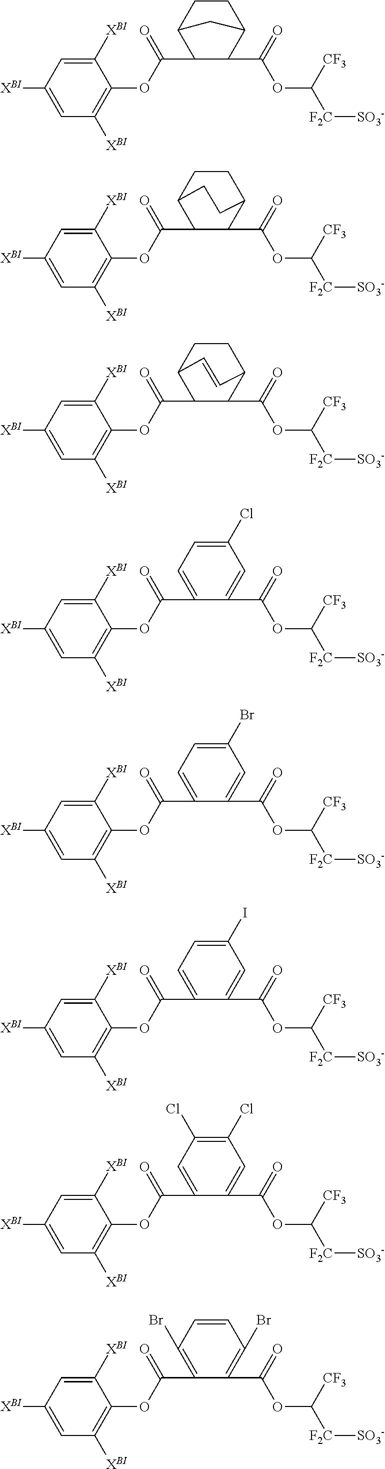

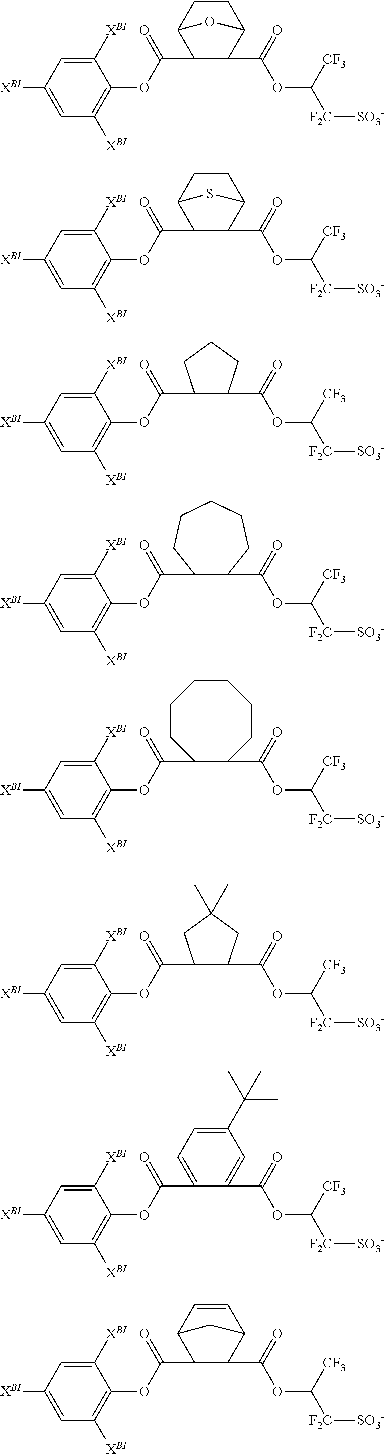

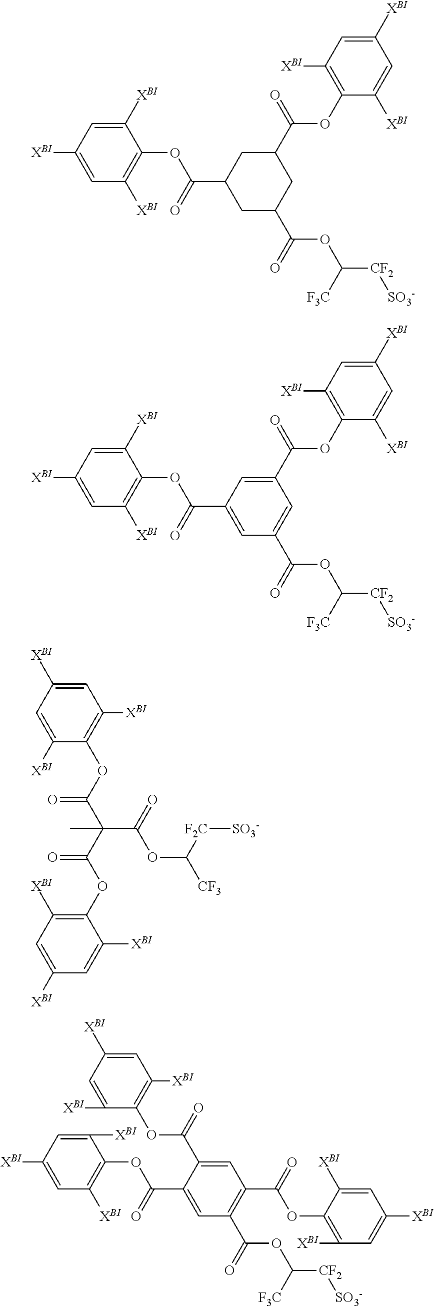

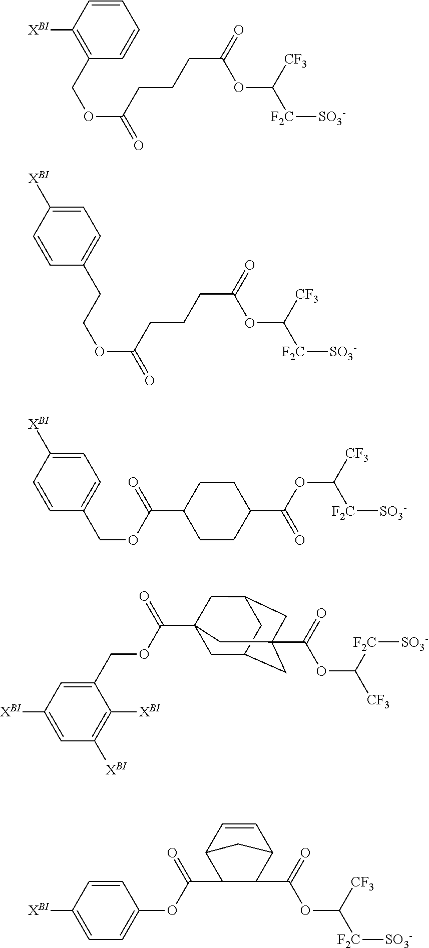

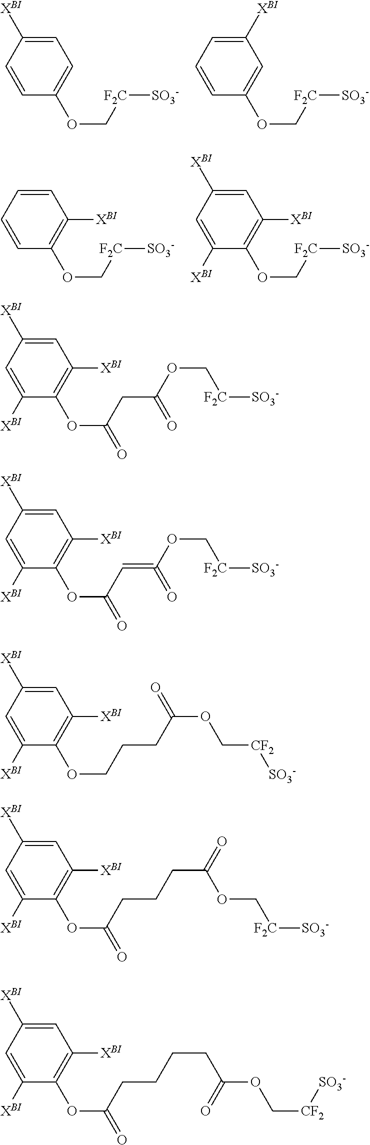

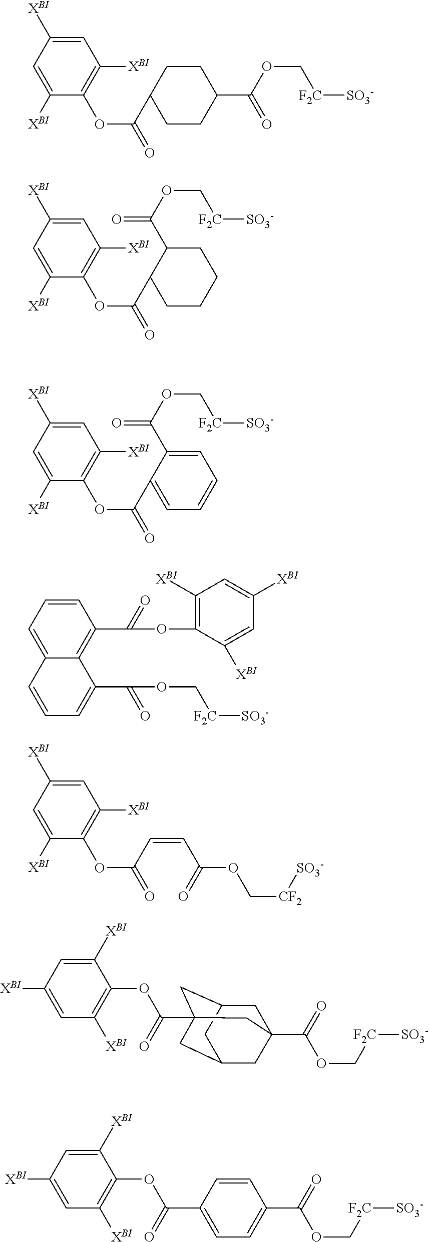

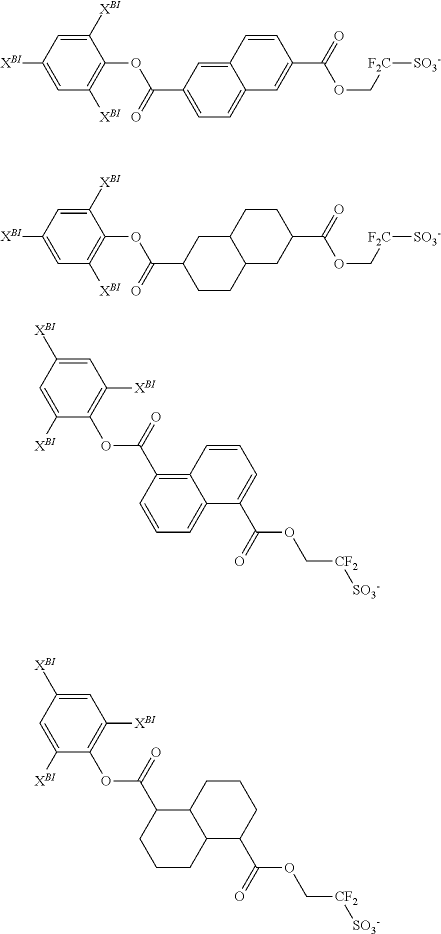

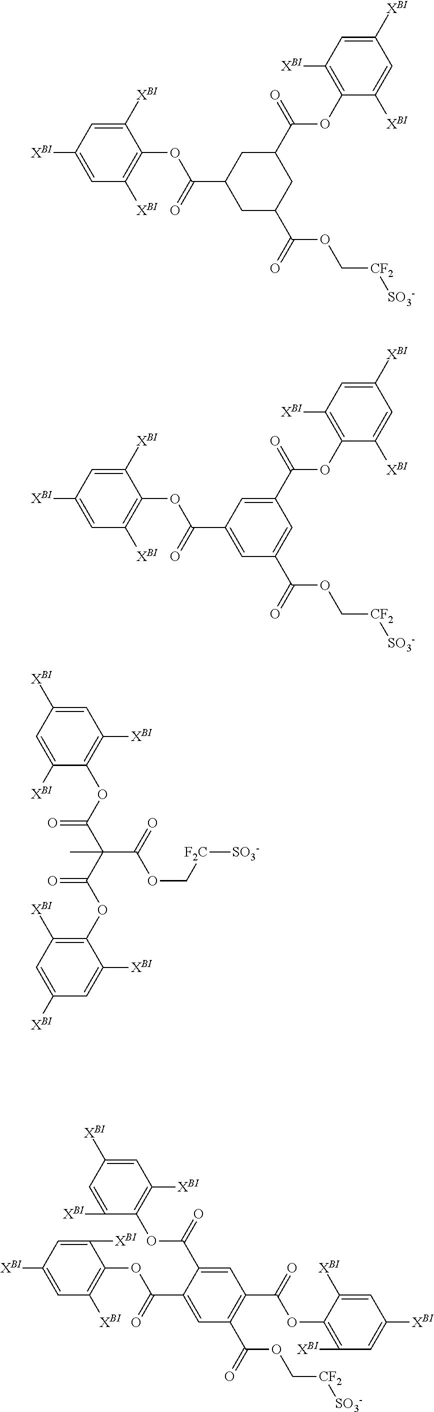

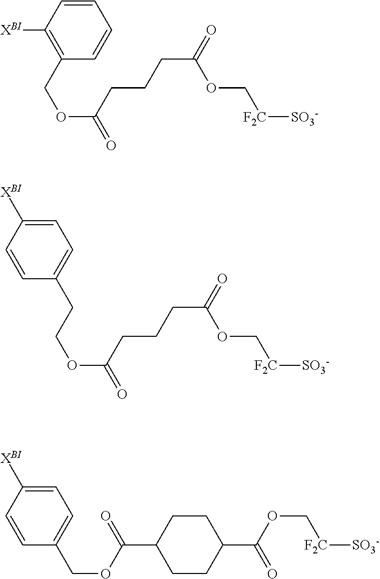

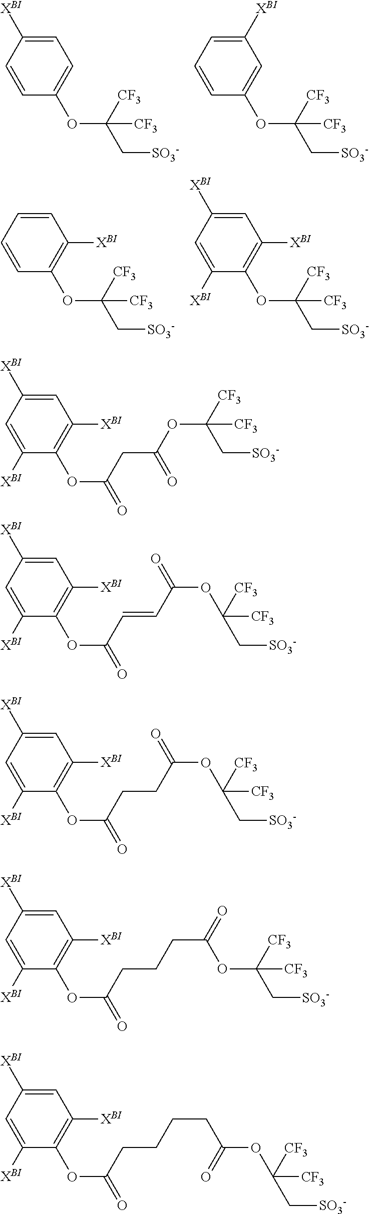

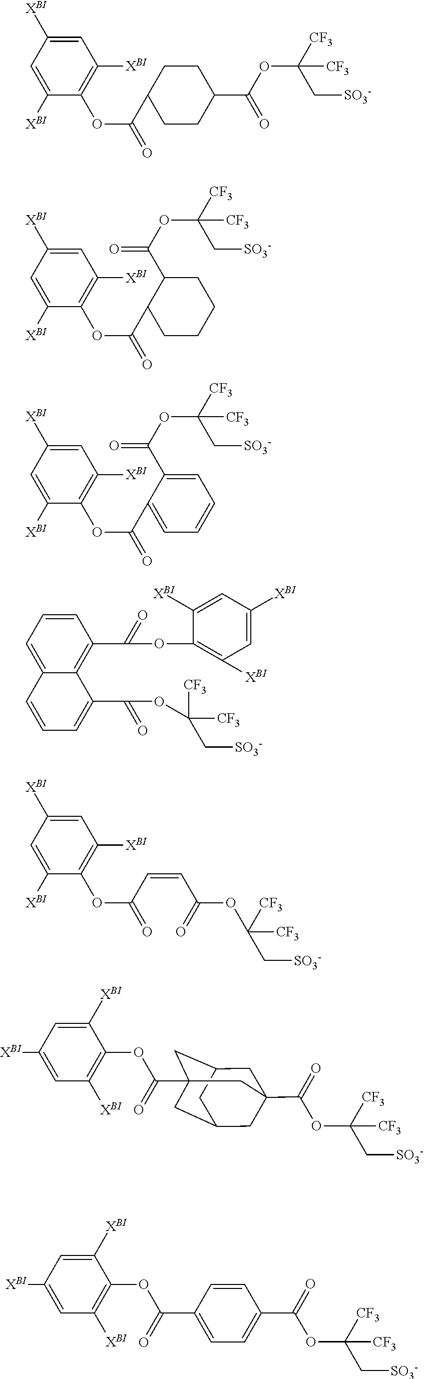

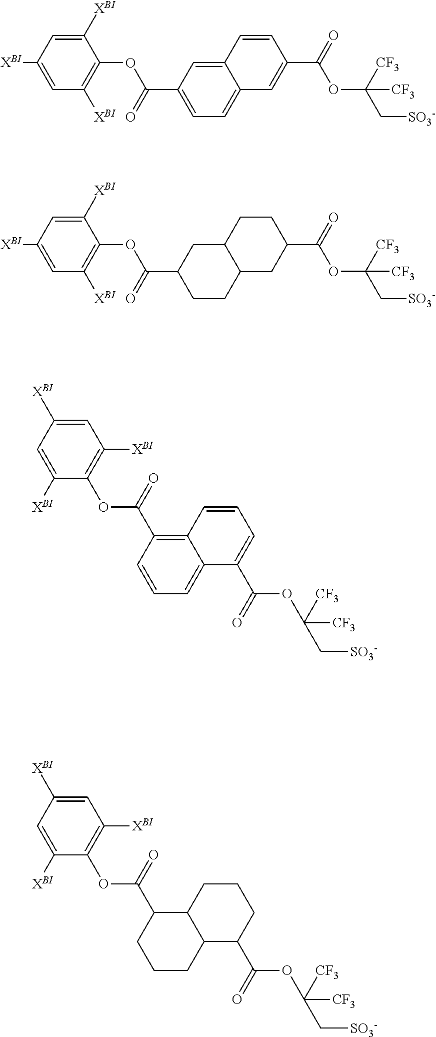

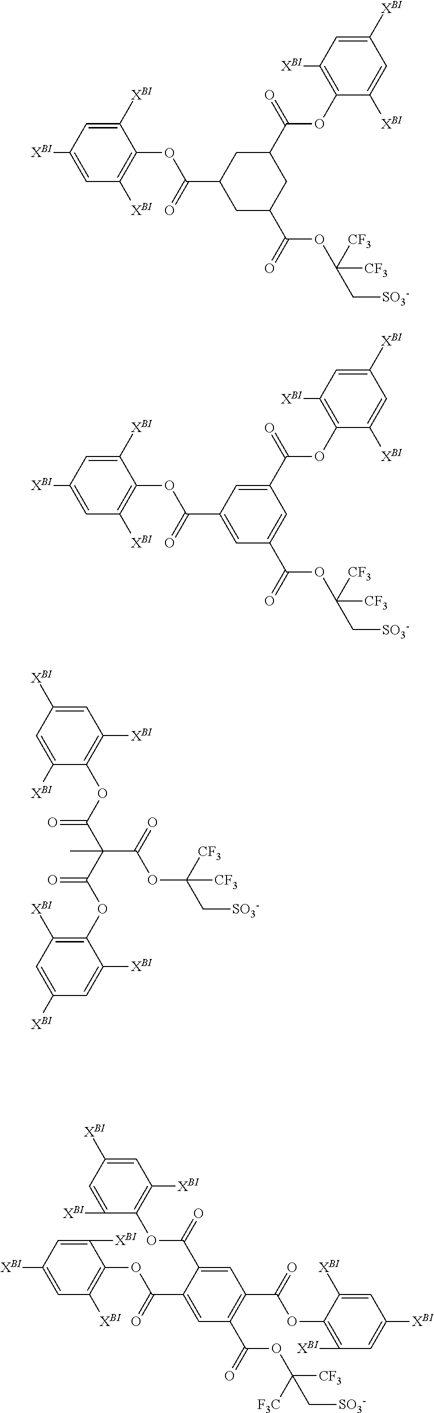

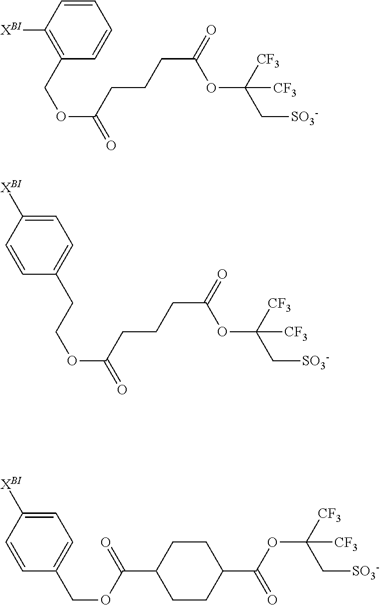

Examples of the non-nucleophilic counter ion include: a fluorobenzene sulfonic acid anion bonded to an aromatic group containing an iodine atom, which is described in JP 6648726 B; anions having a mechanism of causing decomposition by an acid, which is described in WO 2021/200056 A1 and JP 2021-070692 A; anions having a cyclic ether group described in JP 2018-180525 A and JP 2021-35935 A; an anion described in JP 2018-092159 A; and the like.

As the non-nucleophilic counter ion, fluorine-free bulky benzenesulfonic acid derivative anions, which is described in JP 2006-276759 A, JP 2015-117200 A, JP 2016-65016 A, and JP 2019-202974 A; and fluorine-free alkyl sulfonic acid anion and benzenesulfonic acid anion bonded to an aromatic group containing an iodine atom, which is described in JP 6645464 B, may also be used.

As the non-nucleophilic counter ion, a bisulfonic acid anion described in JP 2015-206932 A; an anion having a sulfonic acid on one side and a sulfonamide or sulfonimide on the other side, which is described in WO 2020/158366 A1; and an anion having a sulfonic acid on one side and a carboxylic acid on the other side, which is described in JP 2015-024989 A, may also be used.

Those represented by the formula (5) are also useful as the other photoacid generators of the component (D).

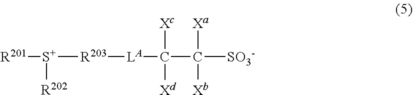

In the formula (5), R²⁰¹ and R²⁰² independently represent a hydrocarbyl group having 1 to 30 carbon atoms which may contain a heteroatom. R²⁰³ is a hydrocarbylene group having 1 to 30 carbon atoms which may contain a heteroatom. Any two of R²⁰¹, R²⁰² and R²⁰³ may be bonded to each other to form a ring with the sulfur atom to which they are bonded.

The hydrocarbyl group represented by R²⁰¹ and R²⁰² may be any of a saturated, unsaturated, linear, branched, or cyclic hydrocarbyl group. Specific examples thereof include alkyl groups having 1 to 30 carbon atoms such as a methyl group, an ethyl group, a propyl group, an isopropyl group, a n-butyl group, a sec-butyl group, a tert-butyl group, a tert-pentyl group, a n-pentyl group, a n-hexyl group, a n-octyl group, a 2-ethylhexyl group, a n-nonyl group, and a n-decyl group; cyclic saturated hydrocarbyl groups having 3 to 30 carbon atoms such as a cyclopentyl group, a cyclohexyl group, a cyclopentyl methyl group, a cyclopentyl ethyl group, a cyclopentyl butyl group, a cyclohexyl methyl group, a cyclohexyl ethyl group, a cyclohexyl butyl group, a norbornyl group, an oxanorbornyl group, a tricyclo[5.2.1.0^{2,6}] decanyl group, and an adamantyl group; aryl groups having 6 to 30 carbon atoms such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; groups obtained by combining these groups; and the like. Further, in these groups, a part or all of the hydrogen atoms of the hydrocarbyl group may be substituted with a group containing a heteroatom such as an oxygen atom, sulfur atom, nitrogen atom and a halogen atom, and a part of the —CH₂— constituting the hydrocarbyl group may be substituted with a group containing a heteroatom such as an oxygen atom, sulfur atom, and a nitrogen atom, so that the groups may contain a hydroxy group, cyano group, fluorine atom, chlorine atom, bromine atom, iodine atom, carbonyl group, ether bond, ester bond, sulfonic acid ester bond, carbonate bond, lactone ring, sultone ring, carboxylic acid anhydride, haloalkyl groups, or the like.

The hydrocarbylene group represented by R²⁰³ may be saturated or unsaturated, linear, branched or cyclic hydrocarbylene group. Examples thereof include alkanediyl groups having 1 to 30 carbon atoms such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms such as a cyclopentanediyl group, a cyclohexanediyl group, a norbornanediyl group, and an adamantanediyl group; cyclic unsaturated hydrocarbylene groups such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group; and the like. Further, in these groups, a part or all of the hydrogen atoms of the hydrocarbylene group may be substituted with a group containing a heteroatom such as an oxygen atom, sulfur atom, nitrogen atom and a halogen atom, and a part of the —CH₂— constituting the hydrocarbylene group may be substituted with a group containing a heteroatom such as an oxygen atom, sulfur atom, and a nitrogen atom, so that the groups may contain containing a hydroxy group, cyano group, fluorine atom, chlorine atom, bromine atom, iodine atom, carbonyl group, ether bond, ester bond, sulfonic acid ester bond, carbonate bond, lactone ring, sultone ring, carboxylic acid anhydride, haloalkyl groups, or the like. As the heteroatom, an oxygen atom is preferable.

In the formula (5), L^{A} is a single bond, an ether bond, or a hydrocarbylene group having 1 to 20 carbon atoms which may contain a heteroatom. The hydrocarbylene group may be any of a saturated, unsaturated, linear, branched, or cyclic hydrocarbylene group. Specific examples thereof include those similar to those exemplified as the hydrocarbylene group represented by R²⁰³.

In the formula (5), X^{a}, X^{b}, X^{c} and X^{d} independently represent a hydrogen atom, a fluorine atom, or a trifluoromethyl group, provided that at least one of X^{a}, X^{b}, X^{c} and X^{d} is a fluorine atom or a trifluoromethyl group.

As the photoacid generator represented by the formula (5), those represented by the following formula (5′) are preferable.

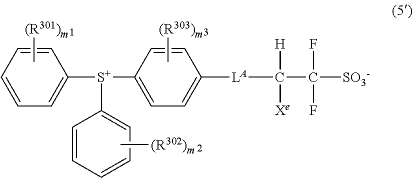

In the formula (5′), L^{A} is as defined above. X^{e} is a hydrogen atom or a trifluoromethyl group, preferably a trifluoromethyl group. R³⁰¹, R³⁰² and R³⁰³ independently represent a hydrogen atom, or a hydrocarbyl group having 1 to 20 carbon atoms which may contain a heteroatom. The hydrocarbyl group may be any of a saturated, unsaturated, linear, branched, or cyclic hydrocarbyl group. Specific examples thereof include those similar to those exemplified as the hydrocarbyl group represented by R¹⁰¹ in the formula (3). m¹ and m² are independently an integer of 0 to 5, and m³ is an integer of 0 to 4.

Examples of the photoacid generator represented by the formula (5) include those exemplified for the photoacid generator represented by the formula (2) in JP 2017-026980 A.

Among the other photoacid generators, those having an anion represented by the formula (1A′) or (1D) are particularly preferable because of reduced acid diffusion and excellent solvent solubility. Further, those represented by the formula (5′) are particularly preferable because of the minimized acid diffusion.

When the acid generator as the component (D), in particular, a photoacid generator is contained, the content thereof is preferably 0.1 to 40 parts by mass, more preferably 0.5 to 20 parts by mass, per 80 parts by mass of the polymer compound (A). Insofar as the addition amount of the acid generator as the component (D) is within this range, desirable resolution is obtained and the problem of foreign substances after the development or during the stripping of the resist film can be avoided. Only one kind of the acid generator as the component (D) or a combination of two or more kinds thereof may be used.

[(E) Surfactant Insoluble or Substantially Insoluble in Water and Soluble in Alkaline Developer and/or Surfactant Insoluble or Substantially Insoluble in Water and Alkaline Developer]

The resist composition, in particular, the chemically-amplified resist composition, of the present invention may further include (E) a surfactant which is insoluble or substantially insoluble in water and soluble in an alkaline developer, and/or a surfactant which is insoluble or substantially insoluble in water and an alkaline developer. Examples of such a surfactant include those described in JP 2010-215608 A and JP 2011-16746 A.

Among many examples of the surfactant insoluble or substantially insoluble in water and alkaline developer described in the patent documents above, preferred examples are FC-4430 (3M), Surflon (registered trademark) S-381 (AGC Seimi Chemical Co., Ltd.), Olfine (registered trademark) E1004 (Nissin Chemical Co., Ltd.), KH-20 and KH-30 (AGC Seimi Chemical Co., Ltd.), an oxetane ring-opening polymerization product represented by the following formula (surf-1), and the like.

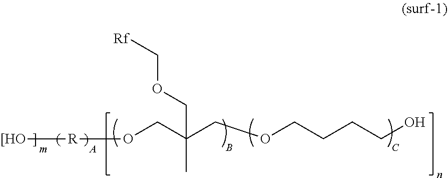

Here, R, Rf, A, B, C, m, and n are applied only to the formula (surf-1), regardless of the descriptions above. R is a di- to tetra-valent aliphatic group having 2 to 5 carbon atoms. Examples of the divalent aliphatic group include an ethylene group, a 1,4-butylene group, a 1,2-propylene group, a 2,2-dimethyl-1,3-propylene group, a 1,5-pentylene group, and the like. Examples of the tri- and tetra-valent groups are shown below,

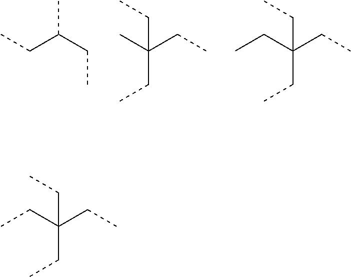

wherein the dashed line represents a bonding arm. These formulae are partial structures derived from glycerol, trimethylol ethane, trimethylol propane, and pentaerythritol, respectively.

Among them, a 1,4-butylene group and a 2,2-dimethyl-1,3-propylene group are preferably used.

Rf is a trifluoromethyl group or a pentafluoroethyl group, and preferably a trifluoromethyl group. “m” is an integer of 0 to 3; n is an integer of 1 to 4; and the sum of m and n, which represents the valence of R, is an integer of 2 to 4. A is 1; B is an integer of 2 to 25, preferably 4 to 20; and C is an integer of 0 to 10, preferably 0 or 1. Further, the formula (surf-1) does not prescribe the arrangement of the constituent units, and they may be bonded either blockwise or randomly. The details of the manufacture of a surfactant in the form of a partially fluorinated oxetane ring-opening polymerization product are disclosed in U.S. Pat. No. 5,650,483 B1.

The surfactant which is insoluble or substantially insoluble in water and soluble in an alkaline developer has a propensity to segregate on the surface of the resist film to exhibit a function of minimizing water penetration or leaching when ArF immersion lithography is performed without using a resist protection film. Therefore, the surfactant is also effective for preventing water-soluble components from being leached out of the resist film, thereby minimizing any damage to the exposure tool. Further, the surfactant becomes solubilized during the development with an aqueous alkaline solution after the exposure and the post exposure bake (PEB), thereby generating few or no foreign substances causing defects. Preferable example of such a surfactant is a polymer-type surfactant which is insoluble or substantially insoluble in water but soluble in alkaline developer, which is also referred to as “hydrophobic resin”, and a resin which is water repellent and enhances water slippage.

Examples of such a polymer-type surfactant include those containing at least one of the repeating units represented by any of the following formulae (8A) to (8E).

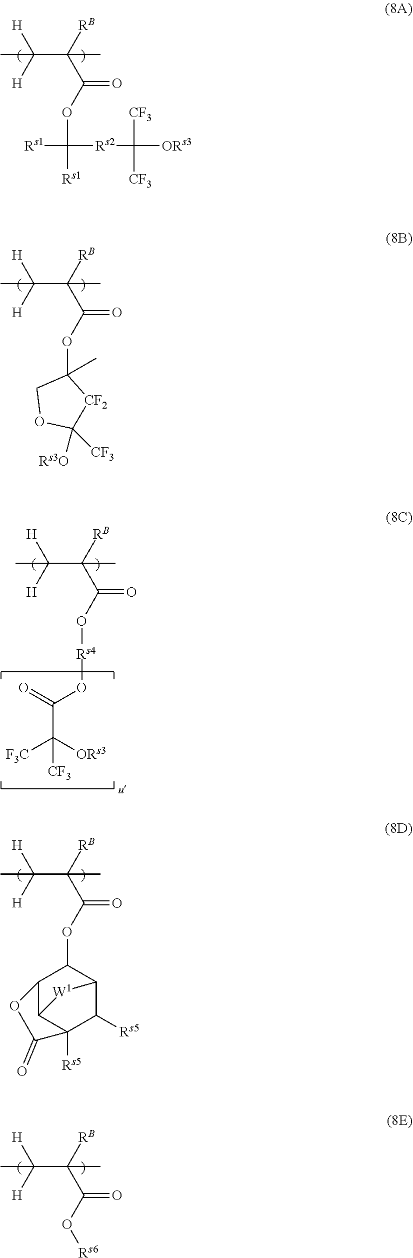

In the formulae (8A) to (8E), R^{B} represents a hydrogen atom, a fluorine atom, a methyl group, or a trifluoromethyl group. W¹ is —CH₂—, —CH₂CH₂—, —O— or two —H separated from each other. R^{s1} independently represents a hydrogen atom or a hydrocarbyl group having 1 to 10 carbon atoms. R^{s2} is a single bond, or a linear or branched hydrocarbylene group having 1 to 5 carbon atoms. R^{s3} independently represents a hydrogen atom or a hydrocarbyl group or a fluorinated hydrocarbyl group having 1 to 15 carbon atoms, or an acid-labile group. When R^{s3} is a hydrocarbyl group or a fluorinated hydrocarbyl group, an ether bond or a carbonyl group may be intervened between the carbon-carbon bonds. R^{s4} represents a (u′+1)-valent hydrocarbon group or a fluorinated hydrocarbon group having 1 to 20 carbon atoms; and u′ is an integer of 1 to 3. R^{s5} independently represents a hydrogen atom or a group represented by C(═O)—O—R^{s7}. R^{s7} represents a fluorinated hydrocarbyl group having 1 to 20 carbon atoms. R^{s6} represents a hydrocarbyl group or a fluorinated hydrocarbyl group having 1 to 15 carbon atoms, and an ether bond or a carbonyl group may be intervened between the carbon-carbon bonds.

Examples of the hydrocarbyl group represented by R^{s1} include linear, branched, or cyclic hydrocarbyl groups, such as a methyl group, an ethyl group, a n-propyl group, an isopropyl group, a cyclo propyl group, a n-butyl group, an isobutyl group, a sec-butyl group, a tert-butyl group, a cyclobutyl group, a n-pentyl group, a cyclopentyl group, a n-hexyl group, a cyclohexyl group, a n-heptyl group, a n-octyl group, a n-nonyl group, a n-decyl group, an adamantyl group, a norbornyl group, and the like. Among them, those with 1 to 6 carbon atoms are preferable.

The hydrocarbylene group represented by R^{s2} may be any of linear, branched, and cyclic hydrocarbylene groups, and specific examples thereof include a methylene group, an ethylene group, a propylene group, a butylene group, a pentylene group, and the like.

The hydrocarbyl group represented by R^{s3} or R^{s6} may be any of linear, branched, and cyclic hydrocarbyl groups, and specific examples thereof include an alkyl group, an alkenyl group, and an alkynyl group, and the like. An alkyl group is preferable. Examples of the alkyl group include a n-undecyl group, a n-dodecyl group, a tridecyl group, a tetradecyl group, a pentadecyl group, and the like, in addition to those exemplified for the hydrocarbyl group represented by R^{s1}. Examples of the fluorinated hydrocarbyl group represented by R^{s3} or R^{s6} include groups in which some or all of the hydrogen atoms bonded to the carbon atoms of the hydrocarbyl groups mentioned above are substituted with fluorine atoms. As described above, an ether bond or a carbonyl group may be intervened between these carbon-carbon bonds.

Examples of the acid-labile group represented by R^{s3} include the groups represented by the formulae (L1) to (L4) mentioned above, tertiary hydrocarbyl groups having 4 to 20, preferably 4 to 15 carbon atoms, trialkylsilyl groups in which each alkyl group has 1 to 6 carbon atoms, oxoalkyl groups having 4 to 20 carbon atoms, and the like.

The (u′+1)-valent hydrocarbon group or fluorinated hydrocarbon group represented by R^{s4} may be any of linear, branched, and cyclic hydrocarbon groups, and specific examples thereof include groups obtained by further eliminating u hydrogen atoms from the hydrocarbyl groups or fluorinated hydrocarbyl groups and the like mentioned above.

The fluorinated hydrocarbyl group represented by R^{s7} may be any of linear, branched and cyclic fluorinated hydrocarbyl groups, more specifically groups in which some or all of the hydrogen atoms of the hydrocarbyl groups described above are substituted with fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl) ethyl group, a 2-(perfluorodecyl)ethyl group, and the like.

Examples of the repeating units represented by any of the formulae (8A) to (8E) include, but are not limited thereto. In the following formulae, R^{B} is as defined above.

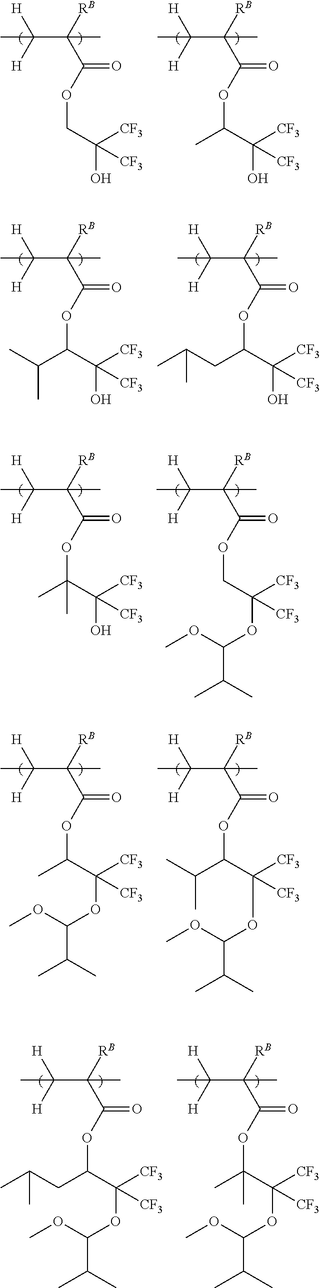

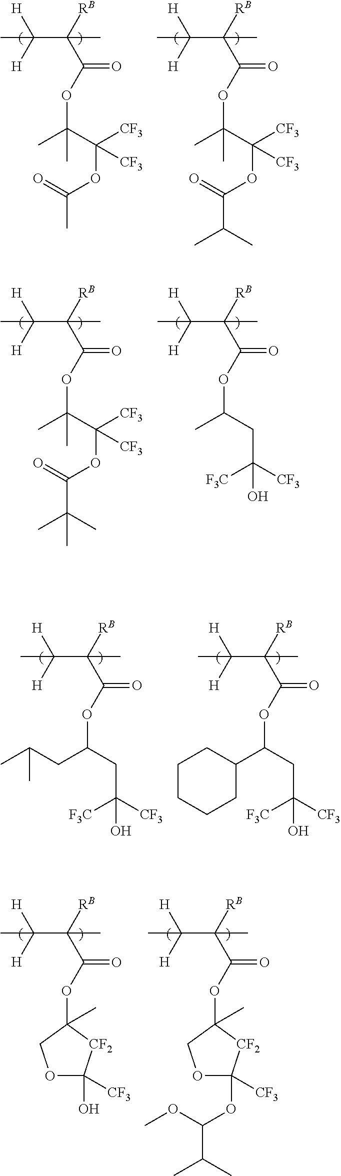

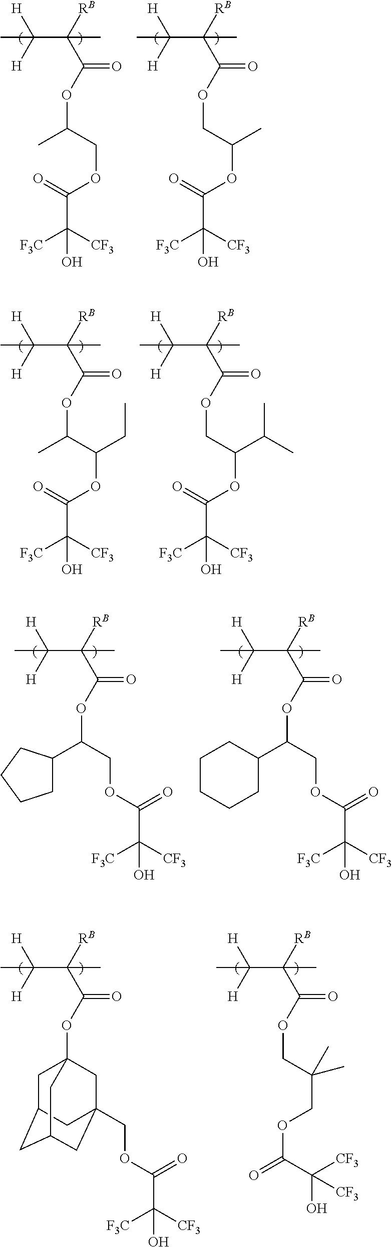

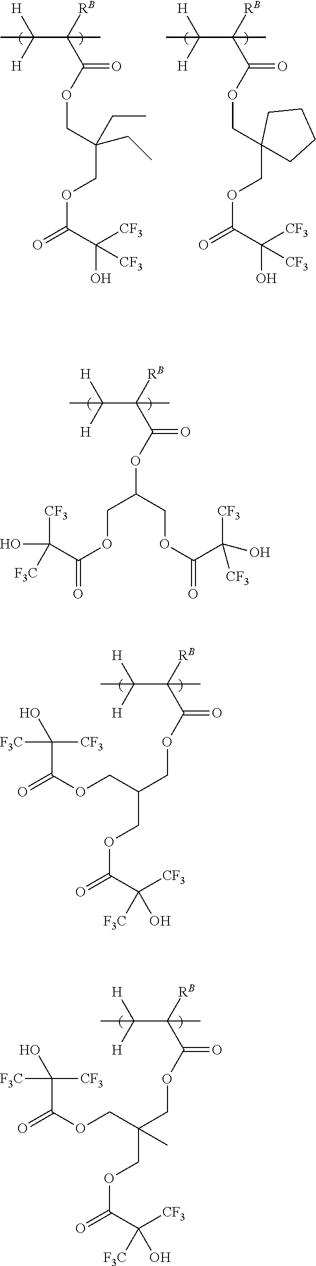

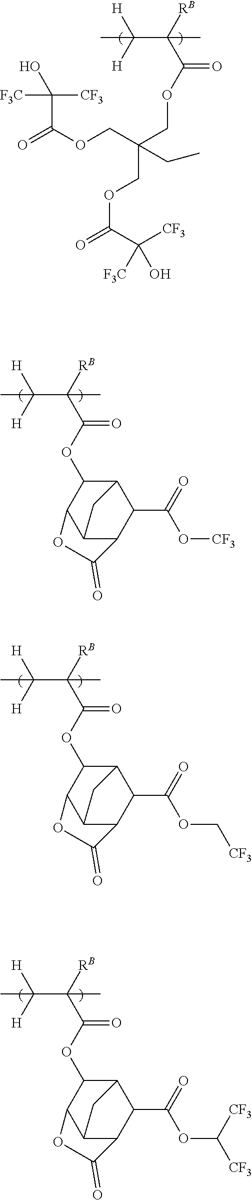

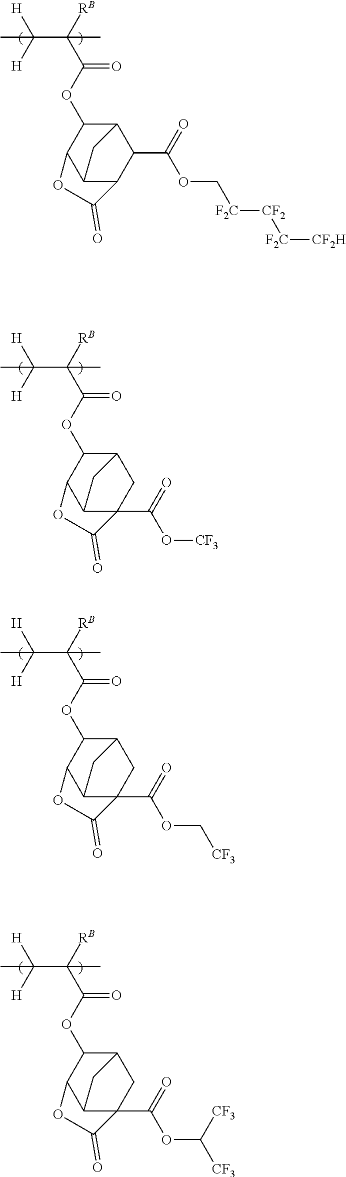

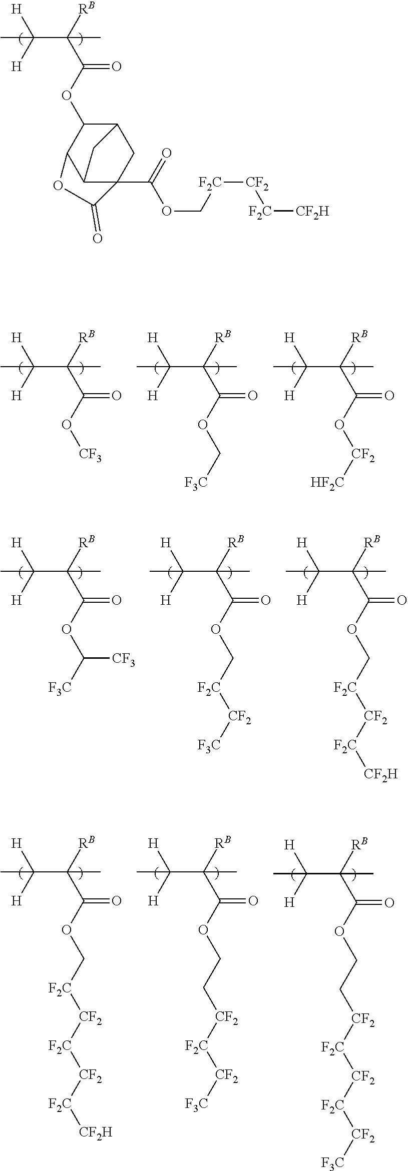

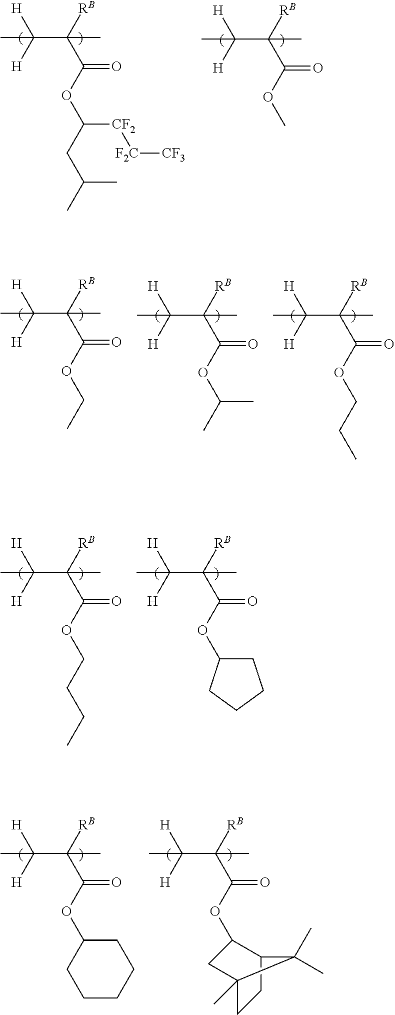

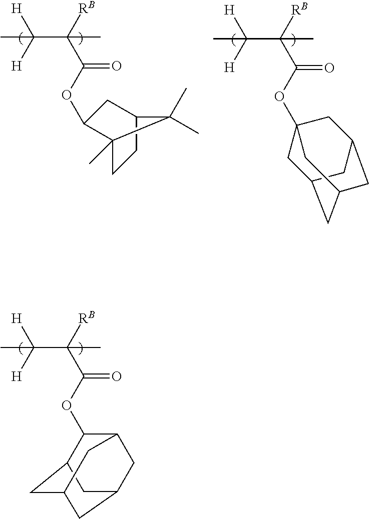

The polymer-type surfactant described above may further contain repeating units other than the repeating units represented by the formulae (8A) to (8E). Examples of the other repeating units include repeating units obtained from methacrylic acid, α-trifluoromethylacrylic acid derivatives, and the like. In the polymer-type surfactant, the content of the repeating units represented by the formulae (8A) to (8E) is preferably 20 mol % or more, more preferably 60 mol or more, further preferably 100 mol % of the entire repeating units.

The polymer-type surfactant preferably has a Mw of 1,000 to 500,000, more preferably 3,000 to 100,000. Mw/Mn is preferably 1.0 to 2.0, more preferably 1.0 to 1.6.

Examples of the method for synthesizing the polymer-type surfactant described above include a method of adding a radical initiator to an unsaturated bond-containing monomer or monomers providing the repeating units represented by the formulae (8A) to (8E) and optionally other repeating units in an organic solvent, and heating the mixture for polymerization. Examples of the organic solvent used for the polymerization include toluene, benzene, THF, diethyl ether, dioxane, and the like. Examples of the polymerization initiator include AIBN, 2,2′-azobis(2,4-dimethylvaleronitrile), dimethyl 2,2-azobis(2-methylpropionate), benzoyl peroxide, lauroyl peroxide, and the like. The reaction temperature is preferably 50 to 100° C. The reaction time is preferably 4 to 24 hours. The acid-labile group that has been introduced into the monomer may be used as it is, or protection or partial protection thereof after the polymerization is also possible.

For the synthesis of the polymer-type surfactant described above, any known chain transfer agents, such as dodecyl mercaptan and 2-mercaptoethanol, may be used for the adjustment of the molecular weight. In this case, the amount of the chain transfer agent to be added is preferably 0.01 to 10 mol % based on the total moles of the monomers to be polymerized.

When the surfactant of the component (E) is contained, the content thereof is preferably 0.1 to 50 parts by mass, more preferably 0.5 to 10 parts by mass, per 80 parts by mass of the polymer compound (A). When the addition amount is 0.1 parts by mass or more, the receding contact angle of water with respect to the resist film surface sufficiently improves; when the addition amount is 50 parts by mass or less, the dissolution rate with respect to the developer on the resist film surface is small, thus maintaining a sufficient height of a fine pattern formed thereon.

### [(F) Other Components]

The chemically-amplified resist composition of the present invention may further contain (F) other components, for example, a compound which is decomposed by an acid to generate an acid (an acid amplifier compound), an organic acid derivative, a fluorinated alcohol, a compound having a Mw of 3,000 or less and a solubility in developer to be changed under the action of an acid (dissolution inhibitor), and the like. Examples of the acid amplifier compound are disclosed in JP 2009-269953 and JP 2010-215608 A. When the acid amplifier compound is contained, the content thereof is preferably 0 to 5 parts by mass, more preferably 0 to 3 parts by mass, per 80 parts by mass of the polymer compound (A). A content of 5 parts by mass or less allows easy control of acid diffusion and prevents degradations of the resolution or the pattern shape. Examples of the organic acid derivatives, fluorine-substituted alcohols, and dissolution inhibitors include the compounds disclosed in JP 2009-269953 A or JP 2010-215608 A.

### [Patterning Process]

The present invention provides a patterning process including the steps of forming a resist film on a substrate by using the resist composition of the present invention, exposing the resist film to high-energy beam, and developing the exposed resist film by using a developer.

The patterning process of the present invention includes the steps of forming a resist film on a substrate by using the resist composition, in particular, the chemically-amplified resist composition, exposing the resist film to KrF excimer laser, ArF excimer laser, EB or EUV, and developing the exposed resist film by using a developer.

The substrate used herein may be, for example, a substrate for the manufacture of integrated circuits (Si, SiO₂, SiN, SiON, TiN, WSi, BPSG, SOG, organic antireflective films, and the like) or a substrate for the manufacture of mask circuits (Cr, CrO, CrON, MoSi₂, SiO₂, and the like).

The resist film can be formed, for example, by applying the chemically-amplified resist composition by spin coating or similar techniques so that the film thickness falls within a range of 0.05 to 2 μm, and prebaking the resulting coating on a hotplate preferably at a temperature of 60 to 150° C. for 1 to 10 minutes, more preferably at a temperature of 80 to 140° C. for 1 to minutes.

Further, the high-energy beam is preferably KrF excimer laser beam, ArF excimer laser beam, an electron beam, or extreme ultraviolet ray having a wavelength of 3 to 15 nm.

The exposure of the resist film may be performed by irradiation while using a mask having a desired pattern when KrF excimer laser beam, ArF excimer laser beam, or EUV is used, so that the exposure dose is preferably 1 to 200 mJ/cm², more preferably 10 to 100 mJ/cm². When EB is used, the exposure is performed directly or while using a mask having a desired pattern so that the exposure dose is preferably 1 to 300 ρC/cm², more preferably 10 to 200 ρC/cm².

In addition to a usual exposure method, the exposure may also be performed by a liquid immersion method in which a liquid having a refraction index of 1.0 or more is intervened between the resist film and the projection lens. In this case, a protection film insoluble in water may be used.

Such a water-insoluble protection film is used to prevent the components from being eluted from the resist film and to improve water sliding on the film surface. There are generally two types of water-insoluble protection film. The first type is an organic solvent-strippable protection film which must be stripped before development using an aqueous alkaline solution by an organic solvent in which the resist film is not dissolved. The second type is an aqueous alkaline solution-soluble protection film which is soluble in an alkaline developer, and is removed simultaneously with the removal of solubilized regions of the resist film. The protection film of the second type is preferably made of a material obtained by dissolving a polymer having a 1,1,1,3,3,3-hexafluoro-2-propanol residue, which is insoluble in water and soluble in an alkaline developer, as a base in an alcohol-based solvent having 4 or more carbon atoms, an ether-based solvent having 8 to 12 carbon atoms, or a mixed solvent thereof. It is also possible to use a material in which the aforementioned surfactant which is insoluble in water and soluble in an alkaline developer is dissolved in an alcohol-based solvent having 4 or more carbon atoms, an ether-based solvent having 8 to 12 carbon atoms, or a mixed solvent thereof.

After the exposure, PEB may be performed. PEB is performed, for example, by heating on a hotplate preferably at 60 to 150° C. for 1 to 5 minutes, more preferably at 80 to 140° C. for 1 to 3 minutes.

The development is performed by using a developer of an aqueous alkaline solution, for example, preferably 0.1 to 5 mass %, more preferably 2 to 3 mass % tetramethylammonium hydroxide (TMAH), for preferably 0.1 to 3 minutes, more preferably 0.5 to 2 minutes by a standard method, such as a dipping method, a puddle method, or a spray method. As a result, the exposed portion is dissolved and a desired resist pattern is formed on the substrate.

As means in the patterning process, after the resist film is formed, rinsing with pure water (post-soaking) may be performed to extract the acid generator or the like from the film surface, or washing of particles may be performed. Further, after the exposure, rinsing (post-soaking) to remove water remaining on the film may be performed.

Also, a double patterning process may be used for pattern formation. The double patterning process includes a trench method of processing a base of a 1:3 trench pattern by the first exposure and etching, shifting the position, and forming a 1:3 trench pattern by the second exposure, thereby forming a 1:1 pattern; and a line method of processing a first base of a 1:3 isolated left pattern by the first exposure and etching, shifting the position, processing a second base formed below the first base by the second exposure through the 1:3 isolated left pattern, thereby forming a half-pitch 1:1 pattern.

In the patterning process of the present invention, a negative tone development method, which uses, instead of the developer of an aqueous alkaline solution, an organic solvent as a developer to dissolve the unexposed portion, may be employed.

Examples of the developer used in this development using an organic solvent include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, 2-phenylethyl acetate, and the like. Only one kind of these organic solvent may be used alone or two or more kinds thereof may be mixed.

### EXAMPLES

Synthesis Examples, Examples, and Comparative Examples are shown below to specifically describe the present invention. However, the present invention is not limited to the Examples. Should be noted that the devices used herein are as follows.

IR: NICOLET 6700 manufactured by Thermo Fisher Scientific Inc.

¹H-NMR: ECA-500 manufactured by JEOL Ltd.

MALDI-TOF-MS: S3000 manufactured by JEOL Ltd.

### [1] Synthesis of Polymerizable Monomer

### [Example 1-1] Synthesis of Monomer A1

### (1) Synthesis of Intermediate Pre-A1

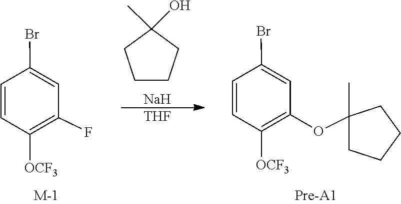

In an atmosphere of nitrogen, sodium hydride (purity 55 mass %, 30.6 g) was suspended in THF (170 ml), and a solution of 1-methylcyclopentanol (80.0 g) and THF (80 ml) was added dropwise. After the dropwise addition, heating under reflux was performed for 4 hours to prepare a metal alkoxide. Thereafter, a raw material M-1 (181.3 g) was added dropwise, followed by heating under reflux and aging for 18 hours. The reaction solution was cooled in an ice bath and water (300 ml) was added to quench the reaction. The target product was extracted twice by using a solvent containing toluene (200 ml) and hexane (200 ml), followed by ordinary aqueous work-up. Thereafter, the solvent was distilled off, followed by distillation purification to obtain 154.0 g of an intermediate Pre-A1 in the form of a colorless oily substance (yield=62%).

### (2) Synthesis of Monomer A1

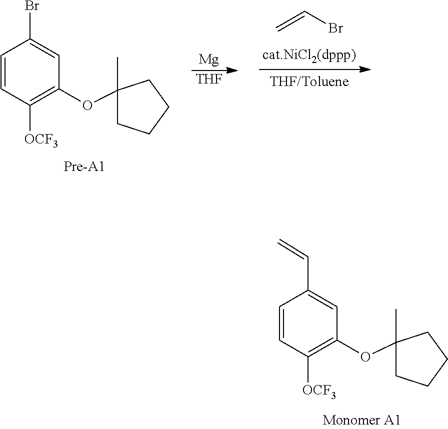

In an atmosphere of nitrogen, Grignard reagent was prepared from magnesium (9.4 g), THF (110 g) and the intermediate Pre-A1 (104.7 g). The reagent was diluted with toluene (55 g), and the reaction system was cooled to 10° C. or less. Thereafter, [1,3-bis(diphenylphosphino)propane] nickel(II)dichloride (1.0 g) was added, and the mixture was stirred at an internal temperature of 10° C. or less for 30 minutes. After the stirring, a solution containing vinyl bromide (50.9 g), THE (55 g), and toluene (55 g) was added dropwise while the internal temperature was kept at 20° C. or less. After the dropwise addition, the mixture was aged for 1 hour at an internal temperature of 20° C. or less. After the aging, the reaction system was cooled, and an aqueous solution containing ammonium chloride (40 g), 20 mass % hydrochloric acid (40 g) and water (200 g) was added dropwise to quench the reaction. Thereafter, the target product was extracted by using hexane (100 g), followed by ordinary aqueous work-up. Thereafter, the solvent was distilled off, followed by distillation purification to obtain 63.3 g of a monomer A1 in the form of a colorless oily substance (yield=74%).

The results of IR spectral data and nuclear magnetic resonance spectrum (¹H-NMR/DMSO-d6) of the monomer A1 are shown below.

IR (D-ATR): ν=2971, 2876, 1599, 1583, 1504, 1450, 1417, 1393, 1379, 1252, 1225, 1202, 1168, 1121, 1031, 986, 967, 913, 870, 832, 801, 754, 697, 628 cm⁻¹.

¹H-NMR (600 MHz in DMSO-d₆): δ=7.28 (1H, d), 7.18 (1H, d), 7.17 (1H, d), 6.74 (1H, dd), 5.82 (1H, d), 5.30 (1H, d), 2.01 (2H, m), 1.67 (6H, m), 1.45 (3H, s) ppm.

### [Example 1-2] Synthesis of Monomer A2

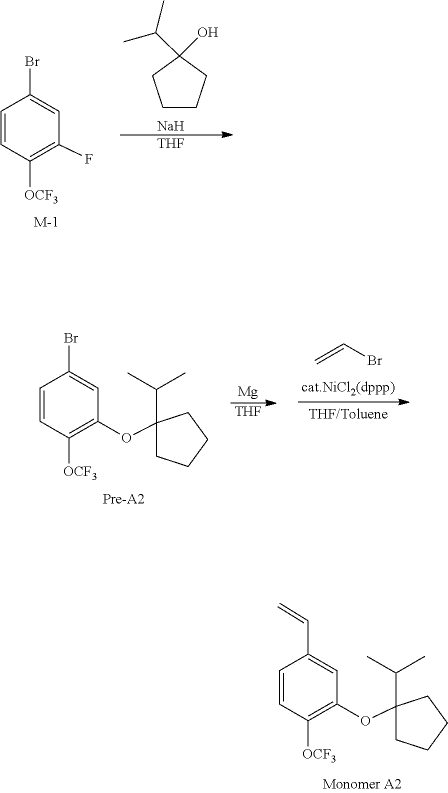

A monomer A2 was synthesized in the same manner as in Example 1-1, except that 1-isopropylcyclopentanol was used instead of 1-methylcyclopentanol (amount=81.3 g, yield of two steps=68%).

The results of IR spectral data and nuclear magnetic resonance spectrum (¹H-NMR/DMSO-d6) of the monomer A2 are shown below.

IR (D-ATR): ν=2966, 2877, 1597, 1584, 1504, 1470, 1454, 1417, 1391, 1369, 1352, 1253, 1225, 1201, 1167, 1122, 1033, 984, 911, 868, 831, 800, 752, 699, 627 cm⁻¹.

¹H-NMR (600 MHz in DMSO-d₆): δ=7.27 (1H, d), 7.18 (1H, d), 7.13 (1H, d), 6.74 (1H, dd), 5.80 (1H, d), 5.29 (1H, d), 2.47 (2H, m), 1.87 (4H, m), 1.54 (4H, m), 0.90 (6H, d) ppm.

### [Example 1-3] Synthesis of Monomer A3

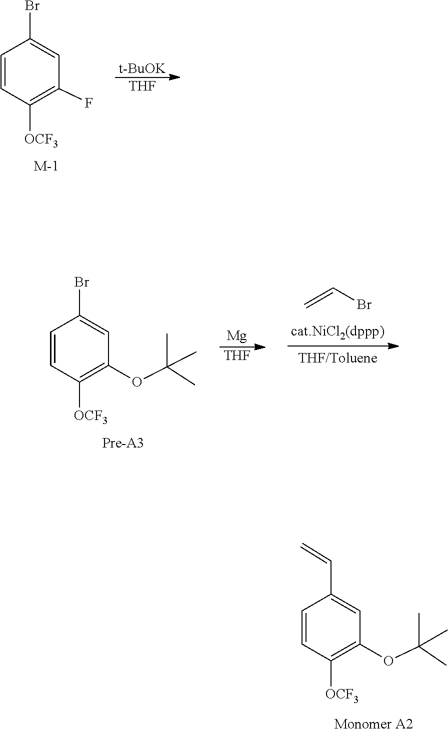

A monomer A3 was synthesized in the same manner as in Example 1-1, except that potassium t-butoxide was used instead of a metal alkoxide prepared from 1-methylcyclopentanol and sodium hydride (amount=92.3 g, yield of two steps=66%).

The results of IR spectral data and nuclear magnetic resonance spectrum (¹H-NMR/DMSO-d₆) of the monomer A3 are shown below.

IR (D-ATR): ν=3092, 2982, 1601, 1581, 1503, 1414, 1393, 1369, 1250, 1224, 1203, 1166, 1121, 1031, 987, 967, 912, 895, 838, 802, 766, 723, 667, 600 cm⁻¹.

¹H-NMR (600 MHz in DMSO-d₆): δ=7.28 (3H, m), 6.73 (1H, dd), 5.84 (1H, d), 5.30 (1H, d), 1.32 (9H, s) ppm.

### [Examples 1-4 to 1-11] Synthesis of Monomers A4 to A11

Various polymerizable monomers were synthesized from corresponding raw materials using various organic synthesis reactions. The following shows the structures of the polymerizable monomers used for the chemically-amplified resist composition.

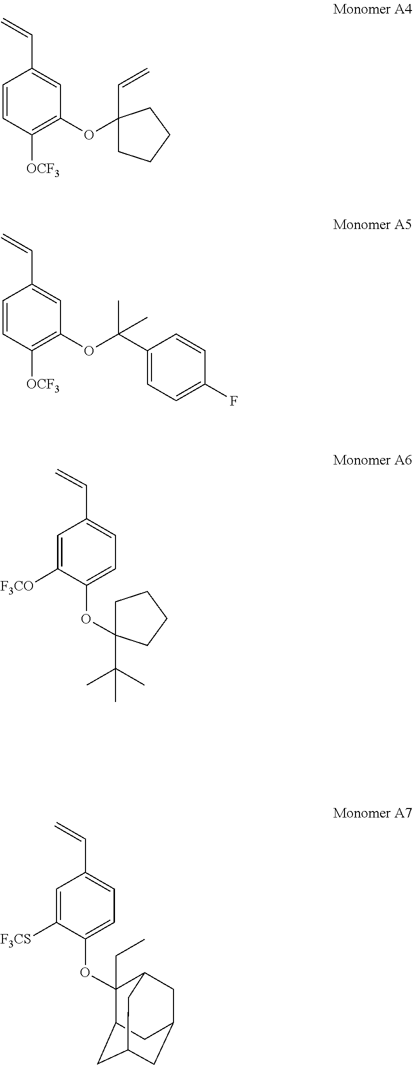

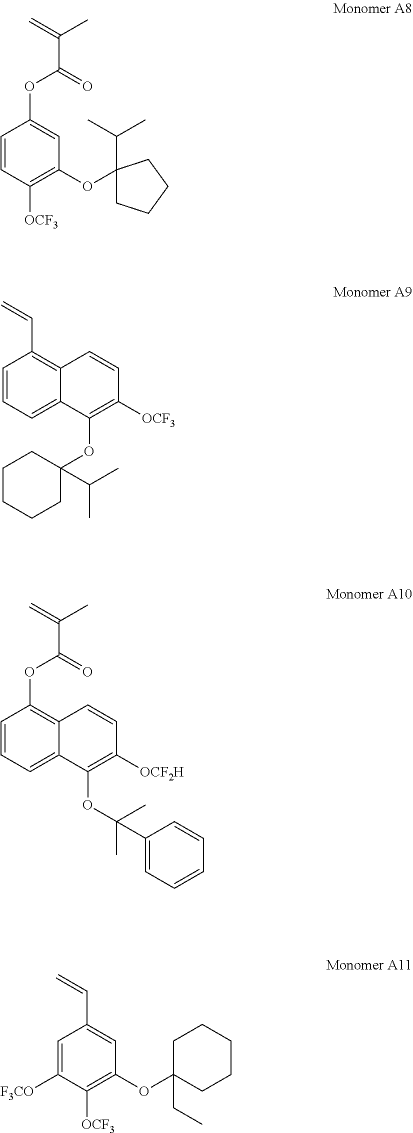

### [2] Synthesis of Base Polymer

In addition to the monomers A1 to A11, the following monomers were also used in the synthesis of the base polymer.

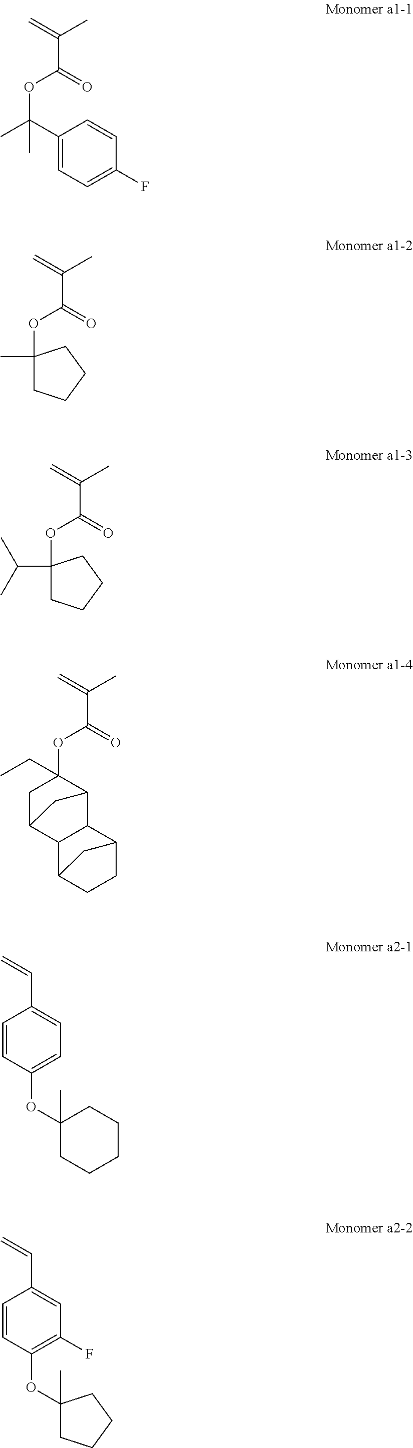 [SMILES: C=C(C)C(=O)OC(C)(C)c1ccc(F)cc1 ; C=C(C)C(=O)OC1(C)CCCC1 ; C=C(C)C(=O)OC1(C(C)C)CCCC1 ; C=C(C)C(=O)OC1(CC)CC2CC1C1C3CCC(C3)C21 ; C=Cc1ccc(OC2(C)CCCCC2)cc1 ; C=Cc1ccc(OC2(C)CCCC2)c(F)c1]

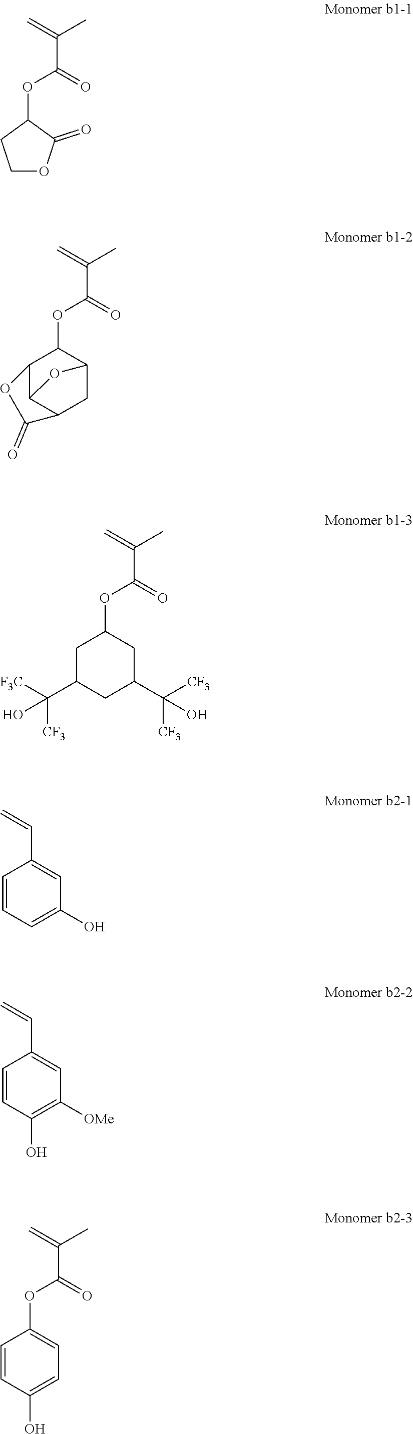

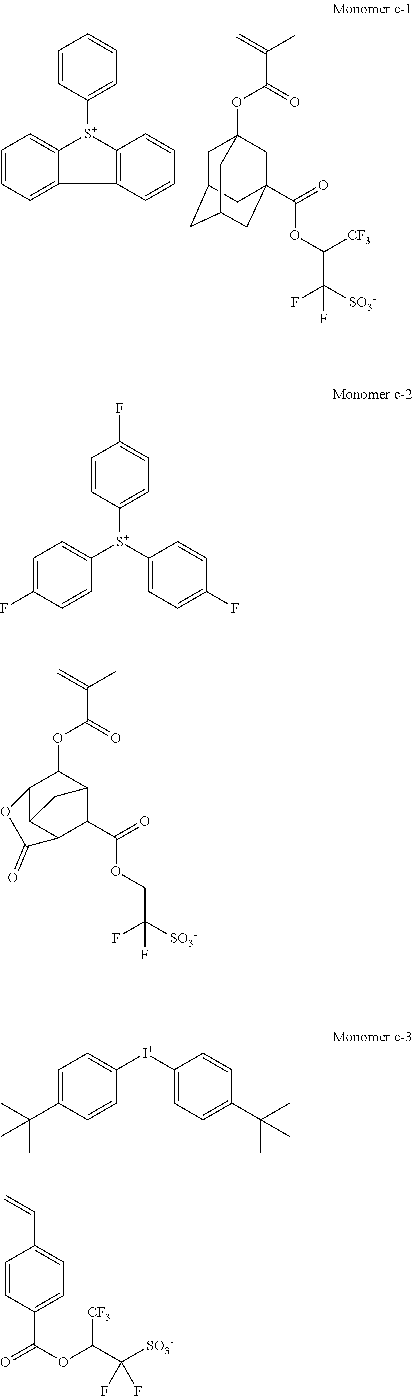

### [Example 2-1] Synthesis of Polymer P-1

In an atmosphere of nitrogen, the monomer A1 (50.1 g), a monomer a1-1 (50.1 g), a monomer b2-1 (24.8 g), a monomer c-1 (38.0 g), 3.96 g of V-601 (Wako Pure Chemical Industries, Ltd.) and 127 g of MEK were placed in a flask to prepare a monomer-polymerization initiator solution. 46 g of MEK was placed in another flask in a nitrogen atmosphere, followed by heating to 80° C. while stirring. Thereafter, the monomer-polymerization initiator solution mentioned above was added thereto dropwise over the course of 4 hours. After the dropwise addition, the polymerization solution was continuously stirred for 2 hours while keeping the temperature at 80° C., and then cooled to room temperature. The obtained polymerization solution was added dropwise to 2,000 g of hexane under vigorous stirring, and the precipitated polymer was separated by filtration. Further, the obtained polymer was washed twice with 600 g of hexane, and then vacuum dried at 50° C. for 20 hours to obtain a polymer P-1 in the form of white powder (amount=98.1 g, yield=98%). Mw and Mw/Mn of the polymer P-1 were 9,600 and 1.81, respectively. Mw is a polystyrene-based value determined by GPC using DMF as an eluent.

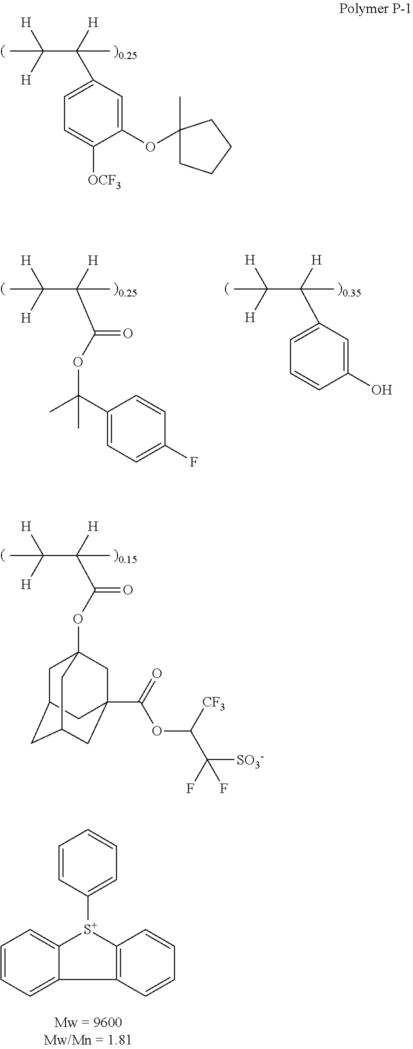

### [Examples 2-2 to 2-30, Comparative Examples 1-1 to 1-15] Synthesis of Polymers P-2 to P-30, CP-1 to CP-15

Base polymers shown in Tables 1 and 2 were synthesized in the same manner as in Example 2-1, except that the type and the incorporation ratio of the monomers were changed.

TABLE 1

| | | Introduction | | Introduction | | Introduction | | Introduction | | |
|---|---|---|---|---|---|---|---|---|---|---|
| Polymer | Unit A | Ratio (mol %) | Unit a | Ratio (mol %) | Unit b | Ratio (mol %) | Unit c | Ratio (mol %) | Mw | Mw/Mn |
| P-1 | A1 | 25 | a1-1 | 25 | b2-1 | 35 | c-1 | 15 | 9600 | 1.81 |
| P-2 | A2 | 25 | a1-1 | 25 | b2-1 | 35 | c-1 | 15 | 9700 | 1.79 |
| P-3 | A3 | 25 | a1-1 | 25 | b2-1 | 35 | c-1 | 15 | 10000 | 1.80 |
| P-4 | A4 | 25 | a1-1 | 25 | b2-1 | 35 | c-1 | 15 | 9800 | 1.82 |
| P-5 | A5 | 25 | a1-1 | 25 | b2-1 | 35 | c-1 | 15 | 9600 | 1.82 |
| P-6 | A6 | 25 | a1-1 | 25 | b2-1 | 35 | c-1 | 15 | 9700 | 1.83 |
| P-7 | A7 | 25 | a1-1 | 25 | b2-1 | 35 | c-1 | 15 | 9900 | 1.78 |
| P-8 | A8 | 25 | a1-1 | 25 | b2-1 | 35 | c-1 | 15 | 10100 | 1.79 |
| P-9 | A9 | 25 | a1-1 | 25 | b2-1 | 35 | c-1 | 15 | 9600 | 1,80 |
| P-10 | A10 | 25 | a1-1 | 25 | b2-1 | 40 | c-1 | 10 | 10200 | 1.83 |
| P-11 | A11 | 25 | a1-1 | 25 | b2-1 | 35 | c-1 | 15 | 9700 | 1.77 |
| P-12 | A2 | 25 | a1-2 | 25 | b2-1 | 35 | c-1 | 15 | 9600 | 1.81 |
| P-13 | A1 | 25 | a1-3 | 25 | b2-1 | 35 | c-1 | 15 | 10100 | 1.77 |
| P-14 | A4 | 20 | a1-1 | 30 | b2-1 | 35 | c-2 | 15 | 9600 | 1.82 |
| P-15 | A5 | 30 | a1-3 | 20 | b2-3 | 35 | c-1 | 15 | 9900 | 1.76 |
| P-16 | A6 | 25 | a1-4 | 25 | b1-1 | 20 | c-3 | 15 | 9800 | 1.79 |
| | | | | | b2-3 | 15 | | | | |
| P-17 | A7 | 30 | a1-4 | 10 | b1-2 | 30 | c-2 | 10 | 10300 | 1.82 |
| | | | | | b2-3 | 20 | | | | |
| P-18 | A8 | 25 | a1-2 | 20 | b2-2 | 35 | c-3 | 20 | 9700 | 1.78 |
| P-19 | A9 | 15 | a1-3 | 35 | b1-3 | 10 | c-2 | 10 | 9800 | 1.79 |
| | | | | | b2-2 | 30 | | | | |
| P-20 | A10 | 20 | a1-2 | 30 | b2-3 | 35 | c-2 | 15 | 9600 | 1.77 |
| P-21 | A11 | 15 | a1-1 | 15 | b2-3 | 35 | c-2 | 20 | 9800 | 1.83 |
| | | | a1-3 | 15 | | | | | | |
| P-22 | A2 | 30 | a1-4 | 10 | b1-2 | 25 | c-3 | 10 | 10000 | 1.77 |
| | | | | | b2-3 | 25 | | | | |
| P-23 | A1 | 30 | a1-2 | 20 | b2-1 | 40 | — | — | 7800 | 1.72 |
| P-24 | A2 | 25 | a1-3 | 25 | b1-3 | 10 | — | — | 7400 | 1.71 |
| | | | | | b2-3 | 40 | | | | |
| P-25 | A5 | 25 | a1-1 | 25 | b1-1 | 20 | — | | 7700 | 1.74 |
| | | | | | b2-1 | 30 | | | | |
| P-26 | A8 | 30 | a1-2 | 25 | b1-2 | 15 | — | — | 7800 | 1.72 |
| | | | | | b2-2 | 30 | | | | |
| P-27 | A11 | 20 | a1-4 | 25 | b1-1 | 20 | — | | 7500 | 1.70 |
| | | | | | b2-3 | 35 | | | | |
| P-28 | A7 | 20 | a1-3 | 15 | b1-2 | 20 | — | — | 7800 | 1.76 |
| | | | a2-1 | 15 | b2-1 | 30 | | | | |
| P-29 | A5 | 40 | a1-2 | 20 | b2-3 | 40 | — | — | 7400 | 1.74 |
| P-30 | A1 | 30 | a1-3 | 25 | b2-1 | 45 | — | — | 7600 | 1.72 |

TABLE 2

| | | Introduction | | Introduction | | Introduction | | |
|---|---|---|---|---|---|---|---|---|
| Polymer | Unit a | Ratio (mol %) | Unit b | Ratio (mol %) | Unit c | Ratio (mol %) | Mw | Mw/Mn |
| CP-1 | a1-1 | 55 | b2-1 | 35 | c-1 | 15 | 9700 | 1.82 |
| CP-2 | a1-2 | 55 | b2-1 | 35 | c-1 | 15 | 9800 | 1.83 |
| CP-3 | a1-3 | 55 | b2-1 | 35 | c-1 | 15 | 9600 | 1.81 |
| CP-4 | a1-4 | 10 | b1-1 | 30 | c-1 | 15 | 10000 | 1.83 |
| | a2-1 | 30 | b2-3 | 20 | | | | |
| CP-5 | a1-4 | 10 | b1-2 | 30 | c-2 | 15 | 10200 | 1.79 |
| | a2-2 | 30 | b2-3 | 20 | | | | |
| CP-6 | a1-1 | 50 | b2-1 | 20 | c-1 | 10 | 9500 | 1.78 |
| | | | b2-2 | 20 | | | | |
| CP-7 | a1-3 | 50 | b1-3 | 10 | c-3 | 10 | 10200 | 1.83 |
| | | | b2-1 | 30 | | | | |
| CP-8 | a1-1 | 30 | b1-3 | 10 | c-2 | 10 | 9900 | 1.78 |
| | a2-2 | 20 | b2-1 | 30 | | | | |
| CP-9 | a1-3 | 25 | b1-1 | 10 | c-1 | 10 | 9700 | 1.81 |
| | a2-1 | 25 | b2-1 | 30 | | | | |
| CP-10 | a1-1 | 35 | b1-1 | 15 | c-3 | 10 | 10100 | 1.78 |
| | a1-4 | 15 | b2-3 | 25 | | | | |
| CP-11 | a1-1 | 50 | b2-1 | 50 | — | — | 7600 | 1.71 |
| CP-12 | a1-2 | 60 | b2-1 | 40 | — | — | 7500 | 1.69 |
| CP-13 | a1-3 | 50 | b1-1 | 15 | — | — | 7800 | 1.72 |
| | | | b2-1 | 35 | | | | |
| CP-14 | a1-3 | 35 | b1-3 | 5 | — | — | 7800 | 1.68 |
| | a1-4 | 25 | b2-3 | 35 | | | | |
| CP-15 | a1-1 | 50 | b1-1 | 15 | — | — | 7900 | 1.72 |
| | | | b1-3 | 10 | | | | |
| | | | b2-3 | 25 | | | | |

### [3] Preparation of Chemically-Amplified Resist Composition

### Examples 3-1 to 2-30 and Comparative Examples 2-1 to 2-15

The base polymer (P-1 to P-30) of the present invention, the comparative base polymer (CP-1 to CP-15), a photoacid generator (PAG-X to PAG-Y), and a quencher (SQ-1 to 3, AQ-1) in each of the formulations shown in Tables 3 and 4 were dissolved in a solvent containing 0.01 mass % of surfactant A (Omnova Solutions Inc.) to prepare a solution, and the solution was filtered through a 0.2 μm Teflon (registered trademark) filter to prepare each of a chemically-amplified resist compositions (R-1 to R-30, CR-1 to CR-15).

TABLE 3

| | | | Photoacid | | | |
|---|---|---|---|---|---|---|
| | | Base Polymer | Generator | Quencher | Solvent 1 | Solvent 2 |
| | Resist | (Parts | (Parts | (Parts | (Parts | (Parts |
| | Composition | by Mass) | by Mass) | by Mass) | by Mass) | by Mass) |
| Example | R-1 | P-1(80) | — | SQ-1(7.8) | PGMEA | DAA |
| 3-1 | | | | | (2,200) | (900) |
| Example | R-2 | P-2(80) | — | SQ-1(7.6) | PGMEA | DAA |
| 3-2 | | | | | (2,200) | (900) |
| Example | R-3 | P-3(80) | — | SQ-1(7.8) | PGMEA | DAA |
| 3-3 | | | | | (2,200) | (900) |
| Example | R-4 | P-4(80) | — | SQ-1(7.8) | PGMEA | DAA |
| 3-4 | | | | | (2,200) | (900) |
| Example | R-5 | P-5(80) | — | SQ-1(7.8) | PGMEA | DAA |
| 3-5 | | | | | (2,200) | (900) |
| Example | R-6 | P-6(80) | — | SQ-1(7.4) | PGMEA | DAA |
| 3-6 | | | | | (2,200) | (900) |
| Example | R-7 | P-7(80) | — | SQ-1(7.8) | PGMEA | DAA |
| 3-7 | | | | | (2,200) | (900) |
| Example | R-8 | P-8(80) | — | SQ-1(7.8) | PGMEA | DAA |
| 3-8 | | | | | (2,200) | (900) |
| Example | R-9 | P-9(80) | — | SQ-1(7.6) | PGMEA | DAA |
| 3-9 | | | | | (2,200) | (900) |
| Example | R-10 | P-10(80) | — | SQ-1(7.8) | PGMEA | DAA |
| 3-10 | | | | | (2,200) | (900) |
| Example | R-11 | P-11(80) | — | SQ-1(7.8) | PGMEA | DAA |
| 3-11 | | | | | (2,200) | (900) |
| Example | R-12 | P-12(80) | — | SQ-2(8.8) | PGMEA | DAA |
| 3-12 | | | | | (2,200) | (900) |
| Example | R-13 | P-13(80) | — | SQ-3(7.8) | PGMEA | DAA |
| 3-13 | | | | | (2,200) | (900) |
| Example | R-14 | P-14(80) | — | SQ-2(7.8) | PGMEA | DAA |
| 3-14 | | | | | (2,200) | (900) |
| Example | R-15 | P-15(80) | — | SQ-1(7.8) | PGMEA | DAA |
| 3-15 | | | | | (2,200) | (900) |
| Example | R-16 | P-16(80) | — | SQ-3(7.8) | PGMEA | DAA |
| 3-16 | | | | | (2,200) | (900) |
| Example | R-17 | P-17(80) | — | SQ-1(3.6) | PGMEA | DAA |
| 3-17 | | | | AQ-1(3.6) | (2,200) | (900) |
| Example | R-18 | P-18(80) | PAG-X(8) | SQ-2(7.8) | PGMEA | DAA |
| 3-18 | | | | | (2,200) | (900) |
| Example | R-19 | P-19(80) | — | SQ-2(7.8) | PGMEA | DAA |
| 3-19 | | | | | (2,200) | (900) |
| Example | R-20 | P-20(80) | — | SQ-3(7.4) | PGMEA | DAA |
| 3-20 | | | | | (2,200) | (900) |
| Example | R-21 | P-21(80) | — | SQ-1(7.3) | PGMEA | DAA |
| 3-21 | | | | | (2,200) | (900) |
| Example | R-22 | P-22(80) | — | SQ-2(7.2) | PGMEA | DAA |
| 3-22 | | | | | (2,200) | (900) |
| Example | R-23 | P-23(80) | — | SQ-3(3.7) | PGMEA | DAA |
| 3-23 | | | | AQ-1(3.7) | (2,200) | (900) |
| Example | R-24 | P-24(80) | PAG-X(20) | SQ-1(7.4) | PGMEA | DAA |
| 3-24 | | | | | (2,200) | (900) |
| Example | R-25 | P-25(80) | PAG-Y(20) | SQ-2(7.4) | PGMEA | DAA |
| 3-25 | | | | | (2,200) | (900) |
| Example | R-26 | P-26(80) | PAG-X(20) | SQ-1(7.8) | PGMEA | DAA |
| 3-26 | | | | | (2,200) | (900) |
| Example | R-27 | P-27(80) | PAG-Y(22) | SQ-3(7.2) | PGMEA | DAA |
| 3-27 | | | | | (2,200) | (900) |
| Example | R-28 | P-28(80) | PAG-Y(20) | SQ-2(3.7) | PGMEA | DAA |
| 3-28 | | | | AQ-1(3.7) | (2,200) | (900) |
| Example | R-29 | P-29(80) | PAG-X(18) | SQ-3(7.6) | PGMEA | DAA |
| 3-29 | | | | | (2,200) | (900) |
| Example | R-30 | P-30(80) | PAG-X(20) | SQ-1(7.2) | PGMEA | DAA |
| 3-30 | | | | | (2,200) | (900) |

TABLE 4

| | | Base | Photoacid | | | |
|---|---|---|---|---|---|---|
| | | Polymer | Generator | Quencher | Solvent 1 | Solvent 2 |
| | Resist | (Parts | (Parts | (Parts | (Parts | (Parts |
| | Composition | by Mass) | by Mass) | by Mass) | by Mass) | by Mass) |
| Comparative | CR-1 | CP-1(80) | — | SQ-1(7.8) | PGMEA | DAA |
| Example 2-1 | | | | | (2,200) | (900) |
| Comparative | CR-2 | CP-2(80) | — | SQ-2(7.6) | PGMEA | DAA |
| Example 2-2 | | | | | (2,200) | (900) |
| Comparative | CR-3 | CP-3(80) | — | SQ-3(7.5) | PGMEA | DAA |
| Example 2-3 | | | | | (2,200) | (900) |
| Comparative | CR-4 | CP-4(80) | — | SQ-1(7.6) | PGMEA | DAA |
| Example 2-4 | | | | | (2,200) | (900) |
| Comparative | CR-5 | CP-5(80) | — | SQ-2(7.8) | PGMEA | DAA |
| Example 2-5 | | | | | (2,200) | (900) |
| Comparative | CR-6 | CP-6(80) | — | SQ-1(7.6) | PGMEA | DAA |
| Example 2-6 | | | | | (2,200) | (900) |
| Comparative | CR-7 | CP-7(80) | — | SQ-3(7.6) | PGMEA | DAA |
| Example 2-7 | | | | | (2,200) | (900) |
| Comparative | CR-8 | CP-8(80) | PAG-X(10) | SQ-2(8.8) | PGMEA | DAA |
| Example 2-8 | | | | | (2,200) | (900) |
| Comparative | CR-9 | CP-9(80) | — | SQ-1(3.6) | PGMEA | DAA |
| Example 2-9 | | | | AQ-1(3.6) | (2,200) | (900) |
| Comparative | CR-10 | CP-10(80) | — | SQ-4(6.4) | PGMEA | DAA |
| Example 2-10 | | | | | (2,200) | (900) |
| Comparative | CR-11 | CP-11(80) | PAG-X(20) | SQ-3(3.6) | PGMEA | DAA |
| Example 2-11 | | | | AQ-1(3.6) | (2,200) | (900) |
| Comparative | CR-12 | CP-12(80) | PAG-Y(22) | SQ-2(7.8) | PGMEA | DAA |
| Example 2-12 | | | | | (2,200) | (900) |
| Comparative | CR-13 | CP-13(80) | PAG-Y(20) | SQ-3(7.8) | PGMEA | DAA |
| Example 2-13 | | | | | (2,200) | (900) |
| Comparative | CR-14 | CP-14(80) | PAG-X(20) | SQ-2(3.8) | PGMEA | DAA |
| Example 2-14 | | | | AQ-1(3.6) | (2,200) | (900) |
| Comparative | CR-15 | CP-15(80) | PAG-X(18) | SQ-1(7.6) | PGMEA | DAA |
| Example 2-15 | | | | | (2,200) | (900) |

The components in Tables 3 and 4 are as follows

[Organic solvent]
PGMEA (propylene glycol monomethylether acetate)
DAA (diacetone alcohol)
[Photoacid generator]

### PAG-X, PAG-Y

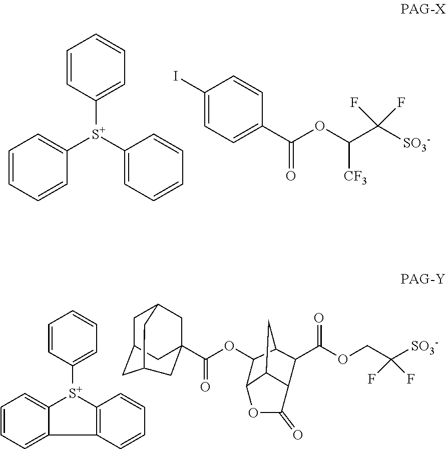

### [Quencher]

### SQ-1 to SQ-3, AQ-1

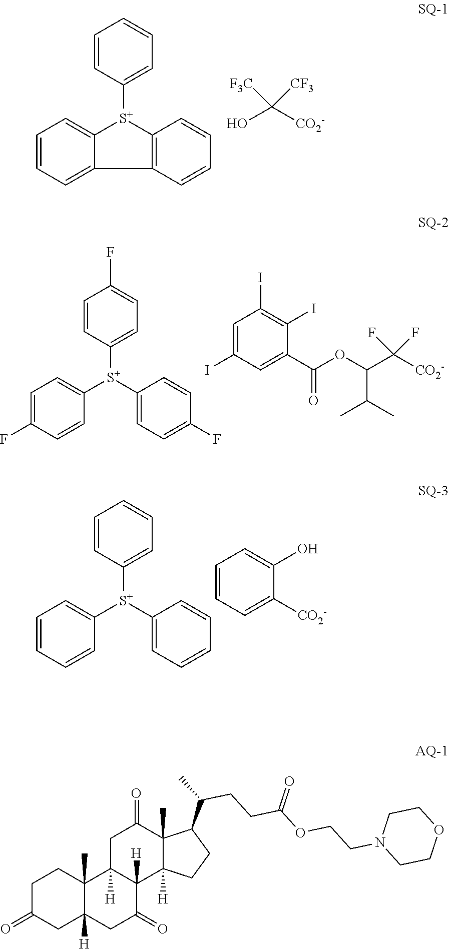

### [4] Evaluation of EUV Lithography (1)

### Examples 4-1 to 4-30, Comparative Examples 3-1 to 3-15

Each of the chemically-amplified resist compositions (R-1 to R-30, CR-1 to CR-15) shown in Tables 3 and 4 was spin-coated on a Si substrate on which a silicon-containing spin-on hard mask SHB-A940 (manufactured by Shin-Etsu Chemical Co., Ltd.) having a silicon content of 43 mass % is formed with a thickness of 20 nm, followed by prebaking by using a hotplate at 100° C. for 60 seconds to form a resist film having a thickness of 50 nm. Then, the film was subjected to exposure with a LS pattern having a 18 nm of on-wafer size and a pitch of 36 nm by using a NXE3300 EUV scanner manufactured by ASML (NA 0.33, σ 0.9/0.6, dipole illumination) while changing the exposure dose and the focus (exposure dose pitch: 1 mJ/cm², focus pitch: 0.020 μm). After the exposure, PEB was performed for 60 seconds at the temperatures shown in Tables 5 and 6. Thereafter, puddle development was performed with a 2.38 mass % aqueous TMAH solution for 30 seconds, followed by rinsing with a surfactant-containing rinse material and spin-drying to obtain a positive pattern.

Each of the obtained LS patterns was observed with a critical dimension SEM (CG6300) manufactured by Hitachi High-Tech Corporation, and sensitivity, exposure latitude (EL), LWR, depth of focus (DOF) and collapse limit were evaluated according to the following method. Tables 5 and 6 show the results.

### [Evaluation of Sensitivity]

The sensitivity was obtained by determining an optimum dose Eₒₚ (mJ/cm²) by which an LS pattern having a line width of 18 nm and a pitch of 36 nm can be obtained. The smaller the value, the higher the sensitivity.

### [Evaluation of EL]

EL (unit: %) was obtained from the exposure dose formed within ±10% (16.2 to 19.8 nm) of 18 nm, i.e., the space width, in the LS pattern described above according to the following formula. The larger the value, the better the performance.

EL (%)=(E₁−E₂/Eₒₚ)×100
E₁: optimum exposure dose which provides a LS pattern with a line width of 16.2 nm and a pitch of 36 nm
E₂: optimum exposure dose which provides a LS pattern with a line width of 19.8 nm and a pitch of 36 nm
Eₒₚ: optimum exposure dose which provides a LS pattern with a line width of 18 nm and a pitch of 36 nm

### [Evaluation of LWR]

The line sizes at 10 points in the longitudinal direction of the LS pattern obtained by irradiation at Eₒₚ were measured, and a 3-fold value (3σ) of standard deviation (σ) was calculated from the results to determine LWR. A smaller value of 3σ indicates a pattern having a smaller roughness and more uniform line width.

### [Evaluation of DOF]

To evaluate DOP (depth of focus), a range of focus formed within a range of ±10% (16.2 to 19.8 nm) of the size of 18 nm in the LS pattern described above was determined. The larger the value, the wider the DOP.

### [Evaluation of Line Pattern Collapse Limit]

The line sizes at 10 points in the longitudinal direction of the LS pattern at the optimum focus were measured for each exposure dose. The thinnest line size obtained without collapse was measured as the collapse limit size. The smaller the value, the superior the collapse limit.

TABLE 5

| | | PEB | Optimum | | | | Collapse |
|---|---|---|---|---|---|---|---|
| | Resist | Temperature | Dose | EL | LWR | DOF | Limit |
| | Composition | (° C.) | (mJ/cm²) | (%) | (nm) | (nm) | (nm) |
| Example 4-1 | R-1 | 95 | 38 | 19 | 2.7 | 120 | 10.1 |
| Example 4-2 | R-2 | 95 | 39 | 18 | 2.7 | 110 | 10.2 |
| Example 4-3 | R-3 | 100 | 38 | 18 | 2.8 | 100 | 10.4 |
| Example 4-4 | R-4 | 95 | 40 | 17 | 2.9 | 120 | 10.3 |
| Example 4-5 | R-5 | 90 | 39 | 19 | 2.8 | 110 | 10.3 |
| Example 4-6 | R-6 | 95 | 38 | 18 | 2.9 | 110 | 10.5 |
| Example 4-7 | R-7 | 95 | 40 | 19 | 3 | 120 | 11.1 |
| Example 4-8 | R-8 | 95 | 41 | 18 | 3.1 | 110 | 10.9 |
| Example 4-9 | R-9 | 100 | 38 | 18 | 2.8 | 120 | 10.3 |
| Example 4-10 | R-10 | 90 | 39 | 19 | 3.1 | 120 | 10.6 |
| Example 4-11 | R-11 | 95 | 39 | 18 | 3 | 100 | 10.4 |
| Example 4-12 | R-12 | 100 | 38 | 17 | 2.9 | 110 | 10.5 |
| Example 4-13 | R-13 | 95 | 40 | 19 | 3.1 | 120 | 11.2 |
| Example 4-14 | R-14 | 90 | 41 | 18 | 2.8 | 100 | 10.5 |
| Example 4-15 | R-15 | 100 | 39 | 19 | 2.9 | 110 | 10.7 |
| Example 4-16 | R-16 | 95 | 40 | 19 | 2.9 | 110 | 11.3 |
| Example 4-17 | R-17 | 95 | 40 | 17 | 3 | 120 | 10.3 |
| Example 4-18 | R-18 | 90 | 41 | 18 | 2.9 | 120 | 10.7 |
| Example 4-19 | R-19 | 100 | 38 | 19 | 3.1 | 110 | 11 |
| Example 4-20 | R-20 | 95 | 39 | 18 | 3 | 110 | 10.5 |
| Example 4-21 | R-21 | 90 | 38 | 19 | 2.8 | 120 | 10.6 |
| Example 4-22 | R-22 | 95 | 38 | 19 | 3.1 | 100 | 10.9 |
| Example 4-23 | R-23 | 100 | 40 | 18 | 2.8 | 120 | 11 |
| Example 4-24 | R-24 | 90 | 41 | 19 | 2.9 | 120 | 11.2 |
| Example 4-25 | R-25 | 95 | 39 | 18 | 3 | 110 | 10.8 |
| Example 4-26 | R-26 | 90 | 39 | 17 | 3.2 | 120 | 10.6 |
| Example 4-27 | R-27 | 95 | 41 | 19 | 2.9 | 110 | 10.9 |
| Example 4-28 | R-28 | 100 | 40 | 18 | 2.7 | 110 | 10.4 |
| Example 4-29 | R-29 | 95 | 41 | 19 | 2.9 | 120 | 10.4 |
| Example 4-30 | R-30 | 100 | 38 | 19 | 2.9 | 100 | 10.7 |

TABLE 6

| | | PEB | Optimum | | | | Collapse |
|---|---|---|---|---|---|---|---|
| | Resist | Temperature | Dose | EL | LWR | DOF | Limit |
| | Composition | (° C.) | (mJ/cm²) | (%) | (nm) | (nm) | (nm) |
| Comparative | CR-1 | 100 | 43 | 17 | 3.7 | 90 | 14.3 |
| Example 3-1 | | | | | | | |
| Comparative | CR-2 | 95 | 44 | 16 | 3.5 | 80 | 14.6 |
| Example 3-2 | | | | | | | |
| Comparative | CR-3 | 95 | 45 | 16 | 3.5 | 80 | 14.5 |
| Example 3-3 | | | | | | | |
| Comparative | CR-4 | 100 | 43 | 15 | 3.4 | 90 | 15.5 |
| Example 3-4 | | | | | | | |
| Comparative | CR-5 | 95 | 42 | 17 | 3.5 | 80 | 12.7 |
| Example 3-5 | | | | | | | |
| Comparative | CR-6 | 110 | 46 | 16 | 3.6 | 90 | 13.6 |
| Example 3-6 | | | | | | | |
| Comparative | CR-7 | 95 | 44 | 15 | 3.7 | 70 | 14.3 |
| Example 3-7 | | | | | | | |
| Comparative | CR-8 | 90 | 40 | 16 | 3.5 | 100 | 12.8 |
| Example 3-8 | | | | | | | |
| Comparative | CR-9 | 100 | 45 | 14 | 3.6 | 70 | 15.1 |
| Example 3-9 | | | | | | | |
| Comparative | CR-10 | 105 | 43 | 17 | 3.5 | 90 | 14.6 |
| Example 3-10 | | | | | | | |
| Comparative | CR-11 | 90 | 41 | 16 | 3.6 | 90 | 13.8 |
| Example 3-11 | | | | | | | |
| Comparative | CR-12 | 95 | 43 | 17 | 4 | 80 | 14.5 |
| Example 3-12 | | | | | | | |
| Comparative | CR-13 | 95 | 44 | 16 | 4.1 | 90 | 13.6 |
| Example 3-13 | | | | | | | |
| Comparative | CR-14 | 100 | 45 | 15 | 4.2 | 70 | 14.2 |
| Example 3-14 | | | | | | | |
| Comparative | CR-15 | 95 | 43 | 14 | 3.7 | 90 | 13.7 |
| Example 3-15 | | | | | | | |

The results shown in Tables 5 and 6 revealed that the chemically-amplified resist composition of the present invention, which contains a photoacid generator, has desirable sensitivity, and is excellent in EL, LWR and DOF. In addition, it was confirmed that the value of the collapse limit was small and the pattern collapse resistance was high even in the formation of fine patterns. Therefore, it was revealed that the chemically-amplified resist composition of the present invention is suitable as a material for EUV lithography.

### [5] Evaluation of EUV Lithography (2)

### Examples 5-1 to 5-30, Comparative Examples 4-1 to 4-15

Each of the chemically-amplified resist compositions (R-1 to R-30, CR-1 to CR-15) shown in Tables 3 and 4 was spin-coated on a Si substrate on which a silicon-containing spin-on hard mask SHB-A940 (manufactured by Shin-Etsu Chemical Co., Ltd.) having a silicon content of 43 mass % is formed with a thickness of 20 nm, followed by prebaking by using a hotplate at 105° C. for 60 seconds to form a resist film having a thickness of 50 nm. The resist film was then subjected to exposure using a EUV scanner NXE3400 (NA 0.33, σ 0.9/0.6, quadrupole illumination) manufactured by ASML through a mask with a hole pattern at a pitch of 46 nm (on-wafer size) and +20% bias, followed by PEB using a hotplate at the temperature shown in Tables 7 and 8 for 60 seconds and development with a 2.38 mass % aqueous TMAH solution for 30 seconds to form a hole pattern having a size of 23 nm.

Using a critical dimension SEM (CG6300, manufactured by Hitachi High-Tech Corporation), the exposure dose that provides a hole having a size of 23 nm was measured to determine sensitivity. Further, the size of each of 50 holes at this time was measured, and a 3-fold value (3σ) of standard deviation (σ) was calculated from the results to determine size variation (CDU). Tables 7 and 8 show the results.

TABLE 7

| | | PEB | Optimum | |
|---|---|---|---|---|
| | Resist | Temperature | Dose | CDU |
| | Composition | (° C.) | (mJ/cm2) | (nm) |
| Example 5-1 | R-1 | 90 | 25 | 2.2 |
| Example 5-2 | R-2 | 95 | 25 | 2.3 |
| Example 5-3 | R-3 | 90 | 26 | 2.4 |
| Example 5-4 | R-4 | 100 | 25 | 2.5 |
| Example 5-5 | R-5 | 95 | 24 | 2.4 |
| Example 5-6 | R-6 | 90 | 26 | 2.3 |
| Example 5-7 | R-7 | 95 | 24 | 2.3 |
| Example 5-8 | R-8 | 90 | 25 | 2.5 |
| Example 5-9 | R-9 | 95 | 25 | 2.6 |
| Example 5-10 | R-10 | 100 | 24 | 2.5 |
| Example 5-11 | R-11 | 95 | 24 | 2.6 |
| Example 5-12 | R-12 | 90 | 25 | 2.5 |
| Example 5-13 | R-13 | 90 | 26 | 2.5 |
| Example 5-14 | R-14 | 95 | 24 | 2.3 |
| Example 5-15 | R-15 | 100 | 25 | 2.5 |
| Example 5-16 | R-16 | 90 | 26 | 2.4 |
| Example 5-17 | R-17 | 95 | 26 | 2.7 |
| Example 5-18 | R-18 | 90 | 24 | 2.5 |
| Example 5-19 | R-19 | 95 | 25 | 2.4 |
| Example 5-20 | R-20 | 90 | 24 | 2.6 |
| Example 5-21 | R-21 | 100 | 26 | 2.7 |
| Example 5-22 | R-22 | 95 | 24 | 2.4 |
| Example 5-23 | R-23 | 95 | 25 | 2.6 |
| Example 5-24 | R-24 | 90 | 25 | 2.6 |
| Example 5-25 | R-25 | 100 | 24 | 2.5 |
| Example 5-26 | R-26 | 95 | 24 | 2.6 |
| Example 5-27 | R-27 | 90 | 26 | 2.4 |
| Example 5-28 | R-28 | 95 | 24 | 2.6 |
| Example 5-29 | R-29 | 100 | 25 | 2.4 |
| Example 5-30 | R-30 | 95 | 26 | 2.4 |

TABLE 8

| | | PEB | Optimum | |
|---|---|---|---|---|
| | Resist | Temperature | Dose | CDU |
| | Composition | (° C.) | (mJ/cm²) | (nm) |
| Comparative | CR-1 | 95 | 28 | 3.1 |
| Example 4-1 | | | | |
| Comparative | CR-2 | 90 | 29 | 3.2 |
| Example 4-2 | | | | |
| Comparative | CR-3 | 100 | 27 | 3 |
| Example 4-3 | | | | |
| Comparative | CR-4 | 95 | 30 | 3.2 |
| Example 4-4 | | | | |
| Comparative | CR-5 | 90 | 28 | 2.9 |
| Example 4-5 | | | | |
| Comparative | CR-6 | 90 | 29 | 3 |
| Example 4-6 | | | | |
| Comparative | CR-7 | 95 | 30 | 3.4 |
| Example 4-7 | | | | |
| Comparative | CR-8 | 100 | 31 | 3.1 |
| Example 4-8 | | | | |
| Comparative | CR-9 | 100 | 29 | 3.4 |
| Example 4-9 | | | | |
| Comparative | CR-10 | 95 | 28 | 3.2 |
| Example 4-10 | | | | |
| Comparative | CR-11 | 95 | 31 | 2.9 |
| Example 4-11 | | | | |
| Comparative | CR-12 | 100 | 32 | 3.1 |
| Example 4-12 | | | | |
| Comparative | CR-13 | 95 | 33 | 3.4 |
| Example 4-13 | | | | |
| Comparative | CR-14 | 90 | 30 | 3.3 |
| Example 4-14 | | | | |
| Comparative | CR-15 | 100 | 29 | 3.5 |
| Example 4-15 | | | | |

The results shown in Tables 7 and 8 revealed that the chemically-amplified resist composition of the present invention has desirable sensitivity and excellent CDU.

The present invention encompasses the following embodiments.

[1]: A polymerizable monomer represented by the following general formula (1);

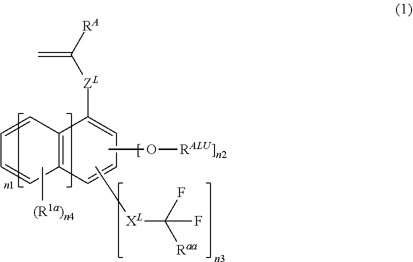

wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a linear, branched or cyclic hydrocarbyl group having 1 to 20 carbon atoms which may contain a heteroatom; n1 is an integer of 0 to 2; n2 is an integer of 1 or 2; n3 is an integer of 1 or 2; and n4 is an integer of 0 to 4; when n2=1, each of —O—R^{ALU} and —X^{L}—CF₂(R^{aa}) is bonded to each of carbon atoms which are adjacent to each other on an aromatic ring; when n2=2, one of two —O—R^{ALU} is bonded to a carbon atom adjacent to the carbon atom on the aromatic ring to which —X^{L}—CF₂ (R^{aa}) is bonded.

[2]: The polymerizable monomer according to [1], wherein, in the general formula (1), R^{ALU} is represented by the following formula (AL-1) or (AL-2) together with the adjacent oxygen atom,

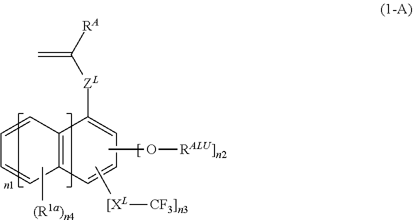

wherein R^{A}, Z^{L}, X^{L}, R^{1a}, R^{ALU}, n1, n2, n3, and n4 are as defined above.

[3]: The polymerizable monomer according to [1] or [2], wherein R^{ALU} is represented by the following formula (AL-1) or (AL-2) together with the adjacent oxygen atom,

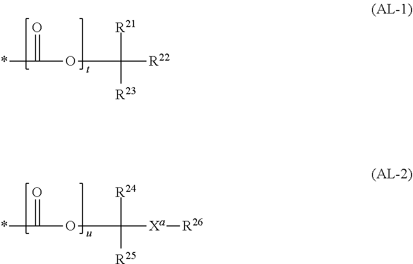

in the formula (AL-1), R²¹, R²², and R²³ independently represent a hydrocarbyl group having 1 to 12 carbon atoms which may contain a heteroatom; any two of R²¹, R²², and R²³ may be bonded to form a ring; and t is an integer of 0 or 1; in the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

[4]: A polymer compound comprising a repeating unit obtained from the polymerizable monomer according to any one of [1] to [3], wherein the repeating unit is represented by the following general formula (1a),

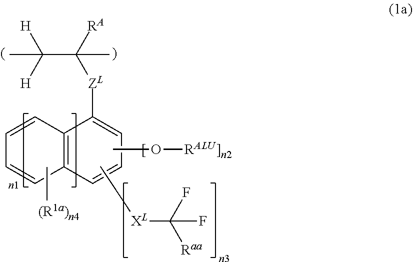

wherein R^{A}, Z^{L}, X^{L}, R^{1a}, R^{ALU}, R^{aa}, n1, n2, n3, and n4 are as defined above.

[5]: A polymer compound comprising a repeating unit obtained from the polymerizable monomer according to [1] to [3], wherein the repeating unit is represented by the following general formula (1-Aa).

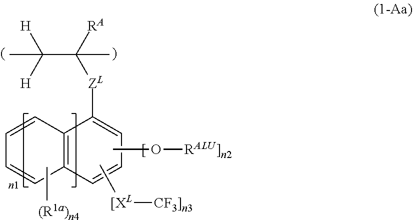

[6]: The polymer compound according to [4] or [5], wherein R^{ALU} is represented by the following formula (AL-1) or (AL-2) together with the adjacent oxygen atom,

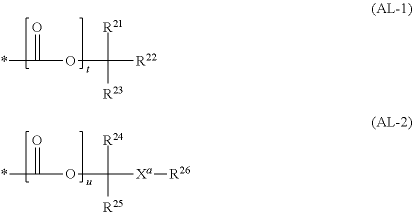

wherein R²¹, R²², R²³, t, R²⁴, R²⁵, R²⁶ X^{a} and u are as defined above.

[7]: The polymer compound according to any one of [4] to [6], further comprising at least one selected from repeating units represented by the following general formula (a1) or (a2),

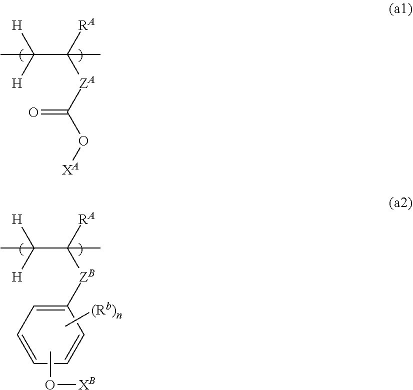

wherein R^{A} independently represents a hydrogen atom, a fluorine atom, a methyl group, or a trifluoromethyl group; Z^{A} is a single bond, an alkoxy group optionally substituted with a halogen atom, a phenylene group or a naphthylene group optionally substituted with a halogen atom, or (backbone) —C(═O)—O—Z^{A1}—; Z^{A1} is a heteroatom, an alkoxy group having 1 to 10 carbon atoms which may contain a fluorine atom, a linear, branched or cyclic alkanediyl group having 1 to 20 carbon atoms which may contain a hydroxy group, an ether bond, an ester bond, a lactone ring, a thioether bond, a sulfonyl group, or a sulfonamide structure, or a phenylene group or a naphthylene group; Z^{B} is a single bond or (backbone) —C(═O)—O—Z^{B1}—; Z^{B1} is a single bond or an alkanediyl group having 1 to 10 carbon atoms which may contain an ester and/or an ether bond; X^{A} and X^{B} independently represent an acid-labile group; R^{b} represents a halogen atom, a nitro group, or a linear, branched or cyclic monovalent hydrocarbon group having 1 to 20 carbon atoms which may contain a heteroatom; and n is an integer of 0 to 4.

[8]: The polymer compound according to any one of [4] to [7], further comprising at least one selected from repeating units represented by the following general formula (b1) or (b2),

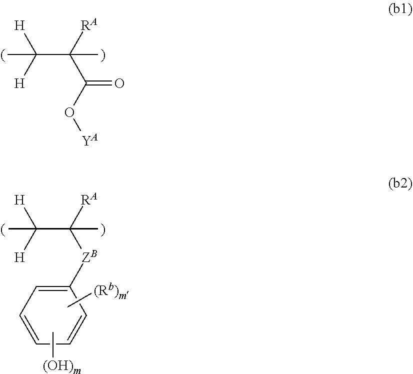

wherein R^{A} independently represents a hydrogen atom, a fluorine atom, a methyl group, or a trifluoromethyl group; YA is a hydrogen atom, or a polar group comprising at least one or more structures selected from the group consisting of a hydroxy group, a cyano group, a carbonyl group, a carboxy group, an ether bond, an ester bond, a sulfonic acid ester bond, a sulfonic acid amide bond, a carbonate bond, a lactone ring, a sultone ring, a sulfur atom, and a carboxylic acid anhydride; Z^{B} is a single bond or (backbone) —C(═O)—O—Z^{B1}—; Z^{B1} is a single bond or an alkanediyl group having 1 to 10 carbon atoms which may contain an ester and/or an ether bond; R^{b} represents a halogen atom, a nitro group, or a linear, branched or cyclic monovalent hydrocarbon group having 1 to 20 carbon atoms which may contain a heteroatom; m is an integer of 1 to 4; and m′ is an integer of 0 to 4.

[9]: The polymer compound according to any one of [4] to [8], further comprising at least one selected from repeating units represented by the following general formulae (C1) to (C4),

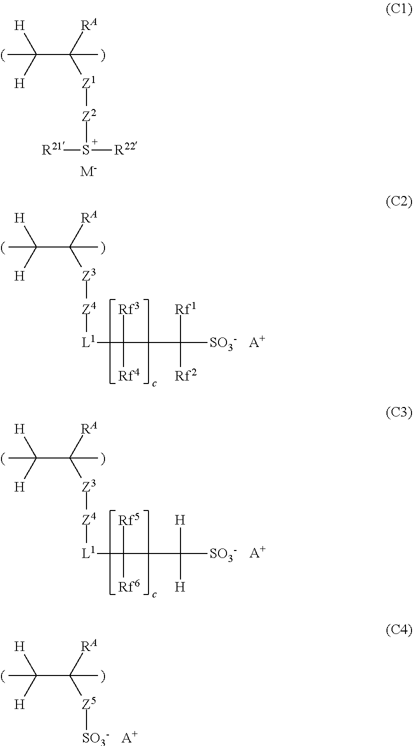

wherein R^{A} independently represents a hydrogen atom, a fluorine atom, a methyl group, or a trifluoromethyl group; Z¹ is a single bond or a phenylene group; Z² is a single bond, *—C(═O)—O—Z²¹—, *—C(═O)—NH—Z²¹—, or *—O—Z²¹—; Z²¹ is an aliphatic hydrocarbylene group having 1 to 12 carbon atoms, a phenylene group, or a divalent group obtained by combining these groups, and may contain a carbonyl group, an ester bond, an ether bond or a hydroxy group; Z³ is a single bond, a phenylene group, a naphthylene group, or *—C(═O)—O—Z³¹—; Z³¹ is a single bond, an aliphatic hydrocarbylene group having 1 to 14 carbon atoms which may contain a hydroxy group, an ether bond, an ester bond, or a lactone ring, or a phenylene group or a naphthylene group; Z⁴ is a single bond, a methylene group, or *—Z⁴¹—C(═O)—O—; Z⁴¹ represents a hydrocarbylene group having 1 to 20 carbon atoms which may contain an ether bond, an ester bond, or a heteroatom; Z⁵ is a single bond, a methylene group, an ethylene group, a phenylene group, a fluorinated phenylene group, a phenylene group substituted with a trifluoromethyl group, *—C(═O)—O—Z⁵¹—, *—C(═O)—N(H)—Z⁵¹—, or *—O—Z⁵¹—; Z⁵¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond or a hydroxy group; * represents a bonding arm to a carbon atom in the backbone; R^{21′} and R^{22′} independently represent a hydrocarbyl group having 1 to 20 carbon atoms which may contain a heteroatom; R^{21′} and R^{22′} may also be bonded to each other to form a ring with a sulfur atom to which R^{21′} and R^{22′} are bonded; L¹ is a single bond, an ether bond, an ester bond, a carbonyl group, a sulfonic acid ester bond, a carbonate bond or a carbamate bond; Rf¹ and Rf² independently represent a fluorine atom or a fluorinated alkyl group having 1 to 6 carbon atoms; Rf³ and Rf⁴ independently represent a hydrogen atom, a fluorine atom, or a fluorinated alkyl group having 1 to 6 carbon atoms; Rf⁵ and Rf⁶ independently represent a hydrogen atom, a fluorine atom, or a fluorinated alkyl group having 1 to 6 carbon atoms, provided that not all of Rf⁵ and Rf⁶ are hydrogen atoms at the same time; M⁻ is a non-nucleophilic counter ion; A⁺ is an onium cation; and c is an integer of 0 to 3.

[10]: A resist composition comprising a base resin formed from the polymer compound according to any one of [4] to [9], and an organic solvent.

[11]: The resist composition according to any one of [4] to [10], further comprising one or more selected from a quencher and an acid generator.

[12]: The resist composition according to any one of [4] to [11], further comprising a surfactant which is insoluble or substantially insoluble in water and soluble in an alkaline developer, and/or a surfactant which is insoluble or substantially insoluble in water and an alkaline developer.

[13]: A patterning process comprising the steps of:
forming a resist film on a substrate by using the resist composition according to any one of [4] to [12];
exposing the resist film to high-energy radiation; and
developing the exposed resist film by using a developer.

[14]: The patterning process according to [13], wherein the high-energy beam is KrF excimer laser beam, ArF excimer laser beam, an electron beam, or extreme ultraviolet ray having a wavelength of 3 to 15 nm.

It should be noted that the present invention is not limited to the above-described embodiments. The embodiments are just examples, and any examples that substantially have the same feature and demonstrate the same functions and effects as those in the technical concept disclosed in claims of the present invention are included in the technical scope of the present invention.

1. A polymerizable monomer represented by the following general formula (1); 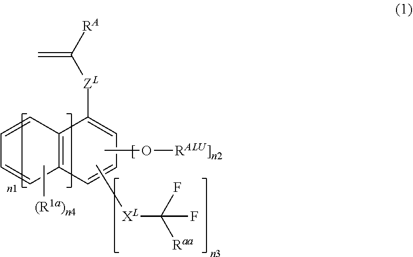
wherein R^{A} represents a hydrogen atom, a fluorine atom, a methyl group, or a trifluoromethyl group; Z^{L} is a single bond or (backbone) —C(═O)—O—; R^{ALU} is an acid-labile group formed with an adjacent oxygen atom; X^{L} represents an oxygen atom or a sulfur atom; R^{aa} represents a hydrogen atom or a fluorine atom; R^{1a} independently represents a linear, branched or cyclic hydrocarbyl group having 1 to 20 carbon atoms which may contain a heteroatom; n1 is an integer of 0 to 2; n2 is an integer of 1 or 2; n3 is an integer of 1 or 2; and n4 is an integer of 0 to 4; when n2=1, each of —O—R^{ALU} and —X^{L}—CF₂(R^{aa}) is bonded to each of carbon atoms which are adjacent to each other on an aromatic ring; when n2=2, one of two —O—R^{ALU} is bonded to a carbon atom adjacent to the carbon atom on the aromatic ring to which —X^{L}—CF₂ (R^{aa}) is bonded.

2. The polymerizable monomer according to claim 1, wherein the general formula (1) is represented by the following formula (1-A), 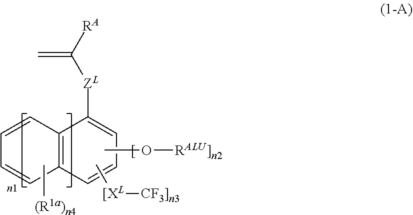
wherein R^{A}, Z^{L}, X^{L}, R^{1a}, R^{ALU}, n1, n2, n3, and n4 are as defined above.

3. The polymerizable monomer according to claim 1, wherein, in the general formula (1), R^{ALU} is represented by the following formula (AL-1) or (AL-2) together with the adjacent oxygen atom, 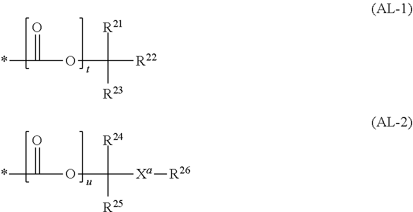
in the formula (AL-1), R²¹, R²², and R²³ independently represent a hydrocarbyl group having 1 to 12 carbon atoms which may contain a heteroatom; any two of R²¹, R²², and R²³ may be bonded to each other to form a ring; and t is an integer of 0 or 1; in the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

4. The polymerizable monomer according to claim 2, wherein, in the general formula (1-A), R^{ALU} is represented by the following formula (AL-1) or (AL-2) together with the adjacent oxygen atom. 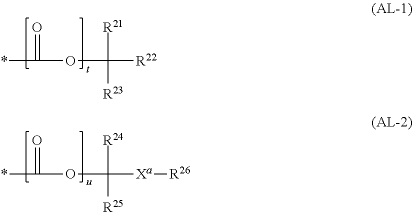
in the formula (AL-1), R²¹, R²², and R²³ independently represent a hydrocarbyl group having 1 to 12 carbon atoms which may contain a heteroatom; any two of R²¹, R²², and R²³ may be bonded to form a ring; and t is an integer of 0 or 1; in the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

5. A polymer compound comprising a repeating unit obtained from the polymerizable monomer according to claim 1, wherein the repeating unit is represented by the following general formula (Ia), 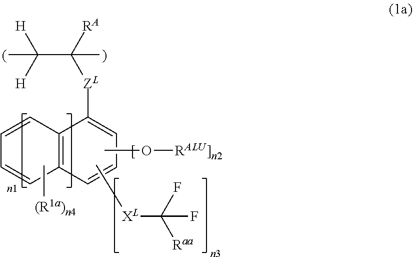
wherein R^{A}, Z^{L}, X^{L}, R^{1a}, R^{ALU}, R^{aa}, n1, n2, n3, and n4 are as defined above.

6. A polymer compound comprising a repeating unit obtained from the polymerizable monomer according to claim 2, wherein the repeating unit is represented by the following general formula (1-Aa), 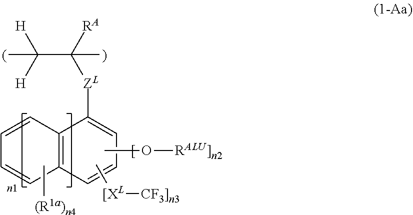
wherein R^{A}, Z^{L}, X^{L}, R^{1a}, R^{ALU}, n1, n2, n3, and n4 are as defined above.

7. The polymer compound according to claim 5, wherein, in the general formula (Ia), R^{ALU} is represented by the following formula (AL-1) or (AL-2) together with the adjacent oxygen atom, 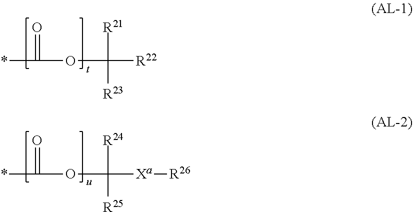
in the formula (AL-1), R²¹, R²², and R²³ independently represent a hydrocarbyl group having 1 to 12 carbon atoms which may contain a heteroatom; any two of R²¹, R²², and R²³ may be bonded to each other to form a ring; and t is an integer of 0 or 1; in the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

8. The polymer compound according to claim 6, wherein, in the general formula (1-Aa), R^{ALU} is represented by the following formula (AL-1) or (AL-2) together with the adjacent oxygen atom, 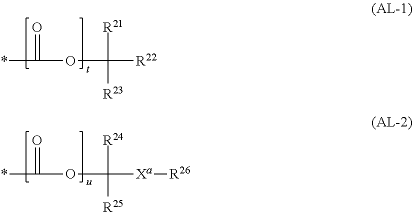
in the formula (AL-1), R²¹, R²², and R²³ independently represent a hydrocarbyl group having 1 to 12 carbon atoms which may contain a heteroatom; any two of R²¹, R²², and R²³ may be bonded to each other to form a ring; and t is an integer of 0 or 1; in the formula (AL-2), R²⁴ and R²⁵ independently represent a hydrogen atom or a hydrocarbyl group having 1 to 10 carbon atoms; R²⁶ is a hydrocarbyl group having 1 to 20 carbon atoms, or may be bonded to R²⁴ or R²⁵ to form a heterocyclic group having 3 to 20 carbon atoms with a carbon atom to which R²⁴ or R²⁵ is bonded and X^{a}; —CH₂— in the hydrocarbyl group and the heterocyclic group may be substituted with —O— or —S—; X^{a} represents an oxygen atom or a sulfur atom; u is an integer of 0 or 1; and * represents a bonding arm to the adjacent oxygen atom.

9. The polymer compound according to claim 5, further comprising at least one selected from repeating units represented by the following general formula (a1) or (a2), 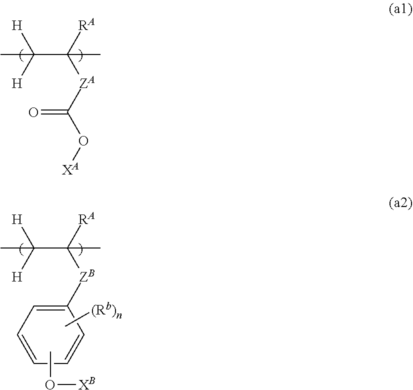
wherein R^{A} independently represents a hydrogen atom, a fluorine atom, a methyl group, or a trifluoromethyl group; Z^{A} is a single bond, an alkoxy group optionally substituted with a halogen atom, a phenylene group or a naphthylene group optionally substituted with a halogen atom, or (backbone) —C(═O)—O—Z^{A1}—; Z^{A1} is a heteroatom, an alkoxy group having 1 to 10 carbon atoms which may contain a fluorine atom, a linear, branched or cyclic alkanediyl group having 1 to 20 carbon atoms which may contain a hydroxy group, an ether bond, an ester bond, a lactone ring, a thioether bond, a sulfonyl group, or a sulfonamide structure, or a phenylene group or a naphthylene group; Z^{B} is a single bond or (backbone) —C(═O)—O—Z^{B1}—; Z^{B1} is a single bond or an alkanediyl group having 1 to 10 carbon atoms which may contain an ester and/or an ether bond; X^{A} and X^{B} independently represent an acid-labile group; R^{b} represents a halogen atom, a nitro group, or a linear, branched or cyclic monovalent hydrocarbon group having 1 to 20 carbon atoms which may contain a heteroatom; and n is an integer of 0 to 4.

10. The polymer compound according to claim 5, further comprising at least one selected from repeating units represented by the following general formula (b1) or 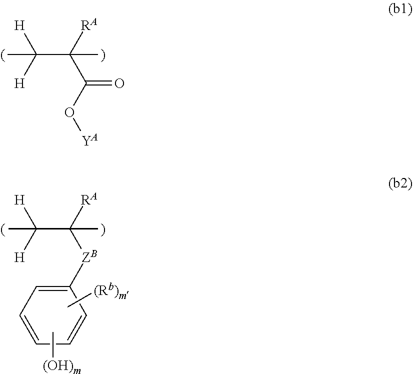
wherein R^{A} independently represents a hydrogen atom, a fluorine atom, a methyl group, or a trifluoromethyl group; Y^{A} is a hydrogen atom, or a polar group comprising at least one or more structures selected from the group consisting of a hydroxy group, a cyano group, a carbonyl group, a carboxy group, an ether bond, an ester bond, a sulfonic acid ester bond, a sulfonic acid amide bond, a carbonate bond, a lactone ring, a sultone ring, a sulfur atom, and a carboxylic acid anhydride; Z^{B} is a single bond or (backbone) —C(═O)—O—Z^{B1}—; Z^{B1} is a single bond or an alkanediyl group having 1 to 10 carbon atoms which may contain an ester and/or an ether bond; R^{b} represents a halogen atom, a nitro group, or a linear, branched or cyclic monovalent hydrocarbon group having 1 to 20 carbon atoms which may contain a heteroatom; m is an integer of 1 to 4; and m′ is an integer of 0 to 4.

11. The polymer compound according to claim 5, further comprising at least one selected from repeating units represented by the following general formulae (C1) to (C4), 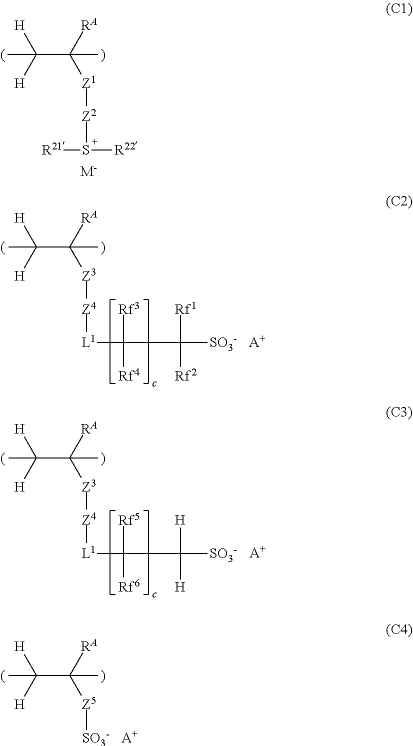
wherein R^{A} independently represents a hydrogen atom, a fluorine atom, a methyl group, or a trifluoromethyl group; Z¹ is a single bond or a phenylene group; Z² is a single bond, *—C(═O)—O—Z²¹—, *—C(═O)—NH—Z²¹—, or *—O—Z²¹—; Z²¹ is an aliphatic hydrocarbylene group having 1 to 12 carbon atoms, a phenylene group, or a divalent group obtained by combining these groups, and may contain a carbonyl group, an ester bond, an ether bond or a hydroxy group; Z³ is a single bond, a phenylene group, a naphthylene group, or *—C(═O)—O—Z³¹—; Z³¹ is a single bond, an aliphatic hydrocarbylene group having 1 to 14 carbon atoms which may contain a hydroxy group, an ether bond, an ester bond, or a lactone ring, or a phenylene group or a naphthylene group; Z⁴ is a single bond, a methylene group, or *—Z⁴¹—C(═O)—O—; Z⁴¹ represents a hydrocarbylene group having 1 to 20 carbon atoms which may contain an ether bond, an ester bond, or a heteroatom; Z⁵ is a single bond, a methylene group, an ethylene group, a phenylene group, a fluorinated phenylene group, a phenylene group substituted with a trifluoromethyl group, *—C(═O)—O—Z⁵¹—, *—C(═O)—N(H)—Z⁵¹—, or *—O—Z³¹—; Z⁵¹ is an aliphatic hydrocarbylene group having 1 to 6 carbon atoms, a phenylene group, a fluorinated phenylene group, or a phenylene group substituted with a trifluoromethyl group, and may contain a carbonyl group, an ester bond, an ether bond or a hydroxy group; * represents a bonding arm to a carbon atom in the backbone; R²¹′ and R²²′ independently represent a hydrocarbyl group having 1 to 20 carbon atoms which may contain a heteroatom; R^{21′} and R^{22′} may also be bonded to each other to form a ring with a sulfur atom to which R^{21′} and R^{22′} are bonded; L¹ is a single bond, an ether bond, an ester bond, a carbonyl group, a sulfonic acid ester bond, a carbonate bond or a carbamate bond; Rf¹ and Rf² independently represent a fluorine atom or a fluorinated alkyl group having 1 to 6 carbon atoms; Rf³ and Rf⁴ independently represent a hydrogen atom, a fluorine atom, or a fluorinated alkyl group having 1 to 6 carbon atoms; Rf³ and Rf⁶ independently represent a hydrogen atom, a fluorine atom, or a fluorinated alkyl group having 1 to 6 carbon atoms, provided that not all of Rf⁵ and Rf⁶ are hydrogen atoms at the same time; M⁻ is a non-nucleophilic counter ion; A⁺ is an onium cation; and c is an integer of 0 to 3.

12. A resist composition comprising a base resin formed from the polymer compound according to claim 5, and an organic solvent.

13. The resist composition according to claim 12, further comprising one or more selected from a quencher and an acid generator.

14. The resist composition according to claim 12, further comprising a surfactant which is insoluble or substantially insoluble in water and soluble in an alkaline developer, and/or a surfactant which is insoluble or substantially insoluble in water and an alkaline developer.

15. A patterning process comprising the steps of:
forming a resist film on a substrate by using the resist composition according to claim 12;
exposing the resist film to high-energy radiation; and
developing the exposed resist film by using a developer.

16. The patterning process according to claim 15, wherein the high-energy beam is KrF excimer laser beam, ArF excimer laser beam, an electron beam, or extreme ultraviolet ray having a wavelength of 3 to 15 nm.
